SEQUENCE LISTING (24 sequences)

SEQ ID NO: 1 - RNA, 3467 nt, Homo sapiens
  source 1..3467: mol_type = mRNA
cagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 2 - DNA, 296000 bp, Homo sapiens
  source 1..296000: mol_type = genomic DNA
gtactgtaaaaagacacagacaagccagaacacatttagagaatggccaaggtgtggaaggaaccagaaaccatgccattatgcaactgttgaaggaagtgcctgttttaccttgtgaagagaagactctagaggaagaagtagcatgaaaaccgctggcaaatttgtaaagatctgaagtgtggaaaagaattattctgcttggtcactggggatacaaggatatctgagtgggagtttaaaggcgggggatgtgagctttaaatgggataagaacattctagtaaccagaaatgcccaaagatagaatgcacagtctggagagccagtgaatatctcacaaatggagacacttgaaactaggatggggatgctgttgtaggaattccagcagacaagtggttgttggttccttccccaactttgtagggttataactagggatgttcctgcgttttctgcttggaggatctgcaagacacctcagggcaggaaatggcattaaatgcagaacagagctagtggctgaaaagcaaaaagccatcaggatctctgagtagtgaaggaaccagagaacatgcaggcaatgtccatcattctgacgcaatcagcagcataatcatcttcccccaggaacatcttgaccagggaatgtgtcagtgtggtgaatttcaacagtggaaagagaaactgctaaatctaagaactttaatttttatagattatgatctcatctctacaattttgaatttcatgctcaataaaagttccttactctctttttttttttttgagacggagtctcgctctgtcgcccaggctggagtgcagtggcgcgatctcggctcacttcaagctcagcctcccgggttcacgccattctcctgcctcagcctccccagtagctgggactacaggcgcccgccacgacgcccggctaattttttgtatttttagtagagacggggtttcaccgtgttagccaggatggtgttgatctcctgacctcgtgatccgcccgcctcagcctcccaaagaaaagtccctcactcttaaagttgcctcctccttcccagggctggcttcatgggcatgcaaccctggagagtctcacaggccctgcggtgggaggagccccatgcttggtttaacgctctgccattgccatcttaaaattcttaatttaattttttttcttttttttgaggtggagtctcgctctgtcgcccaggctggagtgcaatggcacaatcttggctcactgcaacctccgcctcccaggttcaagcgattctcctgcctcagcctctggagtagctgggattacaggcaggagtaaccacgctcggctaatttttgcatttttagtagagatgggggtttcaccatgttggccaggctggtctagaactcctgacctcaggtgatctgcccacctgggcctcctaaagtgctgggattacaggcatgagccaccaggcccggccttaaaattcttaataatgtaacaaagggtctcacgtttgcattttgcagtggactctgcaagatttgtagctttggaccacgtttctctttgcattcagataccttcttttttgccttatttgctcatgcagacccggaacaaatacggaattgcggtgggtaaatgtggtgcagaaagtgaacaactgggtttgtcctgtcactttaggcttttccctgctgtcccagcttcatgtcacttacttgctattagatttgggagttcattagcttcattttcctgatgtataaataggaataatagtaacagcctctttggcttttgtaggaagtaaatgacatgaagcgtataaacaaatactgcatgacaataaatatttgtccttatttgttgaggacatccaaaggacattcaggggcaaaagtaatccaagagtcaagactgaatgcctagtgcgggaaaagacacacaagacaacatttaggggagctggtacagaaatgacttcccaggaaggaagtctgtaccccgctggctgagccatccttcccgggcctaggcacccttgtcagcgcaatgagcaagggagagaaggcaggctgcagtgcagccctcagaagggccagagcactccctggcttcagtccttcgctccaagccctgtgtggagtgggctgtggcttggtaactaaatgctacttcaggtcaagagcaggggatatatctgggcagttctagagcattctaaactatctggacactaactggacagtggacggtttgtgtttaatccaggagaaagtggcatggcagaaggttcatttctataattcaggacagacacaatgaagaacaagggcagcgtttgaggtcagaagtcctcatttacgggggtcgaatacgaatgatctctcctaatttttccttcttccccaactcagatggatgttacatccctgcttaacaacaaaaaaagaccccccgccccgcaaaatccacactgaccaccccctttaacaaaacaaaaccaaaaacaaacaaaaatataagaaagaaacaaaacccaagcccagaaccctgctttcaagaagaagtaaatgggttggccgcttctttgccaggtcctgcgccttgctcctttggttcgttctaaagatagaaattccaggttgctcgtgcctgcttttgacgttgggggttaaaaaatgaggttttgctgtctcaacaagcaaagaaaatcctatttcctttaagcttcactcgttctcattctcttccagaaacgcctgccccacctctccaaaccgagagaaaaaacgaaatgcggataaaaacgcaccctagcagcagtcctttatacgacacccccgggaggcctgcggggtcggatgattcaagctcacggggacgagcaggagcgctctcgacttttctagagcctcagcgtcctaggactcacctttccctgatcctgcaccgtccctctcctggccccagactctccctcccactgttcacgaagcccaggtgggccgtcggccggggagcggagggggcgcgtggggtgcaggcggcgccaagggcgcgtgcacctgtgggcgcggggcgcgagggcccctcccggcgcgagcgggcgcagttccccggcggcgccgctaggggtctctctcgggtgccgagcggggtgggccggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtgggtgccgcgcctcggaaggcggggggaggctgcacggtggggacgcgataccccccaagaccttaacccaagtctttaatgcagagaagccgggggtccgtcaatgggacccctctcctctccgcccccgcttgcggacgtccagcgcatccccgctttcggcccagccctgccccagggagtcgcgctccggcccgctgagagggagcgggcgaggcgctggtctccctggttccgcgccagcccggggcgagaagggtagggggcgaccctgagcccagaccccgacttagtccctgccttggaagcgggggtcgggggaggcgagagacattcagacaggggggaagggggaaggagcacgtggggaaaaccgaaaacgcagcgtccctaaagccagtccttcgctcttgaatcgttgccctttcctttctcttgggccctggggaggaggaggaggagtcgggacagcccaggaagctaggagcccctcgcttttccctgcgttctccgcctcgtttcaccttcctctctcacccccatcccccttccttgctccaacctcttttcgcggctgctggcggcggcagcctgggtgcgaccctagcccgagccggcgtcggggtcgcgcaccgccttccacaggcaaactttgtgcatgtccgcgtctcccttttgtgtaacttgcgagaaatgggaggggtcggagacccatagccaccccgcgccttccccaagtctggacgtcccgggagttcgctcgcaccctaagctctgtctgagaggcagaaggtccgcgggaacaaaagccgcgacaccccgccctcgccgtgggccgagccgcgttagtctcagtgcgccaccgaggctcccgcccggctcccttcccggggctgcgcgtccgcctgggagggggcagagacgctgccggggcttctggcggagcctcggggcccctctgcttcccaccctcggatcacctccgaggcttaattctgtgctgggctgtaaagtccttggcggggaggaaataaggcggagagtgggatgtgcgtctggaaagggcagtggcggatggtggggaggcacgggctctgtcctgtgcatccaaggaggcgtttgtctgattaaggcgcggcctcttccctggcagctctggggactctggtttagttcccctgggggcacaggatgctggggagggtccgaagggtcttttttttagggtgcagataaaaggatcgaattgagtgaagattaagacggagaagatggcgcctctgcagtgcagcaaagaaaagctgtgtggaggctgcagcctagtgaaatccacccaccactaggtgtataacaagtcttccccattcatccaactctcgaacattcagctaacgcctgtgcacaatgactgtttttctttacatctgaagatgcttagaagttgtttgcatgacagttgggccagaggaggctgtatttcacttcggacttcggttgggttcctgtttggcatcttaacatttgcaaccttccttacccgtttttcccctcttttcccccagcctcctagtaattttccaaaagaaaaaaagcaatttccgggcagatgttttcctgtaattccactgaggctgtacagactatcacaccattagttttttagtttctaatttgcaaggtcattgaacttttttctcttctgatttttacccacaagtggctcaggaatcaccttagggctagttaagacaggttgttttaagagttaaagaatttcgttttccatgttttaaaaaaatgcacttaactaagagaattctgatttctctcatgacttacttacatttatttgtagaattattccagttttcccgaatggtcttactacagattaaaaaccatacatttaaggaattaaaagcatatgataaccgtatttttgaagcttttgtcaaaacttaaatatgtaactttgttgagcaatatttctttctgtataaaaagaggaagcatgtaggcagattcgaatttagtttttggtctgtttaattgcattaatatgctctttctgccatcttttgggaccttttcagtagctattgttaactcctttaaagggtggaggtgagattcacattatgccattttgcgaagtgttggtagaagggtaggtaattcagtctactgaggtttgagggtggtttatctggttctctctgtgtccactgaaaagatgtaattacgagttgactgtcttttatagagcttagaaaataggtctgctgcaggtggctgtagaaaagaaatgaataagggcaatgataatggggccttggctatcgggggaaacatggttattatctgggttgatgattttggctggtatgttgatagaggtgtgagtttaacaaaccctaatctatatgatgtttcactaccctaatatttatttatttatttatttttaaatctagcttcctcttaagttttatttccctctgttgagggtttaacctagtcacaatcatcttacctttgtggtagtgctaatttatagtttatggaaacctgctgctactgactagtacctcgctaaaggtagaagccgacacacagaatagtaaagctcaggtttgatggtgatctgtaaggagtcacggttcttgctaatcgatgagaaaccatgataactggcagactgcagaagcttttctcttgtcaccggaaccagcagaggctcctgaacgacatttgcaaggctattagtactgcggagaagttactagatggaataatactggtcctgactgaggaaatgaaaaggaacatgcaagttcacgaggttaaatgaggaatggctgattcaaaaaaattaaaaagaaataacgtgatcaacaatattggtttgaaggaatgggagaggttgtctttatgttaagtcttgttgctagaggatctctattttatgttactgttttttgcatactagagtcttctacagatatttgtatgaatgttgtcagtcattctggaaataagcatagctattacttgcatttcacgtggccactaaactatcagtgagaagtgcattaaattcagtagaaatgtacattgtttctgcaaattgtattaccttgagaatcttacaaagcatctttcattaatacatttcttttaaaatctctatcaattacaaatgtaaagataaacctgtacagatgcattttaaatttaaagcttccagaatgtaaggtttagttcgactgcatacctcattacctccccttccctcctccctccccttactcccaggaagtttgcagggtaaataaaacatgcacatgataataaaacaaagtggaatgtactaaaataccaaaaggaaaatacagattaggtatactgagtaaatatgttacagagaaaacttcctgtatttaaaaagtaaggaacaatgggatgagatcatagataatcctggagcatttcctggtagtaaatttccagccaggtttatatcccaggtctggggattggaggttaagtttctgcatatacaatatttgtttacattcctctctgcctctaccagacagacagacagacacacacacacacacacacacacaccctctgtctctttctctctctttctctctctctctctctctctctctctctctctcacttgagtgacagatgctggtgcagggtattctcaacaggagtagggaatttgaaataaggagttgagaggtcaaaggtaaggggctgaatgcaggaccagagagcttcagtagcatcttgatgggcagagtatgagatgaacagtggcaacgaggaggatagggaaaaatcacattttctgtttgtgttttgtttttgtctggtggttttcaacctctctctgtttcacttgactatgcttgattatgaaatcttttttttttctttttttaatttaaagatctcaactggctttgttgtgattctacattcaggcaacactattctgtaaaatagtataaatgttccgatgagttgaacagaggagattggctttgtagacagaaaagggctgaagaaaacagaaataaaaaaatcttttccttttcatagtgagagctcagatgcaaatgtatggagtggggatggatgggaaatgccgtcttttgcaaacccttggaaacgctgtcaccctgtgggtctcacagccagtgtacagataacaccctcatctgtacacagaagacagaaggtggcaggggaaggaatgagcacaccccagaatgagtagcaagtttaatcaataaaatatgtatttctttttttattgttgtataagaagaaaagcagacagaactcaaaccctgaagctgattttgcaagtgaaaagcttagtaaagggggaaatgttatgtaactattttttcagtttagtgtggatggcttaagaaataatcattactgtgaggacactagtatttttcttggaaaaaaaagtttggcaaattttcctctttagattaatcttggttatagattattattatgcttgagtggtacctcttcagaagtagaaatctggcaaaaactggagtacaatgattgctctaggaataaattcacatatttaaatttaaaagcctggatatgtgcatcaattaatagctttatgaaacagggatgcttagtagattagctagctgagtattcagagagggccacagaagtctcaggttcacatgtattaaatctgctgaaatagaaacctaagagatcattttatttaggtttattttgtgtggctacatgttgaagaaacatgcaatagagatttagcttgaaccttgcgagactcacagtatcttacgaaagaagatagttaaaatggggaagatgagaggtttttaaaaatgtcacctctgtagaacgaaccagagttgttctcaatttataatgttgaatttgatgccaggcgtgatgttaggccaataacactgagtcattgagtgtttctcaggtatgtagagaaggcagcattgcacactaatcaggagagtttagaccagggcaggaatgcatgggcataaattctgtctttgtttcttttgagctagtgaccatgggcaagtttctgtgcctcagttttttcctttgtaaaatgggatgagtacttctagttattgaggatcgaattaatatctttgttaaatgcttgaaacagtgtgtgagttaatatatatgtaacctgatggcatagcataagcactgtgtgttagttgctgctatgtatggtgatgatgttgatgtacaatgggtatatgtaagtttcctgcatagttgaaagttaaaaatctagatgatgtttcacttattgacaaattacttattggccaattaagactattgcaggaaattgacagattgttgtctatgagtttgatctaaatttaaaaagcacatggtatctcagatacaatttttagaatacatgttataaaatttcatttattaaatatgtattgtgtatttcaattggttttttgaaaaatgaaaataattcatgctcattgaaaaatttaaaacagtacagaaatgtgcaaaaagcataagcattttttacacaaatgggaaatacctagtttttttttttctttttttccacataacatggatgtagaccttttttttttttttttgagacagaatctccctctgtcacccaggctggagtgcagtggcgcgatctcggctcactgcaacctccgcctcctgggttcaagcgattctcctgcctcagcctcctgagtagctgggactatgggcactcgccaccatgcccagctaatttttgtattttttaagtagagatggggtttcaccatgttggccaggctggtctcaaactcctgacctcaagtgatctgcccacctcggcctcccagagacctattttttaatgttgtacacactggcagcttgccacagatgtctaatattgagcaagtttatacactggcttgattattaactattagttggctatcagctatgtgcaaggaactgtgaacagacttggtttagttttcagtagaatctttgctagcaaaacgaataaacaggctttgccaatctgggtagaaatggaagatttcaaatgccagcaaaagaattttggtcctaatatggaagcttgaagttatgttcacccaaggacagggaaaccaaaaagcagattgcttgagaatgcggcatttctctaggaaatcagtttcaaaataaaatgaattttttgttttgttttgtttttgagacggagtctcgccctgtcgcccaagctggaatgcagtggtgcgatctcagctcactgcaatctccgcctctcaggttcaagcgattctcctgcctcagcctcccgagtagctgggattacaggcgtgtgccactatgcccagctaatttttatatttttagtagagacggggtttcactatgttggtcaggctggtctcaaactcttgacctcgtgatccgcctgcctcggcctcccaaagtgctgcgattacaggcgtgagccaccgcgcccagctgcagtaaagtgaatttgtgtaaccagccatgttatggctgttacatctttaaaactttatagtgatctgctgtttaattgaaattatatattttttttaaaaatcagcatatgttgctcaaaaataaaaggtttttagcttcaaaagcaggccaaggatatttgaaagccttgagtgtatgaaactggtgatactgtggttaaatgatgatgaacgcagggaaaaatcaagaagcagagtcccaaagaattcacatgggtttttgttaaccagctcatgtaattataattgtcctgtccaactaaagacaagaagacaagaagtgggttcacatacgaggatgtgtaatgacagaagccatatttgtgataaacatcctgattacagtagatacagtgaggagtgattaagatggttccagaagcaggagtggagggttcatgagcagggatgaggaataaataggacaaacattttggaaaagggaaaagatgaataagcctgttttgtaaattaatggatcctgatgaatgaagtgcagtgattccttaaggtcttattaatgatggaccccaaacagtgtttgaatctcttaatttccagatggttcctttttttcttcacaaagtcagaaggttttcttatgagaaatttgattcaacaagaataattgtcacttcttttgttttttggtgatttttttttttttttttttttttttttttgagactgagttttactgtgtcacccaggctggagtgcagtggcgtgtcttggcttactgcaacctctgcctcccgggttcaagcaattctcatgcctcagcctcctgaatagctgggtttacaggcatgtgccaccacgcctggctaatttttgtttttgttttttttttttttagtagagacagggtttcactacattggccagggtggtctcgaactcctgacttcaagtgatccacccacctaggcctcccaaagtgctgggataacaggtgtgagccactgcacctggccaattttttggtgattttgataggaatttttttttttgcctgatgctttttttgagagaatctaataatttgtctcttatttaataatttggctttcattagcttccagaaagccacatactgtcttcattgaaaaatgtaaggcatcctagatgatacagactgataaattacttttcagccataaaagatacttatgagaataagagatctgttgcataaagaaaagtcatttgtgttaagtgaagaaatgcattattactcttgctgctttgctaaatttcaaacattgaataactaatatattttgaccccctggagatattttacaggaagaaaaatgagaggtttaatcctactgttgagatagacttaacgtgttttagacctgcccctcatcccaagttactacaaactcatttgtttagtttttccttgtctgtgtgctcgttatttcacagtaaaattagtgaggcaacataaattttatttttaagagaaagtaaagaaaatggctttatccattaaaggtttattcatcttaaaaaaggacttgttatatctactcctactattggatgaattttcaaaagcatattgatactgtgtttggttttgtttgttttcgggacaagtcctcactctgtcacccaggctgaagtgcagtggtgtgatctgtagccgtggcctcctgggctgaagccatcttcctgccccagcctccagagtagctgggactacaggcatgtgccaccacatctgactaatttcttaaatgttttgtagagatggggtccccactatgttgcccaggctgatcttgaactcctgggctcaagcgatcctcctgcctcgaccttccaaagtgctagattacaggtgtgagctattgtgcctggccaatgttgtttttataaatatcattactcagttattttgtttataattgaattgataggctcatagaataaagatggataagcactgtttcttaactgtatttattgatttttttatctttcaaaaaaaaaatcctcatctacttcaaagattaagcctttaaaaccgatgcaaagtttttgaagtcttagcttaagtcttcagagactaacattaaaacccatcatatgaaaagtgatatgtagtagcaaatattctccatcctgatgctctcaattgcttattatggtttaacacaaatataattttgtcattacagtaaattagtttgaggatataactgaagagtacctaagaaactcccatagaaatgtgaaaatatgcagactaattattctaagaataattaatcatagatacttacatattaaattgattagcaatttaaaattaaagccaaatttctgtggaaattagcttattttcatacacaaagaaacctagattcttaggggtacaaccaagaaagggccactccttcatttcacaaatattttcgaggtccttgcccccaagagggtagacagaataaaaaggaggaaggaagtgacttggaaaaaattaagttgggtaaggaatataagagaatgctacgtagaaggatgtgtgttgtgaatgcaatagtttctgtagtattgtcaaagttctctgatcatttcagtatgtgaaggtaaacttagataactgggggaaggaactatatatttactacattgtaaaagaataattgtagacagaagatctactttgggtgattaaatgttgaaaacaagtggtgggagagagaagttagtgttgattgtgatcacacacttttctatacagtaatttgtttgatcactcatgataatgccttacattttgtatgtactaaataaatgagttctctagaaattctatgttgtggtgataaagaattacgttcagattgaagattcgaacagctgtgcaaccttctcaacatttaagaaactgcgaacaagcatagaaagaagattaaagccttttaaataagattcttttttttcagcctctttttggaacattttattgtgaaaaggtttcataaatactgtcaatacatcacgttcagtcatttgcaattgtatttcaaaaattcagttggcctgcaaaaagaaaaaatgcctcatttttgaaatatttcatccatgccctgattgattgggacactttcaatagagggtgcctagacataattttgtgtgaagaagttagtagagtttggaaagaaaatgtgtattggaattaaatttagtggcatgaaaatgaaaatttctctgtcattgtctttttttttggctccttttttggccattttacagctagtgtttagtgtgacaaggatcaaaattggccctttaaaaaaatatattgaattctattacctctgttatctaaaagaattgtatttttcaatttgaagagcttgaagccaatactactatataccattaacttgacaggaattgaaaatctaattgaggtcattatgattagccttgtatcttgctagtattttctcaatcttattttgaatgttttgtgtcaagtgttaaatcctcttataccacagaatccagaaaatttaaatgcaacagaagtaaaacgaatataaaaattctatggttgttgtggaggggcgttcttggctaacccatagggtgtttgaatgtaaaggcagcttacagaactgctgctagttgaattgtggagggtttcacttttaagagtacatttgagacaaaattgcaattttggaatcacttattcctgcttgttcagtgaaataggagaggggggaattcctgagggaagctgggagatttccaggtgatgatagagtagccatgtgagtatttggctgaaacagagtagaagccacctctgtctgttggacagaggtggccttaacactgagtatcaatcagcatggccattagagctgtccaggacaaacataatgaatgttgtggtggcagagagggtatacttaaatgaacaaaattggaggctgactttccttgtttcttggtagttcctttcttcgtatttgctatttggatatttcacttcacttttgtgagctagactcaaaggttaaatttatatcattgtgaattttctgggtcatattgtagggacacagatgggaagcatcccttcagtttattgagagtatttcttatgcgcatgtgtttttaattgtatttattgatttaataaatagccgatgagatgagaaaaaaatattagtgtcttgttgttgcagagaaaaagacattggatgcattcttagaatttattatcacatgcgaagttatttagatttacaaagcaatctgacatgttgacttcattgctcgtttgctcatttctatcagaggttgaacacaaagccaagcctaagcccgtgattcttctgtgtgcagatgagggggaaggtttaatgcagaatgtttagctgaatatttggattgtgcattccccttttatgtctctactgcaagttttctcagggcatttgtgattgacttctgtgatgttgacatttatttggagttagatgtttaaaaaattactaatttatggcctggtgcggtggcttacacctgtaatcccagcactttgggaggccaaggcgggtggatcacgaggtcaggagatccagtccatcctggctaacatggtgaaaccctgtctccactaaaaatacaacaaattaaccgggcgtggtgacaggcacctgtagtcccagctactcgggaggctgaggcaggagaatggtgtgaacctgggaggcagagcttgcagtgagccatcgtgccactgcactccagcctaggcaacagagcgagactccgtctcaaaaaaaaaaaaaatttactatcttattgtttctctcattttgaatttgtgaaattattattcattttatggacatactgcatggattctgtgtttcagttatgacaggagatctagaatctgggtggggctgtgagtaaatagaaaggtaactcagtctgtgccttgaggaattccgcagtccagtggaggaagaaagagacggagaaataattaggtacagtgtgccaaaggctataatagaggaggtacaaactcttcccagagaaggcaggcagaatttgaactaggtttcaaggatgtgtgaaggtgaactggggtggggagggcttcccagacagaaaaaacagcgagggtgtagagtttcaaagggggaagcccaggggtataataagtgaaggagacagtgtgaaattataaattttggatgcccttaatgagtttgaacttcattttcttagaaatggagtacttttaaatgttcctaaaatggggacgaggcattgatattcatggttcagttaggtttaggagcttttgtagaagtccttttcaaggtctgcttaatctcatgggccagatagtgacacctcgatgcaagggagcatgtaaatgtgctctattagctgggagcattgctatcagaaattacatttccggctctcttcttaagggtaaaggagaggtaggatattgagtggcaactggcattctgggatgtagaagaaatacagtaccataaagggatagaggaaaatgggtagacttaacgttctgaaccaaagctaagattctccgtgagcgattagagtaggcttttgggatttgcttaccttctcttatctgctagccagccacacaggaagcacatctttgtggagtgacagaggaggaggtaggaaatgctttctcatttttatggtagaaacatccaagagctgagccttcattctggctgagagaactctagatggttcagacttgcattcctgggaaggccttccagctagagtgatggaacaaagcaatgatttctgtggccctgtgtggacagcagtggcagtcagtttactctccctgttggttaactgtctttcgtttgatctagagacttcctcaaaacaattttgttgtgcttgttctttgaaagcaacagcagacaaaacaagctttgaggtttttagaaaagagcttgacactgagcacctcttttgagctagatctgtctgtcatgctgaacctgttttctgtggctgcgtagcttttcaaatctgtttatgcttgctggtgttattgaaacagaatgggaagatctatgcaaaaaggaacattgatcttaataaatttatgtcagtaagaatgggactgccattttaaaatttattttaattccttacctattttcttgttcaattattctaaaagtgattaaatgaagatacgtcacagaaagtatgctttgaaatcaagatgtggggaaaacctgtagttctgagtaggcctgatagaatcatcattgttagagtatttggaggttttgtagtagggattttgtcttcacttaaatatttagttgaaatatgttccaatagtttgacacattacagaatctgtctgaaagaaatgtctaaaatatgattcctcagaagctgctcttggttttactttctgctgttggttttactttctaacatttcattttgatcccgcagttttaagagtaatcaaaaaggccgggcacggtggctcacacctgtaatcccagcacttcgggagactgaggtgggtggatcacttgaggtcaggagttcaaggccagcctggccaacatggtgaaaccccgtctctacaaaaaatacaaaaatcagccgagcgtggtggtgcatgcctgtagtctcagttactcgggaggctgagacaggagaatcccttgaacccaggaggcggaggttgcagtgagccgagatcataccactgcattccagcctgggcaacagagccagactctgtctcaaaaaaaaagagtaatcaaaaagctacattatttaaatcttggcatgtctttaaaaaatgttaaactgtcaggaaaattatcacttctggataccaccttcagaaaaacacatgaaaatttgtgcatgtctcaaatagttgttaatttatgaaaaattagaaagagtgtttgaatgtatatttctgtcatattatattgctcatatttttaaattatactttttttcacccgtgaagcccatatagttaagtttgaaaattttaattatattagaatttgaaaattaaataaatctcctttaattcatgcataattttctaatagtcatatagtttgtattatataatttgagaaacttggttctaaaaaaattgtctgccaaagaatgtatatttctgaattctgttctgctttcatacatctgaaaatatttgagctatcataactggctatgcttcaaacccagtctgaaatgggtgacctttgctggctttggattgcagagctgggggctgttgtttgctttatctgatttaaaatagtcagtagtttattttgcaggcagttagatgtatacctttgcataaaatcattgaactgaaaaggttggcactccaagaatacgttgtgagaaaagagaacttgagtactcactctcgactcacctgaatcttattaatattttattgattaatatctgttgtatttttaagtaaataagctctctagaaactatctctgcttctttgttcatttaaaatatattaaacaaattgatacagattgcaaaaattagttgcaacgttcctagttagatatcaatgaaataaaaatggtctattcatacgagttcattttagttattcctactaatatcaaatgtgttttaaaaaatgggagattataatctttttatgaagtaagataaattattactttgtatatttaattcaaagaaaacaaaaaggccacaagtgcctgcataaaggcacatccacttaggattattgttaaggcctacagttgataattaggattcaggcatagcataaatgaagtggtatctttttttttttttttttttttgagacgaagtctcgctctgttgtccaggctggagtgcagtggcacggtctcggctcactgcaagctctgcctccctcctgggttcacgccattctcctccctcagcctcccgagtagctgggactacaggcttccgccaccactcccagctacgtttttttgtatttttggtagagacagggtttcaccatgttagccaggatggtcttgatctcctgacctcatgatccacccgtctcggcctcccaaagtgctgggattacaggcatgagccaccgcgcccagccagatatcaattatttttagacccaatgatatttgctgttgatttattgttggtgcacaaaataagaaaacaaaagcatggttttaaaacagccctttacacatttaatatggaaatggtaacctacaggtctgtattccatacagattcctgggctcattttatctgtgaattcctctgggcgtcatgcatgcccaggactgactttaaagcagaattatccagtagaggtataataggaccgttagatctaatttaaagttttcaagtagccacataaaaaaataagcaggcgaaattaattttagtctgtcactaaattcaaaatgttatttcaacagggggtcaatataaaagctatttatgaaatacattattttttcatattatgtcttcaaaatgttcacatttcacatatttattttttattttattattactatttttttgagacggagtctcactcagtcggttgcccaggctagagtgcaatctagcttgctcattgcaatcttggctcattgcaacctccacctcctgggttcaagcagttttcctgcctcagcctcctgagtagctgggattacaggcgtgtgccatcacacccagctaatttttttatttttggtagggatgagatttcaccatgttggccaggctggtcttgaactcctgacctcaagtgacttgccgctttggcctcccaaagtgccgggattacagttgtgagccaccgcacctgattacatatttcacatattttaattcagaccagctgcatgtcaagtacttagtagccacagatggctagtggttactgtattggacagtgcagctctaggagagtctcttcaacctgcagctgtctaggaagagaatcatagtttctggaacaggtagaatctataagacctcctttttaagcttctccactttacagatgaagaaagtaatatctggagatgttgagttgttcaaaatcacccacattttaaggatcaagagcccagtccctgttctctaacttctaatctctttcttcatcactttttctgcttcatgttgctgcatcccctttagttactaaggtactaacgttaggttgttgctatcagttaaatattaagccaggagctttaagacaatctggcccacttgaaggcatggcctgatttggacaaaacttgtggggtaggatgaatagagttaccaggcatatcttcaacctctctgtggactagtattatacatatatgagatgatgtagggtccagtgatcagttcatctttttctattgtgagcactgttggaataggaatggggggaccttttgggacccaggatatgtgattctgaaccctgaaatgtcagatgcccactaaaaggaaggggagagatggacctgtcattaccttacacattttattgtctgtatgggtatgtaaacatgtacagattataaggaattgcacttcatttctgggcttctggaaacacaggtattaagatttcatttacctccccttgttttggaaggcttattgtgtgtgtgcccatagttcttacccagttacacttaaagtatgatactttttgacagcttaagcctattgcattcctggcatgtaaaatagttattttcagaatgtaagcaaatgtggaaagcttactgcctgacatggagggttggagaagtgggattggtgttcttttgttgtaaactcatttttgtgaaatatttgctttacaacgtgaagcatgcatgatggatgaacttttagaattgaaagttatttttcatttcattcctgaaatatttaccagcatggatgttgactgagtgctttgaagagtttgcttagtcatataacttaggttttatgattttagatttaattaaacttaaagttttcctgtttgatcctcaggacttctcatctgtggcaaggctttactcacaaaggaaatatcaaccttgaagttaagcatgccctgtgtttctttatattctgatgaaaacgattgaagtttgccagtgctgtgtctgggacaatgcttagaggattcatggcttgtaggaatgagttaaaagtacaaggatattggctggcatggtgactgtcgcctgtagtcccaaaactttgagagtctgaggtcagaggattgcttgagctcaggagtttgagacaagcctgggcagcataaggagatcccatctctatgttttttaaaaaagtgaaaaacaacaacaacaacaaggtacaggggtatctgcgtttccttaggcagctgtcttatatacctttaaccttaaaaaaatccaccatttttaaaacagtgttttctgttgtttttcttagtaaatataaacaataggaactgaagtttagggcttaaaatttttgagagaataatttttatttcagtagcttttggggtacaagtggtttcttgttacttagttgaattacgtagtggtgaattctgagattttagtgcacttaagtagggtaccttgtacctaatgtctagtttttcttttaatccctagtcccctattaccctcctcctcgtgagtctctaaagtccaatatgtccctctgtgtgcctttgcttattcctaacttagttcccacttataagagagaacatacggtttttggttttccaattcctgaattactttacttagaataatggcctccagctctatccaagttgttgcaaaagacattattttgttccttgtaatggctgagtagtagtccctgatatatatatgccacatcttctttatcctctcattagtcagtggccacttaggttggttctgcatctttgcatttgtgaattgtggtcctgtaaacatatgtgtacaagtgtctttttcatacaatgacttcttttcctccgggtagatacccagtattaagattgctggatcaaatggtagatctacttttaactctttaggaaatctccataatgttttccatagaggtggtactagtttacattctgagagaataatttttgtaaaattatgtttaagctagatttggaattgggaaagaattacattctttataggaagcttttgaaaaatatatttaaaaatatataaaaatatgcataatcctaccacccacaaatgtctgtattattatgtcatttctaaaccatttttgagattatgctatagattacgttttagatcctacattttatttcttaacattaaatctcatactattttaagttctttgtacatgtaatgtttactgaatgggtaaatctgtttagggaaactggagctatgtagttgtggacttgtgcttttgttttgttttgttttgtttgtttgtacatttgctttttagaaatgtctattcagatccttggcccatttttttttttttttttttttttgagatggagtcttgctctgtcgcccagactggagtgcagtggcgcgatctcggctcactgcaagctccgcctcccgggttcacgccattctcccgcctcagcctcccgagtagctgggactacgggcgcccgccaccacgcccggctaattttgtttttgttttgtatttttagtagagatagggtttcaccatgttggccaggatggtcttgatctcctgacctcgtgatccccccgcctcggcctcccaaagtgctgggattacaggcgtgagccaccgtgcccagacagacatgtgcttttttatttggcatttctagcctgaacataggctggaagtagtgacttagagagggattgtttgccaaatatcaggtaatattttaattatttattatttattgtaatgctgtgaatcaggagtccctaacccctgggccatggactggtaccggtctgtggcgtgttaggagctttgctgcacagcagtaggtgagcaatgggtgagtgagtgttgctgcctgagccctgcctcttgtcacatcagcagggcattagatgctcataggagcgcaaaccctattgtgaaccgcatgtaagaaagtgagagctgcgttgcacactccttattagaatctaatgcctgatgatctgaggtggagcagtttcatcccgaaaccaaaccacaaaattgtctttcatcaaaccattccctggtgccagaaaggctggggaccactgctataaattgctgctgtcaagtagtgttacgcatggtggtctttaacataaaaaaaaaaggtaatgaacgtacaccacacagattagcaaaaataaggaaaggctgatctagtagttcaaacaagcaatcctcttatttaatggttctgtgattgctcgtgtaggcagaagcttaactgtaagttgttgcatttgacaggcaaatgtagctggagctggaatgtagatattagtgaaattgtgatttcagtttactggagccattcagtgggagcatgaagtgggttacatgattacacggatataaataaagcgtcctccttgccacttcactgctgttaaagtctctcctgatgttatgtggtgaaaaacaagtcaaaaacattaattctgtcaagtttccaaagatataagaatctttgaccagaaaagagacggtatttatcttcaaatttaacaaattgtgatgaaatgctgctgttacagaacctagtgtaaggtcgtggagtccattcagccagacacatgtaattttgattattaactgccgtttctgtggtcagtcggccagttttatatatttcacattataggtatttggagcaaagacagtgttgtttattgtttactttaaaaaaatacaatactgtaactacataatgagtaaatgttgatactgttgaaaactaataaaaacaaattcttacactatcaacatcacagaaattctaattgtaagaaaatattgatataatatggaaatccgttcatttatttggtgcattaattgaacaccaacactatgccaagcattgggatacaaccatgaaagaggccagcttcctgatctcacagagcttgtactttgttgggtggggcagagagcaaatcagtcaatctgtctgtctgtctgtctatctatctgtctatctatctgtctatctagatgtcaatcagtatactctgtgccacttggtggtcagtgctatgaagaaaaattaagccagtgaggagctggatagagacacagtctggaagggtatgaaggggatcagaatatgctacccccaaatatgccatttcagcaaagtactaatttgagctgaagacaatcgagaaatagcagacccaggacaagctttaccccctttttataaaaattttgctgtgtgaaggtatcctccttcccctctccatccctggagagaccactagaggtggcaccatcgcctgcagaaacaaaccttattgaaataacccttatcttctattagtttcccccatatatttaccttaccacacttcccctggaagccctaatcccctctcctttgtcttgtgacttctccacagtttattgctctttgttaaaatggtgtgtatgctctcaggcctaactgcttcttttgggtttttgcttttctgtggagctccctcctctcccctccctcatgtaaaaataataacattaaataaaatttgtgttttttcccctgcgaatctgtctcgtcagtttaattggcaaggcctcatgtgctgaacctaacagggtagagaaaaaaggtttttttccccttttctacaagtattaaacctggagagaacaatacagtgtaaaggctgtgaggtgggagcatgctggggtgttggaagaagcacatgagcttacaaacattccatcaatttgcattcattccaaaaattgtatgcaatttttcagtgacgattcccattgatcaatcatttataaaaactaatggtgagggtcttccatactcagtttactcccttcttggaaaagaatgtacattttaatcaagatgatttcatgaaagacattctgtactccaaacatttaaaagagcgtttagagtatacagcaattcatcttttcttctgtattttaaaatttaagacagggagaattattgtattatacttagttcagatttggtggtggttttgttttttggttccagtgaaaagaaattgcatcagtaacctggcggtcaaccgtgccatattgtttgtgatttattagtctggaaaaaataactgtatttctggcatatgtaaaaatagtttgctgagaggcctccttggttgtggcaatagtaatactaattaccaggtactgtatgtagctcattctatgaaccaggccttgttgaaaagtgctttcacttaattttttcttttcagcagcctgtgaggtacctactgttatccccagctgggagggagctggccagtggactgggtttaaactatgttttcattgtctttactcttaaccacttatcttctctgcctccagcaaaataagtcaaagagttcagccagtgtttcttcttggactaagcagtcagcctcctaagtaagcaagccttggaatgcaattgattggtaagatactaaagtaatcagtaaggccctcagttcctggctgccagcttctttcaccctctcctgcaggaagttctgaatgctgatatctctacttggtgtcttctctccccctcccaattccattatttccctttttctcaatcccaagtgaaggaagttctgagtgtggatgttggagtcattcagaccttggatctgagtcacaactctttcaatttactagaaatatgactgcaagaaaatgatgtaagcttccctaaaccaatttccacatctacaaagtatctaaatagattgcttaggagaaaaattgaattaaattgaataaagtgcaaagcacagaacatggccaggagtaggccctggatgaatgagggaaaaatggttctgatttttatttttgagcctctttgtaacatttgttggccccttcagaataatccttagagttcatatcagccacccttcatccctcctgggatgggccaggggagaggcccagttgcctgtgacactgcttcgtttcacatttcactgctttagcatctcaacatttcattgataacatttttgatgcctgaaaacagaataacttatttgtaattctacatacattttgtttgtgatcaagttgttagagtttgaaatgagcctcatttccttgggttttgtccctccccatctgaccttcctcttctcagtgaaccctgaagaaaaaacactagcaaaggaatctcaagaatgttggttcgggacacagctctctccatctactctcctgccctagtcctgcccctccttgtgttttcagcggtgaccaggcactggtgacagccttggctgcttggaccagctggaggtagctgcaaaatgcctgcctgctagctttgaaaaatctctgccaacctcattgaaataattgtttaccacatatcagaggggactgggaagtgggtaattgagagatctttttgaaggggtttgtggcttctccacttgcagaggcttcctgaaatggtgggtaatgctcatgcctcagggagaatcaatatccttctcctaaaacctgctcacacgtagctctaagtattagacatgcatttttgttaacattttatcagtgcatttttctacctgtgtcatttggaccttagattcgggagaaaatcactgtgtatctttggtggtttcaggtggctcttaaatttcattaattcacaaaatctgaattgaatggaatagtctgttttgggaggtttttttgggggggttggggagggattggatttttcagtccttccaagagacttcaggttttttttttgatgttctgattagcccggtgtgtgtaaggccctatcaggtgttcgcctgtgtgatcctatcacacccacatcctgtagctgcatccaagtgctttgaggccgacttaatggggattaacttaacagttgctctgtgggaacagatgctctttgccgcttatttctaataaattgattgagccatttaaaagattttttttttcttttaagttacatcttcttaagaaactgtagacaataaagcaaaactaaaatacttgggtctaaaattagtgtattatatttaaaacactgggcttcagactgattatatcatgctcttttactgactttctagtcagatgcttatttataaaaataaagcttatataaagggcagcattcaaatgttacaccagtcggttttagcagtttgaccttccacagtagctaaatattttttacactgagagaagaattctttaacattatatgctatcagcatgtcaagtgaattctgagcttgcatgcaacagatgagaccctaggtggaaacataaaaacataccagtgtgaaagagaaaagcaggctctctgtctttgggaaggagttctgcagggaggggtgtttataagtgtagtagatacttaggaaaattaatacaattattataaaagtcatctgggaaaaaccattaggtaagaatagccaataaaaaaccagaagcaaaatgtaaagatatagaaattaaaactgcacatcccatgcatagataggtcaatggggcaaactggaagtctagaaataaagttgtgcatatattggaaattagtgcaagataaaagtgtctttttacatcagaaaggggaaagatggattatttagttaatggtgttgggttatctgactgtacatagaaggaaggtgggtaattatttacccagtaccaatactggattacctagtattatgtacccagtacagtttgtcagcaacaaaaggcaaacttttttattccatgaagtgttcttacaaatcagtaagaaatactaaatatgaacatcttcagagaaaaatgaacatttttgaaaacttattatgaaaagaattaaacatacacaagagagaatagtgcgctgaatgctcagcaatgatcagctctcacccagtcttgttttatctgtattcccctttctcaacctgctcaattctcttttccctcactgggttattttgaaataatttcaattctataattttatgtataaatatttcagtgtggatgtctaacagaatctctattagacgtggacaaaaaaggcaaagaattttatagtacacaggaaaaaatacacagccaataaacctatgaagatatgctcaatctcatagtcgtcagggaaatgcaaagtaaaacagtgatataacattgttttttaaaaccctgatgggcaaagactaaaaaggcttgccagattggcattttcatctactgtcaattgataacagatatttctttgcttggagtacagtggcacagtctggactcactgcaacctctgcctcccaggttcaagtgattctcctgcctcagcctcccgagtacctgggattacaggtgtgagccaccacgcctggctcattttttgtatttttagtagaagcagggtttcaccatgttggccaggctggtctcgaattcctgacctcaagtgatctgcccactgcaggcatgagccgctgcatgcagctggtacagaaattttttagatcagtcatttagaattttatgtacatatcccttgatctaggaattctagaaactaattagttcatagtatattgtataaatgcgcacagatgaggatattatgagagcattatttgtatcaactaagcaagcctttaaaaaactcttatacagtagaatattatgctgatacattaaaagtgggcataaccatactgatgtatagaaatatctaaaatcataccgatataagtgaaaacattcagaatgcaggcataatcacagattaaacagtaatttatataaaaaacagaaaatttatatgtgtattatgtcagtagcagattcaagtgaatctgacaaaatctgactctgggcaatgagaacattggaatggaggagaaggtagtacatatttactttgtatattatcaccttttgtactatttgagtctttttaactgtacgcagatactttagagcaggggtccccaacacccagccatggacaggtactggtccatggcctgctaggaaccgggccacacagcaggaggtgagtggcaggcaggtgagccagtgagcattactgcctgagtgctgcttcctggcagtagattctcacaggagcacaaaacctattgtgaactaaacatgggagggatccaggttgcctggtccagtccttgtgagaatctaactaatgtctgatgatctgaggtggaagagtttcatcccaaaaccatcccatcccatccagtctcatgccatcccacccccaacaccccatggataaatggtcttccacaaaaccagtccctggtgccaaaaaggttagagatcactgccttagaggctgctccctcttggcacatcttgaatgtaggactccttggttctgccattcctggtaggtagtcagccagcttttgatagttctccccagagacttcatcccacaggcaagccatcccattccacgtaaaacatctctaatgttaggaaggcacgtattttgatgtgatatctagaatcctcccagctgttcttcagaggaacattacttttataaagttgaaaagaacgttttattgtattataaaactaggtcatttcagtgaatttcatctattttcatgggtaactgattcttatatcaaaacttgcttttgaaagtactggaagcataaattattacttcaggcccatttgctttactttgttcagcacattgaacatcaaggagcaactccctcgatctaaaatttctagcattggtgcttgtaatatcatttttcaagttgacattaatgctatctttggaattctcatggtaagtcagaagaaagcagtttagtggaactgttctttctcccactgaattgtatactgagatctgttatattttctgcattttaacccagaaagttgttcaagagaattgaccatgataagacatctgtagcttattttgtatttttttaatggaaaattgtaactgacttttaaatttaagattgagtgtagcctttcccaaaccaatagctaatgtttttataactatttagtgtaggagattataataaaatccacattgcttaagatataaaatgtttgtgcagtaataggctgtataaaaacagtaaattgagattgtatcaggaataatgtattataaaccatatatgaaaccaggtactaacttggtattctgcatagctttatcagaaaggcagagcaaatactatctcaaaatgaagtataaacaaaatgaggttcagtgagtgatgtggtttggctccatccccacctggatctcatcttgaattgtagtccccataatccccatgtgtagtgggagggaccaggtggagataactgaatcatagaggcggtttcccccatcctgttctggtaatagtgagttagttctcacgagaactgatcactttataagggacttttccccccttttgcttggcacttcttcttcctgccaccatgtgaagaaggacgtgtttgcctccccgtctaccctaattgtaaggttcctgaggcctccccagccatgctgaactgtgagtcagttcaacctgtttcttttataaattactgagtcttggatatgtcttcattagcagcgtgagatcagattaatacagggaggctacataatttgacccaagtagcaataaacaataggggtgaggttgaaatccaggcccctggtacttatggccttaagctaagtgaaataagccagacacagaaaaacaaatactgcatgatttcatttatatgtggaacctaaaacactcaaactcatggaaacaagagtagaagggtggtgttaccagggtttggatgtgggggaagtgaggcgatgttggtcaaagggtgcaaacttgcagttatgagattaatacctaatgtacagcctagtgactagttaataatagtgaattgtacacttgaaaacgtctgcgagagtaaatgttaaatattcttaccacacagaacaaaaaaagtaactctgaggtgatggatatgttaattagcttgattgtgataatcatttcacagtgtatacatatatcaaaacatcacgttgtatacattgaatatacataatttttgtcaattatacttcagtaaagctagggaagaataaaagaagtgacctaaccaggacacctatctgtagctctatggtaaagccaaaagggataataacttttgattatcaaaacttcatcatcattattattttttaagactgggtcttgctatgttacttaggctggtctcaaactcctaggctaagtgatcctcctgccttggcctcccgacatattaggattataggcatgagccatcacacccagccagttttgaaggtatctttattacatttttttgtttaaatatttaatagtaagtgtttaacttgaagcatttttttctgccttaaaattgattttcccactcattttatggatcaaaactattatcagattttaattcccactccaccacattgtttataaaagttgacaattatggaaagtaaagtaatactgaatttttctaatgcctatctttgacatggaataaagattataaaatttcttataacttgaatgagtttttaaaaattggacatctttttataaaaatctagcattgaccatttcaacataagttttacttatctctgaaatacaggtagtacctatttttattaattttcttaagtgatgctgagtgacactatttttgttttttttcactttttctttactttttttttgagacaggatctcagtctgctgccaaggctagagtgcagtggtgtggtcttagctcactgcagcctcggcttcctgggctcaagcgatgctgccacctcagcctcctgagtagctgggactacaggtttgcaccaccaggcccagcagttttaaagattttttgtagagatggggtcttgctgtgttgcccagactggtcttgaactcctggcctcaggcgagcctccgacctctatctcccaaagtgctggaattatagacatcaaccaccgtgcctggccccagtgacactgtcttaattttgaatagggaagtatctaatggtattaaaatggtctggcagagaaggggatatgaggcttggtgaaaataagcacaggatgcaccatctttgagtaagtgttagtttctgcagtgagccagcagcaacattcttagcagagcctagaaagatcgagtgagggctgtgtggtggtgcagcctcttaagtctctctggttccacaacatgctcagattggtggctttgatattttctccatccaggacgcattgtgctaagcttagggaaggtccagtggatatttaccaagcctcttatactgccccgcatgttgccatgagacagcagcgttatcaccttaaatggtgcaaatactccttgtgcataccctacccctgagatctaacatctggattctggatactgtttcctattcttccatcttgctgcatggttcccaggttatgtatatagtgtggctaggcaccacacatggccctggctccttgcagccatacagaggtgacctaactggatgtaattgggtatgtaccgggtgttggctacctgtgcaggttgagggccttccacaggtctagcaaagcaattcattgcctttctgaatttttgaatgcttcatttgtgaaacccttgatgtaataactgttgatattatttctttttgaaaactccccttactcttccctgtttcttcatttgtaaactggaaccgatagaaaagtaccaaacttagggttgttgtgaggcgtaaaagaaataatacagcccagagaacatagtatatactcagcaagtatttattaatatgactactaagccagcattggtgattgattggctcatagtgtcttctatttgccagatgttttcagaggctctgttcatgttcatacccatgtttgtaaatgacagaatgtaaatttcttaatcctctgtcttctcctttagcatcagctcttgtccccaaagtaaaagaactcagtcatccagaacccagttttgtagatctatttttcaaaattgggcagttaatagaaaattaaaagcagtctagggaaaagcctttcctttgggtttgttgctttcttttctgtcataataagactgtgcccaaactgtagggaaaaatcaagaaactgtaaaatacatcatcagggtgtttatagtaaatgtaaaacgttaagtaacatttgagttaatctgtgcttgtacaaacctcagaagcttaaataatgaaccaatgacatataatttagaaagttttcattagactctttgcgttggtatttgcaatatcagatttgcatgtcatcattattagccttcctctattttgagtgttgactaggtgccaggcgtcatgttgagaaatttatgtgcttatttaattctcacactgactccatgagatagatactccattttacagatgaggaccccacatcataaccttttggccagaattacagttagtgttagaatttaaacctatgcagtctcattccaaatcctctgtctttactcctccagaatgctccattaaatgaattgtttcttataatccggttgttttaacgttggaaaagaacttgagaacatttagcccaaccaccctatgaagtttggcagtacttcttgccattctttcaatagataatttatttgagtttaagggagagtctatgaattaacactagtaccaaaactcccatgagaaaataatgtagacagcttttgttaaaaaagcagaatttttaaaggtgttcattcagttatcttcttgcctaacaagttcctgtgttgtgatagactttgaatttgttaaatatgaattggcttcctgattctagctgttttgccagaggcctgatttagagaagggattaaaccactggtcaaccttggatggaatttgggccagcagttccttctccacagcccctgacacttggctttgtgccaagaaaatcctacaaaaggaaattgatttactctacccaagacagagttccggaggctttggttgtccattgttagcttagaagttggcgcagtgtgcgtgtgatccacgcctaaatagcacagccttgctgtgcgtggtagaagttgggttagtgttgacatgctgttgactcaccctcccgaggatggaagctctggcctgggtcaagttgtggtcactgcagttaacagtttgttgatctcagggagtattccacagttgctgatgtaattgacaatgattggagccagctcttccccagattcaaatggaccaattagaggacttgttggttctgtttatcaactatgtaagtgagtactgtgaagttggcattgtttttagtctgtatttgggtaagatgaaactctgttctcagacagtaatatctttcaaaagtacaatgttacttattgtgaccaaagtagggatgaagtttgcaaattatttggtgtaatgatttaatttggccatccatgcagtgcagttctctaccagtgtgattgttctctttgtaaaatactcttactttgccagaaaattgtttgtttgtttgtttgtttgtttgtttgtttgtttgttttgagacggagtctcgctctgttgccaggctggagtgcagtggcccgatattggctcactgcatccttcaagcaattctcctccctcagcctcctgagtagctgggactacaggtgtgtgccaccacgtccagctaatttctgtatttttaatagagacgaggtttcatcatgttggccaggatggtcttgatctcttgacctcgtgatctgcctgcctcggcctcctaaagtgctgggatacaggcgtgagccactgagccctgttgaaaattcttattaataagatgtttctcatagaatgtttcaaaaaattgtgactggtttcaaaatcagatgatttgcaagaattagccatagtataaacacatagttctcaaaccgttttctgggagtgggcccagagatctatttaacaaaaccttgagtagagtctggtgcaagttaaaattggagaaccactaatgtaaccttaccttatctgtggaccacattttgagctttggaagaaataaccctttctatccttcacaaggtaacagatgatttttttgcacaattatttgataatgagcttatatttgagattaaggtatgcacacgataatgtacattctcactctaggtctggggaccttgattctttgactcttctgtccttttctctccagttacttggttttgtgcattaacaaacttttcaatttacttatttttttttacttatttacttatttctcatcccttacgtaatgttgcatcattgttttttgtagcctctgcagttcaagatactaatcacccctttctaccacattaaaatacagactctacctaaaatcatgtatgggagtaacttgatatatacgaaagtgtaaccgtgtcacaattccccgccccaccccgccagtctacataaaactgcaaacactttattgcttaccaagaaagtagacaataatatcgaagaaagccctccttttcctcttctttgaccaacaagggagcagttcctgacaaatgtcagtttgctttttgagttccttaggtttctgtgttgtgtttgcaatggataaatattgagtaacaggtactgactgtattgtatacattgaaaactatccaccaacagaatatttgcaggaggattttgttttgttttgttttgttttgttttgttttgttttctgagacaggctatctctctgtcacccaggatggagtgccatggagtcatcatggcttactgtagccttaacctccttgtctcaagcgatcctcccgcctcagcctcctaagtagctaggactacaggagaggaacaccatgcctggctaatttttttttttttttttgtagagacagagtttcgctgtgttggccaggctagtgttgaactcctgggctcaagaaatctcccaaagtgctgggattataggcatcagccactgtgcctggcttgagaatgacattctataagtagtttcagtcccttagtttaatcccatattttgatcctataacatgtacaatatttgtgtatagtatatgatctgccaactctctaagaagattttaaaacttattatgttaaatttcagacatatacatcatccagcttcaaaaattgaccacattttgctgttcttgtttcagcacctccccctgccccattttcttttctgaagcattttaaaataattctcaggtatcatatcattttacttctgttcataggtttctgaaaggatttgatgcagaatgttattattaaaccagtatacagagagaaaggccactggatattagtacatgttttggtttcagacggtccttttaagagtatattaaatcacataaggattttctgagtacccattagaaccacaaaaaatacctatgatacccttcttactgcagtagaagatgagtttcctagggtagccctttgttgatggaacaaacatgtattttgtttcatcaggaaaatcattggaacagccatgactcagatccactaaatgaaggaacaaaaataggacctgcctcacctcctgatttttttttaaatctctaggatgagtaaccagaatatttttatgttgtgctaagtaggtttgtttgtttgttttttactctgacttatattatttgaattgtagaaatatattgtttttacttttttctttaaaattctatctggagtttctaaatgagagattgtagctcctaaaacctttcttagttttggtgtcttgctttctgttcttagcgtctgtggtcatttctaactcccagtgaggaaagagctctctggtcttgtttctttgttaaatgaaggagcaaacctccgtctcgttgatccactcccaacccacttgttttgtaacaatgaaagttatctcatacttaaaagacaacttgaaattataacacacctgtaacaaagtaaagggtatgggtttttacttaggtttaaaaaaagggcagtaacccttttcataaaaaagcaaaatttagatgggatagaacccccgtttatagaaaagattttaaaatctcatttgctctcattgaaaccttaaccttctctcaaatactggcgcacctctgaggaacttcaaatacggtttgaaaactaatggtttaaggagggccaggcgcagtggctcatgcctgtaaccccagcactttgggaggccgaggtgggcggatcacttgatgttagcctgggcaacatggccaaatgctgtctctactgaaaataggaaaaattagctgggtgtggtggcgcatgcctgtagtcccagctactcaggaggcggaggtggaggcgggaggatctcttgaacccaggaggcataggtgcagtgagcagagatggatctcttgaacccaggaggcataggtgcagtgagcagagatcgcgccactgcactccagcctgggtgacattgagactgtctcaaaaaggaagacgaatggtctaaggaaactagtttgtggtttctcagcggagaatgacattctagtttctacagatctggcaacagaagacaggcacataaactggcctgctttggagtcagggccaagacttaaatccagatttaaaaccagctttttttttttcttcttctaaaacaaccaacctgtgttttaggtccagtgtctgccttgtaagttgctcaaagctcagatacaaggaagtaatcagcacacatttttgtgataacattcctgaaaagaaaaagatgaggtggactaaggtaaatacactgctccttaaaaatccatatttttagattatttataaatcaaatgggcatggttaccaaagatgaattctagaggtgtaaaactccagtcttatacctcaaatttcagttttccttagggagcaaaacacttccatgatcttactgaggacctaaaaaagcctgtacatattttaaaaatagcactccctagataaagcctgatggatccagggtacagtagaatcatgaaattgcctgtttctttttaaaagatgtatatgattgctcggtggctgtatattgtccatcgtgtgcgtgcctgtgtggtatgttggttctttcagagaggcagtggcgtgcagagtgagaggatcacagatgtgtatgtgacaaaactatttcttttctttcttttcttttctttctttctttctttctttctttctttcttttctttcttttcttttcttttcttttcttttttcttttttgagacagagtctcactctgttgcccaggctggagtgcaatggtgtgatcttggcttactgcaacctctgcctcctgggttcaagcgattctcctgcctcagcctcctgagtagctggattacaggtgtgcaccacgatgcctggctaatttttgtatttttagtagaggcagggtttcactgtgttgttcaggctggtctcgaacttctgacctcgtgatccgcctgcctcggccacccagcgtgctgggattacaggtgtgagccaccgtgcctggctgacaaaactatttctaaaacacaagtccagatattcctaccaagggctctctggggcttcgttttgatttatatgcaaaacactcctaatgtggtgacattggggagataaagatctgggatgatgagagagaaaggcaggtaggacacatgcccttccagtaactttccagctagggcttgcatttaacctctgatggaggcctcaccatccaggcagatgtacttgcttaggaatgagtttgagataataaggctaaatgtgctgccttctgtatataatttgcatttgtcctaatgtacgactaaggaaatgaaaaatcaaagtttgagtgactaaaataaaagaggtctgtggagtaattggattgaatttgcaccaagtcaaggagaaaacttctgtattttgtatgttttgtcatggttctatgattacttgttagaaagtgatataaaattcaataacttagtttgatatgaacttggctatctttaagttatagaagtcgtgcatataatgtggctgttttcaacccccataccctttctgagtggttggagatgtcttgaatttcgaagtgagaaggtctggtgctgttacatagataatcatgttagccagctttaccttctgataagctctcaatttaagaaagtacttctaatcctggctttatctttggcttgctatcttattatgcacagagcataattgtaagaagggaataatatctcttgctgtctattcactaagaatagtgtgaggacagggctcttaaaaacatgaagagatgggcactattgtatagatatagagatttgatgaaatgttaaaatgttggatgattttaaatgacaagtggagtggtgagaagtttatttttaaccttatgtggataccttggatatttagaaacatcgaagttttaaggaagaacaaatatcttgactccaagaaaatggtcatgtgatactgtggcatctatttggtcttcctgatgtatatgtttcctgacatttagctcctaaaatccttggaatctccagtgctaagaaggtcttatatgctaatgagatgactggtggctgggtaacctaccatgtgattagagggttaggactttcagtcaccctccccaacctccaggagctgaagtttgagttgataaactaatgatcaatgatacctacataatgaagctttcttaacaaccctgaaggaccgcgttctgggaacttccagatagctgaacacatggaggttcctggagggttggatgtcaggaaagggcatggaatctcgttttctcttcagccgcacctcaccccgtgcatctcttccatctggctgttcatctgtgtgtcctttgtaatatcctttaaaataaatgggtgaatgtaagcaagtatttctcttagttctctgaaggtgctccagcaatgaatcaaaacgaagtaaggggtctggggaatggtagccagttggtcagaagcacaggctacaacctggggcttgtgattgacatctgaagttgggggagttttgtggtactgagccctcagcctgtgggatgtgtaatctccaagtacagtgtcagaatccagttgaattagaggacactcagctggcatccattattggcatgcagtattccttgtcatgagcctgggcaacatagggagacctcatctctacaaatatagctgggagtggtggtgtgcacctgtggttccagctactcaggaggctgagtgggaagatcgcttgagtccaggaggcaaacattgcttctcctccaaatgctgtactttaacatgtttggaaactcctgcaaaggaatttcaacataagcgaagtcaatgggttataaaggactgaatgccattttgtaaatgtcagaaatcaaagtcacaaccaagttaagttctagattagggccaggaagagaaatgtattatctggtcaaccatatcaaatctgtattttggtggaagagcattttcttatattggttctgattggtttgatacggacagggaactgttttctttattccagttatttgttttgttttgtttctgagacagagtctcgctctgtcgcccaggctggagtgcagtggtgcgatctcggctcactgcaacctctgcccctcaggttcaagcgattctcctgcctcagcctcccgagtagctgggattacaagcgtgctgtagtttttgtatttttagtagagatgaggtttcaccatcttggccaggctggtcttcaactcctgacctcatgatcccacccgcctcggcctcccaaagtgctgggattacaggcatgagccaccgcgcccggcctattctagttgttaatcagttaacatagccaaaagatatagaataagattgtagactgaatccatagtctaatgatttgtcttcctagtaattgtgaataattcagacattcaaaattagtctggcatcatacaaacattagactagtcctaaaatgaggatattggcaagtgtgttaataaaaattgcatatgcagtattaataggttccttacgtaggctgccgattactcaggaactgtcagatgttttgttttgttttgtctttgagagatggggcagggttttactctgctggagtgcagtggcacaatcttggctgactgcagccttgacctgctgggctcaagcgatcctccctccacagcctcctaagtagctgggactacaggcatgcaccaccatgcccagctaatttttgtatctttttttagagacggggttttgccatgttgcccaggctggtctccaactcctgagctcagggatctacccacccatgcctccctaagtgctgtgattacaggcatgagccactgtgcccagcccccagatttttaatcttctctttgacattgagtttcagaaatgaagtataaatggtgaatttttagtgtagaatatgatgcactaggaagatcacttacccgtttattcactttcttagaccatgtcaacccgattctgaatttagggttctgttgtgatggtgttctttagaagtgattttaacactcaaaatactatagtctgtaacataatttactgattttttaatttttgcaaactaagttgaagtaatcaagtatttatgagttctttgggtgagaccatctgtcaaagaattctgggcttaatggagatacatatatgttgtgaatactaaacagatatccaagtaataaagcagaaatcatatgctgtgtaacagaaatgtaaagttctgaattagttcacgagtaggagagaaacttttttttaaaggtagaggagaactaaaagtaatttcttaaaagagtgcctattttacatcttgaatattaaaagtctaattcagtgccttgggatcttgttctttttgtagattttgatttgaatgaacttcagtttgctgaaatgtctaatgggtgcaaagtacagatttaattataaggttgagctaaaatcagaatttaagcatatggcatatataataccaatttaatctttcccccttaaatttagttatttttagtgagtcccaaacaccatatttgagacatcacttttttcttttttaaagagatagggtctccctatgtgaggctggagtgcagtagctattcacaggctcgatcatagcaaactacagccttgaactcctggccccagtgagtgtaatgtctcaccatgccaaagtacttttatcttaaattgcttatttttttgtttatttttttaactgactctgtttacaaaattaaccttttatctagtgacagctagattgtatcacatttgtcatctatggacaactgatttttagttgtttaatatggtaagtttattattgtttttccttatttaagaaaacaggatctgagtgaaaaaaaaaaaaatcttaattcaactatcacgattccagtaggaagtagacagacattcatctgtatacattaggtgtcaaagtagagttttgtggtttatcattctgaagatttgagattcaagacatgactatctttcagtcatattgtgcaatagataacatataatgtttctcactgatgagaacagttggttagatctctcagatgacttgaattaggtattagttcaagtcagttgtcagtaagtttggctttatcgttcagaggagcttcttatgactggacatttctcaagtattgccgtattggtcatttggctatcaatgcttaaattatactcagtatagtataaattttcttatttcccttatttaagcatgcctccattacactgttgttgtctctaataagagtttaataatctttgttttggcttagtaaaaagagccaacaaatatatgagtgactgactgagtgtattgctctatgcgcaattgtcttaaaggaaaaacgtttatagaatcactgtgaacaataaaatggttgcatttcgctggaaaacctcggtgctcctttggtaaataatgaagtgttttgttttacgggccttttcactacttcattgacaacagacctttttttttaaattaattaattaatttatttatttatttatttattttttgaggtggcgtttcgctcttgttacctgggccggagtgcagtggtgcgatcttggctcaccgcaacctccgcctcctgggttcaagcgattctcctgcctcagcctcctgagtagctgggattacgggtgagtgccaccacgcccagctaattttatatttttagtagagaaggggtttctccatgttggtcaggctggtctcgaactcccgacctcacgtgatccacctgcctcggcctcccaaagtgctgggattacaggcgggagccactgcatctgaccaacaacatatatttatattacccagtaataggcatagtctaaggaagataatagaatttattcttagtagtgaactctttggcttttgaagcagaatttttttttctttgttcttttctgtgatagtaaggaaggaaggaagggaaggctactactaaaaatataaattgctaaatagaagtcaagtctctgggtgttgaagctcacatattggtgctgtccagacctgacgcatctagaaacaggaatttgcccttagcgtaagcttagccaaatggttgagtcatttctaagtattttaacattatagaatgccttaggaaatgtcatgaatatctaaaacaggtcttcttgaggattttattagtttcttaaaacatggaaaggaacttgattctcagtgctggtacctaaaattcaaagacattagttgtgagttgctgtgctcctcagctctaagccacagttgtcatggccaagcctgtctccttatggagagtggccttgctgtttcctgcagagcttctcttgtgcctggcttgtgcctggaagtcagcgtgtcaccacttcagagagacactatagaagcttggtgatcttcaaacaagaacttaaaacaaagaagttttctttcacagatatcttaaaacttgtagctaaataaaacgtgaataactgtgagagcctcattacagcaatttttaaaaaagcagtacatgttttgttgtatcttaaacatcacagaatggtattgtgtgtccagttttgtttgtaaaatgtaaccttcgtgtgaatgaccatctattcctttctttttggaaggttagtttgtcagctgttgtgtgatgctcaacattttatgtaatgacatttagcacaacgtttcaatataacatctcaggaaaacatttaagaaatagtgaagttctctgggtattacattaatcatattaattacctgttgcttttaatttatcaccgtatcaccctaatatcacaaggcatgtgtagtactattttgtaaagattttctaaagaccttccctctataggaaggaaaatttaattcagaaattgggctgaactctctttcacttgtgtgaaaatgggaaaattagaagtaatttgtattttttattgtgaaatcagtgtaggaacctgtgctcgtatcatggaaacaacagcactaatgtttttctcccctcgggtgtgggctttgctgcagatatccatgtcttctactttttctgttcttagttcttattcacttattgtctatacttcgtgtcactttattgaatttcacaaatgcaatttaggaatccagggatgtgataaaatttgtgcatctgcttgtcattagccatctcacgtttatatcgctaagtgaaaatttaacttagtttggaacaaaccaccatgatttagttcacctgtatagtttaaaagacctaagacctgaaatttttttttttttttgaaacggagtctcactctgtcacccaggctggaatacatggtgtgatctcagctcactgcaacctctgcctcccgggttcaagcaattctctgcttcagcctcctgagtagctgggattacaggcagctgccaccacgcatggctaatttttgtatttttagtagagacggggtttcaccatcttggccaggctggtcttgaactcctaacctcatgatccacctgcctcggcttcccaaagtgctgggattacaggcgtgagccatggtgcctgtccaagacctggaattttacactgcattgggaccttataaaattcttgacttgtacccttttggcattttctgaatgctcatgatttgcttctgaaatattttaggcctttggaaacactccttcattctaatcctctcttttcttgctctcaattcttcatgtcatgtaattattttgcctagagccaaatctgatgtgttattataatcatcatttctgcaggagcttggtaacactcctgcttttgttccctttaaggcttcttttggttttgtttattttgtttcactcagaatgatctggttaagttttgcttgatcatccatttcaaatctcttagtacatttttcaaattcagttccaagcatgttaaatgtgtcacaatggaagtggcccacggtcaaatacattttgggaaaccttggttttaaaaaagttaaactgacttcattattacggttttttttttcaaaagcctccagtatccatcttgaatgatccatttcccatatgtgttttatgacagaattctccccactcccaggtaataactgttgacattttctgaaaactgtttttgagaacactgaattagatatgaaatgaggcatttattcatacttcgaaacgaatgtcttaaacctcactgggccattttgagtatacattactagtgatatatttaataacttagtggtataagcatgcaataacaaatttcttacacaagagaagttctgtgtagaaacaaatttaatactgtggctaaagaaatgaaccaaaaaaatttaatatcttgaaatttgcttcttagctctgtttagaatcattgtgattcacggatagtaaattgtcagagtggacttcatgaaccttatatcagacattctgaaaagagctaaaattgccattaaactgcatgagttaaaatatcttagatagtttaaaaatactatgtagacttggtgagtcagatctgccacactgagcgtgtagctggggttgacgacacacgtgttgcagtgtcgttaccagtttgtctcttttagagacaacttggcaggcagacgtcttgcagataaacaaggaaagattgtctattttaaatggcataagacattctttaaaccagtgtgtcatatgtggttatatagtcagcattttctttagtgcaggaaatcctctctcagggttcctgaataaatagcaggtatctttgtagggatagctcgcaggagatttcaaatgctgtgtattcatcagttcaggctccccagatgtcttacgaacgtgctcattttagttttcttttcttttccctcttcctgctagatacaagttgagacaatatgattagctcttaaagtgcttactctccattttatgtatattactgcattttaatcctcataatatcctgtgaggtgggatgttactatgtccatttgagagataaaggaaatcaagaggtaaagttaactggctttctccaaacagctggtaattactaaatacagaattgacttttgccttttgattccaaagatcatattggtactttcatgtgtgggtcccttcagccagttcctacagtgaccctgacccagctgtaagtcccctgaggtctctagagatatagacagtgatggattagaccctgttcatagttgcagatacaagacatagagaggtttctagcaataatttttttagttatatattttcatagggcttttgatttatgtaacctattagtatatatttgttattccagtagagttaatacattattcaaagagattccagtttttaagccttttctccgtttgtctaagttgttcattatgaaatgattggctagaatttgaaagtctttacctacagagagattctgactggcttgcttggtcttggaaccctagttatgagtgcattgtcttgcttcctggtgaactttgcccagaaatagttaccacctcattttttttttttaaatgaaagctggataattaaccctcaagatttcttaattaaaagattactattataaattgacccacagggagttaatacaataaaattatttttgatcattttatatgaaataattatggggcaatcttctgtaatcttgagttgaaggggttatcttgcttaatgttaataacttttgatacaccaactaccttttaaaaattaacgttttttttttttgcttctgtggaagggaggctgtattttgtaggatttacagtttacatagttattgttttatatgatgtcagttggattaattatgttttgagtggaaactctgtttggttcaaccaatatttgaatgctaattttgttctgtgaacaaatagaaatacagagaacagtaagacatgactctttccccctaagtaaaggcttaggttggagttttcaacctgggaattactgacattttgagccaaataattgtttatgttgggtgggggagggcctacagcggaggctgcctgtacattgtaggatgtttagtaacatccctagcttctacctactagatagatgccagtagcaccacccaagtcgtgacaatcatctttgccaagaactttgaacctctacctagaaacagttcatagggctcaaaaagcaaagtggcctaggacccatgtagttaacaagcttaccctgttggagaaccactgcatagctgaacaacatgggtgagactagtgtcagggtaaaggagctcttgactggagattggccgtacacagaaaacaaagtgagtgttatggatatttggggatttaccttcattagggcacagttggttagcgttaggtgtacaccaacattattccctctttagaaacagccatgcacgtaacattttagcacatctgaagaggcacttctattctaattacctaaaattcttattatctagaattacatgctatgaacaacagcagtaaagtcagaagccagaatctatgtgatgttatctgttttgcgatcacagttgtgttcttctcttagtttatttgaacaagtttgtgaaaaaatttttgtgcttatgtaaatactctttgcttagattttttggtagaatgtaaatctttcccacacagtgcaatacccctcctgcccaagggcgagatatagattgtacagtttgttatttctgtgttttagggcttattcctctgtagacatgtcaaaaagacatccgtcgggggtaaggaatgcattagcaattcaagctgataatctgcatggtgagagtccagataattttttccccaggtcacataggcagctctgcacaagtctgttaaacatatgctagttttgaaaattaaaataagttcccatcactcaaggcaatgaaacgtagatgaattgctctgccagattgactcttagaactcctgatgtgatgtggggctagacagaatgggatgtttgctaataaatcagggaggtagtgattgttttccttctttttctcccccacccccgtcaggacaaactcacattctgtagcctggtgcactctaataatgcctgttgaccttggccatgaaagcactctagactcaccccctaaacaccgcaccgaatgtgctcatccttgaaatttacaagccttagaatcttccctacctccactcctgtttactgcctcctgttactcctggtgcttccagtcagaccgtagtcttggctgtgcagtacctttaggagccctcaaaaatgtttaatttcttttaaaatcagaaggaaaaataaagaacaagataataatcatgaatatacaataatgaatccagcctgacttagagttgtctttataccaatcaacagagccctaaaatatagcttttaatgtttttttaagagagaaagaggcccacgaaagttgtcctggggcccccgcatcctcctcatacactggtgggatgctgctcttgcttgctttggaatgtggcagcagctcccagtgacttcacacgtttattacaaattcaaggtcatttggacgctagccttgtgctacctttcatgcttcctgtccgacccatactcagcctgggcatgttttctgctctgatctttggtctgccttccttctccttctccaagtcaaaccagtcactctttcctgagagctcttcggtgtacccttgccagacctctgcattaacactccagcacctttggttagccaatttggccttatttgtgtctccaccattggattatctgtccatcaaggtcaggaatgttttcgggttaccccactgtccccaactttgaatatgttcactgcctggaaaatgtttatctgaatataaggcatcaagccagaacttgcccaaaacttaacctaggactcaaacagctttgaaatcttatctgtgtagagtgggaacggcatgtgtttgataaccatcagcatttgctgttttgcttagccatcaacaatccgtttctatctcttcctcatagtattttcagctgtatttttgtacagcagtcattcacgttaggtgttaggttacactgaaatctctgacaaccagttctgtgctactgaaggcttttagcagcaggattaaaaaaatagttgattgcacattcgaagtctcactgctcacactgtttatgtaattcttggagtccagttagatcatctgatttttaaaatgttgcctgaattacaaaggagttcaggaatgtgttatcaaagaattaggaaattaacattgaaatttattgagcaacatagctaataggttgtcttgctttttaagccttcttgttttctaggaggagcctgaagtccgtggacaagttgaactgtagctggtatttaggaattggtaaagacagaggtgtggatgctatgttttcttttctctgttaggaaattactctcctggtgccacaataaaaaatttcacaggtatctaaagtggaatttattttgtattgttccacacctttgtcaataattacattttctaccttctagtttattactgttattatttttcatgccaggtgtttcttaattgttggcataagtatttattgaatgcttatatgtgggagtgagagagtcaggccagtgctaaagagtagggaataacccctagacagtgaagtaacttttgtcttgtttgcacattaaaaaaaaaaaaagattaggaatccttgctctatatactagaaatgtttgatgttctcttttcttctcttcaaacagagaaaacttagatgaatacttataggctctactgtcaactaactgcaccctcctaatattggcgtctgcatatttttaccattgtcaatatcagattgcctctgatgttgtgtcactgaaactgatagtaacattacatccaagagtaagactgcagtgtgatttttggctcttggtcttctgttctttcggcaacaattttttataaatatagtcaagaaaatagaaataagagtcttcactgctacctccccttcagaattattttctcctagtattgtggttcctgatagcctttattcctatagaaatggcaattcgaaaaattccttctgtgaatctgagggctctacaaagggagagcttctttgatagtgctttggtgtaagttatttaagaaggacaatctacccaggcttatcaagcaaataacctggaatttaaacctggagtattctttagtgttccaagtctgcaggaaaagaagtaatcttggcttaaactaaataagttaacagaatacaatttgaacaatgaacaagaatgctgtttgaaaattatcttgggtttttatgtcaagttaatattctcactgaatttctattatgtttgtaaagcagctttctcctagaccaaaaatcctagtaataaaaccacaacagtatgagagttataagcacacagtaaaggctgatgagtaaatgttatgacactttgaaaggaaataaaccagacgatataatgtaaatatcctttgtgcttatcaaaaataagtgacttcatgttctgttcctgcaatatttctgtaaatctgctaaaaatagaaaggtaaaggtctaagatcataaatctctgatgccctttccttaaaagtaaggaattcttgtcttggattcttatataatatacttatttataatctcacttgttctgattgcaggtggcctcttactatctttacatccatgtgaatgtgtgtgctttgatgtgtacatatgagagacaagaacaaaagtaaatgtcaaatatccgaattcctgtttcagtgagctttcacggagtaaattctttttgtctacttgcctgtgatctcttctaatatgacttgcgttaatgacctttaagaaatcttttaataaaataaaaaattttattaaaatcttttaataaaaaaattttattaaaatcttctaataaaaaattttattaaaatcttcttcatacgtgttacttgaatttactcatggcaaggaccagttagtaccattttatgaaatcacaagctcatgggcttgtgataaaacatataagtgctttaaatttggcattggtaacggcaggccgactcacttgaaaaatgctctgaactgctctgcctcatgctggtgggaagtcagatggaaatgactttctagagagtttagctggcctccaagcttagtgggtactcactctccttatcccccgatagctgctctgagattttaatgcacattgaagacaatatgataacttaacctggaaaattagcattttggtaagggtctttaacttcattagtattttcataccactttggtattagaaattgtaaagatattaaagggtatggacaatgtggaatgtatgtaccagactcagcagtgtatactgactccctcactatagcccagaactggaaatcccgctaactacctctccccatcctagcctgaattgccctctccacctatgttggttttacaatggcctgacgcctgaaagctggtagaaactgtataggaaaatagggttgggataaatttctgggctgacaaaggatagaggaatatgcttctttttccccttgtccaagtatatcagcaactctaagatgcagatttatcagtatggctctactgggcatgttctagttaaatggtagacccactacctcttctggaatcttgttttccattatattcccacctgcagcttttcaaagacttattctcttctctaatggtctcttggttcttttttttgcccatgctctacgttccgagtgggtcaacattgcagtgggtcaatgagaagaaatagaggggaaaaataaaagaaccagaaagaaacctgttaagtttgcacttacttgtgcattaagcatattagcgtaagccttaaactctagaaaactcactgtagtgctgtaaagtaccactgataaagtaaaagttcatttttatgctgtgtcgttatttagtgatgaatatgatattgcataaaagatgaggatctgtaaggccaggcgcggtggctcacgaggtcaggagttcgagatcagcctgaccaacatgatgaaaccccatctctactaaaaatacaaaaattagccgggtgtggtgacgcgcgcctgtaatcccagctactcaggaggctgaggcaggagaatcgcttgaaccagggaagtggaggttgcagtgagctgagatcacgctactgcactccagcctgggcgacagagtgagactccgtctccaaaaaaaaaagatgaggatctgtgtataactgccaggtgttgtgtgaggtaccatgaataacctggcaatatcctaactgtgcaggtatgaggagtgacccccaggaatataattgcaattaaacttgcaaattatgtggggatactctatttcagggcaataaaataaattatgtctagagctcataactcttacacatatttggctaggtctatgcagcaaaacccaggtgataactgaggctttaactttgggatttgtttggtgctgactctgccacatggtgaaatttggtaggatctcagtgcgagtgtcacatgatcagtctgcttgacccaacatgccagaattccatttctcagcccactctcttctctgcatacatacctacctcccggagagggctgggcgggagcctacgtgtggcaagagccctcactgcttttgttacacagatttcaacacatatggcttgatggagtgtgattgcagtagcatgaatgtttggtgatgaaatttgcatgtcctttgctgggagaagtataattataagatttttgtattcacatagccacatgtgaggtatgagcaggaacggggtctatttaatagcattagattgctttcagatttgaaatatttccttttagaaatatattgtgtaaacaaaatatgtttaattttttttaaaaactatgcctttgtacaaattgtttatcatttgcagaggtctaaaaaatgtaggagaaaagaagctcacaaaatcaatcagatctaaagattgcaaagattgattaccatctccttaaaagcttcagtacatgtgtttacagtatcattctatttgcctctgctcagattctttccaattctaggacaacccaaagtgtatagatcataagccataactgatcctaccttacataatacattaaatataaaatatttatggccaggcatggtgaccagttctgtaatcccagcactttaggagaccaaggcaggaggaacacttgaggccaggaagttgagaccagcctcggcaacatactgagaccttgtctctacagaaaaattagccaggtatggtggtatgggcctgtactcccagctgcctaaaaaaaggcaacttgagaaagtttctaccttctgaaacaatctatgccttgattctatcacttagtattttagttattcctatcaaatacatgactgtgttaatttcttttgccacaaaaatgctgcaaagccacaaactcactccaaaatttagtggcttagaacaataaccatttattatagcccacaagctcaagggtcagctgatcccggcttagcttggctacatttgatataaaacagtagaaatttagaaattgaatatatgtagaatatctcttgtaccaaaaatgaagaggaataatagattttgatttttctttgttgtattctgtgctgggtgctaatctgtcctagctgtcagaacaagtagaagccatactggaaaatcatttatttgttactatcaaatttatttcagtgaatgccattcaaaccactctctctgaggagcccagttaccaagtcctccaccttgttagcaacatggacttaaatgcactaattctagaatgagatcttagcaaagtaccagcatttgagatttgtgcaaattgagctcttaaactgtgtgtatatgtgtgtgtgtgtgtgtgtgttcattctagcactcccactacataccatacaagtagctgtatagcaggtatatatacatgtgtatatgtacataggtaggtatatgtcaactgttgaaaaactagaaaatacacattaaataaagtaagggttgataaacattagtagttggaacattgggagatttggacttctactttgcagattcagttggctgcattagatttaaggaaagaactaactttggagaaatttgtgtttgcatttcaggagagatttcttaaagaggaaactaccagaggttagtagatattaacatctagtgatttaaatttgagaaatgcaaattagacatttaagctgtgggtttgcttcccgttttacttgctctaaaagtaatttttttaaaagccagtaacaaagacatgcaaaatagtactgccatgggggaaaaattacttgaaaataatattcaagattgaagaattaaaaaagcttgacatctagtaccaggaatgccagctggcttgttctgtcaaaaagcaagcatgtgctttggaataacactcttgagtagactctaggcgggttttgagcccttctgattatctaaatagatgtggaagtgacttatcttggtttttctgagtcaaatgagataatgagcaaaggccttagtaggtctttgatttttgcctctaaccctttttctatctgcatgttcacaaggtcaaggctgatgagatatggcaatattgagagagtcagaataggataaagatctcaaatactatatatatatttttttttcatttttttttccctttggtgactgtatgagacatgtcagtaaaactttaaagacaggccaggcgttgtggctcatgcgtataatcccagcactttgggaggccgaggcgggcagatcacttgaggtcaggagtggcaacatggcaaaaacccgtctctactaaaaaatacaaaaattagccaggcgtagtggtgcacgcctgtagtcccagctactcaggaggttaaagcacaagaatcgcttgaacccaggaggcagaggttgcggtgagccgagatcaccccgctgcactccagcctgggcgactgagcaagactctgtctcagaaaacgaaacaaaacaaaagacacacagaaaaactgtaaagacagaatgaaagagatgaactttttacatctcgctcatttcatcacatactatatagtatgtgtatggtttcatatatgttagtgtgtgtgtatgtatatatatgaagttaaaagaaaacattcaccttttaagactaaatggacattgactagattgtaaactgtttttgaggctaagagtttgtttgccatgcataaggctctattttttgtaattccacttttatattcatttatatcagtcctagcagtagatgccactcttactgggtcctgcctaaatgcatgcttcaaatgttggttattaatttcacgagtagtgaataacaaagactcctattgagggacctcttgatttcctactgggcaccagcagcaagcatgacttactggccttcttatgagtgggtaggagttttgggtcaacttgagaacatctatcctttgagtagtaaaaaagcttggtattaggttggcacaaaagtaattgcgatttttgcaccaatctaatatcaacagaggccgtgaaaaacatcaagggaggatagcttgtaattagaagccagatcaacagtgacccaaaaagaatttcaggtttccaggagtgaaatatgtagattactcctctgaaaggaattgtttttctcctattcgctttcatggcaaatcattttttaagccgaaactcaagctctgaaataattgtgtagcaaatagtggctgtgaatagatgtggcaactctccacaagggtaaatagaaaccacttgcagtttcagtcatcacattttatttttcttttttaatagcttctctatttttgctcaccacaaatacatgcacacacacacacaggggcaaagccttggaaaattgcaattttccattttaatttgagagacagcccggtcacattaacagaacccttgacggaataggttgaattcccttctctgtccctataaccccatggccttggacatatccttacctggtgccctttcctccctcttatggtccacttttgtgattctgattttaatttcagttctctgttagtgatgaaatgagcaactgcagatgttccgcttacactggaaatacttttttggcatttaaaagaaaaccctacagatgaattattcccctgaaaggggttcactcagattttctagattatgattctagaaaatagttctttacatttgacaatggagaatttaaatgttttaatatgaaaatgtaaagcactcttgtggggtatagttgaattatgcccaggttaagggaatccagtatttatgtcaggaaaccatggatgtgatagaaatagtatcgctacaaagctgggcacagtggctcatgcctgtaatcccaacattttgggaggccgaggcaggaggatcacttgaggccaggagttcgagaccagcctggccaacatggtgaaaccctgtctctactaaaaatacaaaaattatctgggcgtggtggcacaggcctgtagtcccagctacttgggaggctgaggcaggagaatagcttgaacctaggaggcggagaggttgcagtgagccgagatcatgccactgcactctagtctgggtggtggagtgtgacagtgtatccaaaaaaaaatactgagattatgcaagaatctgactcgaagaagtttgaactgattaaaaagtaccaaatacttcattatatgaggccagctatttagactagtcagcgggtggttcaaatgcagaaaacagatggctttcattagccagaaaccttaaaattacataaatccacggatgcaactaagctatggatctgtgccctctccttgccaaacaggacctctcagctagcaaaacagagagctccttgttcacttttccctcctagttgagaccctgttggggaggaatgctgccaagcctgctctataaggaattttgatctcctagatgataacagagagaaactttttggagaaagggaacaccctgtttgaaaggacaacaagagaacttgttcaagaaggtggctttgcatttaattgaaaattgtagcaactgtaggcgtagggtttccagattcacatctgcagactggtccagattaggtatatatgtttatattcgtttatgttatccccttaagcctcttttaaagccatttctaaaaattgctttggaaagtccatttcacttcagtattctttatattgtggctcaaaaatattttttttctggtaacaaaaatattttctttctgtttttaagacttgtagcatttttaagtttgatatgtacgtctctgtagttcagagcacttggtaaacagcctcttgcttttgaggcaatttatgaatgcttcattgtttttagctggttattttgttcattgtcaggaacactgttttcccatgggaaagcatctccagttttatgatgtcagtggcaagtcattcagtttacaagaattaccttcatattcagtcttagaaaagcatagaccacaaacggccagttatatcacagttctcatcttttaaatgttgtcttagatttttgcaaagtaatcttaactcaccttagaatagtgttaagacataaatagaactaaaatgtagtagttaagtggttatatttcccatctactaaggtatttatatttcttttcttttagaaaataaaaatgtttaaaaaattaggaataattatctttaggatgactttgtaagactgtgcttttttactggcagcaaatacctgccagatttcaggtatttggcaggtaaattgaatcttaaaagatttcaattttaagagtgtccatattaacagatcctgaataggaccctgtgtatcatttattaagtcatttcttggaggcttcactcaggaaaagggagttaacccttggatggcaatgtttaatgtggccacatttccttctggctgaatgccagggagtggctgcgcttagcaaccaggctttgtttcataaatgggttatttgatgcaggtgaggatgccgtgagttgtttgtcttgttttaattcagggagacaggcatatgtagtgaaagcatggaattgtttgcagatgggcttcagtaagaataggaaggtttctggttcaatttgtatctccaagctaattgctctgatttccatatacacatctggtgttggttcggggggaaaaaattttacttgagaaacatggcttttgtgagttgatatttatttgtgaagggcctcttgtaatgtcaaatgtaagaagatctatacacacaaccaattttacaatttctaattcaacagcttaccctattattgaggattcgcagtttaaattattaaatgatctccacttgaagaaaatagagcatgttaatcatcacgattctccaaacatttatttttaaagttaatattaaatctcatctggtggaaaacctgacaggaactgaagcatatattttctagtctaaactctcattgttaaaatgaagacaacatgagacagttatagcgccgtaatcaagttatttgaaaaaaatgttttaaaaattaccactttttggtagtcaacatacacacatatgccaacagatggcattaaaaaatggcaagaacatgagacaattatagggtcataataaatttatttgaaaaaaatgtttaaaaaattaccactttttgatagtcagcatacacacatatgccaacagatgacattaaaatagctgtaaagtcaagaagcagggaggatgacaaagtgaaaaatagaagaaatcaaaggaagttaagtcaaaagaggaaaaaaaaatgaagcatttacttggtttgtcaggtagggtgatccagtgtattttgccaaaaactttcctggttttagcaccaaatgtctcagaacatggcagctgatgacctccctcacagtgagtgatcccagagagcaatgaagaattgtgatgcccttcatgaccttgttttggatgttacacatcatcatgcccttcagtgacctttctggtagcaaagggtttgtttttaaaagagttgcagcatctttaagtctaatatatacatgctcggaagttacaccatcatcacttctgctgtactccatttgttgcacagacagaccctgatacattgtggagtttcccagggtccatgaatagcaggaggcagggactattgggggccttcttggaggctggctaccctggggagagaaattgtcaatttcctctccttgcggggaggaaatggaatcaagtgcatttgtattgacaagaaggtagaacgctttccttagacattgtaatgaacagattgtggataaacaaaagtacagagagagtttgaggcagaggaggagactgtttcaacagaatatttaccagctatggtaaaggctcattagtgtctccgtacagttaacaacatgctgccaagactggaaatctttgggacaggaatgaattcggaaggtgaacagaataaagtactgtgggaacaatggcgattccattagtggattgtacattccttacgggcagggccctccatagcatctgatgtagggccttatccacaggtatctgattattatttgactttctttaatgtttttgcagtgttattatctatactacttatttcagtaagattgacaacatttgcatttcgatttctaagatcaagacaatgaatttcctcttaatccctcaggaaagctgtgaaatttaaatttatagagcattctacttagcctgctgctttgcatctgtcagagaatgagaccccccagtttcatggaagtcacaggaagtggcaaggggctcagatggagttgaggggttctagctatttctgcagatgatctggatattttggcatcggctgctttgacttaaccatctggcaatgtaaaggcagaatttcgttaaatattggaaaagttacttgaaaacatggctctagagattttgccagcagcttttgcactcatgacttggttcaatctaagacctgttttgtttttttgttgtaaggacctaagtcctctgttgggttttgacgaagtatgtgtaaaaatgactaaaatggccagaggtatcttattttacatttatgtgggaagaaatgtttggaaactgtttcatttctttaactgaaaatttctcagtgcaattttagttggtgtttttcctcctgtggacagtttttaggaatgatacaagttatcgtggaatcacccagttcttgagtgtgtgaacgaatggcttctgctggaagttaattcttcttggcatgggtgtctcacatttccccagcagctggatttgacaagggagggcctatcatgattaatatgccttctaattgattggtttaataacacctttactcctgtggatatatttttcatttcttgctgaatttttttttctttttgagataccagaacctgttaagtgacacggtatagatatttcaggacttccattttctcttttaaaaatatttttgtaaaggtaatgcctgtacacagaagagtttgaaaatacagaaggcaccctttttcatgaagttattttgtgaagtggtctattgcctgcaatggtaatagctaggaccccttacttactacccagcaggcactatgtagagctccaaatgagaaaatgttggctccttgcagatggttgggagaggagacaccagcactcttaacagacttgttaagagttgagactgataaccagagaatattctgggggaaaccaggagggatcgccaacatactcagttatatttgtggatggtgaattctacagtgaatttttctgttcaaaatcataatacagtcacttgctaactaccaacgccatacttgctctcagtgctgagaggtggcacaaatattattttcagtcttggtcatcaggcagggcctggcaggcatcatgctctgtgtgagtcagtggagtttattataagaatgcggtagcctccacagtgattatatttattataggaaaacgtgtaattttgttgtcagaacctttcagcaggtaccacaaaatctcatatgggaaaacgtgtaaatttttcatgaataaattcctttcggtattggtaatttccttctctgttttagtataaaagaaagcattgccttataggtggctaagaacatctgtagcatacataacacttaatctaaaggagtgttgaagaccgggctgattcctaattgagaatgagcaagaatagaactctttttgatagttatccctgttcttcctccaagcctctgccttggagctatggatactataactaactgaagcttcttctttcaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtaagtcctgtccggtggctagcaattcacgttggatcacatgcatttgttttcaaaaaatttaacttcttgtattttgcatcagtattttaaccctacagtaaaaatcttggttcctaatgattcaccataccattaatatatttatttgcattaccctatgatatacatataaatgtttttaaaattatgatgtcgtattatgaccatcactaaacagtagtttaagatgtcacagcacttttttttttcctcactctgtcacccaggttggagtgcagtggcaagattatggcttactgtagccttgacctactgggctcaagcaatcctcccacctcagcctcccaagtagctggactataggcacatgccaccaggcctgactgattttttttaatttttagtagagatggggtctccttgtgttggccaggctgtctgaaactcctgggctcaagcgatcctctggctggacctcccaaagtgctgagattacgggtatgagccaccatgctcagccctccacactcctttcatcttcagtatatatctcaccacggctctacagtcaaggtcttttggttaaaagtgctagcattaatttttttctgttaccagcttgatacatattttagttcatttgtatctgtttgtattctattatagtttggttttttcctcccttattaattttactttttgactatcatgttagttctatgaaatatactcagaaagctcacttttcactcattctctattgataaacattttcattgttcctgatttatttttttggcaaaagaaaatacatatttttccttcttgtacaaaaaaagtacaaagttcactgttctgcactttgcttttttttttttttttttttttaacttaactttgtatcctggaaatcatcctttgtcatttcgtggagatcttcatttttgtttaattctttgtagtctttttgctgttactatacagggtgctcctttcttattcaatttaaaatagctggagttccaggcattttatctcacacttcttagatagttatctctgcatgtgactgcatcttaagaacattagaagtaattagtcttatgcttgttggcaaagcagaattctcgacactcattataaacagcagatagcctggcaacattttcttttgttgtaatcctttttgtactagaaatttttctttagcctacttggataatgaatgtttgttgtttaattaatattactattctattgagatccttttattttaaattgtaaatactaaaattattattattaaaaggtttaatttgttttgatagcgctctgtttaagaaaggacaagctctaattactaattggtgttttgagaagcaaaaagcatctacattatcacaatatattacatattaagtaatttttatcttgaaaggagtatgaaccatgatgtgaaatgatgaatagtggaaatcactaatacttaagttcattatatgtattttttgttttgttttgtttttttttgagatgacatcttgctctgtcgcccaggatggagtgcagtggtgcaatctcagcttactgcaacctccacctcccgggtataaatgattctcctgcctcagcctcctgagtagatgggactataggcacccgccaccacgcccagctaaatttttttgtatttttagtagagacagggtttcaccatattggccaggctggtcttgaactactgacattgtgatccacccatgtcagcctcccaaagtgctgggattatagccgtgagctaccacgtatggtccattatatgtatttttacaagaacattctgtgtgctgatttactaagtatggattgataataaatcagcttttgcctgggtgtgacttttataaagtgtcaatgttagaaataatatttacattctgctgaagagttcttgtagtacaattgtgtagataagttataaattttataaatcagaactctgtgcatacttatatgtttttgataccatttttttgatatatgcatacagactatgtattctgagtgtcaaataaactttttttaaaggctagaattaaaatttagtttatgacattgaccaaattgtccttttctggtgaatcgactattttgatacatgtgtcatagtttgcatctaagtttttcttcccttagtttcacgtatgaataaccagatgttttcacatttgtatggtggccataccaaaaattactcttcttgaccccaggaacacccctcttttcccgtttcacttagagatcctactaatgcctgggtagctttactttctgtcttagcctgttgtttgtcccagggtgtattagtctgttctcacattgctgtaataaactacctgagactgggtaatttataaagaaaagaggtttaagtggttcacggttccaaaggctgtatgggaagcatggctggggaggcctcaggaaacttagaatcatggcaggaggtgaaggggaagaagacacatgttcacatggccagagcaagaaagagaaagagtgtgaagggagaggtgctacacactttcaaacacccggatctcatgagaacactgtcacgggacagcactcgagggatggtgcttaaacattgaaaccacccccatgatccaatcccttcccaccagaccccacttccaacactgggaattacaattcgacatgagatttgggtagggacacagagccaaactgtatcaaagggttgagcacagagtggttcttcccagttctggactttaccagattgttggtggccaccatgagtggggagaattagccctttgctgtcaccttcctcttgcaattctggtattatctgtcctctgcctaccacccacccacggaagtcttgttggatgatacacaccagccagattcttcaacattaggcgccataagtaagaaagggccaaaacatcataccttgtgagccattttaatggatttgggctttattttgtagcaacaggaagtcactagactttattttttatctatttatttttttctttttttttcttttgtttttgagacagagtcttgctcttgtggcccaggctgcagtgcagtggcacaatctcagctcactgcaacctctgcctcctgggttcaaatgattctcctgcctcagcctcccgagtagctgggattataggcgcctgccaccacgccgggattttttttatttttagtagagacagggtttcaccatgttggccaggctggtctcgaactcctgaccttgcgatccaccctcttcggcctcccagagtgctgggattacaggcgtgagccacccgcacccggcctagactttattttttcagagaagaggagtgattgtagatttgcatttaaacaaatcaccttcttgctttatcactatgtcccctaccatatatcttcgttctcccagagactgccatgctgcagcctctagccgtgtgctgtccagtactgtagccactggccctatgtggttgtttggcacttgaaatgtggctagtggaaactgatgtgtgctataagtgtaagatagataccgtatctcaaagatataatgtgagaaaacctgtaaaatgatcacattcatttttatataggtttaacattgaaatagttatgttttgaatatgttgttaaatacatcataaatgttactgagttgtaactatgtgattaaaattgatttcacttgtttcttttgacgtttcacgtggcagtgaggaaattgaggttccataggcagttgcattctgtttccgtggaacagtgctgttctctgtgctatcccgagccaccagtgactctgagctgtaagggtcttagatcccatgatctggcaagtgggagagggaggtagaaatgagtccctccataccttcaaccagaacttcactgcatgttgcaagtcagggaacaaggcttgtttggggtccttttaaatacatctttcattaaatacatttggatgtaagatgattatatgcctttctagaaccctgtggtgctctcttcctcctcagacataatgtctttggatactaagggtgcattgatgaacagaacacagggcctgttcttcaaagggggagaaaagcttgtaaaatcataattttagcaatctaaaaactacatggtgacagtgacactaataccatcagcattgagcattcacaaagggtcacaaagggtcagctactatgctaagtacttgcactcattttacccactcagtgacacctgtggagaagaaccgaaggaaagcaccttggggaattgcagcattgaaggcaaacaaagagagacgattccccatgagggagatgagcagtggccagagagataggataggaagtaggaggcgttacatgctggaaggcaagccatgtaaatttctaaatgaggggaaagataagagtgcaagcatgatgcaaacggaaagcagttgaagtgtagtttttgacatggtaaaatcatgtctgtcatgcagactgaacacatgtcaaatattttattcaattcattaatgagatgaactgtaagatgtgaaaaatggttcattttgacttggagagatttaaaattccttccccagcacacttgagttgcatggctatgaacaggaatcatctggaattatctggaatatcgtcaaattatttccatggtacaggaacacttgcattgctatggggtgtctgcagtatccatctttacaaagctctaagggtgtggaatagcttgtaaggatgtaagtaataagggtgtaagaattttaagggtgtaagtcgtaaaatagcttatttagtagcaagtcaactggaggaacatgctgtagaatcagataatcctttccccgccaccctcagggtctgcaagacaacacctgcatttcaatgctaggacacccagcaatctcttttgctcatatcttgtttttggacatttgtatgtgtgtgtgacaaaacattgttaggatctggcaaatagaaggtcattgttgaccctgtggaatagcatggggagaagttacgttgtagtccattgcaggaaaaacaacgtgggtcaatggtgagtcagctgtggactcctggaggaaagacctagaggcaaaaagctttgaggtcattgaagacaaaaggaaaggctttgtgattttgatttttaaatggaaggaatttgggcctaaggaagcttaaactaaactgtgggaaggactgatacccatcctaagcactagttgcaggtatctcttaagaactaggttttggccgagctccgtggctcagacctgtagtcgcagcgctttgagtgagaggatcgcttgggcccaagagtacaaggttacagtaagctatgactatggtattcagttctgtaagaactaggcagaaaggagatccagtggatatgtgattgcctgggtcttggaaaggggagggaacatctccttttctgaggaagaatggggagatagggtgggttaattaggcagtttcttttgccaggtgataaaaaccccctaaacaatttgtattttatatatggagaatattttctcccactgatgggaaggatcgtagtgtgggggatgcttacccaatttcttaaaaatgtacacagaaatgagatccctaggtgctggaaccgagggtcagtactgcctagaatttccccattagggtctcttttgtttctgcttaccttcctgctgtgtgtttactccttttctcctgctaagatgggtgtcctcaaggagaccagggatgttggtagccacgacctctggtagtgttagtcctggtttggcatcccaactttgttctgcccatgggcattatcagggagtgcttctggttggcactgctttgagttacacgtctgcccttggagccaccaccgtatctgcagggactcagtatcaggactagcccatcttggccttgtgggtgtggccgtgtgatgtggggagaggagctgttgccagaagagggatgaggaatggcatgggcaaaatagaagaatgaccccagtgctgcaacacagtgggtatggattagaaaaggcttgtaggctggacagggcgaggtgagggagtgcatcatgcattaggattttaagtggctttttggggagtattgagcaggtggcaggggagacaagttcactcagaagttttagacagtgttgacattttctttcaaattgtgcatcttgaattctctagaacattctttatagatatttcaggatcttcctgccactttccacattgtagctattctggtgtttttcttttataattagaaataatgaatagttctcatttgtaggcttggattttcatgcgtgtgttggaaagtgttcgttttgccttttttttttaacttcatgtttttattttttaaaccgtattgatggattcattgctaggaacggcaaggaaaagaatactctcacaatgttaccacgtttccttctcacaggttttataagttgtattatttttatattttcaaagttataatcttaccatctcctctgttgccatacttctcattcatagggtctgtctttaactggactcattgtttacagctagtcttttcatcagatcatctcaaaccctgtattacattaatattttgattccgttttgttggatgactttcatcttaatcattcttattgttgaacaatgtttttcttattattgaacaattgcttcattaggtatagaattcctggttgatgggtgtttgattcagcaccatgaaaattcacggtattctggtatctgtcattgggcaaattcttcttctttttttttttttttttttttgaaaaaaatgcgtggaaaccattagttaccattgacagagctactcatttaatgattttttttttctcccaatagtggtggatgaattttttactttgcgttctcactttgtgtctctcatgaccaaaatattttagagataactagctggctaagtctgaggtggctgttaacagtctggtcctaggatagaacaaaaccaacagaactcatcaccgtcacgtcgttttgactttttgctcacaagtttaactcattgtttaatttgtttttccaaaggattgctttgcagtatacatgttggtgcatagcttaacatgatgataaatacataactttgactgtgaattatctactctgctcctttcatagtctcatgcttctctgaatttgtattttgtgttactgggttgttttgttgttatcattcatagtgttttttttgcccttgattctttgatccaggttttggtcaagggcaaggttcaagagaaaaagtaaaaacacagtcttttatttttgctcttggttataaacccttgtaaagtttgattgagaagataacttggatcttatggtttaaattgagtctttgtttaatcccaagtcttccaaattctttgtgcttatgtattctttcctctttcccaatcgtaatagttttgcttctgcctagtcattggtcaaatacctgcctttcaatgcatttaaaaaatattttgtgttagcaccatttgagatttttccccccttttttttcaagtatgtattgaggactgttatgggatgaatgtttgtgttcccccaaaatttagatattgaaaccctaaccccaggctattcggagatggtattcggagatgatacctttgggaggtgataactgttagatgaggtcatgagtatgtgcccccgtgatggaactagtgcccttataagaaaagataccaaagagcttgcctttttctccctgctcaaacgaagaagtcacataaggacacagcaaggaggtggctgtctgcaagcaaggaagagcggcctcaccagaactcggtcatgctggcaccttggttttggacttccagcttccagagctgtgacaaaagacatttttgttgtttaagccattcacagtaaggcagcattttgagctaagacaaagacctactgtggcttctgtaggcaaaagccctacagacccaaagtgttgtgagaattaacatttctagggatcatgccttacagtggattttatccaacacttagctctgacctagagcacgctagtcgtcgttaggtaaatgcttgtggatggtagtttctggcacagagattggagcggtaatgggcaggagttctgtatccagcttatgctgagagagcttcccataaaatggagcctatactttccttttgcagagaccatttctatccattgatagaattcttttttttctttttcttttttttgacgggatctcactccatcgcccaggctggagtgcagtggcatgatgtcaacccactgcaacctctgcctcctgggttcaagtgattctcatgcctcagcctcctgagtagctgggattacaaggtgcacgccaccacatgtggctaatttttgtattttggtagagatggggtttcaccatgttggctaggctggtcttgaactctagtgatttacctgcctcggcctcccaaagtgctgggattacaggcatgagccactgcacccagccttccatcgacagaattctaaagacatgtttaggcaagagttggtggaatgttggcgtgtagtccagatttgccttcagtttgtttttatccgcttcaaatgcaaaggccatgataattcaatagtaaagtcataaatccatagtatgacttaaaatatttggttgtaatattacaaagtatgcttccaaacacattaagcatgcaaaatccttatattgttctttttgtttagtgtttagggtagtttgtgtgaatcatagatgaccacagtagactgttgagttgttattttcttttgacattctttgatagaaaacagtgcttttcctctgttatttatactagaagcaaaaagtatctctcatagcattatggaaacaactcgaatacaactacgtcaattattcatcttttttttttttttttttttttttttgagacagagtcccactccatcaaccaggctggagtgcagtggtgcagtcttggctcactgcattctttgcatcctgggctcaagtgattctcgtgcctcagcctcccaagtagctgagattacaggtgcccacaccattctcagataatttttgtatttttactagagatggggtttcaccatgttggccaggctggtctcgaactcctgacctcaagtgatccacctgtcttggcctcccaaagtgctgggattacaggtgcgaaccactgtgcctggcagttattcatcatttaacagcataaagtggccacttgctttaataaactattatagtaccaaaattttggcctgaggaatttataaatataaatagaacaaaacaaaaataatattccccttgaaacatattctaaaaccaggggctaaaagttagaattgttagattttattgtcctgtttctttgggatgaaatgaaattttttaaaaaccatagttttggccgggcgcagtggctcacgcctgtaatcccagcactttgggaggccgaggcgggcagatcacgaggtcaggagatcgagaccatcccggctaacacggtgaaaccctgtctctactaaaaatacaaaaatatttgccgggcgtggtggtgggcacctgtagtcccagctacttgggaggctgaggcaggagaatggcgtgaacccgggaggtggagcttgcggtgagccgagatcgcgccgctgcactccagcctgggcgacaagagcgagactccggctccaaaaaaaaaaaaaaaaaaaaaaccacatagttttacttaaaccaaaggctttgggattccagaagaataaaggaagtgtacttcaattctacatctcaagaatctatttcttcccaaatttaatacaggcaaatttagtctatatttattgggcactgttaggtttttgtgtcaggtgttgctgagtggcgggataccattcttggcccataaggaccttataaatctattaatacaattaaattgtgaagaaaatttagaaaattgcaaaggtagaatgaggttgcaaattggagggtcttattctggagataggtggaagctatggcagatttttagcatgaatgctttctggtcaagacacttttagaaaaattagcttgtttccaagagtagattagattggggagagaaggaattcaaattacaaaataatagtgtgaaaagcattgtgggagatcatcctgtccatcccctcattcatgatatcctgaggtgctgtattaagggcatcattgctctttgtcacccgagcagtccaggaaggctgcctgttcagtcttagactgtatcctcacagtcttaaaccatatcctcactttatgatagaaacacacaggcaagtgttgtaagagttacatattgtcagttaaacaagaaattggatgagtccccatactattcttcctcttttaaagagagaacttgggccgggtgccatggctcacacctgtaatcccagcacattgggaggccgagtcgggctggtgacgagatcaagagatcaagaccatcctagccaacatggtgaaaccccgtctctactaaaaatacaaaaattagatgggcatggtggcgcacgcctgtagtcccagctactcgggagactgaggcaggagaattgcttgaactcggaggccgaggttgcagtgggccgagatcacgccactgcactccagcctggcgacagagtgagactccatcccaaaaaaaaaaaaaaaaaaaaaaaggaaaaaaaaagagataacttgaaacaggctcttccagcctctctattcttttaataacatgcagtgcacttcttggagctaggggctagggtctaggggcttcttgaaggtgttgatgtctctggtaacttacactgcagggcaggtctctggagggaactttgaacaggttaagcctcaaatgcaggcagagatttccaattgggaaagttaggtgaacgtggctttatcatctcttttattggtttatgggtattttctctttttcctgttttctttcccaccatttaacattatctgttctctgcctttttaaccttctggctttaaatgaacagttgcttaatctatagattaacaggatcagcaaacaaagcacagcagtgcctgcacaaagcagaaactgcctgttagatgagttgagcaaaaccatttcctttgcccaactttctcctctaaaatacataactgaacatgggattcccagacacgaattgtttctcaacctttctttttccattctacagttgtatgaaattaaaattaaataaaaagcaaacaacagaggtagtctttttaatcttaccgagttggaaggtatactatatacattatcctatggtagaaggtctggtacattcttgaccattattactttgctatgggcacccttcacccttgtaggctgaaaattggcaaatcataggcacgtatacattgtggttttggaatttgaaagatctcaaattcatattgagtgagacagtttttgaatggagggatcaggctgtctatatgtggatatattttatgcatttctggtctgtataaatgtaaggattgctttactgaccgtagatttcttctagggttaaaaagttgaagccagtttggatattttaaaataagaaaattagccctacattgaaagagagaataggaaaatctaggctatgtgtacacatctgcttgtgttttcacaattgtgtcctgtcctatttatttttatttctctcccatctcttccctcccacccacaatttgtttttcattcaacactttgaaattagaatagcttatctatcaaacaaggttctttgacaggattttccaactctgagtatgaaagtgtggatataacaatatatttatatagaatgtcagaagcagagtttggccagattattgggaaatattctaacgccctgcagcattttaccgcctttgcttttacataaggccatcatttggaaacatagcatgctgatacctcccattattgcagtagcgtttcatcagctgcacgctgcctctcagcgcctcattaagttgcaagcagagaacgttttcgttttccagttctctttctcaattaagttagagattgtattttcagtattcctcattattaaaagaatttttgttgagaatagtttaagatgtgatatttctttgtattccaggaaaatttaaagaatgtcattgaaatccagaaacagaagtatatattgtctaggacatgctttgtcttggtttatatgcctaagtccgagtagtttaaaatttatgattaatatactctttgaatgcttagaaatggcatcgattttagttgttctagtttcatcttaaataaattggagtaattactaactcaggattatgatgtgctacacataaaacatttgcagtgatgtctggtacttaggaaaccctcagttatgacatttttcttactttcttcttggatatgttctttgaaaaagggactggtatggaaaagctttgagacgatgaaaatatacaagataggctatctctgaaacaatttggtatagagcatttgattctctgtttcatgtatatattcatatatgctagctgcagtcatgcagacacaatttggatctgggtttttttttttttcctttttcacatgcagttataaatttagaaacctggctgtttcctctggactaaatgtatttatttaaattttcagtgctttaaatgcaaaattacaagcatatgtacaagtagaaagaatagtaaaatgaaccctcccatctatctgtcactgaattgcagtaatgatcaaatctagtcagtcttgttcctgagtcagtctggaatgcaggttttttcccttaactattcaggttctccagcaatcctggcaaaacacatgcgggtttcagctcatctctttcagtgacctgtaaattaagatgtgctaaagcatcacatgttactcattgacgttttgttaatatatttaaaacattttattagtaccacataatttttagcgttttcaaaaagaaataagaatttggctaaaggtgaattttaaaaattgaatgactaagtagaaaagtaggcaaactgttttcagaattagtagcaatttccaattccatttactctgttcattaactttgttttcagccttttgttagccatgtttgattttgtgtttttgaaatacctttggtcattttaggacttttggcattaatcacctgtcttaggtggcctgtttacaaaagggacttttcggttctccatttgcccttaatgttgggaatagagacaaagtttctgtaggactctgacaacatatggctttacaaagtttttttttaatatatacatttctcttcttggctttcttaaatatcatcaatgtcttttcttgctttacgtatttagcaacttaaatgtcatcttgtctgagatcaatatatttccaacttggagagatagtgagaagagagacttcagatttttaaaatgtggaacaaaacattctgatggtgaccaggttttaaggtatttcttcagtagacaaaactacttaggctaccgatgataagcttttaatattaataataattattatatataaaaatattaatataatatgatctaagattgtttctattccccctctttcctggttgttaggatttggaatttcttatgcactctttttttttttttttttttttttctgagacagagtctggctctgcctcgcaggctggagtgcagtggcgcgatctcggctcactgcaagctccgcctcccaggttcacgccattctcctgcctcagcctcccaagtagctgagactacaggcgcctgccaccatgcccggctaatttttgtattttttagtagagacggggtttcaccatgttagccaggatggtctcgatctactgaccttgtgatccacccgtcttggcctcccaaagtgctgggattacaggcctgagccaccgcgcccggcctatgcactcttaaaattatttcatgtactgatacattcactttcaatgataaaacatggctgtatgaccaggtaggtaggctgcttttcagactcctgtactaactgaatgggccccattagcccaagtgagagcttaacttgtcactcttcctaaatgccttcctcagctgagtgcttatttacagtaatctactgcttatagcagatgctatgatttgtataagctgtccatatctacttggaagaacaatattgaaaggcattgagatacttactttccctcaaaagcccataatccaacaactatttagaatatcttatttaagagataggtttataatacccaaagggaacttaatgtatactgaaagtccctgattgaatggattctgaccttaaccagaaatattaattacatgcttatcagacacttactgtgctatcccagggtttgccactgttgagggaacaccagatgaatcatattgtccctgccttcaaggattatatattctaataggacagacagcccatcagcagaataggaggtagcaagtggtcagggatcagataaagtgatcaaggcaagtggaggatgcagtggtaggacagaatggtctgcgcacagagattgagggcatcatgtggtgggaattagtgaactagcatgcaagccgggcctttttcttggcttttgttcaattggaaggagcataaagctactaaaggagcttatgtttagaaatagggcttattgaaggggacaaggcatctcctagaatccaaatgcacatcccaagacttaaaagagttagagggcacaggcaggccctgtcagatccggtctctgccagatccagtcttgtctgtttctcatcttcccttgtctctctgagtattccctgttttcctttcctggactaactgcttcttccagatgcctagtttgagtccacccatggttttttgggggcttttctttgtttttttgttacgttctgtttttgttttttgtttttttgaggcggggtctcactctgttgcccagactggagtgcagtgacgtgatcccagttcactgcagcctcgacctcctcgcttaagctgtcctcccacctcagcctcccgagtagctgggactataggcgcaagccaccatgcacccatgggtgttgacatgccacctgtggctcagacttatgctcacctccagtcaagtttcttccttatctaacgacagcctttgtgtgttcgtgaaagcccttgagggaaagaatgctacgtacagtttggttttcttgggctctaataagctataggggatggtgctgcagagtttgaaatacagtcactaatgagtcctccttcagagagctgagggtgggagcaccacataaaaggagttgggggtcagcagcctcctgatagtctgaaagccaaagatgatggatgtggggctccacggggaggctataacatgccacgttatgttttctgcccttgaggaaagcttcttttcaccaggcaggcagtacagatacaaacttaatagcgatgtgaggcacactgataaataccagaaaacattctgtaagaggggttagcattatttcactgtgtgcagaggaggtttgtccatgtggggggaccctaatgggcgtaggcatttgaagtgtagtctaagggatcttaggtaagagtgtgtttggaacacagtgtacgtatttatacagcaaatgctctgtgtggctataatttaggaaaatttgtaggacttgaggacagcttatttttctactagctgacagaggcttgggtttgcttctgctttaagtataagtatctcgagtccttttttaaaaaggaaaatcaaggaaatagttggaattgggtttcacataaaaaacattttagaaaagaaggggagtatgagagttaggatttgctaatgtttgctgtattagcaaattatttgcaatgcttgtcatattagcaatacatgtatgttttggaaaacacatgcaaattaagcagcttaaggaacttttatctttatttcctatgtaagatttgagatgctgcaaagaaaatattaaaacattgaaccgatacttacagtagtcctcctatatctgcagaggataacttccaagacccccagtgaatgcccgaaaccttagatagtaccaaacccaatatatactatgtttttttcctataggtacatacctgtgataaagttgaatttacaaattaggcacagtaagtaattcacaacaactaataatagaatagagcaattatgacactacgccaccatcactactcttgcactttggggccgttattaagtcaaataagagttacttgaacacaagcgctgtgatgaagtgatcaatggcaggtagcatgtacagcatggagacgctggacaaagggatgattcactttctggactgtgtgggtttgagatttcatcacactgctcagaatggtgcgcaatttaaaacttattgttggctgggcatggtggctcatgcctgtaatcctggtactttgggaggccaggacaggaggatctcttgaggccaggagtttgagaccagcctgggcaacataggatgaccctatctctttcttaaaattaaatataggccgggcacggtggctcacgcctgtaatcccaacactttgggaggccaaggcgggtggatcacaaggtcaggagattgagagcatcctggccaacatggtgaaaccctgtctttactgaaaatacaaaaattagccgggtgtggtagtgggtgcctgtagtcccagctactcaggaggctgaggcaggagaatcgcttgaacctgggagatggaggttacagtgagctgagattgcaccactgcactgcaacctgggcgacagagtgagagtccatctcaaaaaaaaaataaattaattaaataaatagaaaaataaaactcatttatttatagagctttctatttaatatttttggactgaaactatggaaagcaaaaccacagataagggaggactactatagttatgataaaaatcaggaacttaatctagccacgagaacatttgtatttctgatgaaatttttattcttgtctaaaatgttgaactatatttatatttatatctttaaatataaatgaactatatttatatctttataaatgagctatatatttatattttatataaatataaatatttttatatctatgtaaacataaaaaggctttttttcctctctggatgtttactggggttatttaataaaaacctagggatgatattgggaatttttgttggaattttaaattttaacgtgtttgtttctttttttgttttgtttcgttgtttgctttctgagctgggagttggctgtgcatgtggcagttgctttatcaaaagggtcttgctctgcttcagaagaagttttgttaccttgattgctaatgttctcccccttaaataaacctcctgatgttggaacagcaaaattgcattatgtttggaaaatctaaattaataatcccttatctctgaataaaagtaactgaatcaggttgaatttactgcttccattttaataggacagtaggaaaaaactatgcatgggctttatgtggttcaaatttttttttttttttttttgagaccgagtctcgctctgtcaccaggctggagtgcagtggtgtgatctcagctcactgcaacatccgcttcctgggttcctgcgattctcctgcctcagcctccggagtagctgggacttaacaggcgtgtgccatgacgcccagctaatttttgtatttttagtagagacagggtttcaccatgttggccaggatggtcttgatctcttgacctcgtgagccgcccgcctcggcctcccaaagtgctgggattacaggtgtgtgccactgtctccggcctggttcaaattttgtaatctccacatccatctgcatattaacaattgtcattgttggtgttatagagagagaagctgaagcacagaaaataacacgcatgttctagatttcctggtgggaactggaagtaaattctgtgtttggctgatcactgtataaagttctagtactagtctttagggaaaagccccgtcttcatggattttaatttgtcaatttgcaattctacctacttggaaatagtcaacctaatcagttactgccctgttgcaccaactattaccataggaatctgaagatggtatgcaaccaacatgaaaaagagatccttttctcctggtaatcagtttgtaccctacaaacaattcgttggttttaagttgtatagagggaaccatgatgtgaatatgcctgccactgggtttatagaaaaccgatgactcctgaggaaattctcagcagggttgtctttcagtgaaaagaaccctttagaaagatgcagtaactcacactttactgcacataaagtcatgtccctcagggtggactctcaggtgtcatacagtttatgactcatgagtgctgcagctttgtgatctaataaacaagtaaagacggtgggagaattatattcctgtacaaaaagccacttcacctttcattatataggaaaaaagccatgggaatattaaatttttagtggcatttttagagcattttctgtgatctaaagtgcctgcattgagatattaagatacttaagaacaaagtaaaataaagagaagtattaatagcaggatccctttggagctaggttactgtggcagtggtttcaagaataactctctcagcacccaccccaggattctgatttagagatttctttgtgtgttctcttagcatatacctctaccactgagtttatcccctgctgctacactgattgataccttatttttaaatttttgtttttaaaatagacggttttttagagcagttttagattcacagaaaagttgagaggaaggtacagaggtttcccatatacttgctacctgcacacatgcttagcctctcctattataaacatcccccacagaatggtacatttgttacaatcgatgaaccaacattgatacatcattagcacccaaagtccatagtttccctgagggttcactcttggcggtatacattttttgggtttggacaaatgtataatgacatatgtccatcattgtagtaccatacagagtagtttcactgccctaaaaatcttctgtgctctatctgttcatcctccccctgcccccaaccacccaaacccgtggcaaccactgatctttttagtctccatagttctgccttttccagaatgtcatatagtgggaagaatgtcatatagtatggagcctattcagatgggcttctttcacttagcaatattagttaagttcctccatgtcttttcatgggttcatagcccatttccttttagtgttgaatgacattccattttctaggtgtgtaccccacagtgttatttattcattcacagtaccttattttttaactgatttttttggggtctgggactaaggtgaggtaaacaaggcagtcaggaattgagattaagaatttttaatgtaatatttttaaaaatcaaaattactgcaaaaaaactccatgatgaacaaaatatcaactttttagacagagattgagtcacgctcaattgacatcaagccatgcatagttctcctactgccttattaaaatggaagcagtaaattcagcctgattcagttacttttattcaaagataagggattattaatttagatttgtcaagccctctgaatgtcatggacaattgtgagtgaaattccgtgtatcgtctaactccccatggatgttatcttgctcagaaaccttgttctccctccaaggaaggcactacttctcctgtagccttctcaggtcgaagttcttttctcaaaatcctcacagcgttgcccctagaggtgggaatgtcctcttttcctcttcatttcctcttttcctcttcatttttgtttccaaaccactcttcctttctcttccctaaaagcccccctacctcagcttgaagtgcatgctttcagacaaatcactttgctcctccacctgttggtcatctccagacctctttctacctcatcgatgttcaccattcagactttgaagattttagctcctgccttgctgtcatcaggtgtagctgctcatttgctgattcttaagtgatcctaatattgatctcaatgatccttccagtattccaatttgtcagttcatgattctcatccctccagtgaccttgttttcatcattctccatcacccattgcttacaatggtcatattatactccttgcaatgaccaatcactgcaactcctccaaaattttaatttcaattatttgactctccaagtactacttattaccacctcactttctccagtcctgtgcgccatacattctttagcccctggagtgtgatcagtggactcagattccactggtccctaccaccttttacctgtgtctaattcccctcatgttctcacttttgccacttgagattgcatggcccagcattctaatcattcccttgcttataccgtcacttctctattcgttgttatttatctggcaaaacaaccaccctgttgcaatgcaaggtttatgcaagggaatgcacattttcgaaatagatttttatgtttatgtttttctttcaaatctctactcattaataaatagaagtggtttgatatttcacaaatgtgatttaattgatcaagtttcacatttcattctaagtgctcagttaaacaccatgatgtattcacaacagtaatcaaagcaaggggctgtagttagttataggagcaaagagaattcatgagtttatttttatctaaatgacacccttgataacttttcgataagttattttcatgtaaattacttggaataagagcaggatttttatatagaaagctttctatgtgtcatatcctgtaaaatgtttgtccgattagtgagagatagggggaggttgaattttactaaacataatatccccagccatatctactgtaacgtttgtttaggacattacagactattatcctacaccaatttatttcacatcaaaaatcaccccatgcttgtaagaatcctcctttccccctcttgtttctattttagctcccctggaaaaataatattttaagtctaaatgagtacaataaattaaaaagttgcattgcttaatattttgaatggaatagctagatgcttgttcatttgctatctttttctttgtactgaatttctttagcttggtcagaggtagagcaggcttgactcttagtggatagtggtataggttgagtatcccaaatttaaaaatccagagtccgaaatgctcaaaaacccaaaactatttgcatgtcaacatgatgctcaaaggaaatgctcattggagtattttggattttggactttccggattccagatgctcaaccagtaagtatataatgcagatgttctaaaatcggaaaaagtcagagaccttgaaagcacttctggtcccaagcattttggataagggacactccacctgtaccttacagtggaggcttgttagatgcttgtaaatgccagcccctgcctcaagtaacaattgattctttttgtgtgctctcccaggtctaccctgaactgcagatcaccaatgtggtagaagccaaccaaccagtgaccatccagaactggtgcaagcggggccgcaagcagtgcaagacccatccccactttgtgattccctaccgctgcttaggtgagccggccggccgtggggctggtgttgattgggggcctggtcttgagggaagaaaaagaggatgctcctgttaggtcacatacacagacttgttcttcagcacattgccactctgtgttgtactgtgttttggactcttgcagttacattctgtgcactgaccctataggagcagtatttttgagttccctgcctcagaatgaatttacccagggtgtatattgaaattacaaattcctgggccagttccaggactcctgaatgaaaaatgcctatagtagcggatccgggaattcttattttaccgtatcgcatagatgattctcatgaacaggggccttgtgtgtttcttcacatagactttctagaagaaagaatctaatgtgaagctgcagcattttgttaatttctaaaaaaaaaaaaaaaaaaaaaaaaaaggctgttctacaataactactcctcttatttggtgatagtagaggaattggaattgggaaggctttttttctgcttacatcaaactggaggaaacagtctgattactaatgtttaatgtttgttttgtcacaaacctcaaatttacctcaacattttatttaaaaaaattgagtagttttatgtggtatcgtgctcttttcctctggaaactgtcatactccatatttaatctaacttggttattagtctgttcctagttgttgtttttatgtgaccagtcattgcagaagtcaagttctttgctctggtatcttacatctagatgttttccacaagtgtattcatcagtaggcaggttgtaaataaggatatagagattgtctgtggagattgtggacaaatattgatgtatattctgatgacttcttataaaggttaaaaggaatatttcaaagaaaatccaacagcaaacttacaaagtaaggatatttactgaagattccggagatctatgtaatatatggtcaaaaatgaatgtcctaagagcatactgattgctgttcattcatgtgcataatttgagacataaaatgaaaacactgggcattagaaagaagatttcacctggtgaatttacttacaatgtaattgatgaagagagtgtagaaaactagatttcttaaacatctgataattcaagagtttcctttttcctccttattccttgttcctatatgctttataaggcctcaactcttttctggttcatcaaaaagtgcagtttaattagatgaaaataagcagcacttaaattctagatacagaactcctactcaaaaaaatatagaagtcatctgtccccaggaactgtgtattttatactcacttagttattacaccaagaaataacacacagagattgaacaattcagtacaagttaggaagtagatcatgagtctgttaatttgatgggccaggctttgttaatgatttgtttcttttggttcttacttacctgttctatgaagtattccaaatatattttcattagaatgtaagctcactgagaacaggtagggctttggatgggcattattgctcctggtatgacactcaaaactgcagtcatgtttcagaagtgaacttatcattttacatagatttttctgcttcatattctattcagaatgatttgtattaaagttgatggaaagatgaaatccacattaagattgttatttactacctagtgccagttgttgtagcaaatatcctttgaaccccaggtctattttatcctatctgttgatcacttcctttgcccctgctcttcaagcctcctcatctctgaaaagctctccttccactgggtgtggtggctcaagcctataatcccagcactgtaagaggccgaggtgggaagattgcttgagcccagcagttagagaccagcctgggcaacataatgagaccccgtctcttaaaaaaacaaaaacaaacaaaaaaacctccaagtgaacgaacagtacaacaagtaataaaggatataaaagatgttctcagatctcaatatcaagtcagaagattttataataatattataattacacggagactgtccaaatatttgtatttgttgtgctacactgcattatgcaccatttagggaaacaacagcctaagtaatgtggttttaaataattaatctcatctaccaaatacctgtttgatagtaaagttaatgacctgtcctcaccttatgtttttaaccattatggaaagagaaccacttgaaaatatatcattccactgacagataattacccagctgtgaagcacaggctggagactggcaaggagaaatggagagaagcctataaaataattctttctcagggaaggagtttattcctgccaaggctttgccatcaaccattatcagatttctctttcatcctccgaacagattctgggctcctttgatgtggaggtacataattcaaaaagttgggtcacttgtgacatgttaagcaaatttctcagttctggttgtgtctgatgtgaaatgagcacgttgacactaactgttcttgactactatgggactagataatcatttgctatttcctgtaggtacttggcgtgttgaatcattttaccatattttctcattttatgaatcactggtccttttccattggacctgaaattagaccctaatatgacaagtagagcatatgttgtctcttactttgttattaaccttagattagaattaaggaatataggtgctgcttttttcttaggtagtggcaaattttccaaaggtctcaagcccagagtataatacagagttgataaaataacctggtagagtatgtagagcatgccttgtctttcaggaaaaggaactgaaaaacataggttttccaataggtgtaacataggtaacatagggacataggtgtaactgatgtacattttacttgcacttaatagacaggcagtgttagttcttcgtctctcttcacaggctatacaactaccaagatccagagaggttgaatttcttgctataactcacagcaaataaatgggagagtcagcactacagtccaagctctcttgattattggtccagtgttttttacagtatacacaaaaatttgttttaaaacaaatattcactatgaattagagacaatagcactaaaaaagaaactcacagctatcgttgtgttgacagaggagacaaccatgacctagtataggataaatctccatgtgatatgttttctactttatagtagtagtaggtaacattcttacatattattcctgcttaacaaagaattactgttcagcattaacataagtatgctctaaaatctcttgactgtatccaaatatatgttttagacaagagttttttttttttactgacctagcaaacaaaataaaattatcttaaacattaagcgtgttaaaattcaaaacaaagcttgaagagagaaaaatgcttgttatgtgtggcatacaaccttcctaacctctttatcagatgaaggatccagtgtggcactggattatttttctgtaatattttgcctcggacatatgtgctatacatttcttgtctgcatggggaaagtatgtcaggttgctgttgccactttcttcccatggagtggataatttcccttgagaataagttggctttctcatctcaccctttgcttttggtagtacctctggaaccagagggtaagattcatcttaatcatgtgaacattttattaattatctcatgtgtgattcaagcacatcattactgttactcaaggagaatattttctttgcaaagctttcaaccctttccatttgaatgactgctcttcatggtgtaatagatccactgttgaaattggcacttttctgtacttgtctctgaccagcaaaggcaaggaacattgcgtaaaataaattactgttgaattattggggcaaagtcaggttaatagcttaatccaaaaaaggaattacaagtgaaagaattttaccttaaatgtcattctgtttcattttgtaattaagaccttccaaatgtgtaagtagtaatgaccagaaatcttatcagatatggtcaaatgagaaaggtattcaattcatattaattttgaattttattgttttaatacaattttaggaaaagcatgccttgttttctgggtttcttggctgcctttgtatcaattatctacagctatcttctttagcatggaaaggatgtatttgaatactgtgacattttataccagggtaattctttgaatttatgtaaacaacaactctcttgagacacatcagtacaacacaatagtcagaaatgtcacatgagagttcaccaaactttaaagatggcgatgaggatggaaaaacatttttattgtagactttaaggatcttgtaaatccccgtatgtaatccagtgtgctagagaattggtgcccatggcgcctcctctccccaccgacctgcactgtggaaatcctactaattttgaagtcatgggagatgcatgtgtgggacctatacatgcactgtgatgagaatgatgacttcatcattctcattatttaatgagcccagctgctgcatacaagaaggatgcaaattcttgaatgttcttgattgtttacatcatggtctcataaggaatgaccagaattgctgggcatttcttcatcaggaaaggctaatcagccatccttggagagacatcagactcctttatatgtggccttcccatgggacctttgaatggtcaacgttaatcatgcttcagaaccagcatactaagtaaaagaaaacatgtccggaaaagaaatcttatcagtgtttatctcccaacagtgggattacaggtgattttgattttatttaagcaattttctatgactttgtataataaatgagtagcttttataatcagcaaaagatagactatgaaatcagtacatataattttgtatttgtatgtagtaccattgaaggataatagaaaaaagtcaaaagacttgaaagtcgacacagaatgatcattaatttgttttatctcttggtagttgtactttatgagctttatacatacagtatgggaaaatacactttagttttgaaaacaccatgatgccaaaataaaagaaaataacgttcattcatacgatcttcaacaaaatttaagcagcattaatcatcactgattcttcaggaagattcttgatcttccttaattttttttccctccaaaattgcatatggcagccacatttgtttcaggccagaaggaagaatgggaagagcttacatttatctcacagattccttctgggtgaggaagacttagaaagtgaagccctcttccatttgattaagcaagaacacctataaaaatggtgtcttcggccgggcgcggtgctcacgcctgtaatcccagaacttcgggaggccgagatgggcggatcgcaaggtcaggagattgagactgtcttggctaacaacggtgaaaccccgtctctactaaaaatacaaaaaaattagccaggcgtggtgcgggcgcctgtaatcccagctactcgggaggctgaggcaggagaatggcgtgaacccgggaggcggagcttgccgtgagccgagatcgcgccactgcgctccagcctgggggacagagcgagactcagtctcaaaaaaaaaaaaaaaaaaaaaaaaaaaaatggagcccttctaggtaggtcatcctccatggctcactgcagcacttccttggccctgtcccaagttccataattgcctcaattgcattggctcctgcttgatttgctttctaaaactcaggacagaggatgagttgaggagactgacttttttaacaatcgcattttgaaaagaaggaagcgtagtggtgtttcttggctccgtttccagttttgtttcttttcattttaattattgtacctggtattgtgtcctctttctgggacttttaacaactaatgttttgattgataagagctctaatagcatgggtgatttatacttcattacatcttcagttttgtcttctttactggagttgtattcaggtgtcattaagtaacgaggcattaaaaagaaacgataacttttgaaaaactgctgttatggcttatgagtgggctttgctgatattttcttgaagcacagaatttatctatctctgtaggtcctagatgataggtgagaaaaagaaacccagcttatttggcatgatattgtcccaaggcttgttagtaattgctttgaaaaatgaatagcaaggctaatggacatctgatttgtagtacatgctaagaattgcaaatgtgtgctcaaaagaaatttctctgattacaaaagtaacatatattcaacgtacaaagttgagaaaatacaaccactagctaaaaataatcaaaaccatttggcttccttgcagtctacatttttttcatgtatgttttgcttttgtttttagtgttatttaatcgtttcctgttttgtgacttgctttttacacctaacatttttagaatactctacctgtgaaattaaatattcttggagagcataatttttaatgtagcatcacaccagacactgtaatgtgtttactcttttttatggtcatttaggtggttttctttattttgtggtggtgtaatagttgttataaatgatgctgtgataaacatcctggtagataaatttgtgtttagttgatttttatataacagttattggcggaatgccttgcaaatcttatagttggtgtaataaaatatatgcgccacataattgtttatgaaaatatggaggaaaattttaatactgatttatgatcctgagtttaacataagcatggatacccctacatttagctatgatttttttattgatagagtaaaagcgcctgtaatttctacttaaggccatttaaaataattactatttaaatctatttaaacaattttaaatattgcttttccaaatttccaccttaggaagtagttaattgaatattctcagagaaataagtcctacaaaattgaccaataaaaggtcagttacgtattcattgtgtacgttttattgatcacccacagtgtgtgagcccctggaggtgggtgaaatgtgggcctccctcttacagagttgatagtgggcctgaaggggatgggatttcagctcaggaatttaaggaagacttgacaggaagtagcattgtgagccaagatttcaaggataagatttcaaggcaagaaaggagatgtattttaggtattcagacagagggtcaacaaagtctttaaggtaggaactagttcacttttcaaaaagaagaggtaattggtcatgataagagtagtagtttttaaaaatcattactgttttcattcatttataatgaatactattattttataattttaaagtttatatccaaataattgttttcaagatggtaatgaatgtatattgcttttatgtatacacatagtactttttgctttttggtaactaagtttataatttttgagggcaggaaatatttttgtttttcacttacaaagaaactatattttaataagagttttttgacagcagtgctttatgccaaaggaaaataaagcaacatttaacatatcaaagaaagccaatgcaaaacaaggattatttatttatttatttattttattcatttttgagatggagtttcactcttgttgccccggctggagtgcaatggcgtgatctcggctcactgcaacctccacttcctgggatcaagcgattctcctgcctcggcctcctgagtagctgggattactggcacccgccaccacgcccagctaattttttgtctttttagtagagatggggtttcatcatgttggccaggttggtctcaaactcctgacctcaggtgatccacccatcttggcctcccaaagtcctgggattacagacatgagccatcatgcccagccaaaacaaggattttatattcaatcaaactgacattcagatatgatcacatgaaataaatatcaacgtacaataactcagggcatattttcacattattttcctgtagaatctgctagagaacaagtttcagatacccaaaatacctagagaaaaactgatataaggatgggtaatgagcattaaggagaaacataaaaactaagactcaacgatggtaaaaagtatgataatatagtattccacagctacatgctgtgacaataaagagattctgtaactgtcaggcctctgagcccaagctaaaccatcccctgtgacctgcacgtattcgcccagatggcccgaagcaagtgaagaatcacaaaagaagtgaaaatggcctgttcctgccttaactgatgacattccaccacaaaagaagtgaaaatggccggtccttgccttaagtgatgacattatcttgtgaaattccttcttctggctcatcctggctcaaaaactcccccactgagcaccctgtgacccccacccctgccccccagagaacaaccccctttgactgcaattttcctttacctacccgaatcttacaaaatggccctaccccatctccctttcctgactctttttcggactcagcctgcctgcacccaggtgaaataaacagccttgttgtgacagtaactttaatggggaagaatgagaagaagatgtgggtaaagaaacaactgtttttctattaatggtggtagtattagtgtgtaattctgagcctggtgcataagtaatatggtgtaacataagtgaatagttgtgtgatattttaattcaatcatcccatgtatttgagaatcagaattttgatgtggtaaaagaagattcacatgtaacatataatctattaaaaagataccctgtagtcatgagtttgacttttaggtatcatttctcagtataacatattgttgtagatttcagataacattttttccccataaatactgtccgctgcaggcctaaaaataatgaaaaggcctaaaagaccaactcagtagcggtaaacaccagtactgcacctagattatggtctttaggaaccattttccagtaaaagataccagtaatctttggaaagcaggatttttttttctttttttttttttttgagatagagtctagcttcgtcgcccaggctggagtgcagtggtgcgatcttggctcactgcaacctctgcctcctgagttcaagccattctctgcctcagcctaccgagtagctgggattacaggtgcctgccaccactactggctaatttttttttgtatttttagtagagacggaatttgaccaccttggccagtctggtcttgaactcctgacctcgtgatccacccgcctcagcctcccaaagtactgagattacaggcatgagccaccacaacccggcggaaagcaggaaattttttaaaagctattttaattcagtcatgtaaaaaaaatgagcagaaagaaacaaaaaacttaaaaaaaaacttaaaaaatgagcagaatcttaggaatcacctgcgactttggttattcatgcttagccatttctataggaaattccagcactatttgagttagaccactaattatatgtgtgttgtagctttctccaagagataatggaattcacaaatcaaaactttgagattattaagtgttacaagtgaattataaccccagtcatttgctttcaaatgaacagctaaacatattttgtgttaatttcttatggatgctgctttgtttgtttgtttgtttattgagacggaatttcactcctgtcacctaggctgaagtgcaatggtgagatctcggctcactgcaacctctgcctcccgggttcaagtgattctcctgcctcagcctcccgagtagctgggattacaggcgtgcaccaccacacctagcagatttttgtatttttaatagagacagggtttcaccatgttggccaggctggtttcgaacttctgacctcaggtgatccacccaccttggtctcccaaaatgctgggattacaggcgtgagccaccattcccggcctagataggcttttagttggtttccatatacagtgtatatagggagaaacagactaagacagacactggcatccacagaagtaaaaataaaaagtaaggtataatttaaatttcacacggtcatggtcacatttttcagctttttgcacctatgttgtataaaaaagacaaaaatgcctttagaggctcaagttttcaggacttcctgaagctgtgtcatagtttaaaaatgccataaaaatgccgtcagatttaataacgtatcagctgaacactggatttctgttatgttgagtcttcaaaaatttctccctgtatgaggaattaatactgttatgtctgatctggtggtctgaaatgtgaacttttctgggccaaccagtaaacagctgctgttgctttatagctgcgtaattcagtcttggaaattattagataattcccacaccatttcagcttgctgttttaccataaatttaatgtgaacagtatttctagctaacctcaagctgtgtttattgtcgtggaattttctcttttattctgaggtaaatgattacaaatggttatgatgaggaatcaaagaagaaagtggactttcttcaaagttctttttctactctagccgcttctgagcatctttctcaagcactgtgtttccatcagtccctaccccaagcagagaaaggaaacagttttcccctttgtcttcagaaccctcttctcatcgtgtcattttccaaagcaaataaaaatcttgagatattgtcttgagtttgattttttttttttttttttgagatggagtttcactctttgttgcccaggctggagtgcaatggcacaatctcagctcaccccaacctccgcctcctgggttcaagtgattctcctgcctaagcctgccaagtagctgggattacaggcatgtgccaccatgcccagctatttttctgtttttagtagagacggggtttctccgtgttggtcaggctggtctcgagctcccgacctcaggtgatccgcgcctcggcctcccaaagtgctgggattacaggcgtgagccaccacaccctgccgagtttgatgttttttttaaccattcaagccagtaaataactttccagacagctgtagtagaaaacagaatttgtgtgtgacaaaacttaagattttgggattgagagctctatggtttatgccatgtggataattgtagccagatgaagaatatgaaatttaaggaagtgtttagagtggtacagacagctattattacgattcttggggtagaagagtagcttacatacaacataaaataatggcattaactgaattaacttcagcctttgccatgaaacccaagctttcattatgttttaaataatgaactacattaaaaatagtatataaatatgtaaaatatattagacatagacttctcataataaattatgggaacttttgatgtatttcttccctaggaaaagaactaaacaagtgcttctttgtggacttaaaatttttcagtttactattgtgaattaggtacagcatatctttataattccagataatacccataaaagcagaactgtttatttacttattatgaagggaatactgaaatgaaattaaggacaatttttttctcacgtattatatttttaaaaagttatttgatgtttgtgattaaataaagaatgaatttttaaagccagttctcaattagcactgaggaaaagaatatgctttttttcaggtacacaaataagcatcatggatataagaaacgacataaagatgaatctacagtctggattaagacgtattttaatttcagtgtcaaaggctgttaggaaatattcagattttagtacttgacaaagaaggttctcaatatttgtttcagtgttcagtgaaaggataaattctgtcgtctttctcttttatcatattagtcactgagtggtttcattaaacattccctcttccatatcccctgataaatttcaacttgttggtgatgcatcccaggagtgtctgtctttttttttttttttttttccttctcctgtcagtgagtcttaggctttggctttgaaaccctatagtgaagggaaatctcaggcaacagctgtgcaaccgtctggagagcagctagtccagattggaggagacgaacgaggctggtcgcaggagagaggcctccaggaaaaaatggcattgatgcattacctgcgttaaagattttcctgtacttagcatgcttcttagggttctactggagcgtttgggaaagatacagtattgatggatgcatagaaaatgaggcaaattttttaaatggggcaaatattaactgtagacaaagaaaagccaaaagtacaaaataacagcgttgtaagatacgaagataaaaatacgcctaagtatgagcgttctggcagactcacaatacagatgccatatatggggtaaaaccataccagtaggatggtgggaagggaaaggggtgggctcatgttccatagtgggaagacctacagcggaaaaatcaggagaatagttatttaagcacagagtttagggagtaagttgcagaagagacaactaaatgagatggccgtgaatgatttttatgacctcctcttgttatggagaaatcgatcaaggaggcctatatttgatggcaggaagaagttgagtttaaggtttccagacatttgttttaatctggaaaatttctttttttctttctttcttttttttttttttttgagacagagtctcactctgtcgcccaggctggagcgcagtggcgcagtcttggctcatcgcagcctccgcctcctgggttcacgccattctcctgtctcagcctcttgagtagctgggactacaggcgcccacaaccacgcccggctaattttttgtatttttagtggagactgggtttcatcgtgttagccaggatggtctccatctcctgatctcgtgatccgcccacctcggcctcccaaagtgccgggattacaggcgtgagccactgcgcccagcctaaaatttcttttaagcttagaagttgcaagtcaagttactgttaaaggcgtcctaactttgaattagataaagctaaactgtggtttaattgagctgagtttgttgtatgctatatctcaacttcacaagatgtttgctttttcttggctataacctaagattatacaaatataaaattacctttatgcccatttgatagactttagggctgaatttccctgtgtaagatgttgtagaggtttgtacttgtgattgtttttactctagaagcatgtttatcttcactcctttagtgacacgtgtttgttcaggaacttcaaactcaatgtgatcaaaactagactcatgttccctgaatagtctctcctgactacaaatctgttggaaacttccaatcctaattaataattgtaaactttttaatgtaagtcatatgaggggttgttgtgagcataggaggtagggagatggtagtgtgagctggagcatatggtaaaagttgtttgaaagaaagtagacccatgatgtggatcctgagggatacgtaaagaaaatgaagaggtgtagatttaaaaatgtttatggggcctgggcgtggtagctcacacctgtaatcccagcactttgggaggccgaggtgggcagatcacctgaggttaggagttggagaccagcctggccaacatagtgaaaccccgtctctactaaaagtacaaaaattagccaggcatggtgatgggcgcctgtaatcccagctactcaggaggctgaggcaggagaatcacttgaaaccgggaagcggagattgcagtgagccgagatcgcgccactgcacgccagcctggatgacaagagcgaaactccgtctcaaaaaaaattaaacaataaataaataaatatattcatggggatgttccagggaaagtagtttgtgttggtagccttgagaaataataatagaaaattagaatagaactaagttggttcattaaagttggaacacttgtagagatttttgatttggacagacattaattcatttgattatttctcttattcgatgtccagaaaaatatctgtacattcaatgtggttttatttgtactgcacatgtcattaataggctaaagttacctctaagatacctgctttcttagtccttgttacgctgtaaattctgggcatcaggatatagctttttattatgttagctcttttaatagatgatacaattggattggggaaaaaactgattatactctggagtaatactttcatagcctgtagtatcctgcaaatagctcctggtcatcttcgaattttactagcaaacttgtagggtaataggttgggacaaagtattataatcagtggctttgagggcctgataagaggaaacaataattgtgcagctatattgtgaataagtagtgcatgatgctgatggttaaccctttcctgcggtgctcaaacattgggaaataattgctttttgttcaaaatgtaagctcttcgataattgagacttttgtcaattcactgaaatattcccagagcttgaacatgtctggtacatagtacatatttaataaatatatagatttaatgaatgaattgaggatgtagccaaatattggaaaaggaaagggaaaagaaagaatagcccaaacattagaaagacagaagatgtggagcagcaatttcacatgcctaaaagaatttctgagattatggcaaaatcatttactttacttactccttattgatcaacaaaggaagagcaggttttatgtaacaaccaagaagtaacatagggatctcagaagagaagaaaccaagtgtctttggatcttaataaaaagaacattcaactccagaaatagggttaaacctatcctacacatgtctagtaaaaagtcagtgtatcagaggcatctgtgtttagagataaaacataccattagattcatttataatagtttcattgtccttagtttttaaggctaatgataaagatttttttttttttgtgagacacagtctctctgtcacccaggctggagtgcagtgacgcaatctctgctcactgcaacctccgcctcccgggttcaagcgattctcttgcctcagccacccaagtagctaggactacaggcacacgccatcatgcctggctaatttttgtatttttagtagagagggggtttcaccatgttgcccacgatggtctcgatctcctgacctcaacagatctgcccgcctcggcctagtgctggaattacaggcgtgagccaccacgcctggccttaagaattaattttcacagaaatctgcttctaaatattattcagtgataatagattttagaaatttcagattcttatgttgtaccttaaaggttaggatactatttataaatgctaaacatgaactcctgtcatcctattttaatgtctttattaagtgattacatgaatgtggcgtttcagtaggtgtttggtgtattaatagatgtaggtactcccctcttaaacctttaagtattttcatgccatgaaaaatttggggggcattctctttatttattgattaggtacctgtattttcaagaatattactcagcttcagttcccccaaatcttttttagtgttaactaccttccgagaacgtggtataacattttaaaatatattttttgaaggtttggatgtcactgaagatccccctatttattggatggtatctttatcagctgggaagataattagtttcccaatgttgctatatttttatgaataggtcctgtagctcttgctttcttgaaagcttagaattgattgtaagccatggactaatgatattctccaagcatatttaacatgaataggctcatgttcagtatgtcaagaagttaccctgttgtactatttaaataatttgagcttagttttctttttccacctttgttctaaattcttaggtgagtctgaattcctgggcatctgtgcacacagcttcttcagtaggtcctttactctcattgtatctgatctttgacttcataaagacccttggcctactgaccctgtatttaatttcacatctcaatagtatttagccttctcatatttttcctcttctttagagctccctgatccctcactccagtcattcaactctgtgtattcctttaacttccaccctcgctctgtctcttcacagatatgcttctttatatggtagtcagaagttcctgtgtgatcttcgtaacctccagttccctccacagtcacctgttatcaggcatctgttccttgagctccagggaatgagctctttgctatcattctggttgtcggactcactgggcactgttgtgtcctccacagtctctcagtggcatcggttgatgggtgcattctccccacactgatgtcattctgctgcttctgattttctttctgctgtcctccattttcctttcaggctcccttccccgtcctccccgcgccaccttttgcaatatttaggattcagaaattccaaatattgcaaaatatttagaatcagaattcagaaattctaaatatcagtgtttaataggagtaccctggggttgtccattttctcacacaacacatacttgctaggagatctcataagtgcagtggtttttcagaggcctgcattgatgattcctgagtcattatgtccaggccaggcctcatacatactatttactggttctctttctgcgggtgctccaccaagaattcagactcaatggaatatttcttctaagatgtacaccagttccttatcaaaaaatggcaaattcagacttcttaggttaaacagagaactcgtgaacatggcatatgtctgttattccatttttctctgttggcttattgctgagtaccatttgggataaaaatctgtttcaggcaaaggataggattgttttcagcttttattaaattctcctagacccttgattttatgcagagaactaattaaatttgaatgaatttaatgtagaaaccctgctattgatgcagaaatcaacctgattctggactagcaccagaatcaactatttttgttgtttttgctttctttttttttgcttttgctttctctgccggtaaataacaactagtatgtaagaatcaaatgaatcaagcataaatccctaggaaatatttatgcattcatttttaacaatttaaagatgtgaagatatataattaagcataatcctctcaaagtttgacgttgaaaagtatttcagatacttcaacttaaatatattttttgcaaaatatcttttttaaaaaaacagatgtggtctcagcaatgtaattattacagtgtgaggagagcttacagttgtaccaaaatttataactaggaagaagagaatgtgaaataatccggaaacctacaaaactgtcctatacaggaataccaattaaacagctactaaaaatgagtgtaaaatgacaatatggctttatcaagtgcttagggtctggaaaaatgcacatattgcctgttatttattgttatacttaatgaatgttcttttgcacgtgagaactgttataaaagatgtcttttgtacatacttggtacaccagttggctttttaacccattagctgttttcccccaaaggtggcctactctagatgacgttaaaaatacacattttccagggacttatataaatagtcaactgccctattatggaaacttaaaagcgaaggccctgaaatatccagacagtttgaacataaaccaaaaatgaagtgaatctctagaattgctgtgtaatagcgtgagtggggagggggtccctcaagagagggaactgacctttttcttcctcgtgggtgaccttgagtacacactttatttagtcattaaaagggtcacctgaagcttagagaatgaagatcctacagcctgttaattttatcattgttagaaactctctgcctttggcctgaaaatggtaaatcacctttcttagaagattctcatatccagatattcttaccaaatcagaatcttttccagcattttcttcaaaaaaccacagttctgggttaaaacacagcagatcctaactgtaattttcctttcatgtacattttagtcatctttataattttaaatgaaagacaggaactacattatttgaaataagggttaaccccctctaattataagcattaatcacacacagttttctcatttctaaatgttaaatataaaatgggagctatctcttcagacacttttctgttgccttctgtgtgcggcaggaccttacttttaagcttagtttgatttaaaggctgagtttctgcttgcttggaatgcttgctcttcaaaatatttgctccctggagagggaaactgaagatggagctttcacgtgactttggattgctgacctggaagacgtttggggtaataggatgggctagggcatggttccactgctttctgtttgagtggggtcaggaaaggaaggcggggctcgctgtgtgcaaacaggaaaacatagtctgaagatgtttgaagcctgagtgcactttcatttgatgccttgctttattgcttaagagaattttaaactgccgaatccgcaaatgtgctgcagcctataatgacacatatttaaatgatttttgcatctaaacaaatgcattcactctttgcctggtgtgtacaatttgaatgacactcgtgctctgcctggaaggaacagacgtttttcattataaaattcagtcaagcagaaaagtagcaaaggaagcagaaatttccatttacatctatgattgaagttgtagtgataaaagaggatgactaagttgaagagtagccttgttctcattctgctatctgacatgggatcttgtcactttggtaatagtttttaaacttacattattgaggaaggctctgatcagaagtctcaaattatacatgttcttcaaccttagatttttctgggtgttttgtttcaaaagtctcaaattatatatgttcttcaaccttagatttttctgggtgttttgtttggttttttggttatctatagatagatttgagtatctctttttttcttaaaatccgttacttactagtaataaagacttaagtggtagctttacgctccattatcacagagattcacagtactttttagtttttacttgtgagcctcagcaggtgtagacagataccttagttgccactgataacatccagttgccactagatcaagaaatggttcaaaattctccaggggcattttctgctaaagaagagttggtgatcttccttttggaagccttaatgtacttaaaccttcttccttagaagtccagaaattcaattatttcagctctctgtggagtgataagtcctgccaaattgatttacaataatgaaactaagccttgtttacaaatctaagtggtgatttctaagtccacagaggcgtggtaacagagagagagattctgatttatctgggggtctatccgtgtttgaacagattgtgtgatttttgaaacatgattcaaaagggatgttgataaattctaaaatacatttttattgaagagcttgatgaatcatgtatacttactttatttgttattctattagatattgtcttcctcatccctaggtggtgttgctagtacagtgacagaattcaaatccttctttttcctctgctctaacagagctttgtgaactcatagcaatattgtaatgcctggtaaaaggtggtgccagtttcaagtactatatcctaattacttcatggctaactcttgttattgttaacaatattgttaactcttttgttaaatgcagtgacatcttttttctttctcattctctgtgtactttttgccctaaatagttctgcatcttttaagatacaagtttgctttttcttggaggtttttatttggaaaatgcctgaaatatatagaccaacatttgttcctccaatggttaaattacattgtctcccaaagtgaggcattttaagtctggactatacccaggagaatctaaaagaggcagttaataagctcttgaagagcacagtggggtggttgtgtatttattgagtctcacacttaggagagaaataggctggaccaaatgagcctgggcatcaaaagcatgtgggtggtcgttggtgatatgtgcagtgatgagtgtgtgtgctgagggtcctagaccaggctggcagggaaatgaactgaaggcagtgaagcctcccctgataactgcagagagtgctgcagcatccaggacctcaggatcatgatgaacaagctgtagtggatggaagggagaggaactatgggaaggatgagtttctggccagaaagtggtttactggagtgctgtgtctgagtattacatgatctgaggaccatgaggtccagctttgggagtatggagagtttgagtggagtagagttatagtcccatggtcttctgattcacagacattcatgagcgataagtatctattgagaaagtggaagcatgatttttatgacctcctcttgttgtggggaaatcgatcaacggggcctatattggttgggttctacaaccaaatagaatgtgttcagcaggagtcatgttttgggctcctcacaatgttcagtgttagttctgttcctttctcttgcttccacataggaaagactttgacccaatgaggaattagtcattgtcctttgggtacacttccggaatttgataatcagttgggcacttgattctagtacatttgctaaaagtaattaataccctgcaattggaagcatgcagcaagcgactgggaatgctaagtgaaaggtcagaattgaaagagtggaggaggctgttgacccttaggcatggatgatatgtctgagtgagaaagcaagaagagtagaagacagagccttactttataatcagaattatgaaaagaggagccaatgagagggaaagaatgattggcttgagatagattggtggaaactggagaagattgaatttaaagaagaaaagtgggccaagttcagtggcccatgcctagaatcccagcaaattgggaggctgaagctaggagagttgcttgaggccaggagtttgagatcagcttgggcaacgtggcgagacccctgtctctacaaaaaataattaaaaaaaaaattagccaggcatgttggcatgtgccggtagtcccggctacttggaaggctgagacaggaaggttgtctgagcccaggaggctgcagtgagctatgatcacagcactgcactccagcctggttgacagagggtatccccgtctctttaaaaaaaaaaaaaaaaagagaatgtggcagagtagtcagagaactggagggctgagaagagactgatggcatttgttgttcttgaccttgaaagaagagttgcagattgttggagcaaggccagatggtaataggttggaaagaacaagtgaggggcgtgagagtgacagtctacaaccccgttaagaagttatctgtgaaaatgcctcttcctgtcttgattatagcctccctcgcacatggctttctgagtatggtatgtgaaaacagcatgctgtatatttttaccttcccatatgagttaaggttttggtaatgacttgtggttcttttgcctcacggaacaccaacctggttggtattttgtcggcaattatgtggtttttaaaaagccccgtatataaccttaattaattgcgtctctaaaatctatcaggctttgagttttttgttttttttttttaaataaggcagaaagaaaggaaagtgttaggagaaagcaaccagtgagaatacctatattttgaagaaacaaagtagggaatttagaattatctctactatatctgctttttcagctcatctctcgggatacagaggactgaactaagctaaagtaggttcaagtctaaaggaaaccttaaattaatttctgtattagaattaatcacacacctataaatacaatttatgatatgagaactgcaacaagagaaatggggggagatgagcagtttgctgatcaatagggttaatgatggtaacatagaatcctagtgatatgcctcccattgactaaaatgtatgcgttaagatagcatagcgcataaaatggtgtactgcagtctcagtgtattcaagcttattatggtttctctgcctaggtttgtgtctttggctttagattttctcccataaccccaacctgtccacattcaagtcactactccttgaaatatattattgatctctaaaatctgatgctaagtctgtcttgcccaacggtggccactagccacccatggccattgaatatttgaaacctggctagtccaaactatgctgtgagtataaaaaacccaccagatattaaaaagttagtctgggagaaaaaaacaaatgtagaatgtcttaatgataattatttacattgattgcatgttgaaatgatgaaatttaggatgtactgggttaaaatatattaataaaattaattttacctgtttctttttacctttttatgttaatgtggctacaggaaattttaaattagatatgtggcctctattatatttctgtgagacaaagctgctccaagctgttttggtccagaactatatctcggttcttttgtatcctacatggacctgcatagaatggtatttagtaatactcagcagtagcagagatgatctcttatcaaattatgttaagttttaatttttattcttgtctctcaccctatgtgcacctctctggccaggaggcatttatcatatcaccttttaatatttggaaatcacttgatctgcttgagtcattgtgttgttttcaaatctagaactctttctaccttattttgtaacttctataattaaacaaaaggcgaaccaggagcagcttggctcttagggatgtgtgaaaaggcgctcagtccttaagttggtgccaaaagtgacaaatctataggatgagatgcaggtttatggaagagaagcatgaaaatttttattttttaaaaggacacagcaagctagtcaccaaaagacagatacttgattctacttatatgcggtacctttttgaatttgaagagtcaaattcaaaaagatggaaaacagaatggtggtttccaggttctggaggtgggggaatggggagtggaggtgggggaatggggagtggaggtgggggaatggggagttgtttaatggtgtgcagggttttagtttagcaagatgaaaacagttctggagatggaaggtagtgatggttgtacaacgatatgaatctacttaacactaaactgtataattaaaagtgggtaagcaaagagtgtcttacaaagcaataacttgtaacttaaaatttttaaaaaccttaatgagattagttgtaatgggttatcttaagtagacccttactgccggaaggaacagctgaatcacatcacttgggagcttgttagaaaagcagactcagtcccaccctagacttgtccaatcaaaacctgcattttaacaaggcccccaggtgattcataggcactgtaaagcttgagaagccctgctccaaatatcatttctcaaagtgtgatccatgagccacctgaagcagagccaccctgggaaggtctgttttcaggcgcgtcccagaacacgtaaatcagatctaggctgtggagctagggagaagatgaatctgcagactgatcaggatcccaggtgattttgaagcatggtttgagtttgaaaacccctaatctggaaagctttaacaactattactcttacaattccatcttttaacgaatgttggaattattaaatacttgtaacaagaagtcctgttattgataaaatcaagatatgactaatagtgcagtcaaaaagaggcaacaaaaaagcacctataacttatcttgggttggttaatgaggaagctgtcattcaaaattagtaagactactgtaacatttcttggggtcctgaacattggaatgagagtcggttttcagagttgagcccacctctgtcattagctttgtgcaaacctcatcaggctcaagattcctcatttgtaaaggggaatgaattctctgaccagttagtttacattcctaagacaaggttaaatttggaaagcaaagaattttcggtatatatgtgataattttgaaattcttgaatttttaaaaaatttaaatgccacattacttatatttttttctaaaaaaaagtcaaaccaagcgctgtctagttattaatagataccacagatgagatgcctgagttggtaagctctttttaattctagttcttttcaactacaataattttatagacttttttttgaaggctagaatgttgatgtggaatcaaataggtatgcctctatgtatggtagatgcacctgacagggatcacttgacttgaacatgccctgacaatgatcctgtgtggcaggtgcacctgattctgagttctgagttaagaaatccgggagtgggccgggcggggtggccggtgcctgtaatcccagcactttgggaggccgaggcaggtggatcatttgaggtcggaagtccaagaccagcctggccaatgtggcaaaaccccgtctctactaaaaatacaaaaaccagctgggcgtggtggcgcaggcctgtagtcccagctactttggaggctgaggcaggagaattgcttgaacccgggaggtggaggttgcagtgagccaagatcacgccagtgcactccagcctgggcgacagaatgagactctgtttcaaacaacagcaacaacaaaacccaaaacaaaaaaacaacaacaaagaaatccaggagtggctaacccagagagccattccttatctgtaaggaacatctgagccctcatctccttctgtggaacatgggcctcgccggggattgagaccctttgttatgagttaagtggaagttgccaggtggagattgttagggggagggtgttaaatgagaatgctgtagagaccacatgccttttgcaagcggctgcggttcctctgtccaacccaccaccactggactctctaccctgtatgcaagcccccagtaaaaccctatgagacatacggttcactagctctgggcctcttctggctcttgaacctggtgccatccacattgcagtcagtaggagtttggcatgacaccttattatgtgaacttttatatcaagccattaaatttttttaaaatagactttatattttacaacagttttaaatttacaggaaaattgagaggatagtccagagagttcccataggctttgcatccagttttccctattgttaacatcttatattaatatggtacatttgttgcagtcagtaaaccaattttgatacatggttattaactaaagtctatactttattcagatgtccttagtttttgtctaatgtcctttctgtgttccaggatgccatccaggaaggcgcattatatttaattgtcaggtctccatagcctcctttcctcttggctatgacagtttctttatacttctctagttttttaataaccttaaaggttttgtattggtcagataatttgtaggattcccctctgtgggaattttctcatgctttgcctggggttattggttttaagaaggaagaccacagaggtaaaatgcccatttcatcacatcatatcaagggtacgtgtttatcaacatgatacagccctgttggcattgaccttgatcacctggctcaggtggtggttgttgagtttctccacttgaaaattactcttcttcccactttatgtgctctactctttggaaggaagtcagtttgcacagcccacaattaaggagcagagaattatgcttcattttcttgagggcagagtatctacataaattatttggaattcttctgcgcaggagatttgtctcttttttcctatttatgtattcattcattcagtcatttagactcatggatatttacattgtactttgatttctaatccagcactacttcataatttttattgctcaaattattccggctttagctattagaagccctttcacttggttcctatgctcatttgacatagcctcttttttttttttcttacaagatgctgcagtctcatcttgcatattttcagccctaatcctagaatcagctgttcctccaaggagcccttgttccttttattggagagtaatgttattggagaatagtattaaaatctggatgttaaggatgctcgttgccactggagtttctttgcttttaagacttctcagttgatagagcaaggcaatgtatttgtccatgttcacccttatgttatgcagatctataaatatttctatagctaactagttgtatttatattaagctataatatctagtctatattaagctaaatacaagttcatccggatgtttcctactctaacgtattaccacatggacactatatctctcctccttggttatgtataaattctcactccatccaccatctatgtacttaattgttcagtttcagtatatgtgcgtagccgtatcagaattgttaacccaggctgggtacagtggctcatgcctgtaatcccaatactttgggaggctgaggcaggtggattgcttgagcccaggaatttgagaccagcctgggcaacatggcataaccccatgtctactaaaaatacaaaaaaattgctggtcatggtagcatgtgcctgtagtcccagctacccgagaagctgaggtgggaggatcacctgagcccaggaagtcaaggctgcagtgagctgtgatcgtgccattgcactccagcctgggagacagagtaagactctttcttaaaaaaataaaaaataaaaaatataaaaaaatttgttaacccacacctatatggatctttttaaaagctgcaagactgctaacaaatatttatttatttatttatttatttatttatttgagacagagtctccctctgtcacccaggctggagtgcaatgacacaatctcagcccactgcaacctctgcttccccggctcaggcgattctcctgccccagcctcccgagtagctaagattacaggcacccgccaccatgcccagctaattttgtatttttagtagagacggggtttcaccatgttagccaggctggtctcgaactcctgacctcaggtgatccacttgcctcggcctcccaaattgctgggattataggtgtgagccaccatgcctggccataaatacttattttgttcccgtggttttttttttcttttctcttttttaaagtaaatcctttgcactatttttattattttaagaccagtgttattcagagcctgggttaaacactattctgtgtgtgtgtgtgtgtgtgtgtgtgtgtttgtttgtttgtttgttttttgagacagagtctcgctctgtcacccatgctggagtgcagtggtgtgatcttggctcactgcaacctccgcctccctggttcgggagattcttcggcctcagcctcccgagtagctgggactataggtgcgcgccaacatgctcagctaatttttgtatttttagtagagacggagtgtcaccatattggccaggctggtttcaaactcctgaccttgtgatccacctcggcctcccaaagtgctgggattacaggcatgagccaccactcccggccagcactattctgtcttatactcactctgccatctgtgtgtgtatatggtacgtatgtgaggctggatctgtgcacttggaaaagttggagagatgatttaggtaaaacttttagttttcacttcttttgcctttttgcttatcttgatccatttagttcctaccatctggcaaatagttctcatctgaagaatcccatctgtcagttctcactattatccggtaatagttacagtgaaatgctggtgacggcaacagccactttttaaaaggatgtgtaattcaccctcatgtcctctttgagaacccagtaactgcccacactctgaagctaaaaccataacagttttatcacatggaaagtttgttgagaaatgtttggctgtttctgtgaagtaatcgtcccgtctcttgagtacagatgcctcccaggaaaattttcccagcctatttgttaatgtgaaagttacaaaagatagcatataatgagcacattcaagacaaaaaaatttgtggagtaagagtctgaaatggtaaaataaaaaatcacttaagaaaacatttatctgtagctgtcattttcatatacacatatctagtgtatacaagttagtaagtacataattctcaaaagttctacctgtctacacatctacacagaaaaattaggaaagtcttgtccagacttttccatcatcttcagtataaagcatcttctagctgttggaatagaaacaagctagattcttgactcctttctatttgattatttagatctgaccttcagacgacatccagaatctgagaacacgcaccacctctactgctctcatcctggtccaagtcattgtcatctcccacctggattaatgccagagcccccttcctggtcttctcacttctagtcttgaccccctagggcaagaataattagcttagaaaaccaccccagagccttccagcattactctgagtactggccagagttggacaatgtttaaggccccgtgcagtcagctgtccctgtcctgtcctggctgtcactcactcgcttgcccactgatctcttccacatcattcctccacgacagcactgatctccttgttcttctgtgagttcaccaggcatgcatctgcctcaagactttgtgtaatacttgttgcttcctctggctagaatattcttccctttgattctgaatactttgttccctcacttcctcaagtcttcactgaaatgtcatattttcaagtaggcattccctgaccaccctatttaaaatttcacccctcagaaaaaacaaacaaaccacccctttctctgctttatctatgtaacatttattaccatccaacagacactgtatttttatctgtattttttatcgcttccccgctaggatcatatatcgttttatctgttttgttcactgctgtattcttagcactgagacataattgtaagtatgcagtgaagagttagtggataaaggacagtggctgcaagaggggatttaggaaacaccccctcccagccaggaccagcactcggtgcaagagttgaaagagttgatcactcagattctctgcttctccagtcgtagggactccaccccgctgtgttgattgctgtcatgtattcaccccatcataagcagtactcacatgtaaaacagtaccatgtcatctctgtggggctacaagtaggtggtgatggtgcaatgaacatataaaaccccagtgtcttcagtaggtgcgaaataggtgattagtgaatgatgaagagtagctgttgctgttttgattactatgattattattatgagaaactactgtggttgatgagatagatactggggagaaggatactattaatataatttattgagcaactttgttctgggcactagttgagtactttagggatattaattcatttaattctcccagttctgccatttactctctatgtgacctggactgttacatagtctctcagtctcaccctctctttttttttttttttttttttttttttttttttgagacggagtctcgctctttcgctcagtctggactgcagtggtgctatcttggctcactgcaacctccacctcccaggttcaagcgattctcctgcctcagcctactgagtagctgggattacaggcatgcaccaccacgcctggctaatttttttgtatttttagtagagacggggtttcaccatattagccagactggtctcgaactcctgaccttgtgatccacccgtctcggcctcccaaagtgctgggattacagacttgagccactgtgcccggcccttgccttctcttataaagtgaaatcattgtagctgctttggtgtggttctgaggacggtgagagaagacgcacgtgaagtattgagggtggtggcaggcacacagttcagagttactaacaatactggtgttttatcttcacaaggcagatctgggcccatagcacaaggaaaccgaggctttggcattttacagtaattacattgccagaacatttcccccactttcttgaatcatctttacctcttgagagatgaggaagagagggcacacctcgggcagaagtccaaagacctcaggcatttttggcctttcattcccatgattctggccaacatcattttagtccccagcagttcatagaaatattgtcttttaatctgatattcaaataatctgtgccttgaagaacctgtgtatggctctagaatcttttacattaatctactcaaccccactctgatgctcaaaacttcagcagtctgttttttttttattttaatgtctttattattatttttttttttgagacggagtctcgctctgtcgcccaggctggagtgcagtggcgcgatctcggctcactgcaagctccgcctcccgggttcacgccattctcctgcctcagcctcccgagtagctgggactacaggcgcccgctaccacgcccggctaattttttgtatttttagtagagacggggtttcaccgtgttagccaggatggtctcgatctcctgaccttgtgatccgcccgcctcggcctcccaaagtgctgggattacaggcgtgagccaccgcgcccggcctaatgtctttattttaaacatttatttttagaggtagggtctctctaagttatgcaggctggcctcaaattcctgggctcaagtgatcctcccaccacagcctcccaagtagctgggatgacacccccaggtgccgccatcctggcagcagagtcagttttgatacatctttcccttctcattcttcccaatgtcatggattgggaagttactgtggttttagttgcagtactttttactctgaattctgtttgcttcctggaattctgtctgtcttttgaaaagacaacataaagaaatcgaaaaatatactttaaattccatagtgtgggagcctctgctactcgcctgtttgatattcattcccattttttcctttctaaaagaattccagattttttttgttcagggtgacaatgtgctctgctgaaaatactttcacagattcccttgcaggtgcatgtgaccagatcacagtaatggcacatatggcctcagtggaatttgctgaggagtatgtccctcccaagtaacagcaaagctttattagaggaaaaaacaacaacagaaaacctttcacctttgtcttgtttctcctgccttctgtctggaacttggacacagagtcttgaatttctgtgaccataaaacacgaggataaaaccaacgtgctcaggatggcagtacagaaagataggaatacgctggtccctgatggcatcatggaatatctgcagtggccctagcttcagtgtcacttcttaggtgagatacataatcctaccgtgtgtttaagccacggtttgacatgtggtctattctctgccgctgactgcattcccagctgttctatatggcttgctagctaatttaagtctagttttattgtcaaattgaccacattgtagattataatattaaagtacttcacagtctaaaagtgaactacttttgtaaaagccaaaggaaatgggccaaatgaaccattttgaacagagtttaaatgagtgagttgagttaccactcttggatggagaactggagacctggattttcagtctagttcttctgccagttggctctgtgggcaagtcacttgacctgagctcttttcctcccatctgcaaaatgaggtaatttgagctagacagtcctcccaaagaccaccaaagagctagcagattgaggcctgcagactcctattgaatttgccatccttgttcaccttgccaagacttcatagccaagaagccagacccagattttgttcattagctttgaaaacaccctttcagttgggtgtgtgtgtgtgtgtgtgtgtgtgtggtgtgtgacatgcgttttgaatataaattgtgaattgttttagcaagaaaccaggttgtatgtttctcaataaacatgcatactgtatattgatgaatttatccacttgaaataaatggtgccaaattgcctaaagtttgctgtaaacaggaacatgatttctcattatgatgtttaatttgttttagtctttgatctcttacatcagaattctgttaagaactctttttacctgaaagatcaaagcaaataagcagaagcatttataaaaataaggtatacgtggctgtggagagaaactccaggtatatacgatcataggctttttaaaattaataaactttgtttttaaaaacaaagcagtggaagttcacagcaagtttgagtagaaagtacagtttccatgtaactgctgtcctagcaaagatttttaagtacaatattttttaattaaaaagagaaaatataaaccgtgctaagccagttggtgaaaaaggcttcattatacttttagcactcttaacgcttattaaaagtttttaaaaacagatttttgtggttgaatcgcttacaatttctgatgtgtaaccttctaacatgtttcatcaaattttaaaagtaatgtaagcagtgctgtccactgctaaattttattttaatgttagaatacaatgaggtttctattttgccattagtcctgagagcttattaagtaaccagttttaatcctttaaaaaaccattctcattttgagggacacatatttcttagctttgtgcaatataagaagctaagaccatcttggcagtatcaaatgaaatgttttgcaagttagaaagttcagtaaccacttataaacataatgaacaattgttatagtcagtctaatgcagaaatacaggtgttgggggcaggggggtggtgggtgggcagacttggtccacaagtagtagtttgccaacctctgtcctaaacacaagttcaagaatatggttctcttggatagattttctgccagatgaaagtaagagtaacatccttagctaatgaactcctaatatccaaggcaacttttaattaattcaggcttttaagaaaagcttgtttaccaactcctcattaaaaatgttcagactggctggtcgtggtggctcatgtctgtaatcccggcactttgggaggctgaggcaggcagattgcttgagcctaggagttcgagaccagcctgggcaacatatctagaccctgtctctgcaaaaagtgaaataattagctgggcatgatggtgcacagctgtagtcccagctactcaggaggctgaggtgggaggattgcatgacccccggaggtcgaagctgcagtgagccattgatccaaccactacattccagccagggtgacagagtgagaccttgtctgaaaacaaacaaacaaaaagattaatgcagtacatgtccttttggtgaataagtaaatatgttttggtaactttagctttgcattataatctagtcgtcagcagattgactaagggcagataaaacaagatgatttggttttgtggtttggcacttactgtctgatttatgcccccaagaactcttgcaggccaaggtcgagtgtatctgatacctgctctggctcttctcacgaactgatttagtagctttgggccagcattagattttttaaactctttgacctatttgtacatgacaggcatttaatctttccagtagttacttgtcaaatagaaaagcatcacttcttagtttggacatttgcaaatatgactgagcagagctgacagatgaggtgggcatcttcaggccatagggtacttctggcctcctgtaacctagaaaattgggatgcctcagatatcaggtattcttccaagcagctagggttagtccctgacctcagaaaccctgagagccctcagaaaccctgagaaatccacctgggaatggtgtatttgttttggatgagattacctttactttccattctccaggtcttcttgtgttttttgtttttgttttgttttgttttgttttatatcattctatgtaaaagagaaatgtggtatcatttgcattatcatgttatttctttccttataatttatatcttaatgcttatctctggggctaggttataatctcctgagttaggtagccttaggtttcttggcggagattcctcccctgggaagtgaggtttataaaagaacccgtgatgtggccctgtggagctgcatggggtgtgtgctcagtactcactggtttcttccctgcaaagtgacacctgcagcctttggggtaccatgtctcataatttttgcattccctgcaacacacactatactgctatgggcatcattgatcattttaaagatttaaggaatgacaagatacagtgcttattctcccctatgatttgcttagatttttatctaaatagtacttattgaaataagtacttcagtatcatgctacatcttacatgtttgagctagaaacagactaaagtttaaacccttttatgagaatgaaaagttacttccttccccttcttagggcttttaatccctctttatgttgcatttcagaaaatgccctatagcgtactatgaaagaaagacattcattcctgtctttgctgctgtgtggacgatctgtattccttgctccgtgggagttgtgggtttatgtctattcttgcatgttttgcctgaaggaccacagtgacctacttggtggtctccctgctttcatggtttcactatgctgatctatccttcacttaattatagagtctatcttaaaaaaaaaaaaaaaaaaaaaaaaacttttggcctatgttgcttcttccaaaatggcatccaaaacctaagcctctttaaaattggagtccagcatgcttccccagtgctacttccttctggactccctttgccacttgctttctgcttccacccatatccagatgggctgcaaatatgccttgctggttctttactctgtctttgcacaggaggctccctcattttgaagggtcttttgctacccatccatccatccatttggattctataatagagttcttgatgtttgacagaactaggccaggaattgtgggggtataatggcaagcaagacagagtctatccttacgttatggaagttcaattcatttcccatgtggcatactacttctcaccattcagattcatctcgaatattgtttcttctttgaggtttttcccactgcacccaagatactggcctgacactttccttatctctattacagaatttaacttgttcattcattaacagatatttattgcctactatgtgcaagaatctgtgctaaaagctgggaatattgcagtaaacaaaaattaagtttacagcttacatataatgtggtaattatccatcttttaccttcattagactgagttcatgtaggactttatgtaatatctttatatcttcaataccagaaatgcctagaaataatttgcataatatacattttctttaaagatttaacaaaagtctgagggacagttttcagaataaactgaattaaactctctctctcttatgtgtgttatgtaaacatatatataaaatacacttttgggttttttgcttaggaagtagaaaaatgctaaaatttctgttacttacaagcagattaaaatgtccaagaaagaaaagtagggatttatattgattctgctcaaatgcttacctagacaatgtcaatatttatattttactcccctgttgtagtagaatggtaattggttctctagttaagcagcttttttaggattaattttgaagcaagaaattcagattaattaagggacaatttcacttgcagcatttaggcattattttaaccatcaaacttgaagtggcaagaaacggagttgcttaaggaggccagacgtaacattctaaaggtagtagggtcttgattgggttgcttaggcattaaaaggctgtttaacttgtcttgaagtctatctttccttgatgtcttctgcggtaagaacactgtgatacagatggaatgacgggaagtggttttcctttctttcagttggtgagtttgtaagtgatgcccttctcgttcctgacaagtgcaaattcttacaccaggagaggatggatgtttgcgaaactcatcttcactggcacaccgtcgccaaagaggtaccagcccataaattctttcttattgcaaagtgaagatttcctggggacgtgcttaatggcattttaggggtattttgaggcaacagctatgttgataagttatttaagaaaaaaacccagtgctactatccgtaacccagattttagtttttagagtgattttaggttcacagcaaaatcgaacagaaaatagagttcccgtgtaccccctgtccccacacatgcgcagcctccccatccatgaatatttttaaaaagtcaaaaatagggccagttgaactagattcattatgtgcctgtgccgtgtttcgcaacttaaaaatataagttgtctgtgccatgccttataacttaaaaatataatctctaatagttataactttaaagtttacccttcagattatttttatgcacctgttgttttcttttgtcatatacaattttttaataatttagccttttcccctgttatctattagtgaatacagtggtagaacagtgaagtagcttttttataccagcctgaaagctccagttagaattcagtttctcagttataccatccagttttatattcagcaggccatgcctaccatcttgatttttccacagggtgagtctgaccgagcacttggttaacctctgcttttggctaagccaataaaagatgtaagatacttagcatgaaaaggtcactgtgtggtaagaccaccttcacagctgctgtgtccccttgataggtttcctaataaatttatataggtttatttgtggcaagatcagtcttaggctaactagaattaggctcctttatctagctcatagtgcttcacttgagtcaattttgatcccctgtccctcaccggaacatgtctggagacattttttgttattacagcttgagggagtggtgttactggcatctgatagtgcccagaggccagagattctgctaaacatcctacagtcaacaggataaccacccgccacccccaagaattaggcagtccaaaacatcaatagtgctggggttgagaaatctgatctagcttagcttaaccattgtatcatagctttatagctgttagacagaaaataccactgaagatttagtattattttatgcctcttataaaatagtatatttggatccactgtagaataaaatgttttttatgtaaaaatgtgttttggaggtttaagagcttgctgagtttaagaaaacaaaccaaataatttgaagtccagctacctatttgataacctcattttttattccttctgtgttttcaaaagaaaaatcctttgtctctaagaaacggaaatgactcccataaggtcaaaatacagtctgtaatattttcattgtctacctttatgtcttgaattattcatccttcatttatcactagtggatgctctcagaactttatgctaatctagatttgtgcaaatttagcaaatcccagaaaagcaaaaagacattcaaggagaaatctctaatgactgtaagttctgattcttagtggctgcaggggataatttcttctcttcccaggattgttactctgaaagtgaaccaagagagtcagaatttctaggataaggcaggtccatgtgagataaacttccatctgtaagggaggtaggagtgagctggggatctacctggaatgcgcacaatttctggatacacgaattaagccagaaggcattaattctctggtatctgttcaccagtggagactgctacatcttatttcccaattagtttccattccagttgttcgtataaacctctattatataacatcgtggtcttattaaataaataataaacaagaataaaaagagtaccaaagtgtaacccatgctaaaaaaaatcctgttgtaaattcactcaaaaagcaaataatcttttcctttgtgaaattggttcctaatatattgggtctgcatgttgattattttatgtggagttttcttacaatgaaacacatctactctaccactcactgttttctccttacactttgtagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggaggtaaggtggcctttgtgttcagcctcagagatgctgaaacatcttgtatggagtatttgtatcctgtaaattaatctttctgtttatcactgaaaaggtctctgcccactcccatcagagtctgctgttatgcaaaaatctgaactatgaatttttatggcatcctgttgaattaataatatcagtcacccatcacagagttaattttaactatttaatattaaacttgggatcaaaatcccactgataactatcataggttactggtagttctaacagggagttgaaataataatggcgttctgttttggtcattaatttaaaaatatttttaaatgctctctgggattgtcagtcattagtttaaggtgaattttcgttaaaggtttggtttgagattttgggcgttctctgtgtggatgtgtaagggtttttttttggccttttatcatttcttttccttcatagtggaagtcaaaggcattaataaatgcttgttaattttttttaactacctttgatttgttgattgtaaagaaacctattttcgcctctttttcttagggagcttagtcccgttacttcttacctgattctcattcttaactgaaggcaaacatctatattcaaatcataaaattataaaatgagaatctttgggttggaaatgccatctggttcaggcattgattttatgctgtctcatcccccgaagggtgatgattgtatcacttatcttattccttcagtattctcttagatgttccctagagctacagatttccaaacagcacattcaataggagttgtaagtttataatcggcaaactacaggatgcttgtttttttttcttttaatttctggtgtaatctgctactagggcccccatagagtttattgtctatatcagctagaacactgggaccacagtcataaactgaaggatccaggcaaactgggtcatatggttacccccaccaattacagtcttcagtgatgatgtcactatccaggttgatattcagttgaaatgctagccagctccttcctagttaccttcatttacatcccatttcagtacctgaacacagtcatgctttagactctgaaattccttagaaatagtaccataaggtttatagctttgatattcctctctgatcatgatcttccctcataatcacctccccagttcaggcttttagtccattatcctctttttccctgcctgcctttctaatcaggatggaatcaatggataaccattcttacctattccattgaacatccttgacttggctgaaacccttcacattttctaaacatatttttgtcaattctccatagctaggaaaaattaatttctttctttagttgcttctgggatttctaaagtatcaagtcatgtaaccttaccacttaattccatttcttattcatgctgtaacatcagctgggatcttaatgttactagatgcacctaattatctctgtcttgatgacctatcatcttccccatagcagttgatccagcccaacttcattctccttggttcccagcgtaactctctctcatgctctacagatgacccctccaccgtcatccattgtgaatttctgcaacatggtacctgacatttggtaggtgcccagtacgtgtttgttgaataaaccattgaattttctcctgtacttcagcaactcttgtaccttcttctatccctttgtttcctgtcttcccttttatgattatgttctgctggactctcaatcatctcctctccctttcttgcattttcagtctccctctcccccacacccccaggcttcatccaccctcctcccagataattctttcccatgtgttactatctttctactatgtgtttttaatcctgttgacagttggtaggaaatatttgatggtttattctacctgtagaaaagtccaaattctcttgcagggcattcagaagtctcttacatggcttcagcctgcattttcagccttatacatgtatgtgactctcctggcatcctaaaaccattgaccttttctttctctaggctttcattcccaccgttctgccccatctgaaatatttttcctttttttgtcaaatggggttaatgtatgataaagatatttgtcaactctaaagtgccacttcttatgacccgtcaaaatctatatatttataaatgttcacctgaaaagatgcagcctttacaagtctttacaggtgccttgctctgctatactacccttgctttacctttttcttataagaccataacttatcttccaattaattgtagtcatatctctttttatctgttagaatccaaactatagtttatgtgtctggtatgtcttgcacataggtcctcaataaatattagacaaagtaaattgtatattttaaattttttttcatagcttttggtaaggacttcatggtccacgattggatattgtagaaagcttatccagcccactgcccaggacgactttgaatgcggaccaacacaaattcataaactttcttaaaacattatgaaatttttttgcctttttttttctataggccctgttatagagcctcccatcttttagcaaagaactgtctaatacaaatgtaatgtgagccacatactttttcgttttctttttttaagatggagtctcgctctgtcacccaggctggagtgcagtggcgtgatctcggctcactgcaacctccacctcccgggttcaagcgattctcctgcctcagcctcctaagtagctgggattacagacatgtgccagcatgcccagctaatttttttttttttgtacttttagtagaggtggggtttcaccatgttggccgggctgatctcgaactcctgacctcgtcatccaccaacctcagcctcccaaagtgctgggattacaggcttgagccaccgtgcctggccttactttttcattttctaatagacagtttaaaaatagtaaaaagaaacaggtgaatttaattttaacaacattttatttaatccagtatatctgaaatatcatgtgaccaatatgaaaaatttaataagatagttcacattctttttttatgtttctttctttcgtaagaagtcttaacacttcttttaacatttaacagcacgtctcaattcatactagccacacactgtgtttgatagttacatgtggctggtgactactatatttggacagtgtagttcctggatgacttgaaatttcttatcactctttatttttccattaagacagacggtttgtataacctagtaatgttttctaaggctgagccactgaatttttattattgtttttaactattcttgtttttaaaatttaaaaaaattggtttggattcagtataattagtatatcaggtcacttgcaccattttattttttaacttcttttcctgtagccctgcaggcgggcagggcttctttcatgtggacaaaaccagaaaggtgttgcatcactgatccacacacaaggtgggctcctgccaactggcagcctccttgttccaggaaccagccctccttttgacacttgaaatgtaaaatcagaatagaagtttaacgtacctgacttaaaacatcttcaggggatgctacgtgacaattcttgaagaaggagaaacgagctaccattttcctggaaacaatatttgattcacagtattttgttgtttggtagttaatgaggcttgttttgatagaagatgagtgtttttctatcgctaatggggcatttagagaaacattccttgtaggatgagctctgttttatgacctcattgaaaaagaggtatgaaaaagaggtaaatctttaacataggcaaaacaggtagtgtttcagaaatttatgtgtaaattaccgagtatatttttctccagtgttgtaatggtcatattctcagatcacctcaaaatacctatttatgcagtctgtaatgtagcataatccagactgttactaacattatgtagcataatgttaataataataattacgtacataatgttaataatacacagactccagccatcgtgggtaagatttttttccatgggaaaattcatttggaaagctaacttgtaagactagccaggtaggcagcctagccctggaggcagagctgctcctcacctggaactgcagaatctgattgaacccagtggtccatgggggttgctgaattcgtaaagcaaatgaaatgttagaagaccctctaattgttgtagagtcacacttttcctcttgcagaggaggtactcacattttttttaagaaacaatctttaattatcgatgtattcttttaggtttaggtttgcagacactggtgagcgtgtgctgagaggtaacttgatgtatctttctggataattagatgattttttttttctatttctttgaacattttttttttaaagagctgcttaagatctggaggagtgattccttttgaccgtgtcttattttcattaagactgtataacttggaagagtttttttttttctttttctttttcttttttttttaagatgaagtttttgctcttgttgcccaggctggagtacagtggtgtgatcttggctcactgcaacctctgcctcctgggttcaaacgattctcctgactcagcttcccgagtaattgggattacaggtgttcaccaccacacccagctaatttttgtatttttagtagagacagggtttcaccatgttggccaggttagttaatgttactctctggtttggggggcagtgatatgttttggcttttttcccacccagatctcttcttgaattcccatgtgttgtggaagggactcggtgggaggcaattgaatcatgggggcaagtctttcccatgctgttcctgtgaagtgaataagtctcacgagatctgatggttttaaaaagaggggttcccctgcacaagttctctctctttgcctgctgccatacatgtaagacatgacttgctcctccttgccttccaccgtgattgtgaggcttccccagccatgtggaactgtaagtccagttaaacctctttcttctgtaaattacccagtctcaggtatgcctttatcggcaacatgcaaatgaactaatacagctcggggtggcttgtgtgggtgcgtatggccatgtatgcaggtgtgcgtgcattggaatatacaattccgtattgaacccagtttaaacttcatcagttctggtgattgctggagtgtcacatatggcaagacgtcaccttgccctctaactacaccagaaaaccaggccagcaccattgactatctagatacagtgggccgtttatattattattgtttcatatttttagattatttggtaaacaatttacagaactaagaataaaggaatagtgagaaataactattagcaattttaataagagttaacatagtaaatattttaattttacgaagaatttaaatatggtaaacccacatgtttattctgtattttcccagttttgaaggtatggatttctgaaatgagcattatcctttggaatgcagctagctgctaatcacctgccatgcacgaacctgaaatatatccgtcttacaaatactggttctacatttcaaatacatctctattaggttcatcatatttcttggtcttttttttttatacttgcaatttcatttaattattgtaaaaatttaagatatttaaaacattgacagctcataattttattgaagagagaatatagccatcttccctcaggaataaaatgttttcaagtttggatttgtaaacagctattaaaatgatcaagccagtctttttttccccctttttttctgcattatctcttctaatcaccttagagagattcctgccttgggcatttgtccacttcctaatggtatcaaataaatgtgttgcacagacataaaatgtgttgcataagcatcataaaatggcaaggaatactgtcacatcattttagcaatgatggttaatgtttttgtcacataatattgagtgatgatatccttcctgttgaatgactaagtggatgagaataccatatttcctttttattcttagaaatacattgctcagccgggcgcagtagctcacgcctgtaatcccagcactttgggaggccgaggtgggccgatcgcctgaggttgggagttcgagaccagcctgaccaacatggagaaagcccatccctactaaaaatacaaaattagctaggcgtggtggcgcatgcctgtaatcccagctactcgggaggctgaggcaggagaatcaattgaaccagggaggcagaggttgcagtgagccaagattgcaccatcacactccagcctgggcaataagagcgaaactccacctcaaaaaagaaagaaagaaagaaagaaatacattgctcacttgtcaattcttgagcttgagaaaattcatctaggatatcatgtgaaggctcatagcctaaggttttacttacgtgatcaatttcattcttcttctggtttatctaattggtaaaatgcctccttctgtcagttgttcctatttgccaatatgtggaagatgatttttgactatattcagtgagtctccagacttctttttatcatgctttttaaagagatgggacctcactctgttgcccagacttgagcgcagtggctattcactggtgtgagtatggtgcattacagctgcaaactcctgggctcaagtgatcctcatgcctcaacatcccaagtagctgggactatgcagactttttttaaaccatcttgaaagatgatgcagtactttgggaacacagcaataaaaatgtgataatttccctattgcatcacctttctaggcaattatatcattctttcatgctcatattattttgtcttctttagcacagtttttaaatagttgatggctaatgatgaaataattcttcggatgataaatattttgtaacataggaacacaatataggaaaaagatcgaggttcataccatatctcagactcatggttaagtacaagctagaatgagttttagtcgattaaatttctttttctctatgaatggataaagtgagaatatcagcccacaagtagttagaagtgctcagaactcaaaggtaaaattgtgtccaagacctgatggtattctaaaggttaacagcctatcctacttaccaagaatttacatttattatgaggcctgtacaacctgatgcaggaaaatacttggcataattttctataagttaggtagttaggtgctcaacaagtatttgagttgaatcacattggcattcacatcaaacacatacacctacgtcatcatgttaacttaaggttaatagtgtcaccaaaactaactggtctttaaaaaagaaaaactaggccaggcatggtggctcatgcctgtaatcccagcactttgggaggccgaggctggcagatctcttgatgtcaggagttcaagaccagcctggccaacatggaaaaaccccctctctactaaaaatacaaaatagccaggcatggtggcatgtgcctgtaatcccagctactggggagactgccagagaatcgcttgaacccaggaggtggaggttgcagtgagctgagatcgcaccactgcacttcagcctgggcaacagagcaagactctgtctcaaaaaaaaaagaaaagaaaagaaaagaaaaactaaggaatatcattctagacatgcagtaattaagtaaaattgcatcaggagtctaataacatagcatgatgggtcccatgtttcattatttccatgggcagaagagggatggctacattataaataaaggaaaattgtaagaataagaaaagatgaagtatgaaatatttccaactcaaaaacaaactgtatctgagcatgcagaggtacaaaatgtcacctttttcctccagtgtaatgaagggcactaaacctcttgagcatttgatatgttccagatactatattaaagtgatttttttttttaggagacaggttcttgctgtcacacatgctggagtgcagtattgcaaccatagatcagtgtaaccttgaactcctgggctcaagtgatcctcctgcctcagcctcccaaatggctaagactacaaacacattgccaccatgcctgcctaatttattttgatttatttattttttttgtagagacaggggtctcactatgttgcccagactgttttcaaactcctggcctcaagtgatcctcctgcctgggcctcccaaagtgttgagattacaggcatgagccaccacgccagaccaaggggtgaaatcttgataacaataatcaatgcagtcatcataaaacgcttgcacaggctcaatttgtaggtggcacagtgctacatctagtcaaaagcaaatttaattatggtcattgtgctctgagaacttttctcagaaaataataaccagatggtattatagatggtgcatttcacaaatacatgatttgtagttagtgaaaacttgtcattcaatcatgtaataaattatgcacatgccgtcttccacttagctagtgatgttgaaacattcatagccttacctgatgagctcaggctgtcatgagaatttatgtctattcagtactttgtctttaaaattaggtgagagagaaagagaggctgtatttgactatatatctttttatttgtatctctggagttacaagtttttgaatatataatctttcagctgcctttctgcattgcattttgagaaatgatattaaatgctcaaggaaaaaaaacctaacaactttagagggccgcaaagtgaatcatacaaaagataaattaaagctttaaatctttgccagtggatgagtcaagtaagagtggatttaatgaaatggctcatggctttgagaagcagctgtcataatgctgggttggcagcttccaggactgtgtgaggctttctgactgtctatactgaaataggagaactcatttgaaaatgacgaataggagaatagcagaaacttgcattttcttttgagggaactggctgccagttttgaggtccttatttagaaatgctcgattcctgtattgaataaacttggactacatgtatgtcaaaaagaaaagcaaaacacttttatagccttaagcagattacagatgtgtggccaaaccagtggggtctgtttttgtttccaagcaggtgatggctggaagaggagatgcctctgtagtgcagggcttgagttgctgctgtctgagctacatctggcaatgagactgtgctgcttactgagacctaatggcacaggttgccccagtaacaggatattgctgaatgagaaatactgtaggattcatttgtaagagttatttggagggttggattattcagtgctaaggatcctaagctactgatgcatggactcttttcttccctccagtatcttttaattaaactgtacaaattccctggcttaaaaatagtaggaaaagaggaagaaaatacagattcttatgattttaactggtctctggcaatagtacaccgaaacattaatttcttgttgaaaccatctggatatttgtatcttcagtgtaaaacccctaatttagataggactctccattgtgacatggaaaaataaccctttaattagttaattaattttactgtggcctcaaaatctacatattagcatatcaagccaaaaactgttggctttacttgggagccatgtggcttaaactagtaccagtaacttaccacaaggaactcagcaaaattataagtccatgtttgttttggaaggaaggcaattattacgaaaataatttagagatgttcaaactatttcctttgagtagaggtagagagtgaacaaaatctgtcccaagtgggagctaactagcttccttttttgccctccctgtttacaaagtaacacttttagtttactaggtaacatttgcagtatccccagtgtttcaatgtaaaattgtaggtggtagactaatgccacttagccaaagccatgatcacctgaatttttaatatatatttttgtatggagtgagatactgggagaacagtcttcattagcttaaactctagaatgtactgtttgtttgtttgtttgtttgtttgttttgagatagagtctcgctctgtcacccaggctgcagtgcaatggtgcaatctcagctcactgcaacctcccctcccaagttcaagtggtactcctgcctcagtctcccgaggattacaggtgcctgcttccacactcagctttttttttttttttttaatggagacggagtctcactctgttgcccaggctggagtgcagtggtgtgatttgggctcactgcaacctctgcctcccaggttcaagcgattctcctgccttagcctcccgagtcgctgggattacaggtgccaaccaccatgcctggctaatttttgtatttttagtagagacgaggtttcaccatgttggtcaggctggtctcgaattcctgacctcaagtgatccactggcctcggcctcccaaagtgctgggattacaggcataagctaccatgcccagccaagaatgtacaatatttttaaagaaatgcggtttattaccataaaatacaggttgagcatccctaatccaaaatctgaaatgccctaaaatccaaaatgttttgagcactgacatgacagcaaacgtagaaaattatgtgactttatgtgttcaatgtatacaaacttcctttctgaaacaaaaattatttaaaatgttgtataaaattgccttcaggctgtgtgtataaggtatttttgaaacataaatgaattttatgtttatacttgggtcccatctccaacatatcttatatgtatggaaatatcccaaaatttgaaaaaaatctgaaatctgaaacacttctggtcccaggcagtttggataaggattactcaatctgtaaatacagtagctcaaattgattttgtaaggcagtcaagttattatttatagtattttaatttgtattattcttttaatatagcaattcattttatatatttatataaataaattttttgcaattgagcaaattaaaaaaattaagtgcttccttcagatctacattttattttctatttgttatcaatttaataagtaaaggacacacagatcatctgattgtgttgtcagacatttgaaggtgcaactgatgaatgttcactgttgtactttgttttgtcatgtaaattgttttaaagagaagtgaatgttgagcatatagtaggccacacgtgcctgtggattggctcattgttttaggaatgtgtaattctggaaaagatttgccaagggtattcaaaagtatagtttgatattagaaattatttgaaaataatctttatagcatagcaccattttatttagatcaatagtaattcaagcagtatacataatttgctattgtaagcgtagcatataacctaagttttcttttacatttgagaccactgtttgctgttggtcgaatttggtcttccaagattttttgcttagtgacctaattgccagcctttgtgaagagctgttacttattgtcaaaccaggcagtaagggtttgcttttatctttttatcagttctttcaatgctagtagtcatgagattaatgtagttggatactaaaatgttaatacgtgttagataacaaactgggaaagagatgaatgacatcatgaaggaaagggcagtattgcacttacagtgatttcatgatgatagaactaacctgcctaaaaagacattgggtggaaacagaaaccaggtgtaatagacgcatccaagtagtcaaggaaagaaaagacttgtaagtaaataagataatctttgcttcaaaaatatcttcaaaggtttaaaatgtgctttaccctcatatataatctttttgagcctatttctttacctcaaacatggggaaaattgcaataatctgtatcacggtatttgcttccaaaacgactaattggtgtaaatgcatatcaagccggtaaagtttacagtggacttgtttaaaatgcgtagattggaattgttgggggagcaggccagcagcatgggatgttttttttgtttgtttgtttgtttaagactctgtcgcccagactggagtgcagtggcacaatctcggctcactgcaacctccgtctcctgagttcaagcaattctcctacctcagcctcccaagtagctgggattacgggtgcctgccaccacgcccagctagtttttacatttttagtagagacagggtatcaccacgttggccagactggtctcgaactcctgacctcaggtgatccgcctgcctcgggctcccaaagtgctgggattacaggcgtgagacaccgcgccagctggggtgatttttttcttagtcaaatcacttctctacccatcatctcacactgtcgaatacaaagactctccttggggctcaatgactcatctcccacctctggtttgataatcatggctattataacatgttccctttttaattagttattttaaaattattgtaggtcttgtaatcataatcaataataatacgtaatggctccttaagcacttctatcctagataaggttgagaacattattaaaattagttttggtagtaatcaagtaatgataattcttatttatgaatttaaatatgtaggagtttttttattttagggaagcaaaaaggactagaaagtatgatgtctcatgattagaaaactgctataaaaatgaaggtagaattgatttgggctgttttggacattttaaatagaaatttaaataggtattcaaatagcaaagtcattctactttaaaacttctatacttttttaggatcttcaattttatatttaaattatatttttgaaatggttttagaagaaatattgtatcttaaactctaactactaataagtaagagaagatatatcaaaggtttgatcttttatctcctagaatatactattcattcgtttcccattgtacgtcatttgaatgttaatggttagcctcaaatggttttcaacataattgtatagctactattattggaaaattggagccaaggactcatacacatatcagaagtggtaatgaagcactttggttttatgaaaatatgtttacaaggtagtcacaggatagcaatattgcatttactgtaatacaatgatggtggaactaagtgcagtggaacaggatgctgagaccacagttaccgcatctctaagtgcctcgtctagttatggtccaacctcagatataactcattggtgttgaaacacacctagcaacattttaagcatagaccgttcattaaaaggtacctttttaaaaatttttatttttaaaaattgacatgtaataattgtacacatttacagggtacacagtgatcagatcagactgatcagcatattcatctcaggcatgtatcatttctttgtgttgggaaaattcaatatcctgcttctagctgtttgagattatgtcatatattactgttaactgtagtcatcctacagtgctgtagagcactagaacttattcctcttatctagctgtaatttggtatcctttaacaaatctttctctatcctcagtttctacctacccttcccagcctccatcatcctctgttctactctttacgtctatgaaatcaacattttctagcttctgcatatgagagcatgtggtgtttaactttctgtttttggcttatttcacttaacatatgtcctccagttccatccatgttgcttcaaatgacaggattttattcttttgtatggcttaatagtatttcattatacgtgcatgcacgcacggacacacacacatacacacacacacacacatcacattttcttcatccattagtctctttggacatctaggttattccatatcttggctattgtgagtagtgctgcagtaaatatgggagtgcagatacctcttttaatatactgatttctcttcctttagataaatgcccagtagtgggattgctggatcgtatggtagctctatttttagttttttgaggaatctccatactatcctcaaatggttgtactagaacagagcttttttaccttccttccatttgggaatcttcttacccaacatgtatattagtaacttggctatgtatgttccattctctgtctcaaatactgtgtctaggacatctgcaatctttggctatgaccacgactgtaaagtgcatgacgcttttgctctgtcatcagtattagttgacaggcaagtaagatagtacttctcttcataacccctttacctgcccagcagaataccagtacagacagacccagaatatctctgggctcaagaatccagacatttataggatgggacttttatctataaatcaccttaagattagtaagttcttagaattggttacctttgccatttctcagaagccctttgtctagtaacctgtgtgtcactgtcatttccagcctgtctttcctacacattgtttatgcaggctttaggagtgcccaccccccagttctaccccccacccccaccctagcctttgtggttttaattatcctgtcttggctgattttgttttgttttgacatttcatccattaaattcctgacactggctatctctttacaattattatactatttttctttctggctcgctcttcctgattgctgtaatccatgcccttgactttaaataccatttgtaagtagactaggccaaggtttatttctctaggccagactctacctagttatttcataggcatatgaatcctaacatgtccaaatgagagttcttaattcccctgctacccagtcctcaatgtggttctctcaaagctattctatctcagtaatggtcctatgccctctcctagggattattgctgagaacctagaactcatcatcgtctcttctgcacatcctatgaattctggatttatccacttatctccatcttttccagcactaatctaaactcccattgtttcttgctggagttatttccgtatcctgctaatctctgctctcgttctctctccctgacacatgcgctgctttcttctcccacaccaagccattctctgtacagcagccaggatgtagagtgtttatttacataaagcagatcctggaacccccaaatttaagttgtgttcgtggcttaccaatacgcttaaaaaccaagtgcacagagagaaaacaatgaagccttatttggatatgctgaggataattgataacgggtaacaactaacaaacacgtagttggtgtcaggtttgacctcctacattccttacaggtattagtgtatttgacccagctcctccctacctccgcagtatcaccccatgcctctctttcccccgactctcccctgctcagtgtcacacaagtcaatatccccagcttgctaacccccgggctgacagatctcattgtctataatggatgttccaaggtcccaccttcagcctcagttttgttgccatctttccagaatgaattcccttgatcattctgttttttggagacaaggtcctgctctgtcacctaggctggagtgcagtggcatgatcatggctcactgtagccttgacctccctggctctggagatcctcctgagtagctgggactacaggctgcacctggctaatttttttttaatttttagtggagacaagatctcactttgttgcccaggcttgtctcaaactcctgaactcaagcagtcctcccacctccgtctcccagagtgctagaattacaggcatgagccaccatgcctggccctgatcattcttttctgaagaaaacaccccttcttcattattctcaatcctagcagcctatttacttccattctgaaatgaatcacattttagtattttattggttcgttgtcgttctctccccccatagaatgtaatctctctgagtacagggagcttatctttttcccttttggagcctcggatccagcagaatgccagacacttagcagccactagcatttgatgagtgaatggcttttcatacacaattttagacattgttcctgattggtctctctagtcaataatagatttatcaaatctaatcgataatagatttctttttcttttctttttttctttttctttttctttttttttttgagaagtcttgctctgtcgcccaggctggagtgcagtggcatgatcttggctcactgcaggctctgcctcccaggttcacgccattctcctgcctcagcctcccaagtaactgggactataggcgcccgccaccatgcccagctattttttgtgttttagtagagatggggtttcactgtgttagccaggatggtctcgatctcctgtcctcaagatccgcccgcctcagcctcccagagtgctgggattacaggcctgagccaccgtgcctggccgatagtagatttcttttattgattagagagactaatcagcaacaatgttttaaaattctcacatagatctctgaatgggaaagcagtcttgaattatgcttgcagggagagagaggggaaggaaggaggaggaatggtataattagacttcataaggctagattttgtttctacttaaataatggtatcacagtgggacatattatccatgcagaaagtgagtgggcatctggtctattatctgtggtctttcaggaatggcagctctttagtttaaaaaacaacaaagtgcagcactgtttatttaatggaaatagaaaagcagtgcctgtgtattagtggctcagatttttttgttctttgttttcagctgtctgctgtatgcctctccagcggggtcttacagaatctcaagtctaccatgatcaaagctgaacccattagctccatttcttaattcctcctgtatcctctgttaccattagtaactattctccaactcaaagcatcagattcctcctcttactcactcccttcatccccagtcaccagttctgtgctgttattttatccaagttctcagtaggcttcacgagcaccgccttcacatcccaactgtcgctcctccacctatctgtcctccatgcttctgctagctcatgtttgttgcaatacaactaaccttgccactcttctgcttcaggcttttctactaggctctgcatctagatgatggtccagactcctcagcctgtgaaacaaagcccttcataaaagagcagagctacagctagtcagctcccttcctgcagtccaccccctcctccaccatactgagaattggcaattcctgtagcactctttgtatgagtccattttcacactgctataaagatataaccaagactgggtagtttataaaggaaagaggtttaattgactcacagttctgcatggctgaggaggcctcaggaaacttacaatcatggcagaaggggaagcaaacacatccttcttcataaggcggcaggaaagagaagagcgaggagcagagtgggaacagccccttataaaactgtcagatctcctgagaactccctcactgtcacgagaacagcatggggaaaccactcccatgatccaattccgtcccaccaggtctttctctagacacatggggattatgaggattacaattcaagatgagatttgagtgaggacacagccaaaccatatcactcttcatctgctacgggtgccattccctgtttctacagtgtcctgtttgctaccttctttgtagatttctattcattcatcatgtcccagtttacctgttacctcttcaacagattattcttcagtctgtccctcccaaactccaccaatgaacattctttgttcagcccattattgttaatatgcacttcactgaaaacactgttttatgcttgtttacatcccatctctgcagggctctgagcctatagtcagcagttgggaacttactaggcatagcgcccagtaaaggatagctctttatatatatatatatattttttttttttctaaaataataagccccaagaaaaacattacctgtgaaaaacaaatgatatttttataccattttcaaaacctaatagcaataaaaagaaaagaaatgtcataagtatacatttaagctgtagttaaaaacagtaaataggcacagtatttaactcttatcatgtgctgttccaagtgtgttatttacatcattggctgttcacaccatcccctgaggtggttaatatttccatcccatgttaaagatgaggaaaccaagtcacagcagcctaagtgacatgttcatagtcacattggagctggcaggtggcagcactgagaatgaaacccacgtggctgagctctggaaagtcaccattaatcaccacactagaccttctctctaggatctacataactagaacatacaattaactggagtatatgtaagtgtagtcgttccttgagaaaggaaaagaattcacaaatttcatagcctcatcccttgtgcatttgtggcattgtgctaggggtgtctatttgatatttgaccctacagatttctcagatgccaaccaaccacagatactttaaaaaaagatacttaatacttaccaatgcaagaaagtgggcttgttggaagaggaaaaatgttagctcactaatggaaaatcaactaaaagtcttacctctccagaacatgcttttcctaaatgttgtggctaacccaagaagctacatcatctttacctttatgatacaatttaaagtacttttctacttacttcaagccctcagagcacagaggcaagggaggccttgggctataaaccctgtagcctgagccacatggcttcttctcctggacagatacctggcatattctgccactgaggatcagtggtgcagatgtatctgttttctcctaggaatggggctccacttacgaatctggggcaggacaaggtattgctgcattttggggagcgtgctggaaggaggagcagagtttgttgtttttgcttcctgatatggactggctgtgtctccattcaaatatcaacttcaattatatctcccagaattcccacatgttgtggagggacccaggggaggtaattgattcatgggggctgatcttctctgtactattcttgcgataatgaataagtctcacaagatctcatgggtttatcaggggtttctgcttttgcttcttcctcatttttctcttgccttttcctcatttttctcttgccgccaccatgtaagaaatgcctttcacctcccgccatgattctgaggcctccccagccatgtggaactgtaagtcccattaaacctctttttctccccaatcttgggtatgtctttatcagcagcatgaaaacagactgatacacttccttaacagaaccaattattttttcccaaaaaagagggggagaagcccagaagcatgcagcttctctcttctttctaaggaataggtcttcacaaagtgcccccaaaagaaaaggaccaggcttttctcttactttgtgactttcctaggttgctttgtagccattttggacacagaccacctatagctgtgtagtagttctcatgtgtatggacacagtggaaggagatgccaggcacatagttcttgctgcattgacaccttttcagggcatcttcttttaccatcttctgtagaaactgcaacagcccggccaactaccagccccagcccacccctagctttgctgctttcattttctctgtaacacttgtcaccttgtaatgtgctagtgtaacatcgttgttttagttttcttatatctttccttccctgtagaatgtaaataccaggaaggcagggaattttgtctgctttattctctgcagcatccctagactccagagcagaccctggaactcaaatatttgttagacagccaaaggtcatatacttccaactctccacccaaaatatatatggatcacccatccttagtagcagctaaacctttggacatgaggaatttgctcgttcacagttttctggcctagcatctgattaaccaaactttttcacatatcaagaatttaaaatgactccaaaactttttatgaccttaggtgcttctccttaattctagttggttcataaagctgtcagaacaataatctgcttttgataatatatgacttttcttgcctccaagatagcctttaaaaaagcaatttagccttttactgtaattatattccaaatttaagtcttgccagaatatcattttccccctttaaacatactttttatgatgccaagatattttaaagaaaagttcagataggaagcatatttaagatgtcagtatatgtctctaaaatctaagaacatttttctacagagtccagatgataccatctcacccacaaaattaacataaatttttagtaccattggataccatgtccatattcagattctccctgagttccagaaaggtccttcacaggttatgcatttcacatcctatccggcaccagggccagcatcttacatccttaattcttaggattcagaatagcactttttatatcctgacactgacttgctgaagaagctgtaccagttttcctataaactgtccactccttggatttgtcttatatcatgtcccaggggtattgtttagattgttgctctattgctaggatattcatgttaccctaagtgtgtactgagatagcagagtaaagagagattgtcttttaaaggttctgcccaggaactgctggtttacacaaaagctgggctttagtgcttctcggatctctttgcttgtttcagtaatttttttttagaaattcactaattcactaacgtaagtggggcagggtttacaggagcatgtgttgagagaagttggtttgactttaaatggtcatttttacaatccgtatttaggccttacaggtcgcatacaatctctgtcacatattgctgtcatttttaccccctttcttttctttttataatcctttaaaaaatgcaaagcccacccttagctcacaggatgtacaagaacaagttgcgggttgcagtttgccaagacctgctctgtgccactgctgacttgccattcttggagcccactttctgttcttgtcagccaattccctttcaggtgctcaggtttccctccctcctgtcaccgggcctttgcacatgctctacccaccctgcctagtggagagctatctttggcccctggacttcattcacttcggaccttctttgggtcacatttcagtcatgcctcactagacacctggaatctctgcctcactagacacactagcactcactcttttgggtcttctggctgtatgtaattgttatcagggcagtaattttgcatttatacaggtgctgaggtcagtgtctggcctttggctacttgccttgtgagccacctttgctttccagtgtacccctagctcctggcccagtgtccagcttcccagcatccattcatgttttccctggagaccattctgtgcccagcagccagagacatcattgcaacttaaatctcattgagtcgttccagttgctgtaagaaatgtaaccgtactgggtaacactatgcctgtctgtctccaccaccagatcttatacttcctccctcatctgctactccctaagctcactggccctttgttgtttttgctgaacgtgctaatctcttcctcacacctgggcctttgcacttgctcttacccctgcctcagccatccatgcactctcttggttttgccgtgcccgacttcagcatttttagttcacatggctcttccgtggggcccttccctgaccaccctgtttgaaatgctacccatccctggcacacatctatcatagtcattcattagcaattagccccatctcctttccctatagtacttttccataattttttttcttgctcatattacacctccccaactaatagatagattccaagaaattgagtgctttatcttcactacctggaatagtactggcacttggtaaagcacagcgaatattgttgattgaattactaggggttggcttagagaggtacttaatggaatgattcaatggatgaatgaatgaatgcattgccacttttttgctcattatcatcaatttgtacatatgcctttcaactcaaatatttatccattggctaaagaaaaaaaatacctgaatccccgtgtctctgcagtttacccttcctcactgttaaaactcagtgcctgtgtaaaatttcattatattttatttacttcttattccacttgtgcattatgtggatattcctgcctcatccccacagccacctatcacttccggccaactcagatgcctctggctcgcaacaatcacttctctgttgaatttctgaagcgtttaatctgcagtatgtctgttactcatacagcagttagtgtgaattgtctcactcagaataagtattcattaaatgtgtaagaatgatgatatagcctaatttcagatcaagattgtgatttatagctcattcttaatcagctgtgtcatgactttatttatttattccttttttttttttttttgagacagagtctcactctgtcacccaggctggagtgcaatgcagcttctgcctcctgagttcaagtgattctcatgcctcagcctcccaagtagctgggattacaggcgtgcacccccacgtccagctaattttttgtatttttagtagagacggaatttcactatgttggccaggctggtctcgaactcctggccccaagtgatctgcctgcctcggccccgcaacgtgctgggattacaggcatgagccacctcctcaggcctgagtcatgaatttttggtagtccagtcatgtggttatcttcctaaagctggaggccttaaacctagggccccaggtgggcttatgcctttagctgcaagtaacaagaaacccttttcaacctttttactgatttatttagagagaagtacacataagaggtaatgcaagtaagcaaagttttttaagaccacacaactagatagccagcatccggaccagaacacagagacttgctggcccctagaatcctctctcctggcccctgccagttactggcctttcccctgagttctatttgcctgcagtttgcttgttttcatatttaattcaaatagaattaggtgatgtgtccccttgtgtttcgcttctaacagtgcctttatgcgggagaagtactggctatttcttactttgccctccctccccggaaacattttcctaatttagtagcagtaatttcaaaaactgctttcctttcggacaaactgacttatccagccattgtaccttccagtttagactctggcagtttggagagttgagacagagagaaggagaaccgtctgtttatcttcttgaaaaaaactactgtattttctctctcttctgactctgggagcctctagactcttttttcccattttcatatccacagaggaagagtctgaacctcttcctgctgagctttcccctggttggttgcatttcaggaactgacattggttgtcttccctcttccctcatcagtgcaagtaggttggatctcatgatagtccatattcacataaccatgagaatcagagctttctctctgtcatttcctccctgtttatctgtagcaagaagaaaaaagtccgtcttaacagagactgtgaaagaataaatatagactttattgttttagagcagttttaggttcacagcaaaattaggcagaaggtactgagattctcccatatactccctgttcatacacttgcacagcctctcccattatcagcgtcccccttagagcgatttgttacaatcagtgaacctacactgacaccattattatcacccaaagtccatagtttacattagcattctctcagcattgtttattctgtgagtttggaaaactgtctaatgacatgtacccaccattgtagtgtcatactgagtacttttactatcccccaaatcctctgtgttcctcctgttcgaccctctcttgcctttaacctttccaagccactgatctgttactgtctataattttgccatttctagaatgtcatatggctttcttagaaaatacaaatggtgacttttttggcatgactttaataaacagaaatgaaaattggtgtgtgaatcaaaatctttcactctgtaaagggacttatcaggcggcacctgcatgtggaattatatcaaggtcagctttagttagaaacagaaggagagtcacctttgtgatgtcatttctccttcatccaagtaaaatctcgcatgctgacacataaaattttatcatcagtgatttatttcctcgtaggaatgtccctttgaccttattaaattcagatactctggggatggtcagcattttcagatggttcccttgttctgtaaaaatgtttgttattggtgtttaaaagctgtaaaagccacccactataaattgatagccactaaagtctgcgaatgtggtatgaatgttccactatgttgccaagcgtttattttattgtgagttgttatttgattgtttgtaaggaaattgttttatgatgctcagctttatatttccataaactgttcctcccttgcaactccttaaactgtgatgagcttttcaaaacaagtccacaggacaacagtctgtccagcatttgaagaagtggtggaaaagtcatttttttcctttcagtgtttctagctgttcatgaaaactgcagttaaaagttgtaatgttgttgtgtttggcattctttgggaagatgcagcattattcaaacaaaaccgtgtgctcttgttttaaatagtgtccaaatgaataattgaattcatggacatctaaatttaattccagaaatcaaagctatggtaataagaacttggacctgagttctcttaggccaagtaatttaattccttagccggttgaactatttattacgtgtctccgttcctggttattatgccagtaaattgctcaggcagatgcagggatggatgatgggcatataattcactctttttcacggctagacacataccagaatgcagatcacactaaacagctgtttttacaaacatggtttctattcagagctttaagggccacatccaagtaataacaaagaagcctggagaaggaacctggggatacccagggtcatgttttcaccatcccacctggaaacacggtgctgtccaaaccctggatggtacacttggacatcgtcattccagtcatggttcaagatcacgtgtatacatcatgatttaaagagaatactggtttgatttttcttcctgactttccctgaattttccacttaaattggaaaacagcgctagttagtgcccatgatttgattgaataagtaagagatctcaaacacggtaaattaactgatgacatgcatcaggtcatctttctttgattcaccagtaatacaacagtgagatcctacagaaaaatgcaaaggcagaagctcagggcagctgagcaactgttaaggattcttttgaaatgtagccccacgtagtaaactttcgtgcaataatttgggcagtcaaaaaaaaaagtcagttatttcacctcatttatttctacgaggagataaaggcttccaacatctgattttcagtgccagtgttttctgtttctcttttcacctatttcgtcctagtcttgtggaaatgttacaataactgattaggattttctctgtattaacacttcatttattaacatatgagtaagttgctattaataaattattctgcttaatcagtttctttttattctttagccacatgctaaacttcttcggatgtcttcacttaatatccttacatcatatttaatcagagttagaaaggcgttccacttgggggtcatacacatttttttgaaacttttataaacgccctttatactattcttgtatcctacgtaatcagagtcaggtgttccatggggatcatacacatctttatacaacttgtgtaacacatgctttaaatcctgaggtaatcagagaattcctgccattgatggattaatctctttacatatctttccctttcactctcagcaaaactacttatggcggtcctgttagactttgaaagcctttcaggtttatggaggagtcttcctttctattgtcgtagatcatggaatcatagtgttcgcttgtctcacgtgtttggaatctgatttcctacataatgctgtgctctcactacgagtccaaggcttcctgtggctacaaatcccgggttcctatcacttttcccaatgatgagcttctccttgttggccagaatcagctctgtggtaataattcccttctgctttctccctctcgcttttttttttccttctgggtacattataatgtcagcatagcagcttttaattctattactatggtacttctcgaatagacctgtggtagttgccccagaaaggttgaaaccccctgccacatctaggtctttctcctgtattggttttgtgatctgatcaaggacatctattcctctagtcctgctaggtggtatacgataggcttttactattagatattcaaatgctgtcaaccatgcctgccttgacaggtacatgatttccacaaggatttcctaataggcctttttaattttgttctagacatgcccttcttttgccatccaaatttgcttattttttacaaactatgatgcatcagcatgtaaatgtttaataccataaatatttctgatcctcttcccaatttcactgagttactggttatctcttgccattattctcagcatattgcagtggatctcagaatattttcccctgaccaccagcatcagtattacctgggaatttgaaatgcacattctcaggccccaccaaggcctcatgattcagaaactctggagctgcattagaaactgccacaggcacctggtgatctgtgttggtttttgtttgtttttaaggagacagtgtctttgtctccctggctggaatgcagtgatgcagtcatagctcacagcagcctcgaactcctgggctcaagcaatcctcccgccccagccttctgagtagctggaactacaggtgcacaccaccatgcctggctggtttttgtttgtttttaaggaggcagtgtctttgtctccctagctggaatgcagtgatgcagtcatagctcacagcagcctcgaactcctgggctcaagcaatcctcccgccccagccttctgagtagctggaactacaggtgcacaccaccatgcctggcttttttttttttttttttttaatttttttggagacagggtctcaccgtgtggcccaggctggtctcaaactcctggcctctaggttttttcccacctcagccttccacagtgctgggattacaggcatgagccaccacatttagcaggtaggtgatctgtgtttcaataagccttccaggtgattctgatgctcactgaaatttgagaaccactgaaatatttggttttctacaaaacataagcagttagtaaccatagggtacgcaactaaatagttccagcaggcatccctgaccacagggcattgcagatttacttaagtcagtttcatcttccacctgtgtgagttccagcaggcatccctgaccacagggcattgcaggtttacttaagtcagtttcatcttccacctgtgtgagttccagcaggcatccctgaccacagggcattgcaggtttacttaagtcagtttcatcttccacctgtgtgagttccagcaggcatccctgaccacagggcattgcaggtttacttaagtcagcttcatcttccacctgtgtgtccagtttaagctctcagttgcagtttttaaaccaatttccaccagcatatactgagtatccgctaccgtgcaatgcgttgcggctcttgtatgtgcactatgagaagtaattcccataatacggcaaactcagttttgatatccaccaccccactttttataatggactacatttttcagagcagttttaggtttacaaagaattcaagcagaaactacaaaaagttccctctacctctcacttccccactccccaacagtttcccctggattaacattttgccttagtatggtacgtttgaaccaatatcgatacactattattaaccagtattcctgttttaacatgagaggcgactttttgtgtagtatagtctcttggttttgacaaatgcttaatgctgtgtatctattattacttatgacagtatcgtgcagagtagtttctaaaaatcctctctactccacctattcatcccttccccactccccctgaaccactggcaaccgctgatctttttattgtctccgtaattttcccttttccagaatgtcctgtggttagactcatggcatccccttttagcaactgcagaaactaagaactgtccccaaggataaaaatttttgcagacccactgagctgggatttatacttagacctgctgaattgaagttttgggtgtgtttcttctcaaattgccaaaattccatatggacgacttttcttttccttccctgaaatgtgtttaattgactttttctgttatttgtgtttgccttcacagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgaggtaatccaccatttgcttggattccccccacccccaaggaaaagaaagcgtaataccagagttggaaatatccaccctagcaccactgccttccccaatcaaaaacatgtttttttttccaaaaggcttcttatgcttgtgaaatttttttggtttaacaagcaaacaatttcaaataatgtgaaatctttattatacagtttgttttgtaccttgtatatgctgcttggcaaatcccaagttaattctacaagactccgcccaaccaggtagtcatctaatttgacaaacactgagttaggtgactccttggtattcctttggcagtgagtgtttgttatataatattgtctgttaccacctagacgatgagctccttgagggtaaggacttgattttattcagtattgcacagagtgcctggcttgtagtttcgataaatcttagttgagtgagtagttgtgattagatttcattaaagaaggagatttgggccaggcacagtgcctcacacctgtaatcccagcactttgggaggccgaggcaggcggatcacctgaggccaggagttcaagaccagtctggccaacatggtgaaaacccatctctactaaaaatacaaaaatgagccaggcgtggtggcacgcacctgtaatcccagctatttgggaggctgagacaggagaattgcttgaacccaggaggcggaggttgcagtgagctgagatcacaccattgcactccagcctgggtgacagagcaagattgtctcaaaaaaaaaaaaaaaaaaaaagatttggtgagtatcgcattattttatgctatgaattttaacagaggtgatttatgttgagtttacattgttttattgggaagtacggcagtgctttcttcaagaagtaaatatagtattaatgtcttagactttctctattgaattaatgtttgtgttgcaaatattgtgaactaaaagaactaaaggtcatacttgtctccatgaaataaaaacaaacattcagttgatattaaatgaccaaatgttacagaaacgagtaaactgttggattgagttccttaaagagtatgcatttgttccttaaagagttctgcatttcatatctgccatctgcttggcagatagtaaaagatgcttctagagctagattgaatgaatatgtacttactgaattttaatgtactgcaaatacttataaaggtcaaccacaaatgttacagcatttacaaaaagtggctttctctcactttttgctaaagtttaatgatttcagatttattgttctattagagcttaacatgggctattttgtctccctcaactatattcatagttgtcactatagtattttcttccctttgttgcattttttaccttattaaggtgtaatttacatataagtttcatttgcttttaagtatataattcagcggctttaagaaatttgccaagctgtgcaactatcaccataatctaatcttagaacattttcgtcactccagtaagatcacacatactcactcccagttaagccctctactcacctctagccccaagcaacaactaatctactttctgtctctatagatttgcccttttttggactgttattataagtgggatcatataatacatggtcttttgtgtccgacttctttcacttaacacagtatttttgaggttctacatttctttaacttgagcttttaaaattgagattattctgaattaaacactcagcgttagcttaaaatttattgaaagttttacatgttctttgaaaaaaaagaacagagtgcaagatggattaagtttatcattccctacttaatgacagtttcttactcactttagcacataagtaaatgtgctagtaaatgcgcattttgtttaaaccaccatttttcaaatactgagacatgctttctcagggtgtttttcatacaactgttaggaccaactatggcagatctggtcaaatgaacttgttagcctgatgcctgtagcagggaaggacagcttttcagggcttctcttctcgaaacctaaatgaagtgtgaatacaaattacagcagctagcagcctgcagtgggactgaaagtccttggtgaatctccatcaagctgggctgtttaatagaaccagtttagtgtcccattttacttgaaacattagattttgagcacctgcttggtacaagccattttgctaggccatgtggaatggatgatgagcacacacagaccagctaaccctagtggatgttaaaatctcttaggaaatagaagtcaaagaagtacaaagccatctgtgttgtaagaaatgtgaacacagaacctgcctccgagagtaagagggaacttctggattgatcagagaaggttccatggagagcatgacatttgaactagagccaaaagtgtgggtgggtggtgtgtctaccagtggattttgctgtagagtcatcaggactagcaaaacaaatgtggtgccatggaaaggcatatttaggagacaggattggtccaatttaagctagagcagatgtcttttaggttgggaccaaaatatagagaccttcaatgccaggctggcaaagatgggcattaactgcaggtgattaattaacaaatattaattcagaagctactgtgcgctgcagatttttccaggagacaataaagagattttgttttgatgctagtactgttgaggtaacaggtgaacaaggagtgacatcagttatgcagtacaatgagaggatgccacataaacttaggcccatattctagaacaagacaatttgcttgagataaattggtctcattatcgtctgcattagtccagttctttttgcttgagttttgcaggtagtattatttgatattaagacggaacacagagatgttgtattgttttatttcctgtagaaagacgaaggcagttaaaagaatttcataaacaaaacactccaggctaaaggcaaaaaagaaatgaaccttaaaaaataacaagttaataattttgttggcaagaagagcaacaaaagcaaccttctgaaagcaagatacctggtcattgagagatagcaactgttaatgccccagaaatagaggaatctttaatgcaggtccattcattccacctcaaactcctgaacaaaagctctaattgaggcaagtcttcctgggaaagggggcctaacagaaacatgttatggtcagttgtgcccatgcgagataatttttttgtgcttgtagctaattccagtaaagaagtagatgttttagagggtaacgttaaaggggtcagctgcaaagagtccttttcctcctcctcactgttacaaaaggtgacttgtggttcttccctaattgagagcaatggcaacgccataggtgattactcattttgtaaaactctaattacaagtaggttgcttctgtgttcaacataaaaattagaagtgacttgcaaagatgagatactgcagggaggcttacaatcttgttttcagtgttagtgtttcagtacctggaacaaaatgtgatttacagccatcctttctaagaaaattgtcatcattaccaacatattgctactgataccagtccttaatgtgcttcataaaaaactcaataatttcataacccgttaaaaccggaaagaaatggagggaagaaaaataagacacttttagcgtagtcataatcttctctgccatattgtgttagaatcgatgatacccaaatgagaagatgtctgttctctgtcactgattccagtagaataaaattattgtttaaaagtaagcacatccgctgcactccagcctgggcaacagagtgggaccctgtctcaaaaaaataaaaataaaagtgagcacacgattgcaagatctatctggttttgggggcttattgtcatctttggttgttaaaaaaatacttggatattattggttattggtctagtggaataagccattaccgatttgagtacaacagtggtgtatttagatacttgctcctaattttctgttcttcaagaagggattgtacttcagaaaccagagatatcagggtagttttgattgagccataaattctaaattctgattgaaatgggtattgtaataaatggcataatcttttatttaatctagccaattgtgccctgtccagaggaactggacatttcttccttatgttccatttattcagcaactattcgttgtacccctgctgatggctaggctctttgctactgtggaaggcatcgttggggcagagcagacagcattttacctcctgggagactggcagatttttgaagcctttgttgcagatatagattttcacggtcaccactcttgccctttaatgtaatttaaatttttttttttttttttttgacacgggattttgctatgtttcctaggctggattcaaactcctgagttcaaacctcagcctcccaagtagccgggaaaacaggcctcttgtgctttaaagcagtgattctctgtctttaaaccttgtaactatgctgtgtggtggaaattttacttccccgtttaaagccaagatctctaaggccaagaattttttttttttttttttttgagatacagtctcgctctgtcacccaggctggagtgtagtggtgcgatcttggctcactgcaagctcggcctcccgggttcacaccattctcccgcttcagcctcccgagtagctgggactacaggtgcctgccacgatgcccggctaatttttttgtatttttagtagagtcagggtttcaccatgttagccaggatggtctcgatttcctgacctcatgatctgcccaccttggcggcaccccccgccccccgccccccgccccccacaaagtgctgggaatacaggccaccatgcgtggccgaaaaaaatttttttaattggcctgggctgggcgcaatggctcatgcctataatcccagcaccttgggaggccgaggcaggcggataacgaggtcaggagattaaggccatcctggctaacacggtgaaaccccatctgtactaaagatacaaaaattagccaggtgtggtggcatgcgcctgtaatcctagctacacaggaggctgaggcaggagaatcgcttgaacctaggaggcggaggttgcagtgagctgagattgtgccactgcactccagcctgggcaacagagcgaaacttcgtctcaaaaaaagaataaaataaattgtcctgggtcacataactggttaagtgatgaaatcagaatttggacctagataggccaggatatttctgctacactcatgtcaaaattacactttttattttctttataaatacacacaaattcatttcaacctgtgttcccttgtaaatgcatatacctttgatataggcatatattcatctattattttacactgtgcatttctgaatttttagaatttttacttgaagtgatcaggctggcgctgtggctcacgcctgtaatcccagcactttgggaggctgaggtgagcagatcacctgaggtcaggagttagagaccagcctggccaacatggtgaaaccccgtcttcactaaaaatacaaaaaaattagctaggcatggtggcgcgtgcccataatcccagctactcaggtggccaaggcaggggaattgcttggacctgggatgcggaggttgcagtgagctgagattgcgccactgcactccagcctggcgacaagagcgagactccgtctcaaaaaaaaaaaatagaaaaatgatcgggggtccacaaataagagtaatgatcaagggtcagcaaattggggccaaaaatagcctgttttcctgttttctacacaaaagttttattaggcacagcccatcccatttgtttaaaaattgtctatggatgttttcatgttttggaagtagagttgaggatcgcaaaagagattgtgttgcctgcaaaactgaaaacatttactatctggccttttcagtaaaccatttgctaacccctgattctcactataagacggtgactgtgaagttctttctatttcacatactgccatatcaagttgatttcttttgaggtagggtttgtttttttttggttagagatagactagtatagtgtttgagagaatttttatgaccttgggccagttaagtaacctgttttttatgtgtgtagtgtagatggtaatgttatcttgcatgtttcttgtaagaatgactttaggtaacttatgttaaacgcttaccatcatgcccagtaagtgttcaatttacaaactcaagctgtccagattcaaatctttgccttacaagctactaactgtgtaaatttgtataaatcaaataaccttgaatcctcattgcttctttttgtaaaatagggattaataagacctatccaatatttaactattttgtgaggattaaattaattaatttataggaaacactttgcataattcctggtatacagtaactgctcaattaaccttagttgctctcttagttatggttatatcatcattccgatcctccattgcatttgaaacattagcaaactaatgtttactactaatgaggaatgttaaaggtaaaccaataagttgacttttaattataaaaggcataaatgtaacctatccatatctattggtataataaagctgatatttagagagaacattatgttatgattaaaagaaacatttcttgcttaaattttttttgtgtgaatatacactgtatattgaaatatctgcacttaaattttaaatccagctgaaaattggataagatagtattagatgccactgcatttcaagatagagttttatttcttgcaggtattttaagccttgtgggagttcttctgttaagtgaacagatagagacactgatacagatggctctttcaccgtctcatgtgatgataaaagctaaaaggtttgatccgaatattgcttatattgggctaagtgattgaccttggcagaattaaattatttttcttcttctggtgacaattaatctgggagaagtaccatttgatttattgctatacatttcaccaatatccacaaggcacttgtaggtaccagacatggcattggttgctgggggcacagagataggaacaaagtggcttgctcaggttgcttctagccatatctgcattgtccttgagagggatgagtctctcaggcctgcctaggaaggaattttggcagcatggagaagagaaagtgctcagcccggaacaaaagttttcacctactatgttgcagagaagttggcgtatcagtaattataaaggaagaatagggatttactgggtgatagagcaccaaatagaatgaatgcaatacatacttgaagtaaatcagagtagcttgtattgatctcaaggggagaagaaacaaagcttagcttaattctcatgcctctgttgttacttgtttgtaatggacatgacacttacaaggtatcctataattcactaacgggatttttacttatgattaatatttaactctccacaaagcttggggtaaactgtgaacttaacttgctgtgaacccgaacatgaactcacatacttcagttggaggcctcagtagtagcatgctgtacaaaaatgaaactaaagctccagggagtatttgggaacagggcaaaagggtaatggctcctccttatagagcaagcctcaggcctttacctttgtgttttgaagatggagaggatgcacttctactctcgcccgggttgtagcatgcttgctaatgatgtcaagagaactagtgtcagcactgaccatagatgtgacaggtaacagcctgttattttgtgccccatgtcatggcagccattgattccgaggtgtttgatacacactttgatagaggcagagtgaagcaggacatggacaagcatctttccgtgtagtttaatggtctcaatctagaaagcccactcagataattaggttgcttaaaatgtgatgagactagggaatagagccatatagaagtgtgtgtgagagagaattcagtggtttagtatattcacaaagatacacatttgacaccagcctgggcaacatagtgagaacctggctttaccaattaaaaaaaaatagaatagcagaagtaatcagtgaaaggcattttaatacaattatatctagatttattaaaaataaaattttagtccaatttaattaaaggccagaaactagatttttaaagaatattagtctttctcatattttccccaaatcagttgctttaaaaaaaaaaaagtgtgcagatgcctcaggcacaataactgtactcaactcaagaatttgcaattaagaaaaaaaaaaatcaaacctcaaagaagaaatccagcaactcagaggttcaggttcatttaaacatgcatacatttacatgggcacactttttttgttttgttttgtttttaagatggagtttttgtttgtcgcccaggctggagtgcaatggcgcgatctcggctcactgcaacctctgcctcccgggttcaagtgagtctcctctctcagcctcctgagtagctgggattacaggcgcacacaaccgcgcctggctaactgttgtatttttagtagagacaggttttcaccatgttggccaggctggtcttgaactcctgacctcaggtgatccacctgccttggcctcctaaagtgctagcattacaggtgtgagccactgcaccagctgggtgcacctttttgtttgtttgttttgttttgttttgttttgttttgttttgttttgttgacatggagtctcagtcacccaggctgggattcagtggcacgatcttggctcactgcaacctccgtctcccaggttcaagcaattctcaattctcctgcctcagcttcccaagtagctgggactacaggtgtgtgcaaccacacctggctaatttttgtatttttggtagagatgggtttcaccacgttggccagactggtctcgaacttctgacctcaggtgattcacccaccttggcctcccaaagtgctgggttactggtttgagccaccacacccaaccgggcacacgtttttagccaagccataggtctgattaagtacagtcagattccttgagcaggttacaaagctgtggatttgaatggagatctttagttcccttgcagaacaatgagtttccaaaagcagagtggcttacagcctaaccagctgcttctctcagcctttaaccattttgccctcctgaaaattcatctttggtttccaaccacctcttccaatatgcctgactttttgtgattctgttagtagaagagtaaaaagtcaaggggaggcaagaatgaatgaatctcatataaaaatgatgctcaagagaatcccttaattttcaagacagcagcccaaattgtaaacattgagacttttacagtgttaaaaaaaaaaaagtgtttacaggccaggcatggtatctcacacctgttatcccagcactttgggaagctgaggcaggatgatcgcctgaccccaggagtttgcaagcagcctgggccccatagggagaccaaaaaaaaaaattgttgttttggctgtagtgtcacaggcttatagtcccagctactcaggaggctgaggtgggagggtcgctggagcccaggtgaggtcaagcctgcagtgagccgtgatcacaccaccacactccagccttggtgacagtgagaccctgtccaaaaaaaaaaaagattaaggcagaggaaatgttaaaggttacgctatatttagttgataataatggtaagactgttcaggtgttttaactatgagagtagggttgcctctttcttaaatttatatatgttcttattgggtataatttctagacagaatatgcaagtatcacagaatatggatggcaattttatgttgcttgcacgtgactttaggttcttatgtttcatgataggagcaaagaaaagtcttgcttgccaattcttatgattgcatctgttctggaaatttactagcatttgttacccaggatttctgaagtgctggtgactgctgcccagtagtcttggtgggcaaagttactaaaatgcatcacgaatccttctggtgtttgcttgggctatggcagggtttctggaccttagtactattgatgttttgggccagataattgttgcggatgattgtcctgtgcattgtaggatgtttagctgcatcactagcctctactcgatgccagcagcacccccaagtgatgacaatcaaaaatgtcccctgggaggagtaggggatagactctctctggttgcaaaacactgggctatggtgttgagatctgcagtgatgagcagctttagatatgtgaacagaagcccaggcgtggtggctcacacttgtaaccccagcactttgggaggctgaggtgggcggattacttgagatcaggagtttgaggccaacctggccaacatggtgaaaccccgtctctactaaaaatataaaaattagccgggcatgatgatgggtgcctataatcccagctacttgggaggctgaggcaaagaaatcacttgaacctgggaggttgaggttgcagtgagctgagatcgtgccattgcactccagtctcggcagacagactgagatgccgtctcaaaaataaataaataaaaataaaaaagtgaacagagaaatacccgtgcaatactgtgtctcatcccccaaagacggagtcttgctgtcgcccaggctggagtgcagtgacacgatctcagttcactgcaatctctgcctccatggttcaagcgattcacctgcctctgtctccgagtacctgggattacaggtgtgcctcaccaagaccagctaagttttgtgtgttttttttgttttgttgttttttgcttttttttttttttttttttttgagacgaagtctcactctgtcacccaggctggaaggcagtggcacagtcttggctcactgcaacctctgctgcccaggttcaagcgattctcttgcctcagcctcccaagtagctgggattacaggcacctgccactgcgcccggctaatatattttttttttttgtagttttagtagagacagggtttcaccatgttagccaggctgatcttgaactgctgacctcaagtgatccatccatctcggcctcccaaagtgctgggtttacaggcatgagccaccaagcccggctggtctcattttaatgtaagctctaacttctgtatcactgctttgcacattgtagataaccagtgaacattttttattgtataagaaaaagtaaaataaacaaaaatatgccaaagcaaatatgataaatacacatttagtaaggtaaaagcaagtatttgataaaatgtgtaatgtagtctagtagctttatttagaaacagaaaacaacataaaatgttgacttgaaacattgcgtatgcatatttgccatgtagacagtgagatatggaggtaaactgctttcctagttttgttttgtttttttttttttttgttcaaaaggaattcactctccccttgtccactgttataaaatagcaaatggtgattagcatctgtgctgtaaccagagacactgctgttcccagctggaatctgtaggttctttgcctgctagagattgtgacaagaagccagttaacatggagaggtgtacatgtcagtcttccccagaaaaggacatgaaacagtaggaccatccttccaacccatgacctacatctagaggcattctttttttctttttttttttttaaaccggaacagaaatatcatctaattctttctttacagagaagtggtataaggaagtacattctggccaggcacgatggctcatgctgtaatcccagcactttgggaggccgaggtgggtgcatcacctgaggtcaggagtttgagaccagcctggccaacatggtgaaacccctcgctactaaaaatacaaaaattagccgggcgtggtggcacacacctgtggtcccagctactcgggaggctgaagcaggagaatcgcttgaacccgggagacggaggttgcagtaagccgagttcactccattgtactctagcctgggtgacagagcgagattcgtctcaaaaaaaaaaaaagaaaagaaaagaaaaaggaagtatattctaagcccttgaattgatcttggctttcttcattatttttacaagtaaaaataaaaactttgatatttctgatgttctgggatattataggaatgataccacttgaattcagctttctaagaagtctggtatttcaatagtcttaatatgagtaagaaaattatgatcttggtactggatctaaggcagaggtatcgaagctgttttaatgttgcttgagataggacagaacttcagagtaaaataaatgtgtatagacaatctgttcaatctgttcaaattaagttgttgagctgtatatttgaatcttaagactactaattttgttgtatctgtcttttcccaaatatatgtgtacttcttttaataagccaaaacctatgcaacttttactaattcttccctaactacttcttttggaaaatgattaaatcttatgtctcacgttttgacatatctattgcatttaaaagataaggttgatcatgctgcttctcaaactgagatatccattcgagttatccaaggcccttggaaaacaatcgttgttgagtctgtatggggggcctgagtcccaggtggtgttgactctgctggtccatgatcctattttgagtagtagactctagtagcgtctcctgccttccatatggtgcagatttgcagcattgagacaggaagtaagaaactgaaaaggaaaaccattcttacctttggtgttattgttcttggtatattacagataccaacaagtatgaggcaatcctgagacatctacctactgagcttttctttccttaaattcacacctcaagaccaaggaaaccgaaaaactcttacagtctccctacatgtgttcacttgtgcccctatttcctgtcattaacatggtagtgtagccaactcctgctagcctgtaaaagctaattattacagtatagaggaattttgtaagccatagccattagtagcttgaaataccaatggtgggagtatttataccatggcagttggcatacactgtaactcaagagtgttctatttgtttgtttttctagacagtgccagtttcccagcatactcctgtctctcattcagtaagaaaaagcacttccccttattttccaatacaggacatgatatcccgagttttgagcttcttgttaatagcctgttgcacgctaaccacgcagctcagtgaagagcaagtttattcccataacaaacaggaaacaaaggttgaggaaaaaccacagtggtacccttttgtgagtgttccctaacaccgtcacgggagtcctgattagtctaaatactttacaagtaaatctccttcaggtggcattttataaacaatctttcatcatggagagatgagaattgggtcaggtataattagaatgacagatgtattggctagcctgagagaattaacgcaattcagtaaaaaggctagccaactgcggatatcttctagttacttccgtgacaaattagcaccatcctcttgtactgaagcagtgctttcattagcatgcacagctgtttgcatcatctttgtttgtatttcaatccatatttggagcttaagtgagatttgtcataagacttcagttcaagcttgcttgcctaattttacatgcttctatttagtaaaatatgattactaggtcataaatatttgataatttattgccttattagagtaattttctaaacatgttttttgacgactgcttgccactacgtacaagcacttgaaaaatgttcttttctattaaaagtacatggttccagaattgggaaaatcacgttcttaaaaaataaccagttttctattgtgttggcttttaaaattttttcatcagtagaatcaaatgcaaatattgtctgcttatttttcaacagaatacaagtttgattttaataacttaggctcatgaaatgtcaccacgtagtaatacttagaattacagcaataacttaggctaaaaaaataaagttgttatgaactacattgagctcagacagatatatgtattatgtaaacatttatataaaatgtatataaattgtatattgtattatataattatatattgtaatatatagtatataatatataatttataaatataatacataaattatatttataaattataaagtttttatataatatattataatctaatatattataattatatttataaattataacgtttatttgaaatattgaagttttgtatacttagtggattttaatatttatgtcgttttcattttattccactgttacattaaggaagtaacttattacaaaaataaattatatttgtcttacatttgccctttttactctacttaaaattggagaaaaagagttcaagatagggaatttctgtcagaaaggagaaatagttgtattatgtgaagtttaagtagatattcttgtagagtatggagaaaataatgctataaaatgttgccttttaaaaaaaagttcagaaaatatagttcctagctctctctaaatgactagtttgattgtgactgctttcccaaaatttgtaaatttaaaatttatacatcacatggaatatagttttatttcacatacattccagtaggctgagaatacttgaaattcaatgattaaaaatgaaataataataaccagctttgctttattctaaaaccagctttattctttatttaccactttatttacagcgaattatttgctaatctgttaactttgcttcataagaaggcattataattatcgtacatttgaaagtggagattttctccccattctggttgccagtttcacaactgtgttcattctaaatttattaaaatatgtaaaatcagctttctaaccttggttgtccacaaagaatttcactccaatttgaaatatctattcagaactaattttttgtggttgagcattattttttgaacaatgaattgcataatcattttgtataatccagggagctctggcttagattctgtattattttgtgttatactgatttttagaaaatggaatgtagcgttgacacgagcacaaatctcagcaagggcctaccttacaaagataaaatgcgtggagttttattagaagcacatgattttgctttgtgtttcgtatacaccagcatttcagtgtagtaaatggcattttgtcttgaagagtgagcagagttgttacaaaagaaggcataatgcttccttgccagcagaattcagataagtgaatatcagaaaagctatttgtgatcttgaaagtgtaagaaattcaacatcaataaaggattgtggttccttagatagccatgtaagtctttgcctatgtttctaacatttctctttaggaaatattcttgcttttatttttctgaacaaattttctccattattatctctgatatttactttgaacagaaaggtggattggaaatcaaaaaataagtttgatttcaaaaatgatttcaaaatgttagttttctcagtattttacattttgagaagagaacaaagaaggcagatgaagagttttttgttttttcttaaatttgttttcttttcaactttatttttaattgacataataattacatatagtatacattttgaagatacggttttaattttcacaggaatcttatttgggtttagttatataaggtctaattttgcatcctctacatttagtggctgatttataaatttccactgagtgataatagtaattaatgtgaaacaggttttttgctgatgtttttagaagtttcagtagattaggaaggaagaaattatgctattgagaaaaatagaaaatgtaaacataagcatttgaactctctcttgagatatttttatcttccatgaaactagatgcggcttattgaagatgtgtttcaggtctcagtgctctaaaactcttctacttggtgagcatcttgattggttagatctcttactaatatcccattgctagagagggatgtaaaggaaattttctcaaactgagatttccaaatcttccaatctctgagtcaagtaaagagacaaggttagaactcttaggcaaactgaaaaatcaatattcctatattatcattatttttttctttttttgacaccaagtctcactctgtcgcccaggctggagtgcagtggcactattttggctcactgcaatgtacgcttcccaggttcaagtgattccagtgcttcggcctcgcgagtaggtgagattatatgtgcacgccaccatgcccagctaatttttgtatttttagtagagacagggtttcgccatgttgactacgttggtcttgaactcctgacctcaggtgatctgcccacctcggcctcccaaagtgctgggattacaggcttgagccaccatgcctggcttcctatattcatttttttatgtgggctttttaaaatcttttcaatagcaaagacttgacaccaacccaaatgtccatcaatgatagactggattaagaaaatgtggcacatatacaccatgaaatactatgcagccataaaaaaggatgagttcacgtcctttgtagggacatggatgaagctggaaaccatcattctcagcagactatcacaaggacagaaaaccaaactctgcatgttctcactcataggtggaaattgaacaatgagaacacttggacacagggcggggaacatcacacaccagggcctgttgggggggtggggggttgggggagggaaagcatcaggagaaatacctaatgtaaatgacgagtttatgggtgcagcaaacaacatggcacatgtatacctatgtaacaaacctgcacgttgtgcacatgtaccctagaacttaaagtataataaaaaagaaaaaaaattaaaaatcttttctttgagacaattgtagatttacatgcagttgaaagaattaatagaggccgggcgcggtgcctcacgcctgtaatcccagcactttgggaggccaaggcaggcggatcatgaggtcaggagatcgagaccatcctggctaacacagagaaaccccgtgtctactaaaaaatcgaaaaaaattagccggtgtgtggcgggtgcttgtaatcccagctactcaggaggctgaggcaggagaatggcgtgaacctgggaggcggagcttggcgaagcctgcagtgagtggagatagtgccactgcattccagcctgggcaacagagcgagactctgtctcaaaaaaaaaaaaaaaaaaaaaaaaagaattaatacaaagagaccctgcatagtcttcactcagtacagtatcacaactattaaattgacattaatcccttaacactgctttaacccaagtctatgctgctgcttctccgtgttcacttctgagaggcactggccatttcttgtgtgcttattcatactttgtgtaaaagcattagcatcatacatcgctggtgctgaccctaccctcagtcccagcttctcccagagtaggctaagattctcttttctactacatggaaaattgttctggaataataaagggttagcaaaaaagtcagagaaaacaaacagttgcattccctttccctgtagtagagtaggaatataattatgagtgatagaggatacgttccattcctttcccagtcctctccgaagtcagcttcactgctgtaggaagttgctgttctcattatttccctgagggaaagtgttgggaagtgagagttgaggtgatggggaatcagggttgctgacaattgggggaattataacgagaagctctttttcaccaggagaggggttgcatgttaagagaaggcctatctgctcatcctccatgtcagcagatgagaaccagttggacaaggtagctgtctcttagagattgagaagtaacaaaagcacactgtcagtcctgttcttccagcctcacgtacatcttctccatacattatgtatttgacattaaattaacatacactgttacacccattcctgaccgcattatctcaggtagccttttgtatttgagtattgtgggatctgttgctattgcccatgatgcccacttggactccaccaaggcagtgaaactcttgggggccgttcaaagtcaagagtgccaactgcccctaacaggattgggagaaccaaccaacactccccgaatagggactaactcactcttttcttggcctttatgaaaggaaggagttaaggctcttacagctggagactccagcaagagttgtaggctcaacgtgtagcttatactatttacttaattaacatgtaaatggattttgagttctgaatgcttgttctagttgattagggctcatttagcctggcttctacgaattagctataacaaatggtttcatagctgttatagaatgaggcagaagaaaataaaatgaatgtagcattatgtttaattagtggtataggaaaatgttaatataatgccatttagattctggactggggagattgtgcctttatcataagtaagaaattgttgctaggtttcacataagggcacatagttatcatgaatcatctccttcgatcatcattcttttatcccattacttgacttgcacccatccaaaattctaccacttttatttaatatatagactcataaagacaaggtttgtgcatctgcagagctgtgatgttcactgcgaggcccgtggcacacatctataagaagacacatatttggtactctaagatgatacttttttttaatgccaaaatgtgagggtcagcataaattgccaaggcatggtaaattttgttgccaatgctcccgtgactatggggtgaagggattctgtgcctcccttggtagcaatgaccacggtgcttgcaatcccagtgccaattcccggcacagtgcaggctctggaggcactgctgccttgcagtcttattggaattaacactgcctgagtaagagattaataatgggtattgttgtgtcttagaaacgttaaagagcatgtgacatgaatatgtacacattggtgaaggagatacagagaaggaaagcaaagtgaagctgtagatgtttgttacaactgaaggtaagctctgggagcagagaccactgctggctatagttgatgggatccacaatgtcaatacaaatggggactaactattgttttacttccctatacacgttgctctttcagtagatgcaataaagtactggtaaaaccagaggtggctaccatcacgatgatgtcaacaggagggacagtcagcactaagcccagaaggtgtcaaacactccgcaggagaaatgcgccatgcaacggacatgaagatgatctgacactcttcacgtggttttcagatggaaacgtggctacgaaagcatcaacctcattatcatccatcattaaggccatctcactcagtactgctgctttcaaagtccacgctcccaaagcaaattggatttctgtacacaatactcttacaggatgaaacccaaccaacttgttgacgtaagtatcccatctacttcgcaattttatttatcttccaaaattaaaggactggcaccctgatttattaaaagtgaattgggtctagggaccatatcccctctgagttactgacagagcagcttctggcctgtgaagctcaaagccatgcctagatgtgatggatccttgtctttgattggctaattagaatcaatctgttgaaggaccagtggcagtggctcacgcctgtaatcccagcacttagagaggctgaggcaggtgtatcacttgaggccaggagttcaagaccagcctggcctacctggtgaaaccccatctctactaaaaataccaaaaaattagccaggcgtgctggtacaagggtaatcccagctacttgggaggctgaggcgcaagaatcacttgaactcaggaggtagaggttgcagtgagccaagattgtgccactgggcggcagtgcaagaccctgtctcataaaaagaaaaaaagaatctgttgaatattcctaacttggaagtaatgggtgatgcgtggctttcatcttttagaaaggtgttttttcctctgtcagtaaaactaagaagacatctgtgtgtgctaaggcgtcagtctagcaagggcagggcacaaacccagagagagaattgggctgttgtttcacttccaataaaaatgggatatacatgggaggtagagttataattctactcaggattgactttatacatgcaattggatgggtcactgattgactttgaggcaatcagggatgagtgaaactgatcttggggagggtggcaagacatgttgacaggtaagatgtgttttaatgtataacagtgcgtggtgtgtattaatcagttggagttgattgactgcgttttattgtttggaacattcctgcccaagggtcaggtaaatatacaaaatatacaaataatacagatttttaatacataaatatcaattaagtagcataattaaataatacattgttatagttgtacttaatgtgtatgtaaatatatacaaataatacagatttttaatatataaatatcaattaaatagcagaattaataatacgttgttatagatgtacttcatgcatatgtaattaaattattaatgtataatatataaatacatattttaatatacaaataaaatggtcttaaatcagagttaatttaagaccatttatattagtacatatcttaatatacaaatgaatggtcttaagttaatgtcattgagtataagtcacatttacagatatgacatgtgtttccagataaagtcaatggacagacagcttccaacactggaaaacattcaaaatatatgcaattaattttatatttgagttaaggcaatcatttattaaggaaagcaatcagatgtttgtgagggcttaaactttcgcttcataaccaaagcagtttccttacatgaaagttgacttcttaaaaagagaagtttattttttataatggagaattgttgtgatgtgcagataccttccgtcatttcatactcatctaaaagcagccaaatgatcttttcacctttggccggaatccttttattgctaccgagtcctgcactcattaaaccagatgacagtgcatgctctgagaggtgtaaatctctagtcctggtggccagttaaattcctcagtaaatgtttggtagatgctgcctaataaaccagtccaggttgccactgggaggattaaaagaagtaaacgtgtatacatgaacagagagacagtgccttttcatgctaaatgtggttccccacatctcctctgattagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccagtaagtggacccttcttcgagcctggccacctttcgtctctctcgccactgactctgctttttgtaacagattgattttcctggttcttgggaatgggcctgttgctaccactaaccacatttctgtccacttctctaattgctcagagtctccgcagtatgttcaatcatgagcacacctctccgtctttccctgataaagcatggccatggatgtgttctcttcctagctgtagcacatatgtcttgcaatccagagggacttttgagtgcttctcttttaaacaaagctggagtggctgttttgtcttctgcagccagccgtttctgcaaatgcatacttatgtgtttgtccgtgtacatatgtgttcatgtttatgcatgtgcactacctgtgcagatactgtctaccactggaagcctggctgatgcagcttgagttgcattgataaggtgtatggctgtgatgaatacctttaagttggttcataacatgtcttttattgtgctgatgagggaatgtgattgaattgagctcatgcatctcaaatgccggagggtagaagtgcccgtcttgccccctccgagctcagcacacagacatttgccaggtcaaacttctgcttcttctcttgaaagtagaagaaaacttttgacttaacgtgagaaactttcaatgacataaggctcgaggaacataattttattttttttgtttttttaaagacagggtctcattctgtctcacaggctggagtgcaatggcgtgatcttggctcactgcaacctccgcctcccgggttcaagtgattctcctgcctcagcctccccagcagctaggatcacaggcacatgccactgcgcccggctaattttttgtatttttggtagagacaaggcttcaccatgttgaccaggctggtctcgaactcctgacctcaagtgatccacctgccttggccatccaaagtgctgggattacaggcgtgagccactgcaccccgccagaacataattttcaaagtctttctcttgaacattaagcttttcataattccgggggataaaaatatgaagtcatgatgacttaagtgcatgatgaaattttccccaaagaggtaattctcaggaggtccagtaccagatgtgagacttccatgtcgttaacagacttccaagtgaaattcccaattgtgcttatttatttatttatttatttatttgttgttgttgttcatgagtaggaagtatgaactactcaagaagtaattattcagctcagcttgataactaaaaatgaaactcaataactgtgtattacttttttgagagactcctgcctaattctccgctgtgttgcaagggtggcagccacatttcctgcctgagattcctagggattatataaattgttttgcagcaagatcttaggattaagcaaaacaacagaagggcctgcaccattaatacccccactgtaaaggagcccaggatctaagaggaatgtaatggaaaagctcctgttggtctcctgccatcgccagtgaatgagatgactgcttcagtcccatgtgtgaacaggtgtaggaactgataacctcaagggctttttttttttttttttctggctctgattggatgattgcagcttctggctcaaaggactcccttcttcagtttcaaacaaagcagtcaattaggtcaggtattgcgaacacaatggagaattctgttgcctgcagcctttggaacaaaggccttttgttacagtgattctcactggtttagccggtgggagaggtttcctggcagatttgcaacttttttctccctttccctgggtggattcattttctgaaatatcatgtatagtttttggtaataagagatttatttggttttaattattagcagtgatgaatttgcttcaagagattaaaattctctccacctcaccccagtactgccatcccttcacctaacactctctccctttgatcagtttgggctgggagtgaccagtgggcacctgtccaggtgtcctgggtggcacacagtttgctgagggttttgcaaaaggtcgatgctcccaacagagatgtattagatgaggctagctatgcctgattttagaggggaaaggtgacagaataatgtatgaagaaggcaaaggctgaagatcagttagatgggtggacaagaataagtagagagtataagcctcagcaaacagccagagccgagagccacatgctgtcctatgaatgagtgaaataaatggagtttttgtgaaaaaatgcatcgaagggaattggggatatttggcacaaaaggagcaagaaatcctcttctgttaagaaacctgttgttactcggatatactatcagagacaaaatatccagcattctcaaatgttaacttcttacgaaaatagatcttatgtttatatgttcattttggttttgttggagggaccaaacctaagtgagtgattttgtttgttaggttgtttttttgtcagtggactcgtgcatttcagccatcattcccatgtttctctttttgtttttagttatgttctcttattttttccatagtgtcccaaagtttactcaagactacccaggaacctcttgcccgagatcctgttaaacgtacgttgtcattcacctgagggaagggaagaggggaggaggatgctgcttggttcacataactccagcatcatcaccttctttgcatggttttgtgtttcttgaacacctgtcttagtaaaatgtttcttcccattaccttgcttgtaattacatctgattttgccagacagcttgagatgttgggctaagaacatcattgactaagtttcttctatttctgaccaatttcctttttatttagtctggttttattgaatattatatggacaacatcattgtattgtatttgccattactattttatttcctaaaagctatcagtgtaactgagagcaggcttagcctctcactgcttttgcagaactgaagaacaagggctaggtgcagtggaaggaaagtgactttacttagcaaagctagcaatggggaaatggtccaggctcctgcctcaaagcaaccatctcaaattttggattaaaaaacaaaggcttaaaaaggggagcttggaatgcagggcatgaaggaggggtgaggaggtgctgctatacaggacttgttccaaagacttgagttattatccagttcggtaagtgggctggcgcatcccgcacaatcgggttgtaaattaactgcagccttgaggtgatctcctgatgagggagaattctgaaggtgcctaagtcagtcagtggaacttctaagcaagcattaataattttaataataattataagcgtaattagataatggaagcgctgtgcagggagtgcctggtgggaagaaagagagcaaaggttaccatttcattactaaaactggaaggaaaagaaaggaagaaagatctttaaaatagagtactcggttacatcaagatgccatgatttaacaagagactataatacataagtaacttgtttcctcagaaaaagtacttaatattcatggtattgtagtacatggactaattcgtacacgcagatttcaatttatgtgtgttataagacccggtttcaactttagacatgaagggtcacttttagacattaggaacccctctttctcctctcccagaaggcataatggtaggttagaggtgctcaccttttgactaggaagtagagaaagagcctcctataaagaaatgcattgtccagccactgcaggccttacttacagtgtcttatgttttgtttttatttcagttgtaatatcagacgagtttagttttaaaatgtggttttagtactcatatttgaatttggattccatccagaaatagatttatccagtgttaggttagggatgatatatgccatttatgcttatgattgctggaagaagaggcacaatttcagttgtaacttagatattttataattagcatttgtgtacattatcctgaaaaaaaaaaaccctcatctcaaaactgatgaagccagttatcttttttaaagatgataaattatagatgaaggattaggtattttaaaaaagactgcatcctaaactataaagttttttaatggcctccataaaatattcagaataattcacattatgtataacttttgaaggatgtggtttgtactgctgcaacataaacgtttcttttgctgttgtgaaaaaagcatttttctctaaaaacactgtaagtacagcaagtgggttgacagactaggaaaaagctctagctattccactatagtgttgacataggtacgcatgttgagttgcattgtaccgagcattttcacagatcatttaatttttcttcagaacactttgtgggcattttcctcattccttttgtacatacgagaaaaaatgaggcttcaagagcttagtgacttgtcccaagccatcgaacatggcaggaccaggctttgaaacccaaaatgacgttttggccgcactccacaccaaatggctaagcattggttctgacattgttttgccggttagtgaaatcacatttgcgattcagaagcacattttaagaatacagaaaacagtgaaaagaggatgaagaatgttaacattttttcctaaatgccatgaatagagcacgcatcagtaaattaaatgtctttcccattcttttataaaacagtgattatttttctcttgtttcattttagcatttataagaattctatttttccatttttaataactgatggaaacaagcatcattgtctgataaatttattttcatgctgtacacgtaactaccactttcatcatccactgttccaaacagatactgaaagtctcttttgtaattttctccttcccagtaattttctccatctacttgcagtgtatttgggatcctgacacctttccattctaggaggatgaagtgctttgggcagctatattttcttgtctgcctacagagactattcaatcacagcaagtgaagggttttgcagtgctagtacaaacagacaaggaaaaaaacatataatttctagttatttatagcctgtgcagctatgtggtcttatttatagcaaactgcacatttgatttgaaaaagtcatggtcatctggaggtcaaattcagatggcagactcaattcagagaaaaggagctgaactagaaggtaagcaagcctgttagactgggcaacttggacttgactttgctccttcaacaatctagagccagttggatgatatttctgcacaaaggtggttggttttacaggttgctcagtctaagctgcttctagtcaaataacaaatgtgaaaggagatagaaaaagctcttcagttgaactgataaaagattacacttagggagaaaatgtatgatggtttgcattataaaaacaataatttgccataaatcaggcatttctaggtttcaaacttttgtcataaaaaccaattttgagcattgcaattttagggacatattttattgttgttattttaaataaaaatctaaaccacaaatataaggctgagagctttcattttattcgtgaacataggaattcaaatcattaaaaaaaaaccctttctcatttgggataaagaatcttacgagagctgagttaatttctgacattagttttgcacctgcccatttgggtttaggacagtgcaaaaggtttcacatttaatcttcaaaatatagtatttgtagacaataaaattatataaatatttgtggattcatttatattttaaatatgagagtgtgaccattttttctggtgctgtctccatcagactgcatcccaaaaggcaaacagaccaactccttaatggcatttaatctctcaaagagcatcatgccaatttttagtcagcagattgcaccatgtgatctggccaaagtgtgtcccatctcaggctccttcatttgcgctgacatgtggtacaaacaactaaaccaaggtttctcaacctcagcactattaacattttgattcaaataattaatttttaggagtctgtcctaggcatatgggtgtttagcagtacccctgacccctacccactaaatgccattaacacaactccagctgtgataactaaaaatgtctctagacattgctaaatgtgcccagggggtcacattctggttgagaatcctgttctagagtagtagtttccccacatctgactcagagatagaactctcctggtgtaaacacgtgtactttccacaaatttgggggcatgttgaaatgttagaatgtcagcatcatccaattctcgacagatgtttgaaattgaatttgttagactattgtccaagctctcaatatttgcaaggtaaagtggaagttgctgttgctgtcctgactatagtattgaaggtctcgtagttttcctgctctaatgtaagctactgtggcagcagaagagaaaccagcaaatcccttagtttgaagtcattggttcacctatgcaccacccctttctcctctctgtttcatcttagtttcctagaatcccaggatatgctggaggacaaccatctgtagagatgtcctatagtcttgctacacaggttatttgtactttaggattatgtttgatggccttatagtttgtgcagaggtgtaggcctgaccccatgcaggattgagggagggaaaaagattgatttctaggcacatcagaatggtccagatgacatgaaacagactggtacacttacagagaatatgaaaagaattggaacttaacaagtcaggccccaagttccatccccaggttccaagtttgttgccgcagaagctcataattttcaaatcgtagaaaactcacaccacgcagcctttcaatggaatatgccaccttaaccccagggtcgttgtaaagacaggagccataagacagaagtctgaatgcctaggccatttaatctttggccaaatttgaactattgttcttaatcacactattcttgttcttttcactgtgcagtagcaaatgttagttcccattagcacattctgcaataccattggaactaaaactgccaaattagattaggcaacctgggattacactttgctccccaaccaacctagggagggagaacaattttaaagcattaaaagatgtgatggtgaattaaactacctgtcacgtcagtgccagtatattctatcagattaacaagtgtgaaaagctaaatctttggtatctggaaatgcttagataggaagaatggctttttcttcaaagccataataattagagatgaccctaaaattgaaaggaactggattgcttagcaactcagcattttttaaaaagaagggccatctactttgaatacaggttaaatatctcttctctaaaatgggaccagaagtgcttcagattttggatttcggaatattgatgtattcatgatgaaataacatgaaattcatttatgtttcatgtacaccttatacaaaagtctgaagataattttatacaatttttttttttttttgagatggagtctcgctctgttgcccaggctggagtgcagtggcgtgatctcggctcactgcaacctctgcctcctgggttcaagagatccccctgtctcagcctcccaagtagctggaatgaatgacaggcgcctgccaccacacacagctaatttttgtgttttttagtagagatagggtttcaccatgttggccaggctggtcttgaactcctgacctgaagtgatccaccagcttcggcctcccgaagtacttcgattacacgtgtgagccaccacgcttggcctatacagtatttttgataattttgttcatgaaacaaagtttctgtacgttgcactgtgagagaacaaaggtgttgctatcccagccaccatgatgtcatgtcagtgctcaaaaattatcagattttggagcatttcagattctcagattagagatgttctacctgtggtaaaagaatgttcattatgtctctgctctgttgtttagctatttgccaaaagagggtaagtggatcgtttaataaagtggtcagcaaatatttttcatacaaggccagattatagatattttaggctttgtagatcatgcagtttgtcataactacttaactatgcagtggtagcatgaaagcaacaatagacaataagtaaacaaatagacaaggctgtgttccgataatactacttttataaaagtagccagcagctgggtgcagtggctcacgcctgtaatcccagcactttgggaggccgaggcgggcagatcacctgaggtcaggaattcgagaccagcctggccaacatggcgaaaccccatctctgctaaaaacacaaaaactagctgggcgtggtgatgggcgcctgtaatcccacctactcaggaggctgaggcaggagaatcacttgaacccgggaggcagaggttgcagtgagctgagatcacaccatcgcactccagcctgggggacaagagcgagacttcatctcaagggggaaaaaaaaggtagccagcaagctgatccttggtttaatacagcaggtttttttatttttatttttattttttttccaatagctttggggatacaagtggttttagttacggggatgaagtgtatagtggtgatgtctgagagtttagtgtccccatttcactgagtagtgtacattgtacccaatacgtagttttttatccctctcctccttcccactcttcctcattctgagtctccaatgtccattatgccactctatgcctttgcataatcatagcttagctcccaatttataagggaatacatactgtatttccattcctgattacttcacttagaataatggcctccacctccatccaagttgctgcaaaagacttttattttgtggtttttctggcctactagtattccgtggtatatatacaccacattttctttatccctcattggttgatgggcacttaggttggttccgtatctttgcagttgtgaactgtgcagcagttatttttgaccccaggatgtgcgaggccaggtattttgagagacaccagcaaaggaatctgagtactggctatcaatacctgataattttattaatgagttgagacccagatatgccgtagaggtaaataaccatataaaggaaatatagaagtgtaggagaagattgctctggaaggctggggtaaaatgaaatcagctttatccttaagacctccatagaggttaactgtaccatctccttttactgctacagtttatatgaaactggacaatattgaagattttgacttttcgaactcatctattgagtgttgagcagctatgacaggttatttctttatgtgcctccattaacgaagtcaagaatagcttctgacaaattgacaaaaaacaataacctaaggttaacccattctggacgccagttattgctattgcttcacatatattttttagattcaaacttgaggttgcttttaagttttagtgtgatgtgctgaatagagaatagatgttaataaaaaaataatgccagcattcgttccttcacattcagttcatatatttgaaaaaattcccatgtcagtatgtgtccatggacataaaaaagaaatgtagcttgactagagtaaacacatcatagattatatttttattaccttatgtccatgtgatgcattgatagtgttctccaccaactatgattgcactgactataaaattgtaatacaagggcatattagggggagaatgcctttgttttttcttttgttgttattttgtttttaagagagtctggtattacactggaggatcacaaactgtattttcttgttataaatggctcctctatcttggacttaatggttaggataaatatttgaatgtagaatgcgttaggagttttaactatttttgaaattaggaaaatggagagacactttttttttttttagagacactttttttttaagttataataagcatggcactcggaaagctctgccatgtttcaagattaaagtttcacatgaacgctaacaagtttatatttcactcatatataaatgatcctccttgtctcctttcctaaattatagccatctcaaatgacaacattgctgggtcggtatcaaatagcattttcaatgataaggcaatgatttatataggtcttaatttgttttaatcttctgttaccaaggctgtagagataaattgcttttctgctgatgaaaggagactaacccaattttcttgttagaatctaatattaatatatcgactgaatgccattcataagccttggtttagggaaacattcagccttctggcctgttggagatggccacggtcattgctgtttgaaggtgggtcatttcttcttttgtgtcctgagtccattgtgactgtaaaattatgggtcaccccagagcctgaggaaacaaatgcatatcaaaatgataggggcaatctttcacagtgcctttaattttgaccccacattcttatatgaatgtgaagtaccacattagtatgaatatgaaatggagtgctccgcaatgattacaaaatcagaactaaaatgagggaaatcagtgcatgtacagttcaacttaatggattatcctctaaggagtcttcagacaggctaggagagtgacgtctgcctcatcctgaagatattagcaggttttcccctgttttaaagtaacttcccttcaaatgctaacagttttctcagaggaaacacaggtatggaaaatgtttcgttggtgtcttggccccagtgatggaggagcattttgtgataggaacgttgggtgcagcatcgccatttatgaataaactgaaagctaacctgtgttacttcaccccagaaaacagagctctggcaggtattgagaatagatcacagtgtgaagtggcagtattgtgacaagcatgcgtgtatcagtattaaaattgaaatgagccctcacgcacatgtgaaagcagtttctcccgttttcatattcagttccctgaggtaatacagcattttggtgtgagagaaagaagggaatctgacccttctcgtgtgggaagggggctgagggcaacaacctcctgtgacagccaccctggtccaagccaccttcatctttgtctcctttttttttttttttttttttgagacagagtttcgctgttatcacccaggctggggtgcaatggcacaatcttggctcaccacaacttccgcctcccgggttcaagcaattctcctgcctcagcctcccgagtagctgggattacaggcgcctgccaccacgcccagctaattttagtatttttagtagagacagagtttctccatgttggtcaggctggtctcaaactcctgacctcaggtgatctgcccgcctcagactcccaaagtgctgggattacaggcgtgagccactgcacccagcctgtcttttcctcttaaaagatcttccttctacttttgtcccatagcagccagagtgatccttcaacaaaacgccccatcacatatactccccctcaaacagcccacgctggtccctcagaatccttactgtggctttaaggctgtacatgatgtcccccatttcttccctgcctctcccactacttcctctcttgcttctttagctctggccatgcgggtctcgtggaatttgccagccagcttcaactcttagggcttccgtgctagttttgtcatctgcctgccatgtccttccagcagccagctgggaagatctgaatgtcacctcctcacttaagactctgcctggtcgctcacttactgcggtctacctttcccctctcccctcagctctcccgatccttctgaactctgctctgtttttctttttcctctttttctccataatacaaatcacatattaacaatatgacttactggcttttaaaaaattcttgtggtaattgtttctttccctagaatataattccaccaggatcagggccttcatgtttttctcattggtttctcccatgtgcctcacatataataagggctgcaatgttggtgggctggaggaataaaagaatgaacaggcgcactaaaggcaggatagtttgtccatgtcagagctgctgtctcagctgctttgatacctctgacttcccagggtggcaaagaataaggatgtggcttatgtagaaggcgttagtcatccaaaatgccactgtatatttggaagcaaatattcaccttgaagcagtgatgtactaggaattagaactgcttttctgttacattttttcatttgttatgagtgtttagatacacatgatcactattaaaaggaagggaggtaatagcctatatatcctaccatgcccctcaaggctctgttaggagactaaaatacaaaggtcatttaaaaagttaaaagcttatgcatgtgatgttggtattgctgctaatgactaggcatctagaggtcaaaacctagagctaaggatattgaaccctgtgttttgtctcgttttaagtctaggtttttgtggaggggggaacatacctagccagcttgcttgcagaaggcatgagtgagcaggctgaaggagggacccaaggccagaacgtcctccatattctagctccatgggctctttcacttgatccctgctttatgacccaacttctgttgaaggatttgccagatagtgtttggggcaggaacagctgtttcatagaaaacaagccaacggacatcataacaaagcatatggttgttttgccaagttgtgctgctcattaacaaaacaaaacaacaaaaaccctttacagtgccccagcatgtggatggattggccctttactactctaaaaattagctttacagcctttttatggcttacccaatcttaacagactttatttgtgtgtgtttagggatgcaaatgggtatgttaaatgatagtgttttgcctctacgcaataaaatagggtaattttttcttcccatgtattaaacgagaacacagaacatgcctagcatggtgggctctgatgaaaagaggctgttatcatttggccaacatggaaatcctagcctttatttatttatttatttgagacggagtgccaacatggaaatcctagcctttatttatttgtttgtttgtttgtttgagacggagtttcgctcttgtagcccagactggggtgcactggcatgatctcggctcaccgcaacctccgcctcccaggttcaagcgattctcctgcctcagcctcccaagtagctgggattacaggcatgcgccaccacaccctgctaatttttttattttattttattttagtagagacggggtttctccatgttgatcaggctggtctcaaacttccgtccacctcggcctcccaaagtgctgggattacaggtgtgagccaccccatccggccgaaatcctagcctttatcagaggttccgtggaatctcagaggtctgtgagcaggataaactgctttatggtagtttaaaatgcaaaaccagaaataaaccctcctaaagccagtttgtttccactttctaagtggttacagtgacctgtgttttaagtaccttaaatatatcagtgacctgctgatatacagcttcctgtttcctctgtgtcctgtgacatatcctgacattccttgagccccactcccttcccaaaaggaagtaccttgatgggtgaggacatagggcacacgtgtctctggccttctgtggacataatcacaaaatggtagtcagtcctatgaatttaatctctttaggatataaggatattcacgaaatagaagttgagctctctggacatactccaccttaaaattgctctctccttccgggatcctcaccagctgcagagcagtagtaatgaactattaaattcatttaacaatatcagcatatgctatccttctctgcttgactagacagtaatctggcttgctggttggtatgaatataggataatttcactcacactaattgagatggggagtttgtatgaatggctgaaaaatttgagttaaagataaaaagagacacagcttcctcaactatgggaaataactgggactgttaacaaagatttacttgaagagaaattgtattgctcagggttctccagagaaacaggaccaataggatatatatgtataggaagagattgattctgcagaattgactcacacaacaattatggaggctaagtcccacaatctgttgtctgcaagttggagacccaggagagctggtggtatgatttagtccaaatctaaaggcctgagaaccagggaagacaatggtgtaaatttcagtccagggagaagagaagaccaatatcctgactcaggcagccaggaaataaaaagggtgaactgctccatcctctgcccttttgttttcagaccctcagcagatcgggtgattcctgcccatgttgaggagggtgatttactttactgagtgcaccaattccgatgctaatctcatccagaagcaccctcacaaagacactcagaaacaatgtttaatctgggcaccttgcggcccaatcaagttggcacagaaaattaattaccacagaaatatttctggattcaatggattaagttggtaatatagtattcatattcatttccacgtgcctaaaaagaaggtttttatcctttttaatatcctcagtcctattaaacatacctcacattcatgttccatggttattctcttattatgttgctaagtgcatttctacttaggccaatctgattatagtgattcagattatagtgtttatcttctttcctttatacagtagacagtgttcgcaaaccataggagctgaaaaaatttccttcttatatagtaagttttatgattccagttttccaaaggggtgcttatgttatggttataacagttacagccagatgactatcttcatcagcaggtataaccccaaatatttatacattttcatttgtttcaaacataaaatatggtttgcctaaacatgtcattctgccagattggactatgcaaaagaatgttactgttttattttctgcttttactttaacttgttctcatttgagctctaccagttaagatgagaagatgactaaagctagggaagcacattttaaatgctaaataccatgttcatttcaagttgggaacttgaaatgcaagtatttttaacttaatcattaagttaaagtcagtaagaacgtggtatttagcatttattcattttaacttaatgattaagttaaaaatacttgcatattggtttgcttgaagctatggagaaaaacatttaccttttttccaaaaaggtcttaatattcaaaattattgtttcaatcttacaaattaacaatttacttatttttgatactttcggccttttttatgtgggctacacctaacattttttgaaaaatacatactgcttttcctgtaggttgcactttgtgatcctgtgtgtaattcattttcagtggttctattttatttattttttgttagtttgtattttcttttgtttctttttctcaatttttaaaattttaaaacttgtccttttcttttttttctttattattgcactcttctaccaaaacattttcagtggctttgaataaaagtatcagaactttcccgtggttagcattagctctgagatttgaacaggatgtattaagcagaggaggagggaaggccactaacatgtatgagctccactgtgtgtcaccctctctgccggatgacttacatgtggtatggtatctcattttcccagctctcctatcatgtcctgcggcatgttatcaccacttgataaaaaaattgaaacatggagtgcttaagtttcctgccccaggtcacagctgatctgaaaaagaaatgggattcaaatccaaatgtgcttaattgcaaatccccacatgactctttccttccttccttccttcccccgttgctgccaatgtaagaaggaacaccactccctcatttgctacagagaagacaggggcagctgtgtaaaggcaccaggaatatgccctgcctccttccttccacttgaaagcaacatctggatagacactcaactgcttttattttttgaaaaatgattattaatcaggattgaagtggagggaaaaaatcaaaacgaatgtttctttttaaaatgaaagatactgtttaattatttaacagccttccggttttagttaatatctttgaagactttggattaagattccagacattttcttgaaggatgtagaaaacgtatttaacttcaagtttagtttcaaaatgacttagctatcaattcttctcattgaagtcctctttgagatattatatcccaaatgtaatacacaggtaataattcaataggaatacttggaacattggtgatatttgaataaccttttgaatgaaacaattgatttgatctcaaaagatagtgaaaacgtctcattttgttttttatgttaaatatgcttcctcttaagtcaaaattcttttttctttttgcttttaagctcataaatactctgaattctcactaatcttttggaaaattgcttgtataggtattataattcacttggctcatgagttacataatatagcagtatatgttgcaattcaaatattgcttagcagtattagttgaattcaaaatagaaatcactttctttttgacaactagcataatgaattcattgactattgtcaaatttatgaagacagaaaagtccttttgtgattcaaaaatgtgtttattgatgtttatatatcttttgtcgtgtttccatactgtctcatgaaggttgtcttcgtaattcagttatctaaatacatcatacatttaccttttccctggggtgccgttattctacaacaggctaattacagatatctgtggtttctgcaggagtcttagtgtatgatattgatgatatcatcatttactttcaaaattgcaaaatagtaatttaggaaagcatggggatttggaaaaacatgtcttcagcaccaactgtttttgctctttgcatgcttgtttcgtaaagaactctaatgctataattgcaaaatggaccattttaaagatttttccttcattctgtacttgggagtggtgaaagacatccttactgtgctgcacagtgtctcatggtgttctcttaaacagcattaacgtcttgtatgcgctgctttactaaattctctgttctgagaaataactgaaaatacggctttctattaaacgagtggattattctgttgttgttggcttttttttctcaaacctccttctcttctactttatagttcctacaacagcagccagtacccctgatgccgttgacaagtatctcgagacacctggggatgagaatgaacatgcccatttccagaaagccaaagagaggcttgaggccaagcaccgagagagaatgtcccaggtaagtctggctcttccatcattcagccctacgatattgggaaacctgagcttgcctctgcctcagtctccccacaggcctgctggctttatcaagatctttaaagatgtaaagttctaattttaaatgtttactgtgtgggcacagtttgtgggttttttgcatcctccgtgaacatgctgcctaggaaggatccaggtatagttggattttgtaagcacaaattaaatgatgtctgtaacattctctctggcgaagaaaggtacttggcattattattctgtgtatatgtagtacctgtaatcaacatgttgtaaattaaaatccatcaaaatgctctttagtaaagatgaggttttgtattaaatcccagactgttataattgtgaacctacgattgtttacagtctgtaaaatcatagagacttaaaactgagctgaaccatagtgattatttcatctagtctcctaagtgatacatagtggaatagaggttcgagagagtggcttcccattttagcacaaagcaaaatcagtgagccagagacaacatttagaatccaggcctttcttgctcctagtacagtactcctttcattatactttgctgaaaaaaaaaaaaaaaaaaaaaacctgttttggtttggtttgtttttttttagcaggtccttaattaagcaaattaaaaaataacttatgtctaaaacatggctaatccatatatactatcaactacaattgaagctatgtctgctaccaggcactggatgttgtattggagtttgataatatggaggattttagcccttgctcagattcctcattcattcattcattcaatcaacaaatatggacttattattttccagtcacttttctgatttgtagtctacaaacctaggagtgaatgtggggggaaaagaagtaggacataaaagtgaatgaataaagatctcaggttaaagtaagccattcatttttagtgttaatattctgtggcaaatatcagaagataggagaaatttttgtcttagcacagatgcttaatctcttgagagacattaaatgcaatttatgaattagactgctgcgtattttctaagatcacagtcagtctttagtagacaattaatctttcctgtaatttctgatctgggtccctgctgaccccaggaagattcctccctcccagggccaaatgaatggcccctgagcatgtctttcccatgcaaaccagtcaatcaaaagctccttactcccaaccaccttcttatactctgggccactattcacctgctctaatcaccccaggaccaggtaccagaccactagggacaacctcatgccccatcaccattatttgaactaatcagtcctaaacccgcctaccctgcttcgctcattctttccatgaaaaccacaataaaaggctcttgctcatgttttcccctcctgaccaaccctggtgcttcccatatgaccctgcatgtagtggcatgtaccggcttcttgggagctgagtaacaaactgtcttttccatgataatcatctcctgacctcttggcctcacctgaataataagaaaatctacccttgaaaacataaaactaatagtttatctagaaaggtacaacacaggtactgttttttgtttgtttgttttgttttttgtttgtttgttttgccttaagatgaataacaatacttggatctactttggtataataacataaagaggaaaaaagccttataatcacagtacttttggctctggaatataatgtgcagttctagtggctgcccacgatgaaaacctaacagctaattgaactagaaagctagccacagggaattattaagttttcacctgatgccaagaaaatcagatctttccttaggaaagacacaggcactgttcggaaagcattggaggactatgaatcatacttattgccactgaaatgtcctcatttgaatttacatcctatctcccttctttatgaagctttcagaggcttcctgtgccctcacaaatccacttctgggcacatgaagtcgtaaaaaaagaacaatgacaaaaataaacaccgaatgctcagcttgtcattttcagcgcctaccactgtaccctgactctggtcttcctaccacctcttccttctcaactgctacccttgctttctgtacagcgttaagctgctgttctgatcaggccactctgctcatcgtctgttcatctcatcttgggcttccctttgcccttggcattgagttcttctggaaatgccctcccactcttcttcacgctgacacatctcacccattccttacaccccacttcatactcagaatgagacaggcagtcattgctcttacctttcatttaattctaaatatttgctccccggttttgttagtgatttctggacacacctgcattatctttccaagctggatagaaaccttgagacctcaagtcatgaagcctgctacctgcattcatcgtggacccactgtagcattttttttaatgtgtgtttttttaatcctggattttttctcctagtatatcttgttcatttttgaccttattaagaaaacttatttcaaattctctgttagttttagatgtgacaggtcttctttaaaataataatttaacttttagcacatctgtgtaacttgtgacttagttcatgattttgtagattttgttcatcttttagtcatcagatatatctgatataacctaagagatgttgtcccagagcagagaatgttgtctactagattccagtggtcataggaatgtgagacacctgaaatcaatatgtatcttcatagtctgtggtgctgaaatctagggacttttcaactttaaacttgcctgcctcttaaatctctacttagtagtagtcattttctctgcatgaaacagccaagtctttgaagattgataaatccaaatgaaattgatacctttgtgcctgcagtggaaaattgctttataagaatccatagagccactgcaagggccaggtcctgagaattttaaattttaaaaattgtgcggttttttaaattaagttttattgtagtttttgttatatgttttcttacaaatacagaggattttggaattttttcatattttggtttgttgttgccattgtggagtataactctaaacctgtagatttttgttgctaaacctgtagatttttgttgctatatgtcgtgactagcatctagtcaaatagaaatctacagctgaaccatgtgtgagcacttttattacttataaacttactacttttagtatatgcttaaaatgaagtccttcaaattgaaagctgacccaactcataggtttttatttcttttcttttttggagatggagtcttgctctgtcacccagctggagtgcagtggcacaatctagactcactgcaacctccaactccctggttcaagcgattctcctgcctcagcctccccagtaactgggattacaggcacgtgccaccacgccgagctaatttttgtatttttagtagagacagggtttcaccatgttggccaggatggtctcgatctcctgaccttgtgatccacccgcctgggcctcccaaagtgctgggattataggcatgagccactgtgcctggcctaggtttttatttttaagataagcagcaacattttaagagcgcccctttctcctgtcactaaatataaataaacttagctcacctaatgaattttttattttataataaaaaattgggtaccacagggtacaaaaacatgatctttagagccacagagatcttggtttatattcttccctctcccttttttacctgcatagcctcgagctggtaccttaacctggttcgtaaaatgggatggccctggctaatctcacaagctgttgtgaggaatcaattaaattagatagattttgtaaagctcttaatacagggtcagcagtaatgctacttgttatcagtcccacagtgaaattcactgttcacagggagatggtcagcctacatatagtgtgtattcatttgcagtagagtcttagtgggatggtgcaggtgatttgtttatttgtgggggtataggatattgttaaattgttctgaaaatcaaaatgtgagttaatttctatagtaatgcttatttgcaactcattagctaaataaatctctttaattatttcatggattcccccagcttaaagcttaaagtccagtttcttaaagtgtatgaggtaaaatggaaaaaatgtaaatgtaatatgggaaatcagttctcacattgcagtagaacattgaacttctatctcaaactaattttgagattccttgcttgaagaattgttggtagagtaccaccactgtgaacattcccaaagcaggaaattgaaatatttatattgcaaggcttatctcatctataaagcaaagatggtcttccatttcccccttgcccacaggttccctttattaacttggtagaagcaccaaagcagatgggacaatatgtttcttttcttccatgttctattcttgttcattgcagcagggcatttgtcccaattttgctgtgaaccttgagattatttttgatatgcttttcaagtgtaatgaaatatttattccatcttgtttcctccatggttttacaaaaacaacaagaagaagctaattgcctatcatatacaaagaatgaatgccaatggacaataaaatctgtaattaggaacccacatggtgatttgatatagtattgtctcatagaagtcttctatctatagttatttattccaagcctctttttctttcccatcttgaagtatgttagaagagtctattccttaaggctaagtacctacaaaaaacaggctcttataatacagtaatcaagttttctcatgctttgggagtacacagagtatgcaagtacacatctggcaccaaaccaatattttgtttccttgatctttcttcattttacctagttcagagttgcacttcaaacaaattctacttgtcatacacttgaaaaggcagctgtaggtggtaggaagttaatattttctttgaacaacatacagaggtgcagacttttactttttgtatcaggttaatgagtgaacttctctcagggaaatccaggtgaacatcaaagttgagagcaggaagcattgatttcctacaaaacacattcacctgatatcttagtcagtttgtgttgctataatagaataccccagcctggataatctgtaaagaaaaagaagtttaaaattcggctctcagttttgcaggtggggaagtccaagaacatggtgctggcatctgttgaggacttccttgctgtatcatatatggtggaaggaagaagggcaagaggagggcaaaactgagttttataacaacccacttctcacaataacaaacccactccccccataatgatactaatttactcatgaggacagagactgaatcacctcttacaggacttacccctcaacattgttgcactggggattaagtttccaacacatgctttttaggggttacattcaaaccatagcagttggttactaaattagtgctacttgtaaaaaaatacatgtttgctgcattcagaatattacatgttttcttggagtttttggaaggacttagcgtaatccttgctgttcatgacttaggttggcaagctcaaagggccttaattccccatcagttctttaaagggggataggtcttaggatattagacagcagaagtcttctttttcttcccccattatgtactgctttttggcattgtattcatgacacacacacacataaaatgtcatattttctttaaagtcaaaatatacagtttaaaatggtttcagttgctttcataagagagtaaaacttaggtgttcagtttaatattggaagaccttgttcagttaatgtgtgccttccatgaatagttggtatttgttcacatcaaatcaaatgactgtcagttctcggggtaaaaaaagaccaaccttgactcaaaaagctatattcctgcaataaatatgaacatttatgtttaatatactgctgtgattaattatttatatcctagtgggaggtcaaatattcttcagataggaaggggtatgtaataaattttttaaatatctaaaatgacaaaaatagaggaaaaacataatcatccatcctattaagtctgtattcaaaggatgaactgatgattttaaattcaaatgtttccttaatttataggtcatgagagaatgggaagaggcagaacgtcaagcaaagaacttgcctaaagctgataagaaggcagttatccaggtaaaacctgaacccatttcctaccaaacatcacatgccagcagtctttttaatgagtgtctgcaggtttacctttatctgccagcacctcacgtgtgattgttcatagtcccatgaacttcagctgttcttaatcattgtctagtaattgagttgtttaaatcctgccttatatttgctcccaattatttcaagttataattcattgccataaaactcacattgtaacttgcaatctactttttattaataatacatcttttcagtcaccacaatcttctgctgcttctccctccagcaaaaattatgagaatttgaatttttgatggaaaccagtctcaaaattaccttaagtaaaccaacagtttattatggtcaattacttgaggtactgttagtaagagagggatttttttttaatatgtaatataagagtttcaagagtgcatatatttatccaaagttattagtcaaatcagccttcagagagcctatgaaatactgattttcaatgagaaagtaggtttgatgagggttggagagtgcaagaaagtgaagtaagctataaaacgtagaattagtcaatgttggaatgactatgcagtctttaggatactttttagcactagaagaaaatggaataggatgtcttttttagaagacttgaaattgctgcttcatcctacttattcagtccccatggacatatgtgtttatgatggcagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcgggtaggtctcgctgcagccgagttcacacttcaggtcacagcacagacagtaagggtggggcactgggaactggaagccatacaaaaagaatgaggagaaatgccttgagcactgttattcagaggttcaacccctgtccattccatcttgaaggtcaaagggtcacagggcagctacctccacaaggtcatctctacacagcaggtactcacacttccccacagagcagccagacaggactcccagggactcacattgcaagccctgacttgatttaatattcctgaggtggtgacaattgatggcatagttcaagttcgaacacaaaagctggaaggatgtcttccagtcccttcaggctgctgtaacagaataccatagactgggtaccttatcagcaacagaagtttatttctcacagttgcagaggctgagaagtccaagatcaagacatcagcatacttggttgtctggtgaaagtttgcttcctggttcatagaacttaaattttgtttatcttttatttattttatttttgtttttgtttgtattttgctgtatcctcatatggtggaatgggcaagagagctctctgggccctcttttataagggtactaatgtcaatcttgaaggctctgccctcatgcctaattgtctcccaaagctttcaccctcaaatagcatcacactgtggcttaggtttcaacatatgaattgggggtgggagagacacaaacattcagaccatggcaaaggattttagaagtaaccttgcccagccccctcatcatgtaagtataataatataactaaggtcaaggaaagttgagatttggctgagttgtaagaatttttagtggaccagaaagaattagaactcaaatttccatctcagagcagtggccatattctctgtggggaagcatttgtcagtcatgacattagcaatgatgagacttattcatggttttcagtgatcattgccccaaaaaggcatgttgagtggtacaggcatagatgtgttcaccggcattctgtgggagagcttacttggaatggaaggaactttctgagcatgagtgtgtaacttatgtccacttgttctttttttttttttttttttttaaatgagatggagtctcactctgtcacccaggctggagtgcagtggtgcaatctcagctcactgcaacttctgtctcctgggtttaagcaattctcatgcctcagcctcccaagtacctaggattacaggtgagccacgacacccagctaatttttgtattttagtagagacagggtttcaacatgttggccaggctgatctcgaactcctgacctcaggtgatccacccacctcggcctcccacagtgctgggattacaggcgtgaaccaccatgcccagccatttgttctttctgtttgtcattgagcaagggatattgaaagctctgcctgtgattgtggaccgggccatttttctaaacagtgctatcagtttttattttgtatagtttgaggctgtgtttttaggcgtgtaaatgttccaggctgttaggccctccttgagcctcttcttatgaaatgatctttttatcctttgtaatgttttgctccaaaatctactttaatattaataaagctattccagctttcttttgattaatattaatgtggtgcatttttccatccttatactcttattgaccttgtttgtctttatatttgaagtgtattttttgtaggcagcatgtaattgggtcttgcgttttcctttggattgggaagaccatgtacactgaatgaggttatggatatggttgtcactgaatctgtcatcttgatatttgtttgctatttgtccagtctgttccttgttcttcttttttcctcttttttttttggattaattagaaattttttatgattctattttatctcttttgttggtttgtaagttacaactagtttgttattttactggtttatctcctttgtgggtttataagttataactagtttgttattttactggttgctttaggcttatagaaaacattctaaacttgtcacgatgatgttatgtcagttcaaatgatattataccactccacagacagtactttacaaaagtacttccatttttcatctctctctctctctgtaagcggttgttgtcatactttttccttactacattgttgatgtttttctttaaacagttattttttaatgagttgtaaatttaaaaaaaaaacaacatgtttacccatgaagtcatcattttgggtgctgtaaactctttgcgcagatccagctggtatcattttccttctgcctgagagatttcctttaaaatgtcttagagactgggcttggtggcttgtacctgcaatcccagcaactcaggaggttgagataggagatcacttgaacccaggagttcgagactgcagtgagctgtgattgcatgactgtactcccatctgggtgacaaagggcatcctgtctctttaaaaaaaaaagtttgagtgcacataagttggtgatgaaatctttgtttggctttgaccaaaaatgtcccttttatacctttggttttgaaaaatatttttgctgggtaaaaaatactggattgaggtttctctttcactgcctgaaagatgatactctgctgtcttatttgtgttgatatctttatctttatttctctaaaaataacatgtcttttttttctgtttttacagttttctctatatcactggtttttaacaatttaattttgatatgccatggtatcattcagtctgcccttaattccatctggccgtttttcatcttatcacctcacatactgtagttgttgtatatagaaatttaacctgggctacccccaacacccataatatcgtctgtatcttaacttttgaacctatggaatacagttattatatgaaaactgtttaaatatccatgtttgctaattctaacatctctgtcaactttgaatcaattttgattgcttattattacatgtggcatttccctgtatcattttatgcctcataattttttattaaatgtcggacgctgaatttaaacctgttgaatgctaggtgtttttgtaatattcttgagcttttttctgtcacatagttgagttacttggaaacagtttgatccttttgagctttgcatttttagatttgttaggtgggatagaagcagttatcagcctaggactagttattccttactagtgaggcaagacccttctcagtcatctaccatgaatgttaagttttctcacctgactcgtgggaagaggcattattcctgtccctgtgtgaacaccaagcactgttaatgctaattctttttggtgattttccccctggcttcagtttctttcacatgcatgtacccatcattactcagctatattctcagggatcactctgcacatctctggattctcttttggagcaactctctcctcaccaataccctgtcctgtgaattacaactaccttgatctctccacacacttcatgtcctccactccacgagtcctctaggctctgtgtgggtttccccacctgaaaccatgacctggaaattcagggccatgagtttggcagctgtaagtcctcatttgtttccccttttcaggaatcagtgtccttcacctgatgtctaatgtcttgcagaaatgttgtttatgccaggtgtggtggctcacgcctgtaatcccagcactttgggaggccaaggcgggcggatcacgaggtcaagagatcaagaccatcctggccaacatggtgaaccccgtctctactaaaaatacaaaaaattagccaggtgtggtggcgcacacctgtagtcccagctactcaggaggctgaggcaggagaatcacctgaacctgggaggcagaggttgcagtgagccgagattgtgccactgcactccagtgtggtgacagagcaagactccatctcaaaaaaaaaaaagaaacgctgtttgtatatgaatatattgtccagttattggtggttcaggcaggagggtaaatccagttcttctcactcaatcttggctggaattgtttgtgcattttatgcaacttaaccagaaagtcacccatttgcaccgattttcctcaccattttgatttttctttctatggataaagttacagtgcacagaaagccatgaggagccgttaaagagctcgtggcattcttccaagacgtaagatactatttaccctgttctaccgatgatatcactgcaacctcgggagatggagcggctggttcagggtgacactgctgggaagggagagggtagagccgcgagtcactgtggtgccttctcactagaacgagcttcctctctaaacaaagagcttttgaaaaagaatattcatattagcgttttcattttctatttgccaaaattctctatcttagcaaggcacttgtcaggaaatttttttttatatctctgcagaacaaaagttccttggttcctctcttttttggagaacacgctttcaggagaggagccgtgtgatcatttgccagagaaggataggctggtgggtgctgagaccagaatctttatagcagaaacttgtgaagcgttccagctgaaagtccaatctctaaagtaagatctttgctaaatggacattgatgggactaaaaataagctgagggctgaatggaggctttaacaaatgtacatttaaactctcagtttagtctcatcagccatcacaggaaaggcagtggcaagaatgtttgccgaagtgtcatcttttatcaaaggggataattttagagtggttgtaatcgtgcttattgtgggcctccgcattgcttcatatggaattaggcaacatgttcatttgaaaaaaaaattaaaaactggattaggaagaaatattttactgtcaaaaatcagatcactgttgttgattgatctaaggattcctgttaaattcttaactcggatttatttatttatttatttattttctctctctctctctctttctctgtttctctttctctcctgccttccttccttccctccctccctccttccttcttcccttcctttcctttcctttccttttctcttcccctcccctcttctcttctctaatcttctcttttcttttctcttttcttttcttttcttttgttttcttttcttttcttttcagggtcttgctctttcacccaggctggcatgcagtggtccagtcgtagctcactgcagccccaacctcctgggctcaagcaatccttttgcctcagcctcatgagagttgggtctacagaagctccaccatgtgtggctaatttttaaaaaatttttgtaaagacagggtctcgctgtgttacccaggctagtcttgaacttctggcctcaagcgatcctttcacctcggctttttaaagtgctgatattacaggcatgagccaccatgactcacctttaaatctttcctttgtaataaaatgtggctgggtggtcacaggcctggagtctagggggcattacttgccatttttgttaagttaccatctttaggtgttcatttaagttttaatgttgtatggcttgacctggcacttaggtctgcactttggcaggcacaagaaaattgtaaataccttcttttttttttttttttttttttttttgagacagagtcttgctttgtcgcccaggctggagtgcagtggcgtgatcttggctcactgcaagctccgccttctgggttcacaccattctccagcctcagcctctcaagtagctgggactacaggcacccgccaccatgcctggctaatgtttttttttttttgtatttttagtagagacgggctttcaccgtgttagccaggttggtctggatctcctgacctcgtgatccgccagcctccgcctcccaaagtgctgagattgcaggcgtaattaaatgccttcttgaggacttttataccttcctcttgaagacataaatccagagcttactggtggttgtctgaaaaagatcacatcttgtaataggagtcatacataaatatttctgagatctccctcatacttaaccaccgtgcctggaaaatgcagacaactgtattcagtaaatggtatttgaatataagtaaatcaaaagtgtcgtttccatagattctgtagatgttaccacttagaataattacctgtctaacccttaacaaattctaccgtatattctcagactttgcaaacacgggattccaaaacaatgtggattgttttcagtttctttgaagctcagatttaaatcatacttttactcagtttttaaacctaaacgtggaagtgttatgagatgttgtttttgtgcagctagaattccttgttctactaatgaatttcatttcctgagtccctgtgtctgagtatactgtatgtttcaaatttctgatttagttatgtacctataggcaaatacactgaatggtcaacctaaaaatgcattgtttgactcatatttagagtcatacatatataggcacatgtatttaaaataaatactttaggctagatgtggtggctcacagctgtaatcccagcattttgggaggctgaggtgggaggatcccttgagcccaggaatttgagaccaccctaggcaacatagaaaggccctatctctacaaaaaaaaaaaaaaaaaaagatttttttaattagccaggcatgatggtgcgcgcctgtagtcgcagctactctggaggctgaggtgtgagtgtgaaccaggaagtcaaggctgcagtgagctgtgatggtgccactgcactccagcctggatgacaaagcaagacgctgttccaaaattaaaagttaaaaaatgagataaatacttgaaatattacaattattaaaatagttgttttccccatgggaaaaaaaagttgagcagagctgtgtctttgcactcagaaaaaactcttactctgttgaaggctagtgactcttctaaacaccaaccccttcacctttttactttccttagactttctttggaaaatcaaggagttagcactttttccccctggaagaattaaatgcctctccagaactgcagcacaggaggttaacttctaccacctttggaggcctgcaagctaccagattaaatacttaaagctaagcattcatcatttcttaacctgccctctacagtcatgcacttcctgctttttactttgtcagggctcgtactcaaactattcatagcttcatgaaacaggaactaaatgctgtctgagcctttccccttgtaactctcagcgttctgcttatccttcatagacagaagatgatgatatgtcctgactgttgaagagaaattagacttaatgtgatcctgtacagttaaaccaaaggccagatagggattttttatttcaatcaactttctattgtaggcctaggtttggaaaaccttagcaccccctaatttgtaagacttgaactgttttaaagataagggatacacagccaaagctttctttgtaattctgagatttttggtgtttttacagatacttagtctaaatctttcatcagacagttatcatgattattattctgacttcaacaactgcattttctaaagggaagtgagaattaggaaacattttcctgtacatctgtttatctatcccagttgtgttgtttggattttttcagtgtcttaacgattgaaaccatcttatccatactgaaattcatttcatgcttctatctgctgcttcacattcaggctatcagtttaattgagcaaaaaaaaaatgttggcagtttctatagcatcggcatccttctttggtttctcatttttgaatggttcagccacatgttaaattttgctgagatctctggaaacttcttcacataattctttgatagaacatgaattctgtgagactcttcattaaagagcacggtttcatcactaagttgagattttgctcaaggctatttggtaaattccagcagaagagcaataaatgttcaacaagaaacagccgtcattggagataacaagggtagacattaatgggaggtttgtcacaggtaaacaggtgatggcatgagaaggtttaaaccacctcataaataaatttttcgccactatatgatgtggattacaactaaaccaaactttaaccaatgtgggtgttggtgtacctttcttttctaaagcaaaaaagaaattgtggtactaaacttgagcttttatatgataccgctaattgcccttcacccaacaaaccaattatttctacccatcacttttcgttgcaaaagaaattatcttataaaatatatgtatatagcatatatctccaaagcaaatatgggacatctgtacctcaatgtaactgatagtaagttaaaaattatttgctgagttcttggtatgtggacagatgaatattataatgtttaaggcattccaccagcaggttttgaatttaaccaaaattttatgaagctaatcagaatggaattaaaagttgaaagactgccaggccaaaaagatcatctttcagaaaagtaaataattgctctatacttacatatttattatttatttgttcaccatccgtttatttttagttgagttctgtatacattggtgagcaaagggggactcattagcttgggcactcctaagggcaaacccaataacagatagtgaatgggaaggaaaatgagagaattagcatttagtgaacaaaatcttataaactatgctgaatggtatacatgctctttcagtaaagccactcagtactattacttccctaccttctgaatgtttaccaagttcgataaaatcttgctacatacaaatgggaagtatgtaacaagagatactagtggtttctgggtttaattaagttacatagaacattaagaagtatgttgggaaggcattagcttaaaagctgtcttttgccactgaggccatattcatcacccagattccctccacagtggagcactgctcagtcactgccttcagaatgcaaataggtcttcacaggatgctattaaaacccatagcaccgtatggttttacgctctgctttcttccagcaaacctacgtattttaatgttaatatatatgttcattttatctttgagaatgaaaatgtattatgaaaatataaatatttagtttttgtagacaaatagcacatttaaagatccagtagttattcacctgcctggtatgactgatagagaacagatgggtagcaaagaatcctcaccctggaaacatgtacgcatacacacgcaaacacaatctcgcataagctttaaggggttcattcattttgtgaaggttgaccatggataccagtttaaagatgccaggtgatgtttggcttttaagtatcatcaccttgagtttccttatataaatgttttaatatcaacaaaaaactcagaatttctaataggtcataacactgcctaggtatgggaagaggagagggtaaaattaaaagaaatgcaaatgtacttttctttgaataggctaaccagttagaggatgtatgaagcaggaccaccatagtccaagtatgaatatgcaaaattaaagcagattgctgtaaatctctcttaaaaagtcaagaatatgtcacagttggcatttttcggatgatcagaccgtttgacaaatctctgcaagggtccgagttagtcgaacatatcttttgcttatgatttcagcaacactgtgcgctttgccttctgggttgtattactaattctgttcatcattaagtgtacacatgtttcactgtttgactaaacaaagcttgaagacatacaagtccaaacattttctgtcctttctgtattcccaggtcagaagatcttagctctttatccattctgtgctagagtttgtctgaaagcatgaatcatggggttgtgttgggtgctaaatttcagttctatatgctgccttcactaatttatgactgtggtctgaacaaccaaaataaattatttctgagaactagcaaaataccctggaaaaaatttatgaaacctaccagatttgtaaaaaggtcagccaggcgtggtggctcatgcctgtaatcccagcactttgggaggccaaggcgggtggatcacctgaggtcgggagttcaagaccagccggaccaacatggagaaaccctgtctctactaaaaatacaaaattagctgggcatggtggcgcatgcctgtaatcccagctactcgggaggctgaggcaggagaatcgcttgaacccgggttgcagtgaggggagatcaccccattgtactccagcctgtataacaagagtgaaactccgtctcagaaaaaaaaaaaaaaaaaagaaaagaaaaaaggtcactaagtgatgagagaattaaaaccactcctattttctaccttttataacataaactcttgtctttacttcccaggtttgccatctttataacctgtaactaggagcaagtgatttaataaagggattcctcctattccgtctgcaaattggagttaatttttctcctctttctgcaatttctgttgttttagcttttgttagccataattagaaaatcaaaaggattcattgaaagttgagacaaaattgtgaagtaggaagatgtcttcctccgtaagaggagaaagattgttagaggaagtacatggaggctttggtgacacatccctatgaggaagtgatctgcttccacgggaaggagagaacagatgatagatgactcctagatgctcctgcgtcatgttcagtacaaaatcagagccgcatcagcaaaccggataccctgggaaggaacattgggataactccttggccataaacaaaagttttgctctggccttatgaactaagtggattaatagagattagttgttcaatatccttccatctaatggatccatattgttaaaacttaagaaacaccaaagggggagagtgtgagaatctactgccattttctccattgcttcattcaagacttctgtgtccaccttggcatgggattatcatttgcccttttatactcaccttaaactacaaagatcatttgtttgcctttctgaagaaaggaaggtggataaattttgcaggagactttaaaaattcagctcaactcctggcattaaagacaataaaggtggtgattcagaggtccccatggcagtttccccaaatattacgtttaactaagatcatctcaagcacagagagacttaactctctaaagtgtgatttttaaacctgcacacggaaattttctacattttatatgctcaagatttcttgaaatccagggtgtaaaactgtgaaaataatagttctgaacctatttatgaaagcaggagagaattgctttgttcagttttggaaaccaccagggtgcaatttaatgctccaggagagggttttctgtgacctggaaaggttggggtgattcccgtacagtgagtcatttacttccttcttggcaggattatgcccagggcttttaaacgctcttgacacttcactgccttccataagaaacgggtttagggcacaagtgtctattatgataagatgccagatttctagatcaagcataaagctgtggagtgggacaataggacttaataattttaatacacttcccacgtgaactgtcaagtaataagctacatattcagacgtgactggcattgagtcatcctgccattagtcaaatgtatgagacttttcccaagtcccagcaacaccaaatttgagaaatcgtataaaagaaaaatgaaaaagtaaatacgtctaagaaatggggaaatcaataaaaaatgtactagcaccaggggcggtggctcacgcctgtaatcccagcactttgggaggccgaggcaggtggatcacctgaggtcaggagtttgagaccagcctatccaacatagtgaaacccttctctactaaaaatacaaaaattaggtcggtgtgagggtgcacgcctgtaatcccagctgaactcaagaggctggggcaagagaattgcttgaacccaggaggtggaggttgcagtgagttgaggtggaggtgcaccactgcactccagcctgggcgacagagtgagactccatctccaaaaccgtgttagcttttatcaggaaaggccattaaattttcttcaatattacctacatcaatttgacatcaaactataagaacacaatatgtgagatgttttgaaattaatgaaatgaatcttctctttgagacgagtttgtgcacgctatgaagtccattaaatattttctgggaagagttcccagttaaaactactttctaaagtacccaagcacactatatagatacatcaagtaggtttattaatgtcagtatttcggtgaaaattgggaaggccactattctgagtagagaggtgtttgttttctacttgaagactatgaaggatattctgcactactgggcctatttctgaaattgtatctagctgcaagttactcattaatttaattaacaaatatttattagcaaccaaattgtggcaggaactgtgcctagaggtagaaatacattacgctcttgtttccagagaggaaaatgagtaatgttctaaaatgtaaatcttatgtttctctttcctcttgctgtaaatgatgtctaagccatttcggtggtgcctgttcttaatcctttaataaattattttgaatgattttttaaaaggcagtgtgattgtgaggtgagtttagtacaaattaaaaaatgtcacatcagttgcaaacagacagcacataattatgagctaaacactgtttcagaaacagtagtgacaataaataggattcggtataattagtcctcctgggacagatctcttcctctgttctttgccatatattttaataatgagattagccatctaatgccaatataaccagactgtagtattaatacacagaaaaattattgctagtaacagaagatttaaagacatgagggtgatccggattatatgcctgcaggttaaaaaaaaaaaatgtttcttaaactctctttatcaatatccttttcgtgagccactgaattcttttgtttttctgatcatatgaataacccgtcttaagtatgaatattgtgacaatagtaatagcaattactttaagagaatatgtttctatagagataacagaggggtttttccctaccattcctgtatgaaggtttctgtgtttttaattaaaattttaagaaattcatctttgtcttagcataataaaataaacctcactagacaatcaggacaccttgttattcttagatacattattatagctcaagaaataacaaatgttaagcctggaggatttgtattttggccaaaagttgtgagggcaattatttatttatttgagtgaaatatgcaggaatcatatatcaaaaggagggaggtcttttttttaacctcaacaaaaatataaaagtatagggaactgttatctatgtgaacccagcaaatctgagacaggtctcagttaatttagaaagtttattttgccctggttgaggatgcacctgtgacacggcctcaggaagtcctgatgacatgtgcccaaagtggttggggcacagcttagtgttatgcatttttagggagacatgaggcatcagtcaatacatataagaagtacattggtttggtctggaaaggcgagacagcttgaaacaaaaagcaggaagactcgaagtggggaggggccttctaggtcagagataggtgagatgaacagttgcattcttttgagtttctgattagcctctccagggtagacaatcagggtgattggctctaagcagttcccagctagacttttccctttagcttagtgatttgggggccccaagaattattttctttttacacctgtgttttttctttcttaactaaaaatagtatttgtttctgagggatagagataactaccattttatttggtatgatttatttgcctaccgtccaacaaagaaggtaatctgattttgttttcatttccaagcatgattcctagagatgtgtttaggtggccaaatcaatgaagcagaaccctgcaaattcatttattgggagatatcctaaggtcttcctgaattatctgatgttcccaggctttgcttatccaataggcattaggaagaaaaggcttgtgtttatgtagatgcacacaaaatgcgcatctcagcttacataaacagtgaaggggtttcccaggtctcattatccctggaggagctgaggcctggggagtcctcagagcctcctttgctactctgttattgtggcaggccaaatctcactaatgcaggcctccgtaacagctgtttccgtactgactgagtggttcagttaaatactaaaagctaaaaaaaagccagcgcccttatacagaggctggaatgtaacaaaagcccaccaagagttttgcctaggcctttcctggcaaaaccaaggaattcttaacaggacccatttaggattaacaagttttactgggagtctgaagaaactctacaggcctccaagaacaagtttactggaggtctgcaggaactcctcaaacctccctgatttagcaggagacaagataagagtaatcaccccagcacctagacccatttagattaagtaaatttactgagactccagaggaaggtcttcaggactcagaccttagttatggattaaagaagttaatcacttctgtcatgagatgaatgcacacttacacgtagacatacagcttagaaggcatataagttctggaaactttgtaattttgagttggtctggcactaatttccaggccatctccctgtaaaaggttacaaaaataaaaactctcttcctcccagttcatctgcatctcgttattgggctgtgaggtatagtagcccaaccctcagttcagtccgggaacattattatcaaactctggggattttcaggataagttaggaatatattttgaaggagaatggcagtattaaatgagatcagcaattgaaaacatgtgaatacattcaccatttaattacccatctcaatagaagataaggatgaatttagcactagaaaccatgaatgtcacaaaagcctaactttaataccttgaatcactttccagtaatcctaatcctttatccacatggatagaggacattcctatcttgctctttcttctccccacacattagcttaataattagctaaataatttcatgcattagctaaggaataatggaactccagaaacctaggacagtaattgagagactgaacaagctaaatctgaaccttctgcacaattaaaggagctgatgttagcatggcccacagaatattacctgcacttgtaaaaactagaaagtcaggcaagtactcctggaacagactgtcttttctaaccatgcacttatttctttgtttttaactttgtttttgtttgtttgtttttgagacggagtctcgtcctgtcgcccaggctgtagtgcagtggtggaatctcaggtcactgcaagctccgcctcccaggttcaaacaattctcctgcctcagcctcccgagtagctgggactacaggcgcccaccaccatgccttgctaatttttgtatttttagtagagatggggtttcaccatgttggccaggctggtctggaactcctgacctcgtgacctgcccgcctcggcctcccaaagtgctgggattacaggcgtgagccacggcgcctggcctgtttttaacttcgtaagttgctttgtagaactatcttaaacttggtctattttagattctccttgaagggccagatcacaaagtgattaggagaaggaggctctggagccaagacagtgagactaacccattcttcaaatctaacactaactagctgtgtaaccattgggccagggatgtagcctatgtaaccacaatctcctttgtgcttcatgcctcaccaataaggtggggagagctgctatgactctgtcatagggctgagacgaagatttgatgagttaccagcaaaaagcatttaatatactgtctcattcagaagcagtcaatacacattgaactcttattacttagaggaaagaaaggggtcgatgatttatagataaggcctgagtgaaagaaataaatgtgtattgagtatcagcaatggactacgcatcattctgggtaactatcactgaacacatttaatctgcaaatactatctgaaagatactgccatctgaattgcataggtgggaaaagtcaaggcttttgtgcttatttttaaatgtatgttaacatgaataattaattggccgtgcactggtcgcacgcctgtaatcctagcactttgggaggccaaggcgggcagatcacgaggtcaggagatggagaccatcctggctaacatggcgaaaccccgtctctactaaaaatacaaaaaaattagctgagcatggtggcacgtgcctgtagtcccagctactcaggagactgaggcaggagaatcacttgaacccaggaggcggaggttgcagtgagccaagatcacgccactgcactccagcctgggagacagggcgagattccatctcaaagaataataataataataattagtgggaatatatgctgtttgttgtgataggagaagaaaaggggagatccagatgttggtcatcagaaatgacctgcatcttctctaaattgaaatttagcttgaagtagtaatgccaaggccagcctggggccatggcatcctggtgtacctgcttgttggtgacagtccttttaggacaaaatatcttacaaaaacattcatagatgactaactggaaaaaaagaagttatgatgaaaagcaattcttactgtttttgttttttgttttttgttttttttaattaaaagacaggcagttattctctttcccaggcttgagtacagtcgtgcaatcagggctcactacagcctcaacctcctgggttcaaataatcctcctgaccaagcttcccaagtagctggaactgcaggcatgtgtcactgcacctagctaatttttaaaattttcttgtagagacaggttcttgtaatgttgtccaggctggtctcaaactcttggctgcaagtgatcctcctgcctcagactcccaaagcaatccttacttttttaattgaagtgctttttttttttcttatttaaactcttaaaaattcatcccattttcttcaaaacaataaatcctttacaagggagaaaaatgaaatagagcaactaagaaagcatccatgaaggccaggaaggaacatcatggttaaatgcgattagtcacagccccttctgtctccagtcttggatgaataatggtaacagaactagagagatttctaagaccttggtgcttaataacaagcaagcatcgagggtggttgtgcaagatcctgatcacacaactctgcatatcaaatatccttcacaatcaacaagagtcagcctttaatctctgatgttttcaaatgttctttttttttaattcacatgtataacctatcctcttttcaatatatagagatatagcttatcagtcattacctcttttactacttttcttctgagatttaaaaggtcatcctctgatttgtaaattcaaccagatttcacagttcagatgagagttgtaataagttgccaagaactgttgcaagctgtgatggctgtctttctggggattatagagaagcacatttcttttaggaaaaaagaaggcagatgagcccaagttgttttatttttattgtgtatatgtatttgttttctcaagtacagtagcatggcctctgtggcaggcattcaccaagcaaatacagagcaccctctgggtcaagcactgtgttattggggttccaaataaagaaaaaagaccaaatccagccctgtggatctcacattaaagaggggagtaagaatcatagataatttaatgagttaggtacattaatggggatttgcacagtatttcataggagcacagaaaaagggtacctcgttccacctgagatggcagggagatggctcctaagttaactaagcaaaaggggaaggagaggcacatcccataggatatggataacacaacaaaagctccattaccacccgccccaccaccacacacaccatctctggaataaattttgtaccttccaactggttttcagggtactcatgacacatgggttaataatagctaggtttgcccaaatgaccaaaattctattcccaacatgaattttcacaactggtttttttgtttgtttgttttaatgaagaagaaatgttgtgtttttggaaatcatcttacaaaatcatgtagtggaatgcatatagtgatgaaattgttgaagaccagggaatctttgtccttagtttttattaggatggatgttttcaggtaaaactaaaataaaaatgaaatggaaccaattagaaagaaaataggagcacttaagacgaaatgtaattgttgctgaaagaattagaatctgatagcttagaaagaacttaattaaatttcagagtaaaataaatacagtttagcaatggcctttctcctagaaaaatataatgtctcttacagttcagttactccttggcttacaaaatagtggttaaatttcctgtttttctcattttgatttcgtatgttacatgttactggtgatgggattatctgtgtgacattacctctggtcttcaaaatgtacacgtagcaatgtagtctcacatatgccccttgggtgcgaaaagagaaggacctaacttaacctgacagacctcaaatctgttctctttctatagcagagtactcctatttaaaaagaaaatgttgagtaaggaggacagtcatcatatatgtcgagaatgttaaatgctcttgtggaagtgaaaagcagccgatctaagtaattacccattaagaaaggagtaggagaaatgtttagaaggcagacctgcaaaaaggtgactcacagtacgttcacatgaccggatgcattagtggaacctctaacccatcgccaatggaagaagcagtgttttgcacaaacttgaaaaagagtttttcattttcctcccacagcctcgtcacgtgttcaatatgctaaagaagtatgtccgcgcagaacagaaggacagacagcacaccctaaagcatttcgagcatgtgcgcatggtggatcccaagaaagccgctcagatccggtcccaggtaagcgtggggtataatcatcttctgcagctttgacaatccaggattcttgcttcttgggttggtgacaggatcccacgcattctccgagcacctataggtgttttccctatggcaagagctctttaacttctggtaaattctaaaagcttgtcttagagcaatatgtggtttggcattttctgagttatgaaaataacaaatgtaaaaatcttagtctactgctaactgattgctaaggaactgattaaaaccaatgactaaaatgagaaaaacaaaagttgtataaactaaaatcttgaaaactggttcaactgaagtatggcaggcaatcataaacctactggaatgttgagatttcatgtgagcctaaatgaagcccagtaataatgtattttatactccccggttcccaaaggctgaaaactgggatctaccttaggaaatgtgaaattttacattttcttgaatgaaagagttaaatatatatagcatgaaatagccaatcaaattgaaatacgttgttttctagggggaaatttgcaaagaaatgtatttaggcatgagggtcctgttaagagagcctcagtattaggaatcagaggtggcagaggtagcctttttaattggctgttataagcatacaagataatggtaagtttcaagatcaagtcttaaacctgagctcaaaaagttagagaacagaaataggagaacaagaaaaataaagttgccctttcagttcaacctttttaacagtagcagtgttttgtcagttttcttttggaagtttgtatctacctgcagtgcttggtaagaaaaataacctgggcgacagagcgagactccgtctcaaaaaaaaaagaaaaaaagaaagaaaaagaaccattcctacccccagacattgttgacctggagttgtcatcctttgatgcaggttatgacacacctccgtgtgatttatgagcgcatgaatcagtctctctccctgctctacaacgtgcctgcagtggccgaggagattcaggatgaagttggtaagtaagctgttcttttgatgctgcacatggacatgtattttcccccagaggaaaattgaggaagtgagttatctgtttgaggaatatggaagttaatagaacagcttgccttttacgatgaaactgtcaggctggaactagcttactagccttagcaattgaatcagttcttacctctccatacttgtaagactggaggaatgtcgcctgacttagggtatagttttgtaataattggacacttgttattgaattatacgtggcagcattttttctaatttgcccaatcttgttatggctctcaaattatgatgctttggtactttacaattaatggggaaaaaaactcattcttcatcttttgagtgtcacgattgttccatcttacaaatgaaaaagctcaaacttaaaatttgagtaacttactggcctgtgtcacatgcattataagtagcacagccaaaacacataactgtgacttgtgactttgaggttattgttatattcagtaagtcacaattctctttgatggctgagcagggaagccatgagtgttctctgaaccacatcattgtttaaacttaaaagatagtgggctattaaacttatattctgaaataattgtactctcacaagaacttggaaaaatagtacagagaggtctcatgcatccatcacccacatgcctcaccagtgacatcttacgtaactatagtccattatcaaaaccaggaaactgaccttgtgtgcccactgtcttttatgtccctagttttcctaatgactaatgatttcatttaagatctctcatttctgtttctcttggaaagagaataaaataccacatgagtctctgtcagctgccaaaatgcccataactgtttaacttacttcacaaaggtacatagatgacgattatgttggtgcatctttcaatataagcatatgtacctatactggagatttagtgtgtagtctaccaacatataagcaatatgtatctggcatatccttccccataaagaatcctaccctttgttaattgggttggaaatagatacaggttatcatctggaatcagagcacccaggatgttgtagatggatgtacccattacagtaagtccgactttgaagtgcctgaaacaccgctcaagggaacagctggccctcactttttcccattttatgccagtgttattctgctcattctttcctgatttatgtggccttgtaaatattgtttgtattgctgtgaaatatttggcatgtgtcaagtgatgatgttctgttcagtactgcacattaatggacggaatctgaaattatactttgagctacataacttgcagaagaaacttcatatgtcagggaggtgcagtgtcccagaactttgggaggccaaggtgagcagatcacctgaggccaggagttcgaaaccagcctggccaacatggtgaaaccccctctctaatttaaaaaagaagagaaaaaaagcaacttcatagtgtgccaagcttttgttcagaaggagcaactcagtgttgagcaagaatttatgaagatagtggaggtggagtttttagtctacaccatgaattgagaacagagatctccaattaatttttcactgtatcaagtttctttagcaaacaaatatacttttcaggtatacttggagaatagacatcagtaaggaacctttaaaatactatgtattaaataagtgtaaagaagttctccaaaaagttgtagaaggtacaaaatgaaataatttatcagtcttaatgaactgaagctaaaatcttctagaaaacatggtttgtttgtttgtttgtttgttttttacagctatcttaagcctgttttcacatgtgacttcagttgaagtggcataggtaataactatagggaaataagccattacaaaggactaaaaagttgatgaccaatattccaccctcacccagcaattattgttgactctttataattaaattattagaactttttatttggcactatactactgaaaacattagccactaattagtcacacttccacagaacagatggtggttcagatatcttgaatattacgaaatgcccaagaatagggcagcacaataaacttcaaatctcagtaccagggaggatatttagataactagtatcatggaatctttttctgtaaaaagaaaaaaaaaaacagccattaggttggattaagaacatattagggggtgcagcacaccaacatggcacatgtatacatatgtaacagacctgcacgttgtgcacatgtaccctcaaacttaaagtataataaaaataaagaacatattacgtatgtgctcttaatgtgtgtgtgtgtgtgtgtgtgtgtgtgtgtgtgtatgtatgtaatatgctctcaatccacccattagttgggtttttttaagtaatgacatatatatcattctaaatataccagatgccacattctgaaagataatcttttttaaaaacctttcatttggaatcctgggtgcttccatttttaaatgactgttctctatagaaagagaagtttgcaataacgaaccctgtacactatctgagctgctagtgtcaaagctacagaagtggtttgctggggagaatagcttttctgggttgaacctagttgtcatctatagtcctttttcatttgaaggaacatttaaaacaatacgtccctttttagtaggaatatagttgaaacttcaagattggccaggggtgctaattgcttaagccaaatgttattagaggttcctcatgttattctttttacttttgtatgtgtttaaaatttctcataataaaaagtgttgcttttagcttactttctaaataggttcttcatagattttttttccaggttatgtcttgtagtcgttctaatattctctgattaaatgctttgtcattagtctgagttgcatttatttgctatttctgtatataatcaaattttagatttggagtcagttcagtacaaatagcatactaaaattcagagacactggcaaaaggagaaaattttaaagtccctaaaaatgcatgccttttgtatagaagtttgtgcttataaatacaagcaattaggatgtgcttaatgcagtcaaacatctgtagagagatacaatggtttaattaaataatggtaacagttgctttatggataagaggttttcaccagtctggggaactgccagtatctttagtgggagaggtatatctgcttcagtaaattatgcttccaattagaattctcttttatgggtgaaaatgaggaagaatggagtcatgctgagaagaaggaatgctatcggtgaaggggaaaattgacattaaatttgtgagcagtaaactgtgaccagagcatagggttcatcatgcttgtgagttctggccctggccttgtatcttccattgtgcttccctgaggcatcctgtctgttcatgctactttcccactaacttcagtgaatcccaaattcatgggtctagccacagccagcgtactgaatcccagtgccatattccagaccccaaatatcccatgtgcctctgaacctgcaatcaatgtgtataacactcatgtccttctggaaatttacttgttttacacactctattctgagttctcttggttggcctaaactatccatgatgccttcctctccctcacccattatttcttattagttgcaagttctgtatatttggccttctgtgttcatgtgaaatctctgcacatcccctacttacctgagatcctgtcctctcatcagtgtcattagactcccagccagtcaccctacctcttatctcacattggccccctcccacttcaattcctccttttcggaactatgcaagttcccttaaaaacaaaagacaaaacaaaaaaaacagctgcaggccggcggcagtggatcacctgaggtcaggagttcaagaccagcgtggccaacatggtgaaaccccatctctactaaaaatagaaagattagccaggcatggtggcaggcacctgtaattcccagttactcaggaggctgaggcaggagaatcacttgaactcgggaggcagaggttgcagtgagccaagattgcactattgcactccagcctgggcgtcaagagcaaaactccgtctccaaaaaaaaaaaaaaagaaaaaaaaaaagctgcttgtggtttttccttgcttgaaaagttgcacctttcaagattcaagtaaaatgcccaagtcctataaaccctttctaatttgcatagagaacggagaaaagagctggcatttgagtgacaaccatgtaaccaactaatgagtgcctctttgtgataatgctgatggtcacattctttctttatgaatgttccccgttgaggtacaggaccgccatatagagatgtggcagtggcaggtggtttagggtaagagtggagactgataattttattctacatcccactcaaggagccaagccctctggcattgtgcacacccactggaagtgggctgttcttgcatttcacacaaaggcagcatatgggctagtgaagatactcggaaattcagtgatagcatccccagtttacagttgaagaaattgaactttgaacctaagagagtttgccaacttgcccagcccagatagtaaagtgatgaaagtggggtggaagccaagacctgccagaagtccaaaatctgtgttctttctgctgcctattttcctctctgaagtggaattttcccctacttaacctcaatgtgaaagcactgggtatccacctccaataccatactctaacttggagtaaattatttgtttatatctctactaaccctccatcctgtgaactcttccagatatatgttggtcatccttgcctcctcaagatccagcaaggaccctggtactttttagatattagtaattataaatataaggttgaagccagtccccaggataaaaaggcagatgctccttaacttggactgatgaagtgggctcctccgtaaccctaaagacctgaccaaaactccttccttgacatctagcgtaagcaccacacaaaacaaatttctttcctaggccacagcatcctctaaagctgtgattgatcaagctaactaaatggatgtggttggccaagataacaaagcataaatcagggttctgaggacctttattaataaaattttacaacatggtccttctttgtaccatgaccctctgctatttggtcatctacttgatttaattgtgctgtcctgtttgaccgccaaccacagtctgaagctattcccatgacaaataagtgtgaattttcaagtgtcctgttgtgttataaaaggcccgtgttttattttataatttcatatttttttcattcaattttaattattatgtgcaagttcccaggctcagtcatttattaatgtttttattttttaaaaagttattttcaggcagattatgaagatttcttgcaaagaatggcctcaggcattgtttgtatagagataaaatctcctcttttaaatcactatgtaggtcttcattaatattttttcattcaacaaacgatggaatcattatttcagtgaaactatggctcctcttcagtgttcatattctgctttaattatttattaatccttcaaaagggcagaatcccttcatcctttcaacctttgctttgtcaaattgttacttaaaaatgattggtgttttctttgtcctttgtaatcttccttaaatctctgctaaaaggattctattctactctttgtattggcactgaggttatttatcaaagtgtgcttctctttgcagctacctcaaggctaaaaatgtagctcttttcagagatggctgttttacagaatgctataataaccaatttatgagaaaggcagtaggaaataaagagggaacaaaggcataattgtttcatacccattttttttcttctagcattcaaaattaataaaaacatgaattattataaaccagatgcgggaaagatctctggctgaataaatccatagtgaatggggcaggattgaaagatatggcggggaatcaagaaattcgcaataatgaaaggatgcttattttttattacaattttactgtggcctcttgataaaatgttccactcttcaggaaaaatgcagcatggttatttcaaagacagtgtttccatattctgtttctaataagcctgttgatttgggggtttagtgggtccaaagtagactaggtttaaaatagaatatttatagcacttcccagcccaacaaaatagaaatttaatgttattgagaatttcaagtatccttatgtgtaattgttagtcaaggatgaacatcaaaaaaaaaaaatcataacagcaaatagagagctgtcactttgttcttataagaacataggtgtgactttgtttttcataagaaatgacttttaaatgtatatagatttaatggtttacagctgacttaagtatttggacataagaactttttttttttttttttttttgagatggagtcttgctcagtcacccaggctggagtgcagtggcgtgatctcggctcactgcaacctccgcctcccgggttcaagtgattctcctgcctcagcctcccaagtagctgagactacagacgtgtgccaccacacctggctaatttttttgtatttttagtagagacagggtttcaccatgttggccaagctggtctcgaactcctgatctcaggtggtctgcccatcttggccccccaaaatgctgggaatacaggcgtgagcccccacatctggccaagaacatttatacatattatcaacaaccaaagccttgttcccccattggtttcataatagtctcagagctccttattcatattttccctagtgttattaagtaacagacatgaggcctgtaagtttagctgcctgtgtctaaatgtatatctttgacccaagacaatttctctttcaaaatagaaccactcatttataaatgaaaatgctttgtcaaatgaatggcaaagtacggtatactattaagacagcctcctagttttctgacatttggactgccatgttgaaatgtctccacacagcaagatacaaatgtgtgaatgccagtacacttcataccttaggtccccaactggtctgtctgcagacttgaggttacagacctgcaggtgtcgaactcaccccatagtggtggttagtagagtgctttgtatacgtttagctctctatgccttttttggttgtgatattaggtaagctctctttttcttgctttggagcttaattatggaatctgtaaatatagtgcatatgtcatccagatgatctgtggaagtcattgataatgacctttaaaaattcacatgtaatacagcatccgttttcctgaatgatttttatagttttaccattggcaaacagatactttcttttatgtatttctactccataattttaggaaaaagtaaatccttttagctacattaaaaagaatacactttctaggaataatgaatgtaaaaaaagtttccttttttaaaaaaatattgcgctctctaccattctggcagacaaattgttgaccttaatatgtataattcagtgcagtatattgatcttgtttattgtgctccaaatggacgctggcagaaatttgaggaattaaatatcaaccacagtgttttcttcatgcgattgcaatggtttgttatggattattttgttcagttcaaataaatgatgtacgttcagattctttgcccaattttttaattgggttgtttaccttactttcttaattttatttttatttttatttttatttttatttttggagacagagtcccgctctgtcacccgggctgaagtgaagcggcatgatctcagttcactgcaacctccgccttacggattcaagcgattctcgtgcctcagcctccccagtagttgggattacaggcacatgccaccacatccagctaatttttgtatttttagtagagacagggttttaccatgtttcccaggctggtctcaaactcctgagctcaggcaacctgcccacctcggccttccaaagtgctaggattacaggcgtgagccactgcactcggcctgctgttattattgagttgtaagaatttttatatattctggatactattaaattcttatcaaatacattatttggaaatattttacccattctgtaggttgtattttcacttccttaataggatccttttgacacacaaaaattttcaatttagaagtctattttatcagttttttcttttgtggcagttactatggtatggtatctaggaaaccattgtccgaaacaaggttacaacgatttatacctgttaccttctaagagttttgtagttttagctcttacagttaaatctttgattcattttaattttcatatatggtgtgaggtagaggtttgactttattcttttgcatataaatatccagtgtcccagcaccatttgttgaaaaagactattcttgccaggtgcagtggctcatgcctgtaattccagcattttgggaggccaaggtgggcagatgacttgagcccagaagttcaagaccagaatgggaaacatggcaagaccacatttctacaaaaaaattatccaggcatgataacatctatttgtagtcccagctactcaggaggctgtggtgggaggatctcctgagcctggggtggctgaggctgcagtgagccttgatcacgccacctgggcaatagagcaagaccctgtctcaaaaaaaggaagaaaaagactattatttcccccattgaatggtcttggcactattacacaaaatcaattgtccatagataatatgggtttatttcttaattcttagttcttttctttgatctgtgtgcctgtgcttactgtagtaccacactgttttgattattgtagctttgtagtaaattttgaaatcagcaagtgtgagtccgttatctttgttcttctttttcaatattactttgagtatccggagtctcttgcattacaacatgaattttaagatcttcctcaccccctgcccccactaccatttctgcaaaaaaaaaaaaaaaaaaaaaagtgcggattgcattggatctgtagatcattttggggaatatttatatataatgttaatgaggttttaaacaacacttttatctactagaactgatatttgcctcgaaatgattccaccttgagccccattcaggaacgttagtattttagaagtagcaagagaactgatttagcatgctatcatcttttactccaaattttggtgagattccagagaaattatttgtgagcttttccttaagccatgatgactgacatgtcagtagtttttgtctgtttttatgccattcttttatttttgtgcatgtgtgggcacctcaaccaaatggatggagcctgaggcatggtctcccacgccaaagtctgccgtttcttttgatgatccaatgcagttccaaagctattttctcaactcaccagctcgtttgtatggccctgtaatttgaaacaaaatctaaaaagaaatccaaattaaaatgattgggaagtgaagtcagaccacagtggtggctgtggaactacttcctggcatggaacactagagcagatgtgtcctgctttttttgtagagtcttgggaacaaccaatgattttagactggaacaagggctggttattctgtttttcacaagcatctgccttcaaggttcattttttaaaagaggaggggaaaaaaatcagtgatgtttgccaccctgagtatggagatgaatccaaattgagattaattttgaaatatgtttcccgaattttgagattaattttgaaatatgtttccacaaacactaactgggaatgacatgagtttgtttcatgtgagacggagtttggaagacatgttaatttgcttgttggtgttcaactcagaaacaaattttgcatgtattttctgagtaagtgccattattagttaacagttttcctgccgtatgcactatgggctgctttagaaagtaaatcatagaaggttagttataatatctcatcaaaaatagtggatccatctattaaccggttccaagttatgagacgtgtccaaaggaatgttgacttggtgtgcacctaaacactatttgtgaagaggctttggacttcgagcatgaaacggggtcagagggtcaaggccagggcgttacttaatttcagctcctgttgatggattcattgacttcagatagtgcgtttaaaaaagcctgtttaatgtgtttgtcttttgataattattatgtatttacatatgctcctaaaagcatgagttaaatcttatttctttgaaacagtgcttggcaacaacttgtattgtcttgtacatgccttttgtgtctttgcttttggttgggggggggcattgaaatttgtctgcccatcccctgagggcagatactgaggtgtggacctatgtcttcagcttcctgtatagcattgaagttgctatagctgcatgttctctaaaatttttatacatgcgatttaattgttaaagcttatgatttttaagtatagtattctgatagcaaatagattttgtcaaataaaaactaactttatcatcaccactgagatatcactaggtatatggcttagttctcagtcaccacttttatttgtaaggaagtcttagaagttttcacaagtgtatatgtgatagactgggcacagtggctcacgcctgtaatcccagcactttgggaggccgagtcaggcaggagttcgagaccagcctggccaacatggcaaaaccctgtctctactaaaaataaaaaaattagccgggcatggtggcacatgcctgtaatcccagctactcgggaggctgaggttggagaattgaacctaggaggcagaggctgtagtgagctgagatcctgccacttcaccctagccagggcaacagaacgagactccatctcaaaagagaaaaaaaaaaaaaagctgggtgcggtgctcacgcctgtaatcccagcactttgggaggccgaggtgggcagatcacctgaggtcaggagttcgagatcagcctcaacatggagaaaccccgtctctactaaaaatacaaaattatccaggcgtggtggtgcatgcctgtaatcccagctacttaggaggctgaggcaggagaattgcttgaacctgggaggcagaggttgtggtgagctgagatcgcgccattgcagtccagcctgggcaacaagagcgaaactccatctcaaaaaaaaagaagtatatatatgatgcttgctttagcagcacatatactaaaattagaatgattaaagagattaacctggcccctgcacaaggatgacatgcaaattttgtaaagtgttttatatggaactttacttacatgaggtacctagggtagtaaattcatagatacagaaagtagaatgctgattgccaggggctgagaggatgagggactgtggagttagtgtttaaagggcacagaggttcaatttggcaatattgaaaatagttcagtggggaagatggatggccgtgatgcatcgtccatcacattgcacagcatcgtgaaatatacttaataccctgagcgttacacttaaaatggttaaaacagtaagatttatgttatatatatttaccatgataggatatatatatgtatattcatatcattcgccattgtgaatagcaaggagattgaagaaataaggacacaggatcttcaataaatatgtattaaatgcttcccatgttccacattctttgtcattgccagacacgtcacaggtataagggcccaatctttgttactcaaataatttagaataatttagaatttgcttggggagactttggataaacagttaagaagctgtatccattaggtaaagggacattgcttgctgcgtctcgtagcatttggtagtctcgttagacagccgttcttactcctatcgtttgctgaaatttattagcaaaggaaagaatcttagttttcagctcgttcagccgcatgatttcataggctcaggaaaaccatgtaatatgcccaaggtcagacgtgatgaataagactaaagactgaatctattccagtgtctctgtgtccagtgatacttacttactgctgttgccaagttaaacaaaactgggtttgcagagctgtttattcaccttcagcatcttttcattgaaaatgcggtagatagaagaaatgtccctcttttagcatttgtacctcagtaataagatgcttcccaagctcaaagactccacacccttaaatatgtaagtcagtgttaagatatttaaagagaacatgaatatcagctacattgtgcatgttgcaggatttttatattattttgtatcttctgcttagaagtcataagccagcccatgaaccatgccaacaaacataccttcttccatgtgattattgcagccagatttttcagtgcaggacaagcaaagttttctctgaaagcatgagataataattctcagccaggtcttcgccatttgcaagggatcagacataagagtttttagctctgttgatgttaatagctagagcccctgttagcagatggcatgttagtggcctgttgctgctggctattaaatcatacacattggccaggcacggtcggtcacaccagtaatcccagcactttgggaggctgaggcgcacagatcacaaggtcaggagtttgagagcagcctgaccaacatggtgaaaccccatctctactaaaaataaaaaactaggcaggcatggcggcacatgcctataatcccagctactcaggaggctgaagcaggagaatcacttgagcccgggaggcgaaggttgcagtgagccgagatcacgccactgcactccagcctgggcgacagagcgagactccgtctcaaaaataaataaataaaaatattaaaagtaaatgatacacatcatgagatcatctcccaaggcagcagagaattttgcgtatttttttcttagtttatattgttgcaacagagtaattcttcttgatcacagactttgctttttttgttgttattgtttctaagtttcacttgatcctcaaaataacctgagacagacaaaacagataggttttcccattaatctccttcatgagtctgactttgccagccaggctgatcttatatccatagccctggatgcatttatttatgttattccatctgcctataatgcccatccccaatcttggcctattacatgtgtctaacattactttgatgtgtacatgtcagtgaattaccacttaactgattgttagctttttgagagtatgtatcaaatctcatgtgcatttgtgtgtcctgaaacaaattattaggtataaagtatgtattgcaaataccatgcccagggaatatttcagagctgtggttctccaagtgtggtccttagtctagcaacatcagcatcgcctggcatcacctgggaacttgttggaaaagcacattccatgatcccacccctgacctcctgaatcagcaaagctagggatggggctgagcaatctgtattttatcaagctgtccaggtgacattgataaatgctaaactttgcgaattactgatttagagactaatcaactcattaaaaagttaataaataggtgagaatccattgtagcaccccagtcacctatgctattatcttctaaattcacattatacaaattgtatgccacaaaaatgtttaaggtgaaaacccaaaggaattgatgcattggattaattcacaaaaatttaacagctgcaattcatagaaaaaaaatgcatgcatatctgataattagttgtagattaaaaggtgaacaacttcttattattaataccaatgaaaatgtaattttttaaaaattatggccaattggtagttttttaaaaatctgttatgccttgtcatgataaggataggtgaaaaggaactttcataaactattgctaagtatatgggtgaaaccttttagactatttgggtctacctaataaattaaaatgttcagtcttttctaattttagcaccgtatttctacagatacactggcatatctgtggagaggggcacttctgcttgtcacctgatatcccttactactactaatatttttataagcattattacctttaaaatcatgttaaagggtaaatgatgtgcaacagactcagtttccccttctctgactcctgtttagtttacatctcttttctctctctcatttgggggctcaatacccttaagattatagagaaattgcagtaggtaatgttggctggaaatgctaacttgtaagaaacttgggtggaaatggtttaagccatcagcctaaacgatcagtgaaaatgtcagacattctccgctttcttcttccaagatactatactctactgtggccttagagttcttaattcctatataattctgattaaaaaggaaggagatgctcctaatataccttgctgttgtcccctcctgttcattagggtagctgatgtcagtcaaaataaagtctagaaattcttgagtactttttttttttgtaatatactgagaatgcttgaagcaaactaaccaaatttaagtgttttaaggatattaacagtaacaggagagcccctcgattttctgctaagaatcaacctgaacagtttccagactgctggttggcaatgtcagtatcatgagaaaaactgaaaaaataagaaactttagagaaaaagaggaatcagtgcaggctaggggaatgaacttggtaatccagtagtctttccctgccagcattttgattgataagctaagtgtctgctttttctcatcccttattttttggcaggaaaccagtattttgatacttgaatgccttctttctatcttaagggagaagccttcttaaacttcaagtgtgaagtagatcctttaggtataggatgaaaaattaagtgggactatttgtgaattactaaatcgcaaccattaaagaaactaatgccctaattagcctgtcttttgacagacatggcatccatatgtaagtctaatattgtatgcaaatgtgagacaggttttgctttctagatctctttgatgtgaaaggaaaagctggcttcctggcttccttcccatgttctgtatggacctgccttcatcctgctgccaacagctcctcaagggaacatgaaagtggaattaaaccctcctggttttcagattcagaatcctgggtgattaagtggcttgatcaaaatcagaaactgtgaaagtgctaaaaatggtcaaggcccaaggagttcctcatgtgtggtcaaaagcctttcagaaatcaaagtgtgtctttaaatacctggggtgcaaaaatcagatgttttttatttaaagaaaaataggtatattagagcagaactgggggccctctgtaggagcctgagtctgggtgtttactgtcgtctaaggccttgttaatgtgggcttgaggcttcggaatccacgaacaggggaagctagcgtctctcgctgactccagtcctagctcgagctttccatactgccctacgtgacttctgcacttttgtgccatcagccagtagactgagaaaagtatctggtatttgtcggtgtcttcggtttagctgctttaagtccttggatggaaaaggctgattgtcatcctgaaatcattgtctcctaatgtgtaattggggccaagtgaagcacagtaatttagaggaattctagttaaaaatacagattcttgggctctacctcagacctacagagatctgcatttttcttattcccccaggtgatcctttaaacattcagaggtttttaaaactcatgcttatatgcaataacaaaaatataaggattcttagaaacttgttatatttgtcttgaacatgggatgacagacattgtcattttcattgaaaaaaaaaatgaagcagagactcagaatgtatttagagacaactctaaatacattctggcagagcctaactactggattgcagagatcctccctgcttcccccagtccccatctataaactggccatgtgaatttctagtggtgttcacttccaaagtggtgattttataaaatattacaatgaagcattattaacctcaagaagttcagatgcatagagagtttatttgggtgtggcagcattagtggctaggttttatttatccaagataagacttcttattagctatgaaaagggattggcataaactgcttattaatgtccattaactgtatctaaagggaataaacagtataaaaggccaggcatggtggctcacacctgtaatcctagccctatgggaggctgaggcaggaggatcatttgagcccaggagtttaagaccagcctgggcaacatggggagaccctgtttctacaaaaagtcaaaaaaataataagctgggtttaggggtacacacctgtaatcccagctagtcaggaggctgtggttggaggatcacttgagcccaggagttggaggttgcagtgaggcctggcggcaccactacactccaggctgggtgacagagggacgaccttatctcaaaacaaacaaaaacaaaaaactgtataagcataagatgggcttgagtcacaaaattctgcctttcataactttcccccatttgctgtttgatttgctgtttcagaaattgtgaacctacttctctacttatagaacatttacctccctcttagattcttccctttctatcattgatactgacattctgcttaaagaattatccatttaggctctcttcaaatccaaagtgatattattttattttaataagagatgaaagggaaaattgacaaaatgtgcctggagttgtcagcattgtaaatgatggactgtaaaagcttcgtcttggaagattgtcagagagcctagtgccagctcaaaaagtgtggtctttttagagaattgacagaaatgttacaaagaaaacatgtgctgaataccaactccaggtgcgaatcctgagaagcaactcactctattctgcagtaggagatgaattttgcaggttatacagcagagcaatttaaactcccttgtttcttaaagtatatcctatagtgacttgggatggaagtcatatactatccccttttttgttccttcaaagtaaatacaccacagagctggagatgtcccagacacagggggaagctgtttggatttttcccactgcctgtaatgaagcaagatagggaggaagaacttgggaacttgcgtggcaaatggtcaagtaaagaaaaagaaaagctttttctttacttgaccatttgtcacatgtgctttcaaatcctgaactgagtcacatctgaaataaatcacttccttgtcatttccatgaaaggggcagtacaagaaattccttgcacatccctggtgagccccttgttactctcagattcagctattgccttcaggaaaaaaagaaaaagaagccgaagaaagaaagaaaacagaatcctctagaaaaagctattctgacctgtgctctttcttttgtctgagtgtgactagataaatgcattagtctgctagggctgccataacaaaataccaaaataccacagactgaatccttcaacaacagaaattatttttctcatgcttctggaggctggaagtccaagatcaaggtaccagcaggtcggtttctgaggaggcctctcttccttgatttacaaatggccacctacgcatcatgtcctcacatggtctttctcagagagaaagagaggtgtctcttcctcttcttcttttgttttgttgagacagagtttcgctcttgttgcccaggctggagtgcaatggcacgatcttggctcactgcaacctccacctcctgggttcaagcgattctcgtgcctcagcctcccgagtagctgagattacaggcatgcctggctaattttgtatttttagtagagacagggtttctccatgttggtcaggctggtctcgaactctcgacctcaggtgatccacctgccttgccctcccaaagttctgggattacaggcgtgagccccgcacccggcctcctccttttcttataaggacacctgtcctattggatcagagtcacacccccatgacctcatttaaccttaattatctctttaaaggtctgatctccaaaggtcccttgagggttagggctttgatacatgaattttcaggggcacacagttcgatccatagcagttggcttattcatactttgggtagatcctcttcatcgttcctcagctactgtctatttagtgcacagcttctttatcctaaagctagtcaggggaggcatggaaggtattatttctcctgtttctgcacctaaggcattaaaacgaggttgtggtctggcagcctaaactcggccttgatgaacttcaaatcctagacctccttggtatgtgaagtgcatacacttgtatggagggatttgacaggttttttttttttttttttttttgagacagagtctcactctgttgcccaggctggaatgcagtggcacgatctcggctcactacagcctccgcctcccggtttcaaatgattctcctgcctcagcctcctgagtagctgggattataggcgcgcaccaccacacccagctaatttttgtatttttagtagagacggggtttcagtatgttgatcgggctggtctcgaattcctgaccttgtaatccactcgcctcggcctcgcaaagtgttgggattacaggtgtgagccaccgcgcccagcccagatttttttaaattagtttttagtgtatccacactgttaaagcaggtggatactgctggatagtataacaaagtatagtgtgacaaacccataaccaagcattgttagctatattttctcatgtcttgagccaaaaggtcacagttaacctaatgttgctactaaacactcataccataaaacattctattccagtcttccagattttacacaagaataaaatgaaaacttttagactactggagatttttttttaaatacacaatttgttgaatgtgaacgctaaattgaaattgacatgctaagttttatagattacttcagctgccctgacttcagtaaatttagttgtgacacaggtacaacagctgacttctttctggcattagaggtggtgatgtttcgtcactctggagttagtaaaacctgaaacccgccaggcatttgacacacagtttgctgccagggacagctcagcttctggcttattggagaggaaatgacctaacctctgtgtttacaaagtgtttgaaactgaggcccttccacttccttgctgagtgtaagggttgacctggcatccaaaatccccatgatcttctcaggataagtcatgcagccattggtggtcaagaaaggatttgttccctgtggaagggcattggaagaggaacagagctgaaacacacgggtgttcagagggaaaacatctctgcacttaagagggctagattagactattagactattatcatagagctcgtgattcatctcctttgtttaattccagccttgagtaaaggactcactaagaaaccacattcggccgggtgttgtggctcacgcctgtaatcccaacactttgggagaccgaggcagatggatcacaaggtcaggagttcaagaccagcctggccaatatggtgaaaccccgtctcaactaaaaatacaaaaattagtcggggatggttgcgggcgcctgtagtctcagctactcgggaggctgaggcaggagaattgcttgaacctgggaggaagaggttacagtgagccgagatcacgctactgcattccagcctggtgactggagcgagactccatctcaaaaaaaaagaaaccacattctattccagtatatttattataaccagaaattcttgtcttatcagcaaatagctaatattcacttacatgggcagtgtattagattcctagggctgctggaaccagttaccacaaaccgcatggccgaaaccaacagaaatatactctcattatctcattattctagaggttagaagtcctaaagcaaggtgttgccagggccacactgcctcttacagctctaggggagaatctgtttcatgcctttcgcttgctctagtgctgctgccagcccttgatattcctttggttggtacatgcacccctccaatctccacctctgtcttcacatggtgttactccccatgtctctgtttctgtgtcttctctaatatgattaagggcccacccttctccattgtgacctcatcttatatctgcaaagaccctgtttccaagtaaggctgtattcttaggtgtaggcattaggacttcaacatatattttgggggaaaattcaacccataatgcggttatgtttttattttccttgacttttaagaatgtgaactattgtcaaattttttcaaccactatcaaatttaaatctaagaactacatgtcttaaatttatgacctcttcattaaaaaaaaaataataacatttaattggtaccaaaacgtgaaagagtttaatgctttgcatttgcttgaggctgattaatgagtaaatgtgtcaactgtggctgttttaattagccagggttaattaatgtttaccacacttggaagatgaaaaggcatgtataaatgctctataatgtgaatcattttttaatttgttgttgaagcttatagtgtagccaagtaaggattgcttacatcatacttacaaagggaggttcattaatggatacaaatacacagtttgatagaagaagcaagacctagtatttaattatcagcaaggtgactaatttacagtaatctactgtatatttaaaaatagctaggagaacctaatttgaaagtttctagcgtaaagaagacaggctgggcacgatggcttacacctatagtcccagcacttgggaaggccgaggtgggaaaatcacttgaccccagcaattcaagactagccagggcatcatagtgagaccccatctctacaaaacaaatatttaaaaattagccaggtgtgatggtgcatgcctgtagacccagctgctctggaggctgaggtgagaagatcacttgagcccagcagttcaaggttcaagtgagctatgatcacatcactgcactccagcctgagtgacagagagagacccttcctcaaagcaaaaagaaaagaaaaacatttaagtgatggatatcccattatactgatttgatctttccaaattatatgaatatattaagttttctcatataatctgaaaatatgcacatctattatgtatcaataaaaaataaaattttaaaaatgtaaaaaacgatgttggagcactatggtacttaacgtagaggctctgacttttgaggtacattcattacctgttactgaagttgagtatgctttaatattgtatgtttagaaaaggtagttacatttttaaatctgattgtgtaagaagaaaagagtagaaatgtttggggccaagtcaataattatttagtatatcataatttgagatgaaatgtgtttttttttttcttttttagtttcaagttgaaacattttaagaacagataaggaaaggctaactgtgctgagatgaaaataacatttttaaagtaactatcttaaagaaaatacatttctagaattttgtgtaaattttcaacaaagactcagaagcctggatgaggtcttgtgccaagactggaaagtgtggggattagaaaaagtttgaagagattgttgtgagatcctctcgtatgaggcctgaagctgtacatccatggaaagcagttaccccaggcgggaaaacaggttatccattctcgagtgagtggccgggtgggaaatagtccatcatgtcgaatggatcgtactttccagcaccaagatttgtggttctctaggtgaataagatgacaccccttcacttcacttcacttcaccattgtctgcatggagtacgtatctaaaaacaaattagcaaaaaggttgggtctactttacttttcatctcctacactattttcttctttgttctcggcaggaaaagatgaagttgcattttattattatctcataggattcttattctaaaatgatttgatttaaaacccattgcactttatttggataaaaaatgcttcactctctagttcttaacaataacaccagcctgattttattacgttaagcactgcaattagacaatacaaagaatttgcagcaacaacatgttattttatcatgggtggaactgttcattgtctagcagaatttattaccagataaatcccttttggggaatctaatgtggatgtttttttcaaagcgcctgtggagttctgctagtattgggaacgtttccattttcattctgatgcttccctgtgggctgtttcagcatagaagtgagctctctgggtccatgtttccattggctccacccaatattctgaaagattgcaactcaacatgtgtaaacagaacctttctcccccatattttctcttttcgtagattaaactcaggtcaagaattagaagtttatcctccaccccgatagcatctcgccctatcccaaataccgccaccaaaatgcccactgtttttccttgggccagttcctgaaatatctttctggctgtatcactcttgtgtgcatccttatcggctatgcttttggccattttttactgctgacttgtctggttagtcttttataatctccaggcttctcctccatcctttttttacagtttacctgttcatgaagcgcaggtcacttgacccgtagaattcccataggctgggattttgctaattgcatactcatggtataggtcagtgtattcctctgccctctgtatcccacaaactgaaagcctcttggctaagctgcttcccttttcacttcccttgtaccacagcagccagaagagtcacagatcctgggcagcttgtcctctgtgcctcggtacatgcacttgtatctgctaggacaatgatctctttccctggggccccactgtaatatcactagtcacatccctaaagcccaggcttgtttctttgtcattctgtcggtatccctaccataacacttgtaatagtaattatttttatgcccatcttttcttctgaaccatgagcccttcaagtgaaaagaaattgatgaagttatttctagcactcccctggaaaaaggaaggcaggcagggagggaggggctcactagtttatgataaagtggtttgattttcttggttgcagattatttcttttaaagtgactcttttcagcttctctcactcctgtgcatcgtgaatgctctcctgaagacagacgctttccattcatttccaagccccccatgtgaagtgctttctgttgccaggcttggtttccccacacgttgttaaagaagttaaagaagcttgtgaataagctaaagcagaggagaaagaaccattgctatatctagaattgttcaccagaaagcaagttttgtgttaaagaaaacacgagtaggccactgtgcagaagtgcttacagggtttgtgtgctttttaaaatctgcataaataaacatttcaaatgagctatcatatatacaaggatgatgtgctgtctgtgggaaaaggacccaatcctgtgttgtgattttacatatatatatatatataatttgtgctattacacacttgcctgatccccttataattatctgcataagtctttttaggctctttgaatgaaattcactatgtatggatgttgaaaatcatcctttctaccatgagctcatcagacttgctaaccaattctttgcatcctttgaagtataaaattccatcaattcagcatgtatcaacatgaatcaattagaatgcactttcttcacagcatggcggagtttttgctggctgccgggaggggcactgtttcccaattcattcatttgcttgggcccgccccaggatccttcttatcccggtcagatagacttctctcatgagcttgagagacggggctggtcacacaggacacggctttagggacacgactcactgaaactacaaaatgagtgctgctcttagagttgtacccacagcctgcacagctgttatggcaatgctgtgggcaagtcttgctacggcccattctcctccctcccttgtcctgttggaaggttcaggttggagctgaaattcagtccagcacgcagctggacaagccatgtctcctgtatgtgactcactccccagagaaagcatcatgtggactagtaagggcttggctttatcagaagcaaaggagaggtttacatctcttcttgctctctcttagtgggttcagagctccagcagaaaaaatgggacactgtggattgatctccagaaaaccccaaaaccagcactgaaggctccaggagattatgcctttagattgtatacaggcaccagaatgacttttgcctttataaaaatgcgtgcaagagcatgaaaactaatatttgaggaatttttttttctcactgtactttctggtcctcactgaaagcattggcagttttaaaaaatgaaatgtgatactgttatagacgtagcaaacctcacgtttcattttttccaattcattctatatctaggaacttacttagaaggagaaacaaactgtacccagaaaaaggaatgaaactggagaattcttcctcatgattcctctgtgtcctcaggcctaattatcctcattccaaagactctccattgcctcaataacttcgaaaaatttactctccttaagcatagtcataaaaattgcatagtaaagtgataaagagacgaaaataatgctgtaagaataaaaccattaaaaaaaaaccactactcttgattatttctgctgttagaattgtaaatgaattctcattccttttttctacatttttggcatgaaatttatgttactcttataattagtaaaaacattatattttattaataaaagaagactcacagtgaagtcataacatatgctgcagaggttagactcttcaagtcagatttgaagagagaatttcacgtaacaatcaccttgaaatgtcttcaggtcagccatcaaagtacacatctctcaaccagccaggcagccagtttttcctccagcatgtgaagagtgaagagatagctgtttcttggcttaaagaccatatgaaataattgacttatgtttaaaagatatgagggatcaggccgggtgcagtggctcatgcctgtaatcccagcactttgggaggccgaggcgggtggatcacaaggttagaagatcgagaccatcccggccaacatggtgaaaccctgtctctactaaaaatacaaaaactagccgggcgtagtgatgcgcgcctgtaatcccagctactcaggaggccgaggttgccatgagccgagattacgcctctgcagtccaacctggtgacagagcaagactctgtctccagttaaaaaaaaaaaaaaggcatcaaaattacattcacttctatttttatttttgtttgtagaagacatgcagtttgtatcacattaatgggcatatgatgtaatttcactataaatcgaaccagaggaagacatccaaatatgtataaattaaagctgacaatgcctacagtactaaactttttgttttagtataattggagacttagaaaagttctaaaaatatatttaaaaatttttaaaggaagttgcagacataatggccctttacacctaaatacttcactgtacgtttcctcaaaacaagaagatatgtgtgtgtgtgtttgtgtgtgtgtgttgtaatcttaatacagcatctttcagagattatttctgtagtatataaacttggtttttttgtttgtttgtttgtttgtttgtttgttttgagatggagtctcgctctgtcgcccaggctggagtgcagtggcacaatcttggctcgctgcaagctccgcctcccaggttcacaccattctcctgcctcagcctcctgagtagctgggactacaggcgcctgcaccatgcccggctaatttttgtatttttagtagagacgcgatttcacctcgttagccaggatggtgtcgatctcctgacctcgtgaaccgcccacctcagcctcccaaaatgctgggattacaggcgtgagccaccgcgcctggccataaagttgttttttagtctctactttaagttcctgacaacccaggcaggaccaagactccctaatgtagttggaaatcagatcagcacattggcacaagttctacaggtaaacgggaaaaaggagccctacaatctgcttttagatgccagggagcctggctggtctttatcttgattactgactgatgccactggtaggtctaagttctttgatctatgatgtttgaaagccaaaaggaaatgtgttttaaaggaaaagggacttcttctgtaccctcagtctaagccagacaagtgatatttcaggaaaatgtccctagcaataaggaatattttcacaaatgatgtgaagctttgtttgtgtcccttccagggcagtttgttaccataaggggacaaagcatgcttcatttaaacatgtgtggccaggaaactgttatttttaaacaaaaataaaccccgatttagacatgtagtctgagttttaactctgggagattttcaagattgatagtgtagctaaaaataactgacctaattttctggaggtcaagataaaaactggatttgtggtacagcatttactttgtgataatatgtgtataaattcttccccaagttggaaaatgtggccgcgtcatacagggctgtatctctcttgaccattgaagtgcctaggtcgctacactacaatttgacttagagttggtgaaacttctggccattcacgtagccatcgtattcatcctgagaaagggaaacctaaagacttgggagagtgttctgggaggaaagtgctattgagaatgggctgtccaagcaaagtgacaggcaggcctcacttagctagaagagggagcttttcagccctgctccaccatcaggaatctccgtgaccttggactactgctttaatctcttttatcctggcttctaaaagataccgtcgaattcattctttttcaaacctatcaaaaatttgaaatgtatgaaaaagagggtgcttgtgatgatagatgggtttatgcttccttttttttttttttttttttttttttgagacggagtctcactctgttgcccagactggagtgcagtggcgcgatctcggctcactgcaagctccgcctccggggttcacaccattctcctgcctcagcctcccgagtagctgggactacagtgccaccaccacatctggctaattttttttgtattttcagtaaagatggggtttcaccatgttagccaggatgctctcgatctcctgacctcatgattccaaagtgctgggattacaggcgtgatatgcttctttaatattagtttctaatccaagtaatatagccatgtcctctataaaatggaacttttcatggaacagtgagaaggttgcattaggaattgggatacttgggttcagatttcagttctgccacttgctagttttaagtatacttctcaggatggatgtcagtttgttcaactgtcagtgggagtagtgatcccactaaggaagattattgtggttatcaaatcagcagatgggtatgaaaaggccatgagtgcagttctcatggttatgaaggtgtcataactggcatagtcccatcacagtatggctttggcatcgcagatggctgtagcatatatgctaatcagaacatccctaccgtccttaaagatctcatgggttctctcccaaatcagctgctccaccgacctacaagtaagtccatgcttctttagtatatttactcactagtgaaggcccgtctgctaaaagtgtcttcagtaggggagagctgctcttttctgggaaacccagcaatgaagtttgtcctgtgatgccctctacacttactgacctgtttcctgagaacttcccgtaacaaatggtccagggaagaaggaagtctgtgaccagtctttcctttcctgtggctttctggttcagaaatgtgaccctgatgataagaaacttcaaggcatgactctggtgttttggcaatattcccctggcagtattgtttcttggctcacactcatctcatgccttgctttgtttgctttgtttttgagaattgtttttttgtttgttgtttgtttttggttttggcttgtgagacagagccttgctctgttgcccaggatggcgtgcagttggcacaatctcggctcactgcagcctccacctcccgggttcaagcaattctcctgcctcagcctcctgaataatggggattacaggcacacaccaccgtgcctggctaatttttgtatttttttagtagagacgggtttcaccatgttggccaggctggtcttgaactcctgacctcaagtgaaccacccacctcggcctcccaaagtgctggggttacaggcgtgagccaccacgcccagccattatgtttgtttttctttcatgaagggataaccgaggctgttgtgtggtggtgtcttggtacagctcgtggctgtggtcagtgtggtggagggtaggtcttctcagatggtaacatgcatgagagtcacctggggatcacattaacatgcagattctgattccctggatctgggtgggacctgaggttctgcatgtctaacaaactcccagagaacacccatgtagcaagagtacagacaaaaggattcaaagcgaagggactgcaagaaccatggaaccagcctcccacgattttgcggatgacgaacccgagccttggggagtttcagaggccatggagagatgaagtataataaacagcatagcagttgtatgttgagccaagaatcagacatgcacagaactgctgattggaaaattaaatctgtttctcatagccctggtgtggcaaaatgcctagctggaagtgggtgaccgcttgatttacagtgggcctcctgggtagggaaaattagtccatcccatttacagctacttccaatgcagctgtgtcaggggagagagaagaaaggacatggaagagagcgtagaggtatgatcttcagtcctttcaggcaggcacataagtaaatttttatttaaaagataagaattggtcatatgcgaatctcctgttagataagattaaaaacaaatttccattttgtggagaagttgcagataaatatttttccatgggatccaaaagaatcaaaaaggaaagaaaatagtaatagcagtggaggttcgatcctagacagaagaaaatccagcaagccgagggacttcatattctcactggtgtccaaatcagagctttttttttttttctttgtttactttaagttctgggatacatgtgctgagcatgcaggtttgttacataggtatacatgtgccttggtggtttgctgcacccatcaacccgtcatctaggttttaactccgcatgcattaggtatttgtcctaatgctctccctcccctttcaccccacccctggacaggccctggtgtatgatgtccccctccctgtgtccatgtgttctcattgttcagctcccacttatgagtgagaacatgagtgtttgggtttctggtcctatgttagtttgctgaggatgatggcctccagcttcatccatggccctgcaaaggacatgaactcaacctttttttttttccttttttttttttttttgtatttgcaaattcaaatctttttttttttttattatactttaagttttagggtacatgtgacattgtgcagcttagttacatatgtatacatgtgccatgctggcgctgcacccactaactcgtcatctagcattaggtatatctcccaatgctatccctcccccctccccccaccccaccacagtccccagagtgtgatattccccttcctgtgtccatgtgatctccattgttcaattcccacctatgagtgagaatatgcggtgtttggttttttgttcttgcgatagtttactgagaatgatggtttccagtttcatccatgtccctacaaaggacatgaactcatcattttttatggctgcatagtattccatggtgtatatgtgccacattttcttaatccagtctatcattgttggacatttgggttggttccaagtctttgctattgtgaataatgccgcaataaacatacgtgtgcatgtgtctttatagcagcatgatttatagtcctttgggtatatacccagtaatgggatggctgggtcaaatggtatttctagttctagatccctgaggaattgcgacactgacttccacaatggttgaactagtttacagtcccaccaacagtgtaaaagtgttcctatttctccacatcctctccagcacctgttgtttcctgactttttaatgatcgccattctaactggtgtgagatgatatctcatagtggttttgatttgcatttctctgatggccagtgatggtgagcattttttcatgtgttttttggctgcataaatgtcttcttttgagaagtgtctgttcatgtcctttgcccactttttgatggggttgtttgtttttttcttgtaaatttgtttgagttcattgtagattctggatattagccctttgtcagatgagtaggttgcgaaaattttctcccatgttgtaggttgcctgttcactctgatggtagtttcttttgctgtgcagaagctctttagtttaattagatcccatttgtccattttggcttttgttgccattgcttttggtgttttggacgtgaagtccttgcccacgcctatgtcctgaatggtaatgcctaggttttcttctagggtttttatggttttaggtctaacgtttaaatctttaatccatcttgaattgatttttgtataaggtgtaaggaagggatccagtttcagctttctacatatggctagccagttttcccagcaccatttattaaatagggaatcctttccccattgcttgtttttctcaggtttgtcaaagatcagatagttgtaggtatgcggcgttatttctgagggctctgttctgttccattgatctatatctctgttttggtaccagtaccatgctgtttcggttactgtagccttgtagtatagtttgaagtcaggtagtgtgatgcctccagcttcgttcttttggcttaggattgacttggcgatgcgggctcttttttggttccatatgaactttaaagtagttttttccaattctgtgaagaaagtcattggtagcttgatggggatggcattgaatctgtaaattaccttgggcagtatggccattttcacgatattgattcttcctacccatgagcatggaatgttcttccatttgtttgtgtcctcttttatttccttgagcagttggtttgtagttctcctttaagaggtccttcacatcccttgtaagttggattcctaggtattttattctctttgaagcaattgtgaatgggagttcactcatgatttggctctctgtttgtctgttgttggtgtataagaatgcttgtgatttttgtacattgattttgtatcctgagactttgctgaagttgcgtatcagcttaaggagattttgggctgagacaatggggttttctagataaacaatcatgtcgtctgcaaacagggacaatttgacttcctcttttcctaattgaataccttttatttccttctcctgcctgattgccctggccagaacttccaacactatgttgaataggagcggtgagagagggcatccctgtcttgtgccagttttcaaagggaatgcttccagtttttgcccattcagtatgatattggctgtgggtttgtcatagatagctcttattattttgaaatacgtcccatcaatacctaatttattgagagtttttagcatgaagggttgttgaattttgtcaaaggctttttctgcatctattgagataatcatgtggtttttgtctttggctctgtttatatgctggattacctttattgatttgcgtatattgaaccaagccttgcatcccagggatgaagcccacttgatcatggtggataagctttttgatgtgctgctggattcggtttgccagtattttattgaggatttttgcatcaatgttcatcaaggatattggtctaaaattctctttttttggttgtgtctctgcccggctttggtatcagaatgatgctggcctcataaaatgagttagggaggattccctctttttctattgattggaatagtttcagaaggaatggtaccagttcctccttgtacctctggtagaattcggctgtgaaccatctggtcctggactctttttggttggtaaactattgattattgccacaatttcagctcctgttattggtctattcagagattcaacttcttcctggtttagtcttgggagagtgtatgtgtcgaggaatgtatccatttcttctagattttctagtttatttgtgtagaggtttttgtagtattctctgatggtagtttgtatttctgtgggatcggtggtgatatcccctttatcattttttatcgtgtctatttgattctcctctttttctttattagtcttgctagcggtctatcaattttgttgatcctttcaaaaaaccagctcctggattcattgatttttttgaagggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtgggcatttagtgctataaatttccctctacacactgctttgaatgcgtcccagagattctggtatgttgtgtctttgttctcgttggtttcaaagaacatctttatttctgccttcatttcgttatgtacccagtagtcattcaggagcaggttgttcagtttccatgtagttgagcggctttgagtgagattattaatcctgagttctagtttgattgcactgtggtcggagagatagtttgttataatttctgttcttttacatttgctgaggagagttttacttccaactatgtggtcaattttggaataggtgtggtgtggtgctgaaaaaaatgtatattctgttgatttggggtggagagttctgtagatgtctattaggtccgcttggtgcagagctgagttcaattcctgggtatccttgttgactttctgtctcgttgatctgtctaatgttgacagtggggtgttaaagtctcccattattaatgtgtgggagtctaagtctctttgtaggtcactcaggacttgctttatgaatctgggtgctcctgtattgggtgcatatatgtttaggatagttagctcctcttgttgaattgatccctttaccattatgtaatggccttctttgtctcttttgatctttgttggtttaacgtctgttttatcagagactaggattgcaacccctgcctttttttgttttccatttgcttggtagatcttcctccatccttttattttgagcctgtgtgtgtctctgcacatgagatgggtttcctgaatacagcacactgatgggtcttgactctttatccaacttgccagtctgtgtcttttaattgcagaatttagtccatttacatttaaagttaatactgttatgtgtgaatttgatcctgtcattatgatgttagctggtgattttgctcattagttgatgcagtttcttcctagtcttgatggtctttacattttggcatgattttgcagcggctggtaccggttgttcctttgcatgtttagtgcttccttcaggagctcttttagggcaggcctggtggtgacaaaatctctcagcatttgcttgtctgtaaagtattttatttctccttcacttatgaagcttagtttggctggatatgaaattctgggttgaaaattcttttctttaagaatgttgaatattggcccccactctcttctggcttgtagggtttctgccgagagatccgctgttagtctgatgggcttccctttgagggtaacccgacctttctctctggctgcccttaacattttttccttcatttcaactttggtgaatctgacaattacgtgtcttggagttgctcttctcgaggagtatctttgtggcgttctctgtatttcctgaatctgaacgttggcctgccttgctagattggggaagttctcctggataatatcctgcagagtgttttccaacttggttccattctccgcatcactttcaggtacaccaatcagacgtagatttggtcttttcacatagtcccatatttcttggaggctttgctcatttctttttattcgtttttctctaaacttcccttctcgcttcatttcattcatttcatcttccattgctgataccctttcttccagttgatcgcatcggctcctgaggcttctgcattcttcacgtagttctcgagccttggttttcagctccatcagctcctttaagcacttctctgtattggttattctagttatacattcttctaaatttttttcaaagttttcaacttctttgcctttggtttgaatgtcctcccgtagctcagagtaatttgatcgtctgaagccttcttctctcagctcgtcaaaatcattctccatccagctttgttccgttgctggtgaggaactgcgttctttggaggaggagaggcactctgcgttttagagtttccagtttttctgttctgttttttccccatctttgtggttttatctacttttggtctttgatgatggtgatgtacagatgggttttcggtgtggatgtcctttctgtttgttagttttccttctaacagacaggaccctcagctgcaggtctgttggaataccctgcagtgtgaggtgtcagtgtgcccctgctgggggtgcctcccagttaggctgctcgggggtcaggggtcagggaccacttgaggaggcagtctgccgttctcagatctccagctgcgtgctgggagaaccactgctctcttcaaagctgtcagacagggacatttaagtctgcagaggttactgctgtctttttgtttgtctgtgccctgcccccagaggtggagcctacagaggcaagcaggcctccttgagctgtggtgggctccacccagttcgagcttcccggctgctttgtttacctaagcaagcctgggcaatggcgggctcccctcccccagcctcgctgccgccttgcagtttgatctcagactgctgtgctagcaatcagcgcgattccgtgggcgtaggaccctccgagccaggtgtgggatatagtctcgtggtgcgccgtttcttaagccggtctgaaaagcgcaatattcgggtgggagtgacccgattttccaggtgcgtctgtcacccctttctttgactcagaaagggaactccctgaccccttgcgcttcccaggtgaggcaatgcctcgccctgcttcggctcgcgcacggtgcacgcacccactggcctgcgcccactgtctggcactccctagtgagatgaacccggtatctcagatggaaatgcggaaatcacccgtcttctgcgtcgctcacgctgggagctgtagaccggagctgttcctattcggccatcttggctcccacctcccgaactcatccttttttatggctgcatagtattccatggtgtatatgtgccacattttctttatccagtctatcgttgatggacattttggttggttccaagtctttgctattgtaaatagtgctacagtaaacatatgtgtgcatgtgtctttacagtagaatgatttataatcctttgggtatattcccagtaatgggattgctgggtcaaatggtatttctagttctatatccttgcagaatctcctcactgtcttccacaacggttgaactaacactctcaccaacagtgtaaaagcgtgcctatttctccgcatcctcgccagcatctgttgttgccagactttttaatgatcaccatcccaactggtgtgagatggtatctcattgtggttttgatttgcatttctctaatgaccagtgatgatgagctttttttttttttttcatatgtttgttggccacatacatgtcttgttttgagaagtgtctgttcatatcctttgcccactttttgatggggttgtttttcttttttttctggtaaatttgtttaagttccttgtagattctggatattagacctttgtcagatggatagattcagatcagagctttaacagccagactttacagggaaaaggctgagtggcacctcctttatatgaagaaaatgttcttctccaacccacttgtgttcagtggatggaagaagctgttcagtaattataactgacacttgtaacacataactgattgatttaatgcccataaattttattttctttaatcgtaagatgtgaccgttaaattcttagaagacatttggctagtggattaataattaaaagaatattggatgaattcatctgcttcctattcagcagttcttttatttataaggctatttttctcaaaatgaatgatcatgaatatctaagaaaaaacaaaatatttagctgtttgaagatgaaattattatcttttgatgttgttttctttaaagtgtttacaatgataactaagtggtagggcttcttccctctgttactctaagtatgcctcgtatacatcctcaaagcattttttttttttttttttttggagacagagtcttactcggttgcctaggctggagtgcagtggcatgatctcagctcactgcaacctctgtctcccaggtttaagcaattctcccgccttagcctcccaagtaggtgggattacaggcgtccgccaccatgcacggctaatttttgtatttttagtagagacggggtttcaccaagttagccaggctgctcttgaactcctgacctcgtgatccacccacctcggcctcccaaagtgctagattacagacgtgagccaccacgcctggccacctcgaagcatttgtaaaagagcaaggtgatggggttcattaggtgtttggtatttgaaattaaatacctctgttattgatgaagccatattttaaatattatcaccactaaactcagttaaatatttccactgttaactacaaggttcaaatgcttggatattatagaagatatttgacttcaaagataaacgctacaattttgggtcataacaaatagcttttagaaggggaaaaaataagtcttgggaggaaaataagcagtctcttagtttaatatagcggttggcaaatatagatgcttaaaagttctgaaaatccccattgcaggccatcagagtgttgtaattaagtattaaattaattcagataaaaggataatccatcagggccactaatgtagtggaagtgtggtttcggtgtgattagtctcaccatggaagtcaaagaaagtcccactcagaatgaacaatgaaagaatcatatttgtctctgggtattttgtcataggctcaattgcccagtgccagtgtgtaagccactggtaaaatcctgagcccttagtcatgtctacaggtagctggctgccttagacattttatgatgacgaaattgaattccacttctgataatggtaatccatcatgtctacctctttttttctcttaaaactacactgacggccaggcgcagtggctcccgcctcatcccagcactttgggaggccaaggcaggcagatcgcctgaggtcaggagtttgagaccagcctggccaacatggagaaactctgtctctactaaaaacataaaaactatccggtcatggtagtgtgtgcctgtaatcacagtactcgggactgaggcaggagaatcgcttgaacccaggaggcagaggttcgttgttctgaatgacatagccaggggtggttatttctaaattttctgtgttgaattaagataggaatgtggatttcttcctattttgaacaacgaccaaaaaaaaaaaacgaaggctaagtgtgtttcactctgctaaaaatgtaccttttcttgaatctgtcactttttcacttacattttctgaaaatgaatataattaaccaccatggggctcagcatgctggtggccatttggaggaacttttctaccaacagtgtaacagagaaaagagaaaagggtactacagcgtacctcaacctcccctgactgttctgtgaagtacaagtgtaagatttctcactgtgatcttttgctcctctgcctttttactgccaagggctaagatgggcacagggccctttccagctgcccctgcctgagaaatgcttatttgatgaccaaaagcaataaccattccataggactaacggtgattaagtcagcttacaaatccacaagggtggaagagactgctaaagatattggaacagacatgcttaataatgcctaatttctacagctacttttgtgataatacgagaggctgaaatagataggggctgaacatgggaaggctggtgttatgaagcccaggacttggatttcagcactggtgcttactggccattgggtcttgaagagattggttcctttctcccagcctcagtctccttgtcccatcagattaataatgcctgccttgggagaggagagtcatacaggagagggcatttgagctgggctctaaagagttcaaataacgtagatgagcagagatgggcttccaagatagcaggaatagaaaacaaaagtgtcagaatgggtatgcatggtcttttgtgggaataccccaagctatttagcctgagagagaggtgtggtgtagacagctggagctgtgagttccatggctagaaatacggggtgggcccagaccacagagggccttcagcatctggcaaaggagttggaattaattcggtagaaaatgaagacccactgaaacaaagagggagccattcttgtgtgtcaggaactaagatttcaggaaccaaacgtaaaataaaaaccagcagggagaccattgtctcagtcggtttgtgctgttatcatgataccagagactgggtaatttataaacaaacaatagaaatactacaattctggaggctgagaagcccaggatcaagccgggtgcagtggctcacgcctgtaatcccagcactttgggaagccgaggcgggcggatcacgaagtcaggagatggagaccatcctggctaacacggtgaaaccccgtctctactaaaactacaaaaaattagccgggcgtggtggcgggcgcctgtagtcccagctactcgggaggctgaggcaggagaatggcgtcaacccgggaggcggagcttgcagtgagccgagattgcgccactgcactccagcctgggcgacagagcaagactccgtctcaaaaaaaaaaaaaaaaaaaaaaggcgccttgtggctgcatcctccagaggcagcaaaaactgttctcacatggcagatgcacataaaggggcctgtgctggtttcctctagcccttttagaaggcagtagtccattcgggggcacggggtggaactgtcatgacttgatttccagaagaccccatctctcactactaccacagtggagatttaagtttcaacacatgactttgggggacattcagaccatagcaagcagtgaggagactatcgtaattatccaggcgaaaagtaatcatattcctaggggcggtgagggtggtgtggaaaagcaagcaggaaatggctgagagaattcagaaatagaatgcaaaaggatttgtcggtagtgtgagtactggacacgaagaaatacagggagtcctggatggctctgaaggattcaagcctgtgactgggagggagaatggttctgccatgattagaaaacaggaaaggtggaagggaaagccagggaccttgagtacagttgtgtacctaccaagtgcatggatcaggagggagccctaagttggtatctccagcaaactgttggaaatatacaaatttgagtgggagggagaagtcaaagctggggaagtacatttcagattcacccaaaggtaggagatctttagagtttcaagagtcgatgggatttccaaaggggagaattgtagaaagagatggaaagagatacaagaagaaaatgacagggatgaagagaggagaaaaaaagagtaaaaagagttattcagagatgtcacagggaagaagataagtactgtgtatgcgaaacagtgagaacatttccaaataaaggacatggtaagtgatagtagataccacagacccaagaacagcgtagcccctggggagttcaagagctttctcgctggcaaaggttatttcctgtcaagatgaatgtgtatgcaccaataattattagtattccagaatatcctctctttagcatagtaagtgcttaacagtcacaagcaaaagatttatataaaagaaatgctcatttaatttatttaatcagacattggctaagttattagccgctacctatggctttgtaaaacatgaaaaagttggaggaaatctaatgagggtcttttatctgtaagggaaaggtcatcatgggcatcaagtattgaattttagaaacggtgccacagtgaatagggatatattataactcagattagacttcaaaaggactaatttgcttgggacttccatcatttaaaatcacttgcaataaaactgactacatgaaaagatattaatcacaagctttaatgctattccatcaaattattttaatgggtatatttgaacagaattttaatttttcagatacattttttgtcaatagaaaagagaatttgaaagatgattgtaaaatgttacaaatcaaatttaaatcctgatcttattttcattttctctagcaataaaactaaccagtgatgtgtctgaaatatatactacagcaaactcttgacacagctatgattggaaaatgagacattttggttaatcaaatattattgttaatagtgcagtgctgtctgtcggtctctaatttgtaaaaatcagagaaaaacatggtgaatctttggtgacataagagaagactcacaaatcttcatatcctggggtcagtttttaaagcagtaattacacatgatacaagattatattctaagctctctttttccagtaagaaaagataagtcaatgatgttccataaagctgattcttgtttaatcaatttttaaagtgggggggtaggcggcactgacagcattctttggaaagggaagcaacagaggagaaaggcaggatatttggtgtctgctcacattgggaaatacggggagccaggggtggttaagtccagagaaggctggatgtctgtctgcagggctacatctggaagggcaagggaacagtttccttgttgcctcagagcataggtcatgcagcacaagaggagcatttaggaaatgcagattttttttcaaggcaagacagtgaaaactattcaagaaggtagcaaactactggccctttaatgcttttcttatccttgcaagtgtgcaaggcaagacaggagattgtctgttgtctttcagcttgaatgagattgtgcctcacttactgccgccacaccaaagcctggcgtttattcctgtgcgatcattgtatactagggacgtctaactgtgtcaggaggaaatggcagctgagcccctgtggcctttcacctctagcagtgccacgctgcagcacattggccggctgccatgctcctgcctgcagtcctgccagcagctcttgtgagctgcgactttgcataagtagcttgaatgccatgtgcctcagttttcacatctgtaaaagggagatgataatggtacctatgtcatggctctaaacgcgatcatgcacgtgaaagcagttgaagtcttgcctggcagaagtaaatggtggctgctgctgctgctgctgttgtgattgttgttactcaccaaagagatggttttgtttggtttagatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaaggtaagagtcccctgagccagcaagggcgttctgggaggtatatatacacatacataacgtgtgtgcaagagagagagttatcttttgtatgttcttgagtggtgaattttttttgatgtagtcataaatattttcattataattttactttaaaaagtaaatactaggagttactatgtcattaaaataatatatctgtgtaaattattctttatattttttatgctgcaaagcatgtaaactgccaattgttggaaaagacaacaactccattgtataggtggcaaaacaaaatggtaatgatttacataacatttgcaggtaaagggcctcttgtttcgcacatgtccaggtttgcattctagggaatattatccttcgagtgaacactcccggacgaaggggagccacagtgtattattgaagaggcagttcttcacaaggaaaatgtgtggcaataacattcttctaatttttattttccagttcctcttcttaatataaaatggtacattatataacaaaagtggatacataaagagcatgtactgtatatcatattattatgtttatgactttttcaaatgaggaaatggaggcaccaggagactaagttcttaagtgatgacttttggtaggaattttttagaaatgttttttaactgtgaggtatattgttccctctggaatggaagcacaaaattcaacttgtgtttatgacgtaatgacctagaatgccaccttccaaaaaagcagatagatagtgaaataaggaaaaattacagtttaccaacagagaggagttttgagggcctgcagagatgcagatttgacaggcaccaaagtagtcttcactggctacttattcttacatgaaatttcagattgaattaaatgttaaaggcaaagatagagaactacatataaataaaatttaattttgcttaagacttaatttgtgtttagaaaattaaatttaaggcagctgccaaaattaaggagcacattgctctaaatgttttttcctttggtacctcagagaagtaagattcttctctatcaacaaatttgagcagtagatggcatcctttttcacagggatttcttagcagaaacacctgttcaggcaagaatatctttcagcttcttttatctttttctacatgttcagaagtatacaattacacacaaacaccttgatttctagatacaaataacatgttttttaagaacacaacagcactttacctgattttctttatcaaagcagcaaatgttgatggagatttcatatttataatataccttataggccaggcatcgtgggtcacgcctgtaatcgcaggacttagggaggctgaggtgggcagatcgcaaggtcaggagatcgagaccatcctggctaacatggtgaaaccccgtctgtactaaaaatacaaaaaaaagttagccgggcgtggtggcgggcacctgtagtcccagctactcgggaggctgaggcaggagaatggcatgaacccgggaggcggacgtggccagtgagctgagatcacaccactgcactccagcctgggtgacagagcgagactttaaatttaaaaaaaataaataaataaagtaaaaaatatatataccttataacatgcaattagtactaaatctaaatataatattagaccttaagtgtactggacaagttttttctaaagaaccattgcagaaaatatatagactattttcacaatgtgaatcatcaccccaaatttgtttccttaatttgaaaaagggcaccttacagtaatagcaaacagcatgttactgacaatgtaaaaataattaaaactttaccatttatgaaaataattcttaatcacatagaagaaaacaattttagggacagttcattatcaaagacctggaaaactgtcctgttaaatgtattgagtgcagattgttttgtttctttacaggtagatagattttggtcattgaaggtcctcttcattcagtctctaatgagaatttttagtgaaaaatgtccgatctcattttgtaaattgaatgcacgctaaatttgctcttttactaacagcataccttgttgcatcagccttaggagaaaatctgcgtgcacacgtgtgaatgtacacacacacacatacactgtgaacccttggtaattgccaggatgtccatttgagcaaagcccttgattaagtacttctggatacttgacaaacacgcagctattacattgcctaggtctcatgaaaagagctgatattcttgttggatgattctgtttgaacagtagaatctggtataacaattttgggaggcaatagaacttgcagtgtcagagtcttgagttgcagtactcttcattatcagtcccaacattgacatatatatattgattatagcaaacaatttaaaaagccacttaagcaatttttttttttttttttttttttgagacggagtctcgctctgtcgcccaggctggctggagtgcagtggcgcgatctcagctcactgcaaactctgcctcccaggttcatgccattttcttgcttcagcctcccgagtagctgggactataggtacccgccaccacgcccggctaattttttgtatttttactagagatagggtttcaccgtgttagccaggatggtctcgatctcctgacctcgtgatccgcccgcctcagcctcccaaagtgctgggattacaggcatgagccacggcgcccggccccaattggtttttaaataaaatagacggccatcatctcataggatattaaatactaccccatacccctgaactcctgaatgatacagacccgagtttaaattccagccctgctatgtgcttctgagcaagttacttagcctctctggctgcttcctcgtctgtaaaataaggttgacaataattcctaactcagaggtatagtatgagcattaaataagaaggcataaaaaagctcttagcaaaattcctggaacgtagtaaatgtcagtggtagctttttttttttttaatggagccaggatcttattagagagactttatcagcctgtctcaagatgaatcaccttatggcctcaacaatgtatccttaaaataatgaatataataagccatataatgagatacagtcctaaagccgtctctcttctaacaccgtctcccacctctccccagaaacttgttagtctttctttgtacagacatgtgcacacacacacacatacacccaactactgaaaatatctaaaattctcaaaggatttccttttgttcttcatgcagtcctcattatcatagtttcaaatgcttaccaaaaatttgttgtttttcttaagaacaatcagtgaaatccagaggaggttctgatgcccattttttaaatttacattgaagaaaataagcatatcagagttttccaatgtgctgtgatttgccttaacaattttttattctttggttttcacatccacaaaacctaaatgaatggctaggctgaaatgaagctgttgttacagaattgtgctcacagatcgtgaaatgatctggctggaagcattagcgttgatgacatttgtgtttgttctactttttcctgactgcctgagaagggcagccacaaagagcagcatagcatgcagtgactgccagcaagtctttgtggccaacagtctgcctctttgtagataatacctagatataatatgtagataaaataatatttttaaagagacaaagcaggtcttataagacttgtatggatgtatttgcaaaatgtgaagctgccttctagtatatttttaactagctttatagaggcaatgaggaagccaatttaaatgttgaaatttagattaaataaatatttcattaaacttacaaaccagttcttgtcagcctcttatctatatgtaaaactggtgtactgctgattcctcattacctttggcctggtagagagtttgtacaaggcagcacggttaatagttgaggtttattacaaagaataccagaagtaacagactcaaaaatcccagaggtctgaccattctgtttctctcctcttacagcaaaggatttgattcaaagctactgagagcaagagaataactcatttctgcttacattttattttaactctattaaaagttaaagctaagataatgtatttcttgtttttaaattactaaacagccaatgtaagatgttttttagcacttcattgatgaagttactgtctggttaagtaaaggtcaattggaaaaattggaaagtatttgcagtggattgggaaataacacactactttgttttgttggtgtgtattcttttttttcggcaatgatatcataattgccttttaaaaaacttttaatgtctgctccctgagctacttattttttgagcattaatttatactaaatcagcttcaatcttttaaagcagattagggtttatgtcactgaaactagttttgtttaatcttcctaaaaaatagggttagggttttattattgtttttaaataatacatgcctatttaaaaaaatagaagaagaagaagaagagcggggagaggcagagaaacttataggacaatatcatttgtaagtttctttcacataagtatacgcatttgaaccctggaaaaaccatttactttgtgacacaactgaagttcagaaagaatgtgattagccccagatcacagagaaagtaagtggtgacattagcagtggcccgtgggactaccagctcctcagcctgcatttaaaccacaggtacacttaacttactgccccgagttgcatatacattttctaataacgtaactattgatgtatgtgctcaccaccacttcaaggtacgcgtttgccctcgtgcatgtggtctgttgtgttcttaactgcagttgaagcactattgggtccctcatcatcaggaaattctcacaaagtacaatgacctttcaactaactgcattttacaaggagtaatacagttactcattacaggttgctgcaaataggaaaaaaaatgaagtatagaaaaaccaaacctcattgtcgaaatgtatttgggagttgcagaaggaagtgaactatgtcttcctctaatgcactagtgaaatactagagaatagtcactctcgcatcagtagttactatggaatatttaatcataggatatttaatcagtgcagttctgtagtatctggtagggtctcaaacaggctttaaaggttacttatttgttcctcaccataattagaagatgcagatgttaacatttctgtttccatatggagaaacagccacagaagtggcgcatcccaaggccacagacattgtaaatgttgggcccaggcctgggattagatgcctcaccctccttttgactccaccatgctgccagtctctacatgagaaaggaccaaatttttatttcatccaaatatcctagtgctcccggaaatattttcttatattttggagacttggcttactgccactttttttctccctcactgtttctattaacatgtggcatccaaaatcgatttggactgagaatcccacctttgaatgcctggcctgtgtgcatcagtaagctggatggttttgctgaaattgttagaagccatttcatcgagcacaccgtgtctccttcagcacctgtgctcactttgtcttacctttttccctttacacttgccaccttaccaaagcagcttccccctgcacctacctccgtggcttcatttaccctatgaccattattaatcctaaaaacttcacttttgtccccattccttgtttcttcaaacgccagcgtaaaacaaggaagtattttctgagcatcagaagtgctttggaaaggtcagatgatggtgaggaatatcatgctgtccacactgctttctttgggtcggacccagaacacataaaaagggaaaattgtgaacaatggcaaatataaaatgaaatatcctcatttcacacgtttcagctgtatgtcatctgtcaaatggaagccagcctttttgaaaaagtgagaaaaagactgcggtttccctcttctgccctcatactaaggacaaatttgattcactcactaaatggcagattatatagtaaaactttgctctcctgataaattcatatttgcacttcccctggaaatttaacagagtccttttgtagttaattaactgtaaaataagataagattgggcagttgtaaaaggaagtatctttaaggttttctgctaggctgggaatattttatttctaatcaaaagccaagaaactatctctaaactagctcagagggaaatccaccctccccacccctcttctttgatgttgctgcgtatcctggtgagggcttttattagaaaactaaggagcaggactggcacatcaatagcgataagccatttaaagtggtgcccctcctggtcctcgatgctcgggattaactcactgtggttcttttctggctgctccttttttgtaacttgtttattgcatatgcttttttccctgctcgactatgtttgggagccacgacttaccatcttgatttgtcttgtttgctttctgtgtcccttgcttcctgtgcccagttgagcctgttgatgcccgccctgctgccgaccgaggactgaccactcgaccaggtatcagaaccgcttgacttgtgcctctccgcatcttgggcctaagctcgagcagacgttgtctcctttctgcattttaagcctctctcaattgtacttactgcagagtagaatttgaagtgaccttagaaaccaaacttttaaaaattccccttgactattccttactcttcatttcccaagttccgagtgaaatgattttttttgtttctgagctttccctttgaaacctaaaagagagcccttccaaatttgggtttttttttttcttttttctttaaaacaaacaaaacaaaacaaaaacagagacgattatatacagcctgtccttaaaaacagaagcccagatagatgtagactctcaggtgatgacttggataccagtgtgaacatctctcctttcttgtttggtgtccagatgggtaattttctaactgaactgggctcaaacttcaatcagttactgaagtagacatgatttcctgaaaataccagaattggtttctttccagagcccaaaatacatcttctgttttgtgggttgtatgtacttattctcaatgagtggctaaagtaacacaacagatgtggatgcataattttgcacataagccctttttttatgtattacttaaaaatcccattaacactgaaatagacacttctttttttacagtttcacaaacattaagaagaaacacatttttattttatgaataatgtcatagcacagcagaagcatatatgtgcctggtgtctactctatgaacaacctcacccaactgatagtagccagtgctttcagtgaatcaaaaagaggaagaaagttattgaattgacaaatttgtgtattccagggagattttaatcccaaataacacatacttttccatatttattttcttctttatcctcataatgtccccaggcgatagtcaaagaacagggaattaactttctttgaagatattaatacacagaggtcaacctttgctgcctggttcagatacagtcagcagcagaccaggagacccaccctgtccctcacacttccaccacaggctcgagccatgactgcctccaagcacggtcactgatgaagactgagagtctgcctgtggcatcttcatgcctttatgctgtgattaacagtggcagcagagacagtagtgtgacacgtcttgacagctggtctcgttgatttatgaatttctgtctgtaggcttttaaggggaacttaaattatagtgagtcagagacagtttcagttcttctaatgtcctccttattgtcaatgtcatgtaagggaatctcaaggagccaggagttgacattcctgttctgctcagccaacttgtaaggcctcgggctgagggtcagttcagttctaattgcaaccacagccagctcaggttggtcatttgccttgggacttttttcatggtgcttctctttaaaagacatgctcttcatctacaagcataaaatgtaacttagaaagcgctcattcgactcattcaattttatcaaatttctacattgggccaggagagcctatatgttactattgaaacatccaggaacaggtgacactaccagtcaatgtttttttcttttttaaaaaaattgttttttgattttttttttttttttttgagatggagtctcgctgtgtcccccaggctggagtggagtggcacgatctcaactcactgcaacctctgcctcgggggttcaagcaattcttgtgccccagcctctcaagtagctgggactacagatgcgccccaccatgcccagctaatttttttgtattttcagtagagacgggatttcaccatgttggccaggctggtctcgaactactgacctcaggtgatccacccacctcggcatcccaaagtgctgggattacaggcataagcaccatgcccggcctattcttttaaaaaaaattttttttaatctaaatctaacaaaaaggacacctccaaacgctgtcaaacaaaaacagtgtgttttaaaggaagagctagagaatagttaatggtattacactggtgggagaaaaccatccaaaacctttctcataacagtaagaatatgatgtcacttggggatttcacaaagatcaatccactaaattctttctaggtttgaaagaaattaaacggtagggtccctgctctcaggagcttgaggtataatagaaggaattttgcatatttggaaacatgagatggaatttggaagacaaggaaatacaaagcaacattaagtgctaacaagaatgttctgtgatgggaggagatggagtactaacccaggggcatgttaagtagagaacttactggagatgggcttccagagaggttatgaaagatttgaggcacctggattggcaaagaagaagggaacaattctattcagaagagagggtggcctaagccaagcttcctttcatacacagcgtaggcttgggttgcccagtaagatggctaaagttgagaaagaatgctcagatatgaagtgaggaagattctagttaaggtttgtctatgaatagcagtatttactgcacatgtattaagccatgcgtgtgttctgtttttgtgcttgtagtaagtttatcaaacatttgggctaatttccccgtgttttgtagcatagctttggttcttcttcacattttagagaccacccacttgagtctaatgtgccgaattatatctttagcctttccaaacttagaaagccttcccaagattgtcatgccgttcaagtgatggcattgtttttgaataatcactgttgtcctcagaaacaagttaaaaaaaggtttttgtttttcccccacccagtgctttataatcatgactgaggaaaatggcttctccttaactctcatatctaaaaaaacagaatatggtagcttccagcatgcagtgtcaagtagggaccattgtaataacagtggtgatttgggggcatagtggccattatagctcctcctcctggcttacttccaaagaaaaaatagactctgaaggactaattatacagtttgaccagaataaaaaagaaaaacacattagcgagctcttcacatagggtagaaagctaccctttctgttacaattcatcaccacccttctgcagtccaaataaagggaaaccaggtccttgacccgagccatgcaagaggtgtcatgaaggacatacctcccttagcccccacatgaacaaaaggctctgtccactcatcatgacttacaggaagagagagctggagtcaccagggcttagggtgaaatcagcacaactcaagaaagacctctaagtaagcctgcaaaccaaggtccctgccatttatttgaaactactcatcacattttattacatgcattatcaccttattatgaatatttccctatgtgccagcttactgattttgtcttttagtaagatttgcttaatgtaaaaagtcttccctagatgactattcatgctaaacaaacatgaactattatagcaagagctcttctgccaagagcttctcaaactattgagctgtaaacatgaatttatctgtcatgctgacttgataatatgaatttttttttttggaccaccaaaagtattcacagtatcatttcaaaagtagtgtgggcttgtaaagtttttagtggagagctcttggttttgttttttttttttttttttttttaaaacaggatttgtctctgtttccaggctggagaacagtggcatgataccagctcactgcaacccccatttcctgggctaaagctatcctcccacctaagtttcccatgcagctgggattacagggatgtgccaccacacctggctcatttttgtatttttagtagagacagagttttgccatattggctaggctggtctcgaactcctgagttcaaactgtttgcccgcctcagcctcccaaaggctgggattacaggcatgagtcactgcacctggccgagagctcattctttttttttttttttttgagatggagtctcactctgtcgcccaggctggagtgcagtggcacgaatctcggctcactgcaagctctgcctcctgggttcacaccattctcctgcctaagcctctagtagctgggactacaggtgcctgccaccacgtccggctaattttttgtatttttagtagagacggggtttcaccatggtcttgatctcttgacctcgtgatcctcccgcctcggcctcccaaagtgctgggattacaggtgtgagccactgtgcccggccagagctcattctttacttttatttttcagtataaatatttggctccaaaatctaaaagggaagactcttcaaactgttcaagaaagttttcttaaaaatctcagaaatatagttctaacctattgtgttgctagatcttttatttatttatttatttttggagacagagtctcattctgtcacccaggctggagtgcagtggcacaatctcagctcactgcaacctccacctgccgggttcaagcgattcttatgcctcagccttctggagtagctgggattacaggcacccgccaccacacctggctaatttttttttttttttttttttttttttttttgtatttttagtagagacagggtttcaccatgttgaccaggctggtctcaaactcctgacctcaagtgatccacccagcttggcctcccaaagtgctgggattacaggcgtgagccaccgtgcttggcacgttgccagatcttaataacagaaaaggagtgagctctaaagaaatcactggttatataaggatctcagatttaaaagggctatagaaagattaaaatatacagccagtgagaattaagagagttttagcatcactaccctataaaaataagtgtcagaaaggttcgtttcagcagaatgagttgaaacttaaccaaaaaatacttaacaatattaaagtaagaaaataaataagaatagagaactctgtgggtctcgtagcaagaagttgggggttgagggttaggtcattgagaggaccagagttaaagaatgagaaagggaaactgtcccacttccattgtgccttttatgtctatattaaagagaatgaacttctgatgaaaggaaattaaagcccgagatagatcaacatgagaaaagaaagccctagcccattagttacctattgctacgtagcaaataacgcgagaatttagcagcttaaaacaacaagcattagtttcttacagtttttataggtcaggatttgggatgtggtttagctggatggttctggcacagtgtccgtcatgtggttgcagttaagacactgcctgagctaaagtcatctgaaggtttgattggacctggtgggtctgtttccaacgtggctcactcacgtcactgttactaagagacctcagtatcttaccatgtgaactctgtaatgctgcttgagcgtccctacaacatggcatctcgcacccctgcaccaagaggtccaggaaagagattaaggaagttttagtgccttttttggcccagtctcagaagtcatacaccatcacttctatcatgctctgttcactagagcaagtcactacatcccgaccacattcgaaggaagggaatcaagttctccctcttgaaggaaggagtatcagcaaatttgtggatatattttaaactatgacatcttgctacccaagtccaagttcttatgacccagaccaactgcaggctaagagaacttgaaggggaagtagcaccattgcctgtggtctttgtggaaagctgggaaggtagggataggaaaatgaagttctgaatagcaaaaaggagagaagatagcttcacaatctgtaagccaggtatagagtgacattgattgtgggtctcttttcaaataggttatcaagtggaagctttttgaacaattggatgaggaaatggtgctaggtcacaaccagcgtggatttcctaggttttgtcagattatccttaccttctccttgtataggctgtgctcttagactggcacatcatatgctgtggacattgtgcttttcagtgtcagcaagatgtgtcacaagtttcccagaatctcgtagacaagattgaaagagtgattaggccaagagcaggaagtgaattcccctgggagccaaaggaattgggaatgtgtagcccaagtaagacaagaaccagcaggaacatgcctctccttagggtcgtgatacctgttcaaggttttaatgtggaagggaggattaggcttgctctgtgttgaatcaggctcaaaggatggaagttacagggaagctgattctggcttcatgtaaaaaaaggacagtttgggcaggcaaatctatcaaaaaatggagggaaattgatacattcctctatgttcaaacaggaactgacaatctgcccctgggtgggaacacggtagagaagatgacttcaaaagcccttttcatcctaaaattctgatgtttgataattaaatgttatagcatggacactgacatttacattttttacttatgtttttggtttttaaatgactctgcattttgttttaagcttcaaattattatttgaataatgaaattcatcagaacaattagtgttaagaatcatatagcaatttatagaaaaggaagagttcgtaggttataaattctgttagttgctaagaagcatttttaaaattatgtactatagctctttattcagcagacgaaccaattacaatctgtgtaactagaacacttgactaaaattatataatttttacaacgcttcactgcatagatacatgaacataatttatttgtaattggaacaaagccccaaagtagcagttttgttctaccaggtaattaatgctcatttttaaagccttttattattatttctgaagtaatgagtgcacatggaaaaagacacataataggctaaacaataagcccgtaagccaagccaacatattccaggaacaaatccttgccaacctctcaaccaggatttaacttctgcttttcccccattttcaaaaattatagcatgtatttaaaggcagcagaagccttactttcaggtttcccttaccctttcatttctttttgttcaaaataggtagtaattgaagttttaaatatagggtatcatttttctttaagagtcatttatcaattttcttctaacttcaggcctagaaagaagttttgggtaggctttgtcttacagtgttattatttatgagtaaaactaattggttgtcctgcatactttaattatgatgtaatacaggttctgggttgacaaatatcaagacggaggagatctctgaagtgaagatggatgcagaattccgacatgactcaggatatgaagttcatcatcaaaaattggtacgtaaaataatttacctctttccactactgtttgtcttgccaaatgacctattaactctggttcatcctgtgctagaaatcaaattaaggaaaagataaaaatacaatgcttgcctataggattaccatgaaaacatgaagaaaataaataggctaggctgagcgcagtggctcaagcctgtaatcccagcactttgggaggccaaggcgggtggatcacgaggtcagaaattcgagaccagcctggccaatatggtgaaaccccatctctactaaaaatacaaaaaagattagctgggtgtggtggcaaacacctgtagtcccagctgctggggaggctgacgcaggagacttgcttgaacccaggaggtggaggttgcagtgagctgagatcgtgcctaggcgacagagcgagactccatcccaaaaaaaaaaaagaaaagaaagaggctgtatgtatagttctttcagactacaaggcagcaaagttcgtgcatgactcgggacttaaagtggaattaatttcaatatagcagccactttgacttccactgtgttttctgggaaaataggtttacaataggtttatttgaaggatcaaacacatgcatacactgcttggttttacagaacactttatgtggcttaaattcacatccggaactgtcttcctttacccattcatttctcccccagctctttcttttcattccctcccctacctcccatgatttaacttctcttgcaagagtaagatcatggagtgagcaggaccccatgatgttcccgatagtgttattcatcaaaaggtttgtgcaaagaagacagcagcttccttttcagatgaaatcacttttcccccctaatgttagaattggagtaaatcaaaaagccacatctcctttgtggtcagctctagtagttatataaaatcctttaccaaaagcttagaaatggagataaatcaaatcgtggattatgttagggttccatcttatcagtaggtgcagtaagagggttaaattaatgaagacgacaattttatcacattcagtggtggacagaaaaatggtaagaaaatttccatagcaataatacttaaagttatctcaggcacttcttttgttttgttttgtgtgtgtgtgtgtgtgagtgttacttttttccaagcagaaaatgtcttttcaatattcataaagttgataaatcctagtattaatctctaaaagaaacacctccaaattattatttatgccttacttgactccaaataattgtagcaaataaaaaactgacttgggatttggatttgcattcttaactcccatagttctttttttgtagaaagacatttagctttttgaagcatggttttcattggcaagataatctagtatcagttgttataagatcagggtttctcttgatgaggctgttgctgaagaggttaataaaaactggggaaccactaaagagttgaagagttggtggggtagaaagctgacgattaatgtacagatttgcatttgtcggggcctggggcctgtgtcatataagcccatccccacaattacactaacgcctataatgcgacagtgactaatggcagcggcagcaattaggagaatcagctccctctactggactagttaagataatgtattataattagtgcaatgaatattacaaaattacagtattttcttaaaggcacaggcatatgtccagacttgtatttattcctgattacctcacactagtatattagctaattaatgatttgcttttcataaaaatgttgagctagcatatttgtttagtaaagggaataattatgaacaatttctcattttgttataaaccacgagtaaaacacttttagaagttgcttcatttgctatattttatattgcctttccagattgctagtatgttagtttcagcttagaaaaatcagtcatttgactaccttgaggctaaattgaaagaattttttagggaggtacaggcataccttggaaatttcagaacaccacaataaaacaaattttgcaacaaaatgtcacacaaattttttggcttcccagtacatataaaagttatgttggccaggcacggtggctcatgcctgtaatcccagcactttgggaggccaaggcaggtggatcacaaggtcaggaggtcgagagcatcctggccaacatggtgaaatgccatctctactaaaaatacaaaaattagctgcatatggtggcatgtgcctgtagtcccagctactcgggaggctgaggcaagagaatctcttgatcccaggaggcagaggttgcagtgagccgagatcgcactccagcctgggcaacaagagcaagactctgtctcaaaaaaaaaaaaaagtttgtatttacactatactctagtctgttacatgtgcaatagcattatgtctaaaaaaaaaaaaagccatgtatatactttacttttattagtaaaatattttattactaaaaatgctaatgatcatctgagtcttcagcaagtcctaatctttttgctcgtggagaatcttgccttgatgttcactgctgcaggctaatccaggtggtggctgctgaggtttgtggtggctgtggcaatttcctaaaataagacaataatgaagtttgctgcatcgattgactcttcctttcacaaaaagatgtctctgtagcatgtgatgctgtttgatagcattttgctcacagtagaacagctttcaaaattggagtcagtcttcgcaaatctagccactgcttcatcgagtttatgtgatatcctaaatcctttgttgtcatttcaacagtgttcatagcatcttcaccaagagtagattccattgcaagaaaccactttctttgctcatccataagaggtaactcctcatccattcaagtttgatcatgagatggtagcagttcagtcatgtctttaggctccacttctaattctagttctcttgttatttccaccacatctgctgtgacatcctttactgaagtcttgaactcctcaaagtcacccatgagagttggaatcattgtcttccaaactcccgttattgctgatgttttgatctcccatggatcacagatgttcttaatggcatctggaatggtgaatcctttttagaaggtttgcagtttactttgcccagatccattagaggaatcgctatctatggcagtgaatagccctacaaaatatatttgaaatatattttgaaataataagaggctgaaataataagacttgaaagttgatccacaggctgcagaatggatgttgtgttagcaggcatgaaaacaacagtaatctccctgtacctgtccttcagagctcttgggtgactaggtgctttgttagtgagcagtaatattttgaaatgagtcttttctgagcagtaggtctcaacagtgggtttaaaatattcagtaaaccatgctgttaagagatgtgctgtcatcccagttttgttccatttatagagcatgggcacagtagatttagaataattcttaagcgccctaggacttttacaatggtcagtcttggcttcaaaaacaaagtcgccagctgcattagcccctaacaagagagtcagcttgttctttgaagctttgaaggcaggacttctctctagtgatgaaagtcctagatggcattttcttccaaataagtctgtttcatctccactgaaaatctgttatttagtgtagccaccttcatcaggagtcttatctagatcttctggataacttattgcagcttctacatcagcacttgctgcttcaccttgcacttctgtgttatggagatggcttcttcccttaaacctcatgaaatcaacctctcccaacttttttttcttctgcagcttcttcacctctctcatccttcatagaattgaagacagttgtgggccttgttcttgattaggctttggcctaagagaatgttgtggttggtttgatcttccatccagacaactaaaagttttctccgtatcagcaataaggctgttttgctttatcatttgtgtgctcactggagtagcacttttaatctccttcaagagcttttctttgcattcacaacttggctaactggcacaagaatcttggctttcaatatgccttgcccactaagcttaattatttctagcttttgctttaaagtgagagacctgtgactcttcctttcacttgaatacttaagaggccactgtagggttatttactggcccactttcaatattgttgtctctcaagggatagggagcctgggagttgagggggtggagtggccagtttggggggcagacagaacacacacaacatttattgatttagtttgctgtctttatgggcatgtttcatgacactccaaaacaattaaaatagtaacatcaaaggccactgatcacagatcaccaaacagatataataatttttaatgtttgaatatttaaataattaccaaaatgtgacacagacatgaagtgaacctgtgttcactgttggaaaaatggcaccaatagacttgttcgatgcagggttaccagaaacctttttgttttttttttttaaatactatctgtaagcactactgtacaataaagctatgtatgcctgtattttcattaagttgcagagcaaacgtggtaatatttagctttagttttacttcatctggcataagatcaactccttatataacaagatgataaaagttgtggtgtgcttctataatttcattcaagtagataaagttgaaaaataatgacttgcttttataaacagtatgaagcaatgtagtgcagtaaatgaaattttattccttctttacaatgttctcaaaattatttttatgtttaatccaaataaagagcaagaataaagcaacatttcagattttggtttctggagacaatagttagaaagcatgagttatgagtgacttaaaattcttgttgcctgtacttcactttgaaataacattatgctttaaaaagcattacactgctaaaggttaattagaattctgcagaattactatagctaaaagtaggtaacaagatatctttttttctattgtttaactcctttgtttcagaatgcctattcctgtgcattaaaagtgtccctccaaggaaattaggacatctgcagagttgaaaaacacctaagtctcagtcacttagagtcacacatcagggctcagagtgctatgactaggaaaatgctgacctcctttcattagtatgatcgtgcctttccagcttttgatagatccaagcgctatcttcccaccactcaccaaatgttccacctgtcaaagggtttcaggtccctgcagacttcggttttgacctgtggggaaagtagacttcctcgaactggggaagccacatgttgtacatccttctataaactatgattatcattcttagtaggaaaatatgtgatttctttttttttttttttttttaaagtaagcatcaaatatttgaccaaccagttgggcagagaatatactgaaactttttatataacctcatccaaatgtcccctgcatttaagaaatgaaattcttctaattgcgtttataaattgtaaattatattgcatttagaaattaaaattctttttcttaatttgttttcaaggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggtaggtaaacttgactgcatgtttccaagtgggaattaagactatgagagaattaggcttagctttttgctaagaactagctaagtatctcttttaaaaaacgaatcagtgtgcttccatgatgcttgggttacagttgttctttcttgttttggttttcattcattgcaacttaccgtgaatattctgctcaaggtattgagagtgtgtgttgttatctcaacttacaatttgtgttgaagttatcaaaataatacaaatgataatgcatgactttaaaaaagcatgattactcttagacttttttttttcaaacacaagcaacaaaaaccagtacaagaagttaaaaataaaaatagaagtaggaagttgctttttccaaaaaaactaaacatgtttattcttggatttcttttgtcttaaatagtctggcctctcaatacaaccaggtaatagtaaataatgtgacttttcagtgcagactcttacctttaaagtttctcaggaaatcacactataccaaatggcccaccaaaacatacatgattgttatttgagagttctgcaacctattaccctgcacttccacctgaaatatcttagtttcaaggtgctatactaaaacaagaaagagagaaagaaaatttacgaataagttatttttatgcatttgaaatgccatttggatgggggaaaaagtgttaaaatctcacagtatatgcaactttttaggaaactgtcttgcagagagtatagtgtaaacataacttttatatgcacagggaaactgaaaaatttgtgtgacatttaattgcgatattttaagagttatggaatttttagagctgaaagatttattgatgatgaccaatcaaattgatcttctcatcttgtagatgagggaactgaggccgagacagaagggttcaaattatgcaaggtcaagctcagagtccagatttcccctcatattccccattgctttttttttttttttttgagacagtctcgcgctgttgcccaggctggatcttggctcactgcaacctctgcctcctgggttcaagcaattctcttgtctcagcttcctgagtagctgggactatgggcacacgccaccatgcccggctaatttttgtttttagtagagacagggtttcaccatattggtcaggctggtctcgaactcctgacctcaggtgatccacccgccttggcttcccaaagtgctgggattacaggcgtgagccaccgcgtctggcccccattgctttttccatcagagaaaagtccaggtgtttctagggagttgaagattccactatccccaaggaaatgggctctaaaccattcttgattagaagaggacaggcagagtttaaataaatgaaatttgtggtgaagccagaaatttcattttgctcagcaattaaaacctctgcagactacaacagaagctgctgatggaaattggggtggagcctcatttccccattcatactgacgtggatgagcagtgtgaggaaggaaaccaagagaacagcaggcagcagggtggaaaggaacggactgacccatagatcagagtgtgagcacataaaagcaatgaccccactgtcatgtttggccattgcaccttttgtgatagtatctaagcttatgtccctcaaaacagtgtaagataggttttcattagtatcagcttatttcatttctgctcaaattaatttttaaaaaaatttaattccatatttgagcaaataaagatgtcttcatacttgagtaattcaagaccccttttttgctgctgttgttgttttggtttttttcatggttttgttgttttcttttgttgttgttgtttctttgttttttgagatggagtcttgctctttcacccaggctggagtgcagtggcatgatctccgctcactgcaacctccacctcccaggttcaagcgattctcctacctcactctcagcctcctgagtaactgggattacaggggcataccaccacgcctggctaatttttgtatttttagtagagacagggttttgccatgttggccaggctggtcttgaattcctggcctcaagtgatccgccctccccaacctcccaaggtgctgggattacaggcatgagccaccatgcctggccttcaagagcactttttagtttatttaatataattctctttgttgccggtctgtccgttgaagttgccttctttgatgccagaggccaaagacttggcttcctcgggcttccataacaaagtaacacagatgggtggattaagcaacagaaatttaccctctcacatttctggagtgtaagatcaaggtatcagccaggttggttcctccccaggcatctctccttggtttgcagatgcctgtctgctccctgtatcttcacatgaccttcactctgtttttctgctgtgtcctaacctcttcctacaaggacaccagtcatagtggattaggatcaaccctgatgaccttgtttaactttaataacctcttcgaccctatctgcaaatacagtcacaggtggaggtactggcagcttaggacttcaacatatgaattctagggggacacgttttagcccgtaacagaccccagtgatgctgagcataaaagcaaggcctccacctggagctccttcccctggctgtgcgtgagaccaggtcctacctcagacctaaggaaaaagaatccccaaataggaggctgcgaatctgagtgttgaaaatttccccaaatgaatgagatgtgcagtcggatttgagaatccctgacacatcattttaacactccttccttttagttctccgcgcaagcctcagggagtggaaaatggtatctctcacctcccagataaccggttgccttttaagtgctcttccagctgcctgaattaaaacgctctttttttagtcagtttaccaagcagtgtctcattggacttaaaatgtcagaggagaagttgacactttgacccagattctgacattaatttttaagccaggaggtatccttgcaggagcaaaatcgggaaggatggctgttcagctcttctctcctcattttgctgtctactggagagcaactggaagcctttctagatgaggggctccacctcattcgtgtttgttttgtttatttcctctcagctccttcccctcaagacctttgtgactgtccttcctgaaagcagccctctgtgcagccccatagtgtgtctgtacagcaatgttgaaatagaccacattccgcttttctgtcctcaaaaaagatgtagtttagctgggatggtagaacctgaacacagggagttgcaacccagcctaggcatgttcccaggtgagagagcacggtgggctttcagagcaccatttttcttagcattccagcgtctgcacccacagaagtgatgcttctgcaacatcctcttcctaccttaacaggcactctcaccctcaagtacatggaaatacctttatttccggctctggctaaccttcattaatcagtaaccctacttcagtaagaacaaaaatgagtacatcaatatttccagttggactcacctcagtcactcaggacagaataccattgtacttacataccatcgtgcctgaagaagtcatgatgaaagctttggtttccaggagattttttgtttttagagtgaaaaatgcccgcagaagatcacagagataggaaccctaggaaaaaaactcctgagaacaaatgtataaatgaaacaagcctagaacagtcacataccacttcacactggagagaacgttctgagaaatgtggcatggggcggtttgatcgctgtgcaaacatcataaagtacatgtacataaatctagatggtatagcctgctacacacctgggctacacacctacaactattgctcctaggctccacacctgtgcagcatattgctgcactgaatactatagacagttgtaacacactggcagggatttgcgtatctaaacaaactagaaaaggtacagtaaaaaatacggtattatggggccaccatcatatgtttggtctgtcgtggactaaaatgtcattatgtggcccataactgtatttttattggtagagttgttagcatttggaaatgagcacttgctagttgttgtttttttttttttttttttttttcaaataagcaggaaatattgacttactgaatttacttagccataaggccagagagtcactctgtgagccattggccacgctttcccaaggaggtaactgtgccttattggtgcttcaagcagtgaagcaatagccctttattattaccactccccaaatgtgccctccatgtcagaatagccctctggtggctggaaatctcagcactagctgaattaaccaccttgctcctgttttaggacgttcctgcaaagcccactaatcccaggatctgtttagtagcaagcggtacttttctacattcacatacatagacactggatatttaaaaatacgtattgtagctctttctgctactctccccagacatgtcacctttaataaggagaagttgggggcacagcttctctttattataggatgtgaaaaagttttcctctcacagatctgatcacttgaacaccagaagtctccccgcccccacctcctggttatctcattaatgatctaaataagaagagagttccttttatttggcttttatggaggtatggcatatgactttttctaagtaatatgctgtcacactggtgactgtataaaaagatgtccttagaggtcctttcttcccggccagaagctgacccaccaggatacagacgcactgacgttggcattgtagagtagttggagagagtggtcaggttgaatggaaagccacgataaggaaaagtgagggggggtgcagaacctaggagtggcttacagtgagcagcaggaagagaaattggaagatcaaaggaaagcatcaaaaatgatcacaggccaggcgtggtggctcacacctgtaatcccagcatttggggaggccaaggcaggtggatcacttgaggctaggagttcgagtccagcctggccaacatggtgagaccctgtctctaataaaaatacaaaaaattagccaggcgtggtggcatgcgcctgtagtcgcagctactcaggaggctgaggcaggagaatcgcttgaacccaggagatggaggctgcagtgagccaaaatcacgccactgcactccagcctgggtgacagagcaagactgtcttaaaaaaaaaaaaaattatgaagggatagcaaaggagttcctgaacctgcctgaagaattgcttgatgttggccataaacatagtcaactgccttctagttatatataactatagagagatggcatcaaaattaggaaaacaagcatctatacaactaggtagcaggcagttactgggaggttacgagggataagtcactagcttgaaaacttcctattttctttgcttttattttttttttccgcagacttgtggctcctttgatgtgtcacactattgttgtattgttcgccttagttcccttaccctcctttactcgtctggtccatctcaatgacaggattctgtcgtttggcctgactcagtcccttgtataaaaggacatctattaatccacatcctgctggttactgtacataataaaatgtaaacgtggtgtttcctgcctctcctccaagctcacacacccgcatgtaaatactctaggggcaataggaggagacctagaaactaaggtcacccaaatagaatcttgtccctgcaggcggatagctgagctctaaattactctggatacagtgaaggactaaatggttccaatgaatcaagttcagggagaaaagggtcagggggtatggaggttagccaaagagtgtcaaacgttctcctcaaagctattttagaacaaggctggtcaggctatttgggctcagctcttttcagcgtgtgtcataggcagcagccagttgaagccatgccaccatgcagtgcaggggtccaggtctcaggaaggtgcagcgtaccgagagtcacatgaagacctcagatcatttcctgctgctgtgtaatccaggcagtctgggaagagaatgaatattcagtggttgtatgtatgtgtgtcttcatcccagcaagtgacagatgttcctgcccagttggaaaatggaaattctgacttcattgtcttatcattccttccagaacaagcaccatgggctcaaaccctccctggctctgctttaagggaaataatattgtgtctgtgcagaggccctgtctgcacagggtcgtggtagggctggccttcacatggtggcccatgccactgctgtagcttagccatgaggaagagattggtgagcctgctggagaggaaggctacccttacttggtgctcctcctttatccatagacatgagtactcagattttcgcatctagctttcaaggggctggagattaaagatccaatccatactccttggatgcccttttgtcaagataatcccttcttctgtagctcctaagccctctaaggtctgagggccaagctgccccatggcaggaatggcaaaatgtagctgtcctctcagtggcaaaaggtaataggattatcaagtagcaaaacagaagccaagaaaagacaggagagttagaggtctggattgtgcaccagccttggagcctcgcatctctagcagggcactcttaagcgttaggctttggagtttgaagaggagataaacctatcaaattgccaggtgttcccctgcccttgatgcatgcaaatgattgttgacagccagtgtgatggtagatgtgcctcctggaagaaataaaagtataatattccaacaatcaagcagcattgtggcttttttctacaatcagaaacatagcagctttgtgcttgatctcctaagcaagataattttttttaacctgggcattctttgagttctcagtttcgaggtgatggggagcaaaaatgatgcccaagttcaaatggcgtactgctgcagataggctgtctcctggcacacagatttacagagtgaatgaaatctctcattattggtgctaatctttaaccatggtttttaacttacaagttggaggctaattacacagttaccagttaaagccaggtgacctcatttgttgacaacagtgcacagcaaaggaaagaggagaggataaggaaaatatctgtgatttgctcccagaaggacagtgaagctgagcatccatttgagcagagtcctctgagtactggccttggtcagagcttgttggtgggagtgtgctaaggggagatgcctaggtctgtcattggagtaagctgagggtttggatgcaggggaactcaggcagctctgagccacagggtggtagatgggagggattcacagtggcgcacagtctcttccataaatggaaacgtaagaatcggggcacatggccatggcggctcatgcctgtagtcctagtgctactttaggaggccgaggtaggcggatcacctgaggtcagtttgagaccagcatggccaacatggtgaaaccccatctctactaaaaatacaaaaattagccaggcgtagtggcgggtgcctgtaatcccagctactcgggaggctgaggcaggagaatcacttgaacctgggaggcagaggttgcagtgagccgagattgcctgggcaacaagagcaagactccaactcaaaaaaaatgggcacataatggatggaacattctgtcccaagggatgagggagctgtatatctacctattccagctgccttgggattagccactggtagtcattccacctcactcccagactgaaaagagtcaatcacttcatgaagtggggctatccgaagggaccacaggggacctgattatactgacagatttatttttatttatttactttttttgagacagagtctccctctgttgcccaggccggagtgcagtggcacgatcttggctcactgcagcctcagcctcctgggttcaagcagttcttctgcctcagcctctggagtagctgggactacaggcacgtgctactgtgcctggctaaatttttttgtatttttgttaaagatggggtttcactatgttggccaggcgtgtctcaaactcccgacctcgtgatccacccgccttggcctcccaaaagggctgggattttgagccacctcacctggccttttttttcttggagatggagtctcactctgtctcccaggctggagtgcagtggcacaatcttggctcactgcaaactccaccccccaagttcaagccattctcctgcctccgcctgtggagcagctgggattacaggcgtgcaccaccacgcctggctaatttttgtatttttagtagagacaaggtttcaccatgttggccaggctggtctcaaactcctgacctcaagtgacgcacccacctcaggctcccaaagtgctgggattacaggagtgagccactgcacccggccatattgacagatcttttagtctgaattgttatcagccagtgtggtctagtcatgacatatctagctccatctcgagacctcatgtcttactgctgacactacatcctcgctggtactttttcttccttggcttcaggggcattgctgtgtaggctcagatccatgccagatggggtgtgtagggggaaaagctgcataagggaggaatgtcacttggcaacactcaacaagggggaatgggcctgcagtattctcccagagatgaatgagcagcccattctaggaaatatccctttgaaggaatatcttctctatggtacatccaaacttttctctggaaaacatgcctcttcattctgatgactttctatttattctcttcatcttaccatggttttacaaaagaggaattttaacatactcattcagcagagttaaggacctcctctcttctgcgaacggggagaggtataacaaatatagtaaaacaagaacagccactgcccacgttgtaggggtaagtaccctgcccaacctgcagacagtaaatgccacacacagctctaacagtgacagggggagaaaaacgtcttcagttactggcctgaccttagaggtaaagcaacatattaacagagagggtctgattttaaactttgagctgaacatacttctatagtatcttaattctcagtttttaaaaaattacatcatctagaatggcgtcgtccttgctcttccttttttcttgtgccaaataatagccccctttggtatatgttattgttttaattgattctcaaacctctccttaacattttaacaaacataaaatttgatagaaatgggctagactaaattcagcagtccaggaaatcgagtatttttctacacagatttgagtgtcacccaagatacagtatactttgctgaatctccctccatgatactcttgtggttttatctattttaccaaaacacttccacgtaaaacaggcttccgttgactctgtttgtcctttatggacccaacaggggaggaaaaaataccattgcactgggtttatccacttactgtggagttggggaaccagaaacaaccactggacacgttgcaagcgcgtcaaggtttccaccgccacctggtggctagatggagcacagcatctgtgtaactggtgggcaaaggcgttctgctccaagatgtcaaagtgggggagaaaattatgggtgttctgcaatcttggcaaaagaagagtactgtactccttatctttttactgcttctccatgttcacccttaaaagattgatttttattttttactcagctctcctcttgtttttcaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaccattgtgctggtgtgaatgattccatagtaacaatcttgaccatttactgacgtacagaccagtgagaagtcttcgcatgttgggtacccacacctgttgtgtcttaattgcaagtctgagtaggaagttggggccaacatgtgtctcccagtgctgggaaaatatttcatagacctaatttacagtctttacttgatctaaaacattttgctgccatattttggccctcaagtttgtcccaaatgagagacaaagggaaaagttccagggaaataaaaattaagacagctgattatctgtaaagcatggtttctcatcctgaacgctactaacattttgcagggaataattccttgttgaagggagttgtcctgaccagtgtaggatatttatttattttatttatgttttttgagagtctcgctctgtcacccaggctggagtgcagtggcacaatctcggctcactgcactccagcctgggagacagacgagactccgtctcaataaataaataaataaataaataaataaataaaaggagggcctggcacgaatgacatgcagggaaggcagtgagcaggtggaggtccctgtactcgttgtggtgccttatctaccaggcggttgagttgacgtctttgtggacagaattcgattacaaaggtgtccgaaagtctccaggtgggggagagttttgtagcgggcataatttgggttgtaaattgactgttgtctcctgagcagtctcctggtgagggagagtttcactggagctttgaaacacatgttagacagccttgccctgtagggagtgtctcgtgaaggagaggtaaacaattataattgcatttctaaagggctaagtaggacatgggaaactggaaaatggagaaaagaagagaaaaaaatagtaactcattctctttttcttagaaaaatgaggatacttggtcacatcctcacatggtgaagaataagatggtctcttcatttcccttcaacactagtcccatcgtgaggctctgccctcatgacctaatctaatcctaactgcctcccaaaggtcccacctccacatcccattattggggattagggtttcaacatagggatttgggggaaacacaaacactcagttcatagtgatatctgactgcgattccaaaggtctgaggacacattggccctgttgtttttgctgtgttgtccttggcctcaggtgaacattttagctttagattgcaacattttaagctaaaaacactaagctagaaaattaaacttatctcgtcacattctgcatggtgaacaaactcaaaactcacctgagtgtatcttgttttcatgagaaaaaatgaccggtcatctcccttgccctgttagggacagttagacatgactgctgtatgaagatatgtacatgttaacaattcatatgtacaagctactgttccattttgcctggcccaaggggaggtagctagctagttggggtagcagggcgaattaaccctaatataatagtacatttctaatgccaccaatgaaatgaaaaccagctctcaaacatggcactatatgggatggcattccaaggccaactcagttacagtaactacagctttaactttgatcattttgttaccagaaaggggtcccaatccagaccccaagacagggttcttggatctcatgcaagaaagaattcaaggcgggtccatagaggaaaaggaaatgaagttttaaagaaagtaggaaaataaagagtggttgctccataggcagagtgaacctaagggctgctggttgcccattttattgttatttcttaattatttactaaacaagggctgaattattcatgcctccccttggtagaccatatagggtaacttcctgatgttgccatggcatttgtaaaccgtcatggctctagtgggagtgtagcagtgaggacaaccagaggtcactctcattgctatcttggttttggtgggttttgaccggcttctttactgcaacttgttttatgagcaaggtctttatgacctgtatcttatgcagacctacaatctcatcctgtgacttagaatgacttaaccctctgggaatgcagcccagtaggtcccagccttattttacccagcccctattcaaaatggagttgctctggttcagacgcctctgacaattttaaagctaatgaaaaaaatgtctagccaaatgtatatatacatacatatataattatgaaaagataatgccttataatcttgattcttaaattgaggtatattgatagtattctcccaatggtctggggattaatcaataggtaatttatacaaaactgaagtgctgccaaatagaattataggagtagctgctgcgcaaaaaagattaatgagatttggggagactttgctgtggctaatatgatgggtcaggaagagtacaatctcagtacaattttaagttgtatttatcttatgaataattgggctattctttcatatgcccaagggccattttatagctctatatt

SEQ ID NO: 3 - RNA, 3543 nt, Homo sapiens
  source 1..3543: mol_type = mRNA
cgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 4 - RNA, 3355 nt, Homo sapiens
  source 1..3355: mol_type = mRNA
agtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 5 - RNA, 2846 nt, Homo sapiens
  source 1..2846: mol_type = mRNA
agagttccggaggctttggttgtccattgttagcttagaagttggcgcagtgtgcgtgtgatccacgcctaaatagcacagccttgctgtgcgtggtagaagttgggttagtgttgacatgctgttgactcaccctcccgaggatggaagctctggcctgggtcaagttgtggtcactgcagttaacagtttgttgatctcagggagtattccacagttgctgatgtaattgacaatgattggagccagctcttccccagattcaaatggaccaattagaggacttgttggttctgtttatcaactatgtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattctt

SEQ ID NO: 6 - RNA, 3167 nt, Homo sapiens
  source 1..3167: mol_type = mRNA
gcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 7 - RNA, 2366 nt, Homo sapiens
  source 1..2366: mol_type = mRNA
gatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcgg

SEQ ID NO: 8 - RNA, 3423 nt, Homo sapiens
  source 1..3423: mol_type = mRNA
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 9 - DNA, 21 bp, synthetic construct
  source 1..21: mol_type = other DNA
cccactttgtgattccctacc

SEQ ID NO: 10 - DNA, 21 bp, synthetic construct
  source 1..21: mol_type = other DNA
atccatcctctcctggtgtaa

SEQ ID NO: 11 - DNA, 24 bp, synthetic construct
  source 1..24: mol_type = other DNA
tgatgcccttctcgttcctgacaa

SEQ ID NO: 12 - DNA, 15 bp, synthetic construct
  source 1..15: mol_type = other DNA
tcgccgcttgctgca

SEQ ID NO: 13 - DNA, 17 bp, synthetic construct
  source 1..17: mol_type = other DNA
atcggccgtgatgtcga

SEQ ID NO: 14 - DNA, 23 bp, synthetic construct
  source 1..23: mol_type = other DNA
ccatggtcaaccccaccgtgttc

SEQ ID NO: 15 - DNA, 20 bp, synthetic construct
  source 1..20: mol_type = other DNA
gtttacctttaacattcctc

SEQ ID NO: 16 - DNA, 20 bp, synthetic construct
  source 1..20: mol_type = other DNA
gcattctcttatattcctta

SEQ ID NO: 17 - DNA, 20 bp, synthetic construct
  source 1..20: mol_type = other DNA
gttcacagtttaccccaagc

SEQ ID NO: 18 - DNA, 20 bp, synthetic construct
  source 1..20: mol_type = other DNA
ctccaattttaacttgcacc

SEQ ID NO: 19 - DNA, 20 bp, synthetic construct
  source 1..20: mol_type = other DNA
  modified_base 1: mod_base = OTHER; note = 2'-O-methoxyethyl 5-methylcytidine 3'-phosphorothioate
  modified_base 2: mod_base = OTHER; note = 2'-O-methoxyethyl thymidine 3'-phosphorothioate
  modified_base 3: mod_base = OTHER; note = 2'-O-methoxyethyl 5-methylcytidine 3'-phosphorothioate
  modified_base 4: mod_base = OTHER; note = 2'-O-methoxyethyl 5-methylcytidine 3'-phosphodiester
  modified_base 5: mod_base = OTHER; note = 2'-O-methoxyethyl adenosine 3'-phosphorothioate
  modified_base 6: mod_base = OTHER; note = 2'-deoxyadenosine 3'-mesyl phosphoramidate
  modified_base 7..10: mod_base = OTHER; note = 2'-deoxythymidine 3'-phosphorothioate
  modified_base 11..12: mod_base = OTHER; note = 2'-deoxyadenosine 3'-mesyl phosphoramidate
  modified_base 13: mod_base = OTHER; note = 5-methyl-2'-deoxycytidine 3'-phosphorothioate
  modified_base 14..15: mod_base = OTHER; note = 2'-deoxythymidine 3'-phosphorothioate
  modified_base 16: mod_base = OTHER; note = 2'-O-methoxyethyl guanosine 3'-phosphodiester
  modified_base 17: mod_base = OTHER; note = 2'-O-methoxyethyl 5-methylcytidine 3'-phosphorothioate
  modified_base 18: mod_base = OTHER; note = 2'-O-methoxyethyl adenosine 3'-phosphorothioate
  modified_base 19: mod_base = OTHER; note = 2'-O-methoxyethyl 5-methylcytidine 3'-phosphorothioate
  modified_base 20: mod_base = OTHER; note = 2'-O-methoxyethyl 5-methylcytidine
ctccaattttaacttgcacc

SEQ ID NO: 20 - DNA, 20 bp, synthetic construct
  source 1..20: mol_type = other DNA
  modified_base 1: mod_base = OTHER; note = 2'-O-methoxyethyl guanosine 3'-phosphorothioate
  modified_base 2: mod_base = OTHER; note = 2'-O-methoxyethyl thymidine 3'-phosphorothioate
  modified_base 3..4: mod_base = OTHER; note = 2'-O-methoxyethyl thymidine 3'-phosphodiester
  modified_base 5: mod_base = OTHER; note = 2'-O-methoxyethyl adenosine 3'-phosphorothioate
  modified_base 6..7: mod_base = OTHER; note = 5-methyl-2'-deoxycytidine 3'-phosphorothioate
  modified_base 8..9: mod_base = OTHER; note = 2'-deoxythymidine 3'-phosphorothioate
  modified_base 10: mod_base = OTHER; note = 2'-deoxythymidine 3'-mesyl phosphoramidate
  modified_base 11..12: mod_base = OTHER; note = 2'-deoxyadenosine 3'-mesyl phosphoramidate
  modified_base 13: mod_base = OTHER; note = 5-methyl-2'-deoxycytidine 3'-phosphorothioate
  modified_base 14: mod_base = OTHER; note = 2'-deoxyadenosine 3'-mesyl phosphoramidate
  modified_base 15: mod_base = OTHER; note = 2'-deoxythymidine 3'-phosphorothioate
  modified_base 16: mod_base = OTHER; note = 2'-O-methoxyethyl thymidine 3'-phosphodiester
  modified_base 17..18: mod_base = OTHER; note = 2'-O-methoxyethyl 5-methylcytidine 3'-phosphorothioate
  modified_base 19: mod_base = OTHER; note = 2'-O-methoxyethyl thymidine 3'-phosphorothioate
  modified_base 20: mod_base = OTHER; note = 2'-O-methoxyethyl 5-methylcytidine
gtttacctttaacattcctc

SEQ ID NO: 21 - DNA, 20 bp, synthetic construct
  source 1..20: mol_type = other DNA
  modified_base 1: mod_base = OTHER; note = 2'-O-methoxyethyl guanosine 3'-phosphorothioate
  modified_base 2..4: mod_base = OTHER; note = 2'-O-methoxyethyl thymidine 3'-phosphorothioate
  modified_base 5: mod_base = OTHER; note = 2'-O-methoxyethyl adenosine 3'-phosphorothioate
  modified_base 6..7: mod_base = OTHER; note = 5-methyl-2'-deoxycytidine 3'-phosphorothioate
  modified_base 8..9: mod_base = OTHER; note = 2'-deoxythymidine 3'-phosphorothioate
  modified_base 10: mod_base = OTHER; note = 2'-deoxythymidine 3'-mesyl phosphoramidate
  modified_base 11..12: mod_base = OTHER; note = 2'-deoxyadenosine 3'-mesyl phosphoramidate
  modified_base 13: mod_base = OTHER; note = 5-methyl-2'-deoxycytidine 3'-mesyl phosphoramidate
  modified_base 14: mod_base = OTHER; note = 2'-deoxyadenosine 3'-mesyl phosphoramidate
  modified_base 15: mod_base = OTHER; note = 2'-deoxythymidine 3'-phosphorothioate
  modified_base 16: mod_base = OTHER; note = 2'-O-methoxyethyl thymidine 3'-phosphorothioate
  modified_base 17..18: mod_base = OTHER; note = 2'-O-methoxyethyl 5-methylcytidine 3'-phosphorothioate
  modified_base 19: mod_base = OTHER; note = 2'-O-methoxyethyl thymidine 3'-phosphorothioate
  modified_base 20: mod_base = OTHER; note = 2'-O-methoxyethyl 5-methylcytidine
gtttacctttaacattcctc

SEQ ID NO: 22 - DNA, 20 bp, synthetic construct
  source 1..20: mol_type = other DNA
  modified_base 1: mod_base = OTHER; note = 2'-O-methoxyethyl 5-methylcytidine 3'-phosphorothioate
  modified_base 2: mod_base = OTHER; note = 2'-O-methoxyethyl thymidine 3'-phosphorothioate
  modified_base 3: mod_base = OTHER; note = 2'-O-methoxyethyl 5-methylcytidine 3'-phosphorothioate
  modified_base 4: mod_base = OTHER; note = 2'-O-methoxyethyl 5-methylcytidine 3'-phosphodiester
  modified_base 5: mod_base = OTHER; note = 2'-O-methoxyethyl adenosine 3'-phosphorothioate
  modified_base 6: mod_base = OTHER; note = 2'-deoxyadenosine 3'-mesyl phosphoramidate
  modified_base 7..9: mod_base = OTHER; note = 2'-deoxythymidine 3'-phosphorothioate
  modified_base 10: mod_base = OTHER; note = 2'-deoxythymidine 3'-mesyl phosphoramidate
  modified_base 11..12: mod_base = OTHER; note = 2'-deoxyadenosine 3'-mesyl phosphoramidate
  modified_base 13: mod_base = OTHER; note = 5-methyl-2'-deoxycytidine 3'-phosphorothioate
  modified_base 14..15: mod_base = OTHER; note = 2'-deoxythymidine 3'-phosphorothioate
  modified_base 16: mod_base = OTHER; note = 2'-O-methoxyethyl guanosine 3'-phosphodiester
  modified_base 17: mod_base = OTHER; note = 2'-O-methoxyethyl 5-methylcytidine 3'-phosphorothioate
  modified_base 18: mod_base = OTHER; note = 2'-O-methoxyethyl adenosine 3'-phosphorothioate
  modified_base 19: mod_base = OTHER; note = 2'-O-methoxyethyl 5-methylcytidine 3'-phosphorothioate
  modified_base 20: mod_base = OTHER; note = 2'-O-methoxyethyl 5-methylcytidine
ctccaattttaacttgcacc

SEQ ID NO: 23 - DNA, 20 bp, synthetic construct
  source 1..20: mol_type = other DNA
  modified_base 1: mod_base = OTHER; note = 2'-O-methoxyethyl 5-methylcytidine 3'-phosphorothioate
  modified_base 2: mod_base = OTHER; note = 2'-O-methoxyethyl thymidine 3'-phosphorothioate
  modified_base 3: mod_base = OTHER; note = 2'-O-methoxyethyl 5-methylcytidine 3'-phosphorothioate
  modified_base 4: mod_base = OTHER; note = 2'-O-methoxyethyl 5-methylcytidine 3'-phosphodiester
  modified_base 5: mod_base = OTHER; note = 2'-O-methoxyethyl adenosine 3'-phosphorothioate
  modified_base 6: mod_base = OTHER; note = 2'-deoxyadenosine 3'-mesyl phosphoramidate
  modified_base 7..9: mod_base = OTHER; note = 2'-deoxythymidine 3'-phosphorothioate
  modified_base 10: mod_base = OTHER; note = 2'-deoxythymidine 3'-mesyl phosphoramidate
  modified_base 11..12: mod_base = OTHER; note = 2'-deoxyadenosine 3'-mesyl phosphoramidate
  modified_base 13: mod_base = OTHER; note = 5-methyl-2'-deoxycytidine 3'-phosphorothioate
  modified_base 14..15: mod_base = OTHER; note = 2'-deoxythymidine 3'-phosphorothioate
  modified_base 16: mod_base = OTHER; note = 2'-O-methoxyethyl guanosine 3'-phosphorothioate
  modified_base 17: mod_base = OTHER; note = 2'-O-methoxyethyl 5-methylcytidine 3'-phosphorothioate
  modified_base 18: mod_base = OTHER; note = 2'-O-methoxyethyl adenosine 3'-phosphorothioate
  modified_base 19: mod_base = OTHER; note = 2'-O-methoxyethyl 5-methylcytidine 3'-phosphorothioate
  modified_base 20: mod_base = OTHER; note = 2'-O-methoxyethyl 5-methylcytidine
ctccaattttaacttgcacc

SEQ ID NO: 24 - DNA, 20 bp, synthetic construct
  source 1..20: mol_type = other DNA
  modified_base 1: mod_base = OTHER; note = 2'-O-methoxyethyl 5-methylcytidine 3'-phosphorothioate
  modified_base 2: mod_base = OTHER; note = 2'-O-methoxyethyl thymidine 3'-phosphorothioate
  modified_base 3..4: mod_base = OTHER; note = 2'-O-methoxyethyl 5-methylcytidine 3'-phosphorothioate
  modified_base 5: mod_base = OTHER; note = 2'-O-methoxyethyl adenosine 3'-phosphorothioate
  modified_base 6: mod_base = OTHER; note = 2'-deoxyadenosine 3'-mesyl phosphoramidate
  modified_base 7..9: mod_base = OTHER; note = 2'-deoxythymidine 3'-phosphorothioate
  modified_base 10: mod_base = OTHER; note = 2'-deoxythymidine 3'-mesyl phosphoramidate
  modified_base 11..12: mod_base = OTHER; note = 2'-deoxyadenosine 3'-mesyl phosphoramidate
  modified_base 13: mod_base = OTHER; note = 5-methyl-2'-deoxycytidine 3'-phosphorothioate
  modified_base 14..15: mod_base = OTHER; note = 2'-deoxythymidine 3'-phosphorothioate
  modified_base 16: mod_base = OTHER; note = 2'-O-methoxyethyl guanosine 3'-phosphodiester
  modified_base 17: mod_base = OTHER; note = 2'-O-methoxyethyl 5-methylcytidine 3'-phosphorothioate
  modified_base 18: mod_base = OTHER; note = 2'-O-methoxyethyl adenosine 3'-phosphorothioate
  modified_base 19: mod_base = OTHER; note = 2'-O-methoxyethyl 5-methylcytidine 3'-phosphorothioate
  modified_base 20: mod_base = OTHER; note = 2'-O-methoxyethyl 5-methylcytidine
ctccaattttaacttgcacc

(19) United States
(12) Patent Application Publication
(10) Pub. No.: US 2024/0301411 A1
(43) Pub. Date: Sep. 12, 2024

(54) COMPOUNDS AND METHODS FOR REDUCING APP EXPRESSION

(71) Applicant: Ionis Pharmaceuticals, Inc., Carlsbad, CA (US)

(72) Inventors: Hien Thuy Zhao, San Diego, CA (US); Punit P. Seth, Carlsbad, CA (US)

(73) Assignee: Ionis Pharmaceuticals, Inc., Carlsbad, CA (US)

(21) Appl. No.: 18/443,619

(22) Filed: Feb. 16, 2024

Related U.S. Application Data
(60) Provisional application No. 63/485,807, filed on Feb. 17, 2023.

(51) Int. Cl. C12N 15/113

(52) U.S. Cl. CPC C12N 15/113; C12N 2310/11; C12N 2310/315

(57) ABSTRACT

Provided are compounds, methods, and pharmaceutical compositions for reducing the amount or activity of APP RNA in a cell or a subject, and in certain instances reducing the amount of APP protein in a cell or a subject. Such compounds, methods, and pharmaceutical compositions are useful to ameliorate at least one symptom or hallmark of a neurodegenerative disease or disorder associated with APP. Such symptoms and hallmarks include cognitive impairment, including a decline in memory and language skills, behavioral and psychological symptoms such as apathy and lack of motivation, gait disturbances, seizures, progressive dementia, and abnormal amyloid deposits.

### SEQUENCE LISTING

The present application is being filed along with a Sequence Listing in electronic format. The Sequence Listing is provided as a file entitled BIOL0460SEQ.xm1, created on Jan. 31, 2024, which is 384 KB in size. The information in the electronic format of the sequence listing is incorporated herein by reference in its entirety.

### FIELD

Provided are compounds, methods, and pharmaceutical compositions for reducing the amount or activity of amyloid precursor protein (APP) RNA in a cell or animal, and in certain instances reducing the amount of APP protein in a cell or a subject. Certain such compounds, methods, and pharmaceutical compositions are useful to ameliorate at least one symptom or hallmark of a neurodegenerative disease or disorder associated with APP. Such symptoms and hallmarks include cognitive impairment, including a decline in memory and/or language skills, behavioral and psychological symptoms such as apathy and lack of motivation, gait disturbances, seizures, progressive dementia, and abnormal amyloid deposits. Such neurodegenerative diseases and disorders associated with APP include sporadic Alzheimer's Disease, genetic/familial Alzheimer's Disease, Alzheimer's Disease in Down Syndrome patients, and sporadic and familial Cerebral Amyloid Angiopathy.

### BACKGROUND

Alzheimer's Disease (AD), including both sporadic Alzheimer's Disease and genetic/familial Alzheimer's Disease, is the most common cause of age-associated dementia, affecting an estimated 5.7 million Americans a year (Alzheimer's Association. 2018 Alzheimer's Disease Facts and Figures. Alzheimer's Dement. 2018; 14(3):367-429). AD is characterized by the accumulation of β-amyloid plaques in the brain prior to the onset of overt clinical symptoms. Such overt clinical symptoms include cognitive impairment, including a decline in memory and language skills, behavioral and psychological symptoms such as apathy and lack of motivation, gait disturbances, seizures, and progressive dementia.

Patients with Down Syndrome (DS) can experience early-onset Alzheimer's disease (AD in DS), with amyloid plaque formation observed by age 40 in most DS patients, and Alzheimer's dementia observed by age 50 in more than 50% of Down Syndrome patients.

Cerebral Amyloid Angiopathy (CAA) is a related disease that is characterized by the deposition of β-amyloid in blood vessels of the CNS. CAA is often observed in AD patients upon autopsy, but is also associated with aging in the absence of clinical signs of AD.

AD, AD in DS, and CAA are all characterized by the abnormal accumulation of β-amyloid plaques. β-amyloid (Aβ) is derived from amyloid precursor protein (APP) upon processing of APP by α-, β-, and γ-secretases. In addition to the 42-amino acid fragment Aβ, a variety of other fragments of APP are also formed, several of which are proposed to contribute to the onset of dementia in AD (reviewed in Nhan, et al., “The multifaceted nature of amyloid precursor protein and its proteolytic fragments: friends and foes”, Acta Neuropath., 2015, 129(1):1-19). The increased incidence of AD in DS patients is thought to be directly related to the increased copy number of the APP gene, which resides on chromosome 21.

Currently there is a lack of acceptable options for treating neurodegenerative diseases and disorders such as AD, AD in DS, and CAA. It is therefore an object herein to provide compounds, methods, and pharmaceutical compositions for the treatment of such diseases and disorders.

### SUMMARY

Provided herein are compounds, methods and pharmaceutical compositions for reducing the amount or activity of APP RNA, and in certain embodiments, reducing the amount of APP protein in a cell or a subject. In certain embodiments, the subject has or is at risk for developing a neurodegenerative disease or disorder associated with APP.

In certain embodiments, the subject has Alzheimer's Disease (AD). In certain embodiments, the subject has Alzheimer's Disease in conjunction with Down Syndrome (AD in DS). In certain embodiments, the subject has Cerebral Amyloid Angiopathy (CAA). In certain embodiments, compounds useful for reducing expression of APP RNA are oligomeric compounds. In certain embodiments, compounds useful for reducing expression of APP RNA are modified oligonucleotides.

Also provided are methods useful for ameliorating at least one symptom or hallmark of a neurodegenerative disease or disorder associated with APP. In certain embodiments, the neurodegenerative disease is Alzheimer's Disease. In certain embodiments, the neurodegenerative disease or disorder is Alzheimer's Disease in Down Syndrome patients.

In certain embodiments, the neurodegenerative disease or disorder is Cerebral Amyloid Angiopathy (CAA). In certain embodiments, the symptom or hallmark includes cognitive impairment, including a decline in memory and language skills, behavioral and psychological symptoms such as apathy and lack of motivation, gait disturbances, seizures, progressive dementia, and/or abnormal amyloid deposits.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications, and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyfuranosyl sugar moiety. In certain embodiments, a 2′-deoxynucleoside is a 2′β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a OCH₂CH₂OCH₃ group in place of the 2′—OH group of a furanosyl sugar moiety. A “2′-MOE sugar moiety” or a “2′—O-methoxyethyl sugar moiety” means a sugar moiety with a OCH₂CH₂OCH₃ group in place of the 2′—OH group of a furanosyl sugar moiety. Unless otherwise indicated, a 2′-MOE sugar moiety is in the β-D-ribosyl configuration. “MOE” means O-methoxyethyl.

As used herein, “2′-MOE nucleoside” or “2′—OCH₂CH₂OCH₃ nucleoside” means a nucleoside comprising a 2′-MOE sugar moiety (or 2′—OCH₂CH₂OCH₃ ribosyl sugar moiety).

As used herein, “2′—OMe” means a 2′—OCH₃ group in place of the 2′—OH group of a ribosyl sugar moiety. A “2′—O-methyl sugar moiety” or “2′—OMe sugar moiety” means a sugar moiety with a 2′—OCH₃ group in place of the 2′—OH group of a ribosyl sugar moiety. Unless otherwise indicated, a 2′—OMe has the β-D ribosyl stereochemical configuration.

As used herein, “2′—OMe nucleoside” means a nucleoside comprising a 2′—OMe sugar moiety.

As used herein, “2′—F” means a 2′-fluoro group in place of the 2′—OH group of a furanosyl sugar moiety. A “2′—F sugar moiety” means a sugar moiety with a 2′—F group in place of the 2′—OH group of a furanosyl sugar moiety. Unless otherwise indicated, a 2′—F sugar moiety is in the β-D-ribosyl configuration.

As used herein, “2′—F nucleoside” means a nucleoside comprising a 2′—F modified sugar moiety.

As used herein “2′—NMA” means a 2′—OCH₂C(═O)—N(H)CH₃ group at the 2′ position of a furanosyl sugar moiety. A “2-NMA sugar moiety” means a sugar moiety with a 2′—OCH₂C(═O)—N(H)CH₃ group in place of the 2′—OH group of a furanosyl sugar moiety.

As used herein, “2′—NMA nucleoside” means a nucleoside comprising a 2′—NMA sugar moiety.

As used herein, “2′-substituted nucleoside” means a nucleoside comprising a 2′-substituted furanosyl sugar moiety. As used herein, “2′-substituted” in reference to a sugar moiety means a sugar moiety comprising at least one 2′-substituent group other than H or OH.

As used herein, “stop site” refers to the 3′-most nucleotide of a target nucleic acid which is complementary to an oligonucleotide when the oligonucleotide is hybridized to the target nucleic acid.

As used herein, “start site” refers to the 5′-most nucleotide of a target nucleic acid which is complementary to an oligonucleotide when the oligonucleotide is hybridized to the target nucleic acid.

As used herein, “5-methylcytosine” means a cytosine modified with a methyl group attached to the 5 position. A 5-methylcytosine is a modified nucleobase.

As used herein, “abasic sugar moiety” means a sugar moiety that is not attached to a nucleobase. Such abasic sugar moieties are sometimes referred to in the art as “abasic nucleosides.”

As used herein, “administration” or “administering” means providing a pharmaceutical agent to a subject.

As used herein, “ameliorate” in reference to a treatment means improvement in at least one symptom or hallmark relative to the same symptom or hallmark in the absence of the tneatment. In certain embodiments, amelioration is the reduction in the severity or frequency of a symptom or the delayed onset or slowing of progression in the severity or frequency of a symptom. In certain embodiments, the symptom or hallmark is one or more of cognitive impairment, including a decline in memory and/or language skills, behavioral and psychological symptoms such as apathy and lack of motivation, gait disturbances, seizures, progressive dementia, or abnormal amyloid deposits.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “antisense oligonucleotide” means an oligonucleotide, including the oligonucleotide portion of an antisense compound that is capable of hybridizing to a target nucleic acid and is capable of at least one antisense activity. Antisense oligonucleotides include but are not limited to antisense RNAi oligonucleotides and antisense RNase H oligonucleotides.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl sugar moiety. In certain embodiments, the furanosyl sugar moiety is a ribosyl sugar moiety. In certain embodiments, the bicyclic sugar moiety does not comprise a furanosyl sugar moiety.

As used herein, “bicyclic nucleoside” or “BNA” means a nucleoside comprising a bicyclic sugar moiety.

As used herein, “cell-targeting moiety” means a conjugate moiety or portion of a conjugate moiety that is capable of binding to a particular cell type or particular cell types.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) similar to cerebrospinal fluid and is biocompatible with CSF.

As used herein, “chirally controlled” in reference to an internucleoside linkage means chirality at that linkage is enriched for a particular stenochemical configuration.

As used herein, “chirally enriched” in reference to a population means a plurality of molecules of identical molecular formula, wherein the number or percentage of molecules within the population that contain a particular stenochemical configuration at a particular chiral center is greater than the number or percentage of molecules expected to contain the same particular stenochemical configuration at the same particular chiral center within the population if the particular chiral center were stereorandom as defined herein. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides. In certain embodiments, the chiral center is at the phosphorous atom of a phosphorothioate internucleoside linkage. In certain embodiments, the chiral center is at the phosphorous atom of a mesyl phosphoramidate internucleoside linkage. In certain embodiments, a population of modified oligonucleotides is enriched for modified oligonucleotides having at least one phosphorothioate and/or mesyl phosphoramidate in the (Sp) configuration. In certain embodiments, a population of modified oligonucleotides is enriched for modified oligonucleotides having at least one phosphorothioate and/or mesyl phosphoramidate in the (Rp) configuration. Unless otherwise indicated, chiral internucleoside linkages of modified oligonucleotides described herein may be stereorandom or chirally enriched.

As used herein, “cleavable moiety” means a bond or group of atoms that is cleaved upon administration to a subject, for example, inside a cell, a subject, or a human.

As used herein, “complementary” in reference to an oligonucleotide means that at least 70% of the nucleobases of the oligonucleotide and the nucleobases of another nucleic acid or one or more regions thereof are capable of hydrogen bonding with one another when the nucleobase sequence of the oligonucleotide and the other nucleic acid are aligned in opposing directions. “Complementary region” in reference to a region of an oligonucleotide means that at least 70% of the nucleobases of that region and the nucleobases of another nucleic acid or one or more regions thereof are capable of hydrogen bonding with one another when the nucleobase sequence of the oligonucleotide and the other nucleic acid are aligned in opposing directions. As used herein, “complementary nucleobases” means nucleobases that are capable of forming hydrogen bonds with one another. Complementary nucleobase pairs include adenine (A) and thymine (1′), adenine (A) and uracil (U), cytosine (C) and guanine (G), and 5-methylcytosine (^{m}C) and guanine (G).

Certain modified nucleobases that pair with unmodified (natural) nucleobases or with other modified nucleobases are known in the art. For example, hypoxanthing can pair, but is not considered complementary, with adenosine, cytosine, thymine, or uracil. Herein, hypoxanthine (I) is considered a complementary nucleobase to thymine (T), adenine (A), uracil (U), and cytosine (C). Complementary oligonucleotides and/or nucleic acids need not have nucleobase complementarity at each nucleoside. Rather, some mismatches are tolerated. As used herein, “fully complementary” or “100% complementary” in reference to an oligonucleotide or a portion thereof, means that the oligonucleotide or portion thereof, is complementary to another oligonucleotide or nucleic acid at each nucleobase of the shorter of the two oligonucleotides, or at each nucleoside if the oligonucleotides are the same length.

As used herein, “complementary region” in reference to an oligonucleotide is the range of nucleobases of the oligonucleotide that is complementary with a second oligonucleotide or target nucleic acid.

As used herein, “conjugate group” means a group of atoms directly attached to an oligonucleotide that confers at least one property to the resulting conjugated oligonucleotide. Conjugate groups comprise a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that when covalently bound to a molecule modifies one or more properties of such molecule compared to the identical molecule lacking the conjugate moiety, wherein such properties include, but are not limited to pharmacodynamics, pharmacokinetics, stability, binding, absorption, tissue distribution, cellular distribution, cellular uptake, charge, and clearance.

As used herein, “constrained ethyl” or “cEt” or “cEt sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt sugar moiety.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “deoxy region” means a region of 5-12 contiguous nucleotides, wherein at least 70% of the nucleosides comprise a β-D-2′-deoxyribosyl sugar moiety. In certain embodiments, each nucleoside of a deoxy region is selected from a 2′-β-D-deoxynucleoside, a bicyclic nucleoside, and a 2′-substituted nucleoside. A deoxy region supports RNase H activity. In certain embodiments, a deoxy region is the gap or internal region of a gapmer.

As used herein, “double-stranded” in reference to a region or an oligonucleotide, means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

As used herein, “duplex” or “duplex region” means the structure formed by two oligonucleotides or portions thereof that are hybridized to one another.

As used herein, “gapmer” means a modified oligonucleotide comprising an internal region positioned between external regions having one or more nucleosides, wherein the nucleosides comprising the internal region are chemically distinct from the nucleoside or nucleosides comprising the external regions, and wherein the modified oligonucleotide supports RNAse H cleavage. The internal region may be referred to as the “gap” and the external regions may be referred to as the “wings.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. Unless otherwise indicated, a gapmer may comprise one or more modified internucleoside linkages and/or modified nucleobases and such modifications do not necessarily follow the gapmer pattern of the sugar modifications.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “internucleoside linkage” means the covalent linkage between immediately adjacent (e.g., contiguous) nucleosides in an oligonucleotide. As used herein, “unmodified internucleoside linkage” means a phosphodiester internucleoside linkage. As used herein “modified internucleoside linkage” means any internucleoside linkage other than a phosphodiester internucleoside linkage. A “phosphorothioate internucleoside linkage” or “PS internucleoside linkage” is a modified internucleoside linkage in which one of the non-bridging oxygen atoms of a phosphodiester internucleoside linkage is replaced with a sulfur atom. A “mesyl phosphoramidate internucleoside linkage” is a modified internucleoside linkage in which one of the non-bridging oxygen atoms of a phosphodiester internucleoside linkage is replaced with NS(═O)₂CH₃.

As used herein, “inverted nucleoside” means a nucleotide having a 3′ to 3′ and/or 5′ to 5′ internucleoside linkage.

As used herein, “linked nucleosides” are nucleosides that are connected in a contiguous sequence (i.e., nucleosides immediately adjacent to one another, no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “mismatch” or “non-complementary” means a nucleobase of a first nucleic acid sequence that is not complementary with the corresponding nucleobase of a second nucleic acid sequence or target nucleic acid when the first and second nucleic acid sequences are aligned.

As used herein, “motif” means the pattern of unmodified and/or modified sugar moieties, nucleobases, and/or internucleoside linkages in an oligonucleotide.

As used herein, “modified nucleoside” means a nucleoside comprising a modified nucleobase and/or a modified sugar moiety.

As used herein, “modified sugar moiety” means a sugar moiety of a nucleoside other than 2′-β-D-deoxyribosyl sugar moiety (the sugar moiety of unmodified DNA) or β-D-ribosyl sugar moiety (the sugar moiety of unmodified RNA).

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. A nucleobase is a heterocyclic moiety. As used herein an “unmodified nucleobase” is adenine (A), thymine (‘T’), cytosine (C), uracil (U), or guanine (G). As used herein, a “modified nucleobase” is a group of atoms other than unmodified A, T, C, U, or G capable of pairing with at least one other nucleobase. A “5-methyl cytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “the nucleobase sequence of” a reference SEQ ID NO, refers only to the nucleobase sequence provided in such SEQ ID NO and therefore, unless otherwise indicated, includes compounds wherein each nucleobase, each sugar moiety, and each internucleoside linkage, independently, may be modified or unmodified, irrespective of presence or absence of modifications, indicated in the refenced SEQ ID NO.

As used herein, “nucleoside” means a compound or fragment of a compound comprising a nucleobase and a sugar moiety. The nucleobase and sugar moiety are each, independently, unmodified or modified.

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or a terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences. Each oligomeric compound of an oligomeric duplex may be referred to as a “duplexed oligomeric compound.” As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. An oligonucleotide may be paired with a second oligonucleotide that is complementary to the oligonucleotide or it may be unpaired.

As used herein, “pharmaceutically acceptable carrier or diluent” means any substance suitable for use in administering to a subject. Certain such diluents enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “population” means a plurality of molecules of identical molecular formula.

As used herein, “prodrug” means a therapeutic agent in a first form outside the body that is converted to a second form within a subject or cells thereof. Typically, conversion of a prodrug within the subject is facilitated by the action of an enzyme (e.g., endogenous or viral enzyme) or chemical present in cells or tissues and/or by physiologic conditions. The first form of the prodrug may be less active than the second form.

As used herein, “RNA” means an RNA transcript and includes pre-mRNA and mature mRNA unless otherwise specified.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense agents that act principally through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount and/or activity of a target nucleic acid. The term RNase H agent excludes antisense agents that act principally through RISC/Agog.

As used herein, “sense compound” means a sense oligonucleotide and optionally one or more additional features, such as a conjugate group.

As used herein, “sense oligonucleotide” means an oligonucleotide, including the oligonucleotide portion of a sense compound, that is capable of hybridizing to an antisense oligonucleotide.

As used herein, “single-stranded” means a nucleic acid (including but not limited to an oligonucleotide) that is unpaired and is not part of a duplex. Single-stranded compounds are capable of hybridizing with complementary nucleic acids to form duplexes, at which point they are no longer single-stranded.

As used herein, “standard in vivo assay” means the assay described in Example 2, 3, 4, 5, 6, or 7, and reasonable variations thereof.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be the same as the number of molecules having the (R) configuration of the stereorandom chiral center (“racemic”). The stereochemical configuration of a chiral center is random when it is the result of a synthetic method that is not designed to control the stereochemical configuration. In certain embodiments, the stereorandom chiral center is at the phosphorous atom of a stereorandom phosphorothioate or mesyl phosphoramidate internucleoside linkage having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage or a mesyl phosphoramidate internucleoside linkage.

As used herein, “subject” means a human or non-human animal. In certain embodiments, the subject is Inman.

As used herein, “sugar moiety” means an unmodified sugar moiety or a modified sugar moiety. As used herein, “unmodified sugar moiety” means a 2′—OH(H) β-D-ribosyl moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests. In certain embodiments, a hallmark is a result of the diagnostic testing, including, but not limited to, post-mortem tests. In certain embodiments, symptoms and hallmarks include cognitive impairment, including a decline in memory and/or language skills, behavioral and psychological symptoms such as apathy and lack of motivation, gait disturbances, seizures, progressive dementia, and abnormal amyloid deposits.

As used herein, “target nucleic acid” and “target RNA” mean a nucleic acid that an oligomeric compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “treating” means improving a subject's disease or condition by administering an oligomeric agent or an oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent or composition that provides a therapeutic benefit to an subject. For example, a therapeutically effective amount improves a symptom of a disease.

The present disclosure provides the following non-limiting numbered embodiments:
Embodiment 1. An oligomeric compound comprising a modified oligonucleotide consisting of 8 to 80 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases of the nucleobase sequence of any of SEQ ID NOs: 15-18, and wherein at least one internucleoside linkage of the modified oligonucleotide is a mesyl phosphoramidate internucleoside linkage.
Embodiment 2. The oligomeric compound of embodiment 1, wherein the modified oligonucleotide has a nucleobase sequence comprising the nucleobase sequence of any of SEQ ID NOs: 15-18.
Embodiment 3. The oligomeric compound of any of embodiment 1 or embodiment 2, wherein the modified oligonucleotide consists of 20 to 80 linked nucleosides and has a nucleobase sequence comprising the nucleobase sequence of SEQ ID NO: 15 or SEQ ID NO: 18.
Embodiment 4. The oligomeric compound of any of embodiments 1-3, wherein the modified oligonucleotide has a nucleobase sequence consisting of the nucleobase sequence of SEQ ID NO: 15 or SEQ ID NO: 18.
Embodiment 5. The oligomeric compound of any of embodiments 1-4, wherein the nucleobase sequence of the modified oligonucleotide is at least 85%, at least 90%, at least 95%, or 100% complementary to an equal length portion of an APP nucleic acid, wherein the APP nucleic acid has the nucleobase sequence of SEQ ID NO: 1 or SEQ ID NO: 2.
Embodiment 6. The oligomeric compound of any of embodiments 1-5, wherein the modified oligonucleotide consists of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 22, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.
Embodiment 7. The oligomeric compound of any of embodiments 1-6, wherein the modified oligonucleotide consists of 20 linked nucleosides.
Embodiment 8. The oligomeric compound of any of embodiments 1-7, wherein at least one nucleoside of the modified oligonucleotide is a modified nucleoside.
Embodiment 9. The oligomeric compound of embodiment 8, wherein the modified nucleoside comprises a modified sugar moiety.
Embodiment 10. The oligomeric compound of embodiment 9, wherein the modified sugar moiety comprises a bicyclic sugar moiety.
Embodiment 11. The oligomeric compound of embodiment 10, wherein the bicyclic sugar moiety comprises a 2′-4′ bridge selected from —O—CH₂— and —O—CH(CH₃)—.
Embodiment 12. The oligomeric compound of any of embodiments 8-9, wherein the modified nucleoside comprises a non-bicyclic modified sugar moiety.
Embodiment 13. The oligomeric compound of embodiment 12, wherein the non-bicyclic modified sugar moiety is a 2′-MOE sugar moiety or a 2′—OMe sugar moiety.
Embodiment 14. The oligomeric compound of embodiment 12 or embodiment 13, wherein the non-bicyclic modified sugar moiety is a 2′-MOE sugar moiety.
Embodiment 15. The oligomeric compound of any of embodiments 1-14, wherein at least 2, at least 3, at least 4, at least 5, or at least 6 internucleoside linkages of the modified oligonucleotide are mesyl phosphoramidate internucleoside linkages.
Embodiment 16. The oligomeric compound of any of embodiments 1-15, wherein at least one internucleoside linkage of the modified oligonucleotide is a phosphorothioate internucleoside linkage.
Embodiment 17. The oligomeric compound of embodiment 16, wherein at least 7, at least 8, at least 9, at least 10, at least 11, at least 12, at least 13, at least 14, at least 15, at least 16, at least 17, or at least 18 internucleoside linkages of the modified oligonucleotide are phosphorothioate internucleoside linkages.
Embodiment 18. The oligomeric compound of any of embodiments 1-17, wherein at least one internucleoside linkage of the modified oligonucleotide is a phosphodiester internucleoside linkage.
Embodiment 19. The oligomeric compound of any of embodiments 1-18, wherein the modified oligonucleotide has an internucleoside linkage motif (5′ to 3′) selected from sooosssssszzszsooss, sooosssssszszssooss, sooosszsssszsssooss, soooszsssszzsssooss, sooosszzssszsssooss, sooossssszzzszsooss, sooossssszzzzssooss, sooosszssszzsssooss, soooszssszzzsssooss, sssossssszzszsosss, ssoossssszszssosss, ssoosszsssszsssosss, sssoszsssszzsssosss, ssoossssszzzszsosss, ssssssssszzzsssss, sssoszssszzzsssosss, sssoszssszzzsssssss, and ssssszssszzzsssosss, wherein each “s” represents a phosphorothioate internucleoside linkage, each “o” represents a phosphodiester internucleoside linkage, and each “z” represents a mesyl phosphoramidate internucleoside linkage.
Embodiment 20. The oligomeric compound of any of embodiments 1-19, wherein the modified oligonucleotide comprises at least one modified nucleobase.
Embodiment 21. The oligomeric compound of embodiment 20, wherein the modified nucleobase is 5-methylcytosine.
Embodiment 22. The oligomeric compound of embodiment 21, wherein each cytosine is a 5-methylcytosine.
Embodiment 23. The oligomeric compound of any of embodiments 1-22, wherein one or more nucleosides of the modified oligonucleotide comprises an unmodified nucleobase.
Embodiment 24. The oligomeric compound of any of embodiments 1-23, wherein the modified oligonucleotide comprises a deoxy region.
Embodiment 25. The oligomeric compound of embodiment 24, wherein each nucleoside of the deoxy region is a 2′-β-D-deoxynucleoside.
Embodiment 26. The oligomeric compound of embodiment 24 or embodiment 25, wherein the deoxy region consists of 6, 7, 8, 9, 10, or 6-10 linked nucleosides.
Embodiment 27. The oligomeric compound of any of embodiments 24-26, wherein each nucleoside immediately adjacent to the deoxy region comprises a modified sugar moiety.
Embodiment 28. The oligomeric compound of any of embodiments 24-28, wherein the deoxy region is flanked on the 5′-side by a 5′-external region consisting of 1-6 linked 5′-external region nucleosides and on the 3′-side by a 3′-external region consisting of 1-6 linked 3′-external region nucleosides, wherein:
   the 3′-most nucleoside of the 5′ external region comprises a modified sugar moiety; and
   the 5′-most nucleoside of the 3′ external region comprises a modified sugar moiety.
Embodiment 29. The oligomeric compound of embodiment 28, wherein each nucleoside of the 3′ external region comprises a modified sugar moiety.
Embodiment 30. The oligomeric compound of embodiment 28 or embodiment 29, wherein each nucleoside of the 5′ external region comprises a modified sugar moiety.
Embodiment 31. The oligomeric compound of embodiment 30, wherein the modified oligonucleotide has:
   a 5′ external region consisting of 5 linked nucleosides;
   a deoxy region consisting of 10 linked nucleosides; and
   a 3′ external region consisting of 5 linked nucleosides;
wherein each of the 5′ external region nucleosides and each of the 3′ external region nucleosides is a 2′-MOE nucleoside and each nucleoside of the deoxy region is a 2′β-D-deoxynucleoside.
Embodiment 32. The oligomeric compound of any of embodiments 24-31, wherein at least 3 internucleoside linkages of the deoxy region are mesyl phosphoramidate internucleoside linkages.
Embodiment 33. A modified oligonucleotide according to the following chemical structure

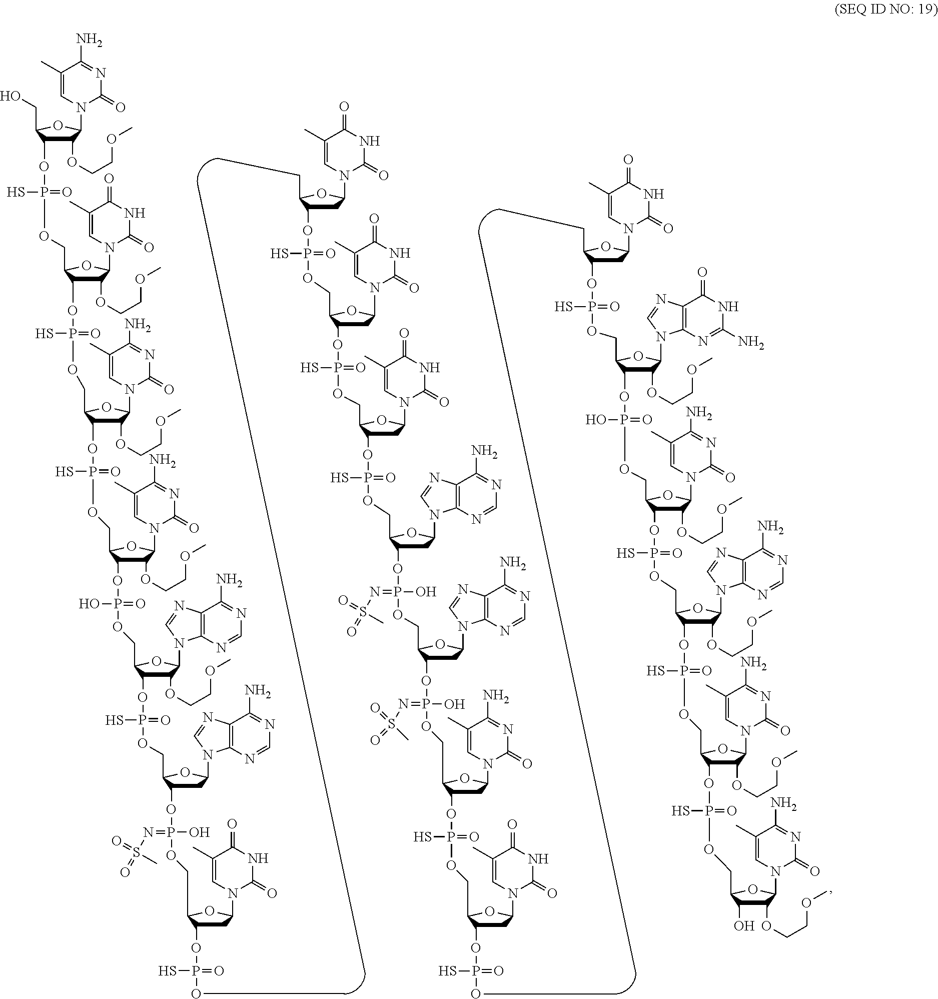

or a pharmaceutically acceptable salt thereof.
Embodiment 34. The modified oligonucleotide of embodiment 33, which is a pharmaceutically acceptable salt comprising one or more cations selected from sodium, potassium, calcium, and magnesium.
Embodiment 35. The modified oligonucleotide of embodiment 34, which is the sodium salt or the potassium salt.
Embodiment 36. A modified oligonucleotide according to the following chemical structure:

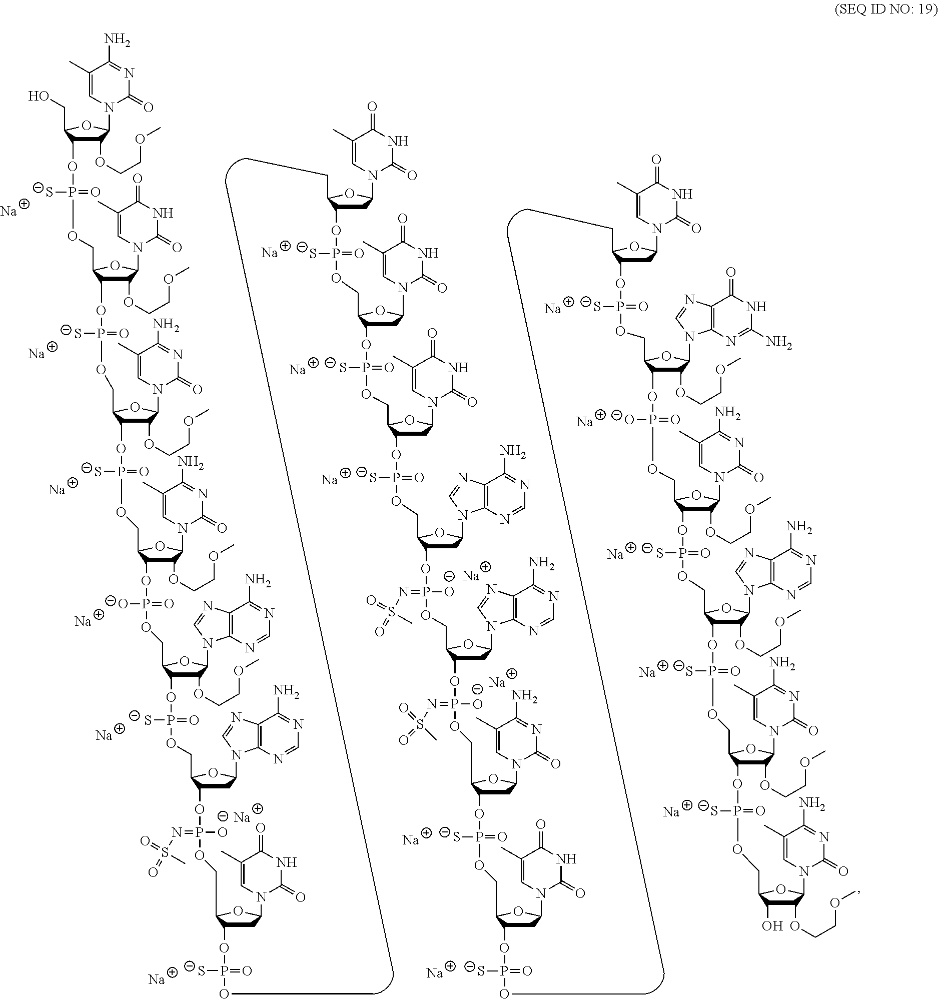

or a pharmaceutically acceptable salt thereof.
Embodiment 37. A modified oligonucleotide according to the following structure:

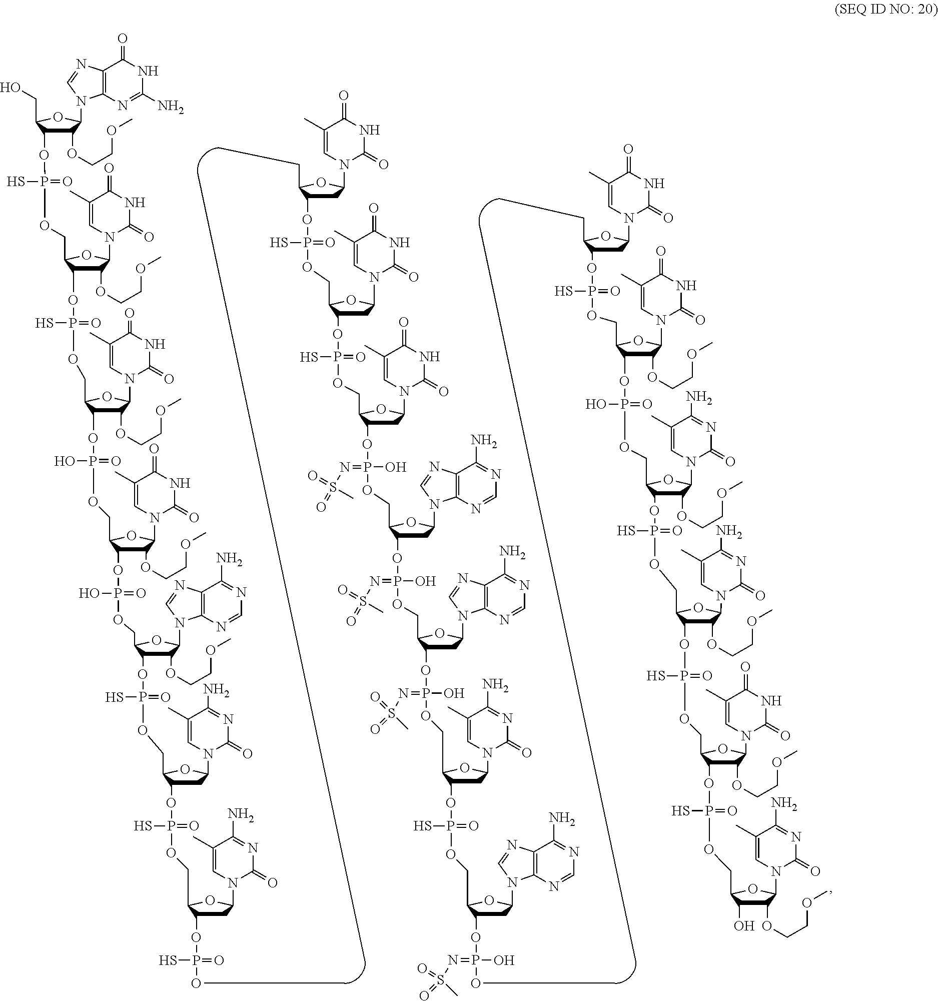

or a salt thereof.
Embodiment 38. The modified oligonucleotide of embodiment 37, which is a pharmaceutically acceptable salt comprising one or more cations selected from sodium, potassium, calcium and magnesium.
Embodiment 39. The modified oligonucleotide of embodiment 38, which is the sodium salt or the potassium salt.
Embodiment 40. A modified oligonucleotide according to the following chemical structure:

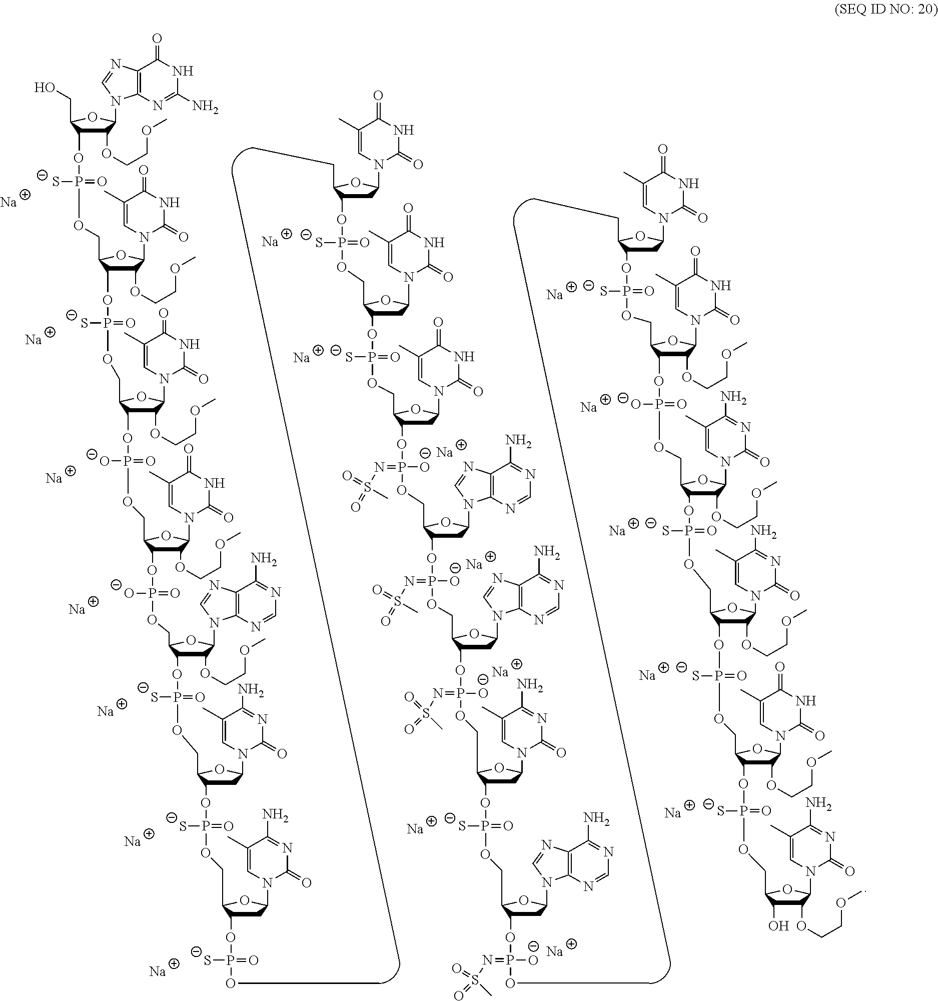

Embodiment 41. A modified oligonucleotide according to the following chemical structure:

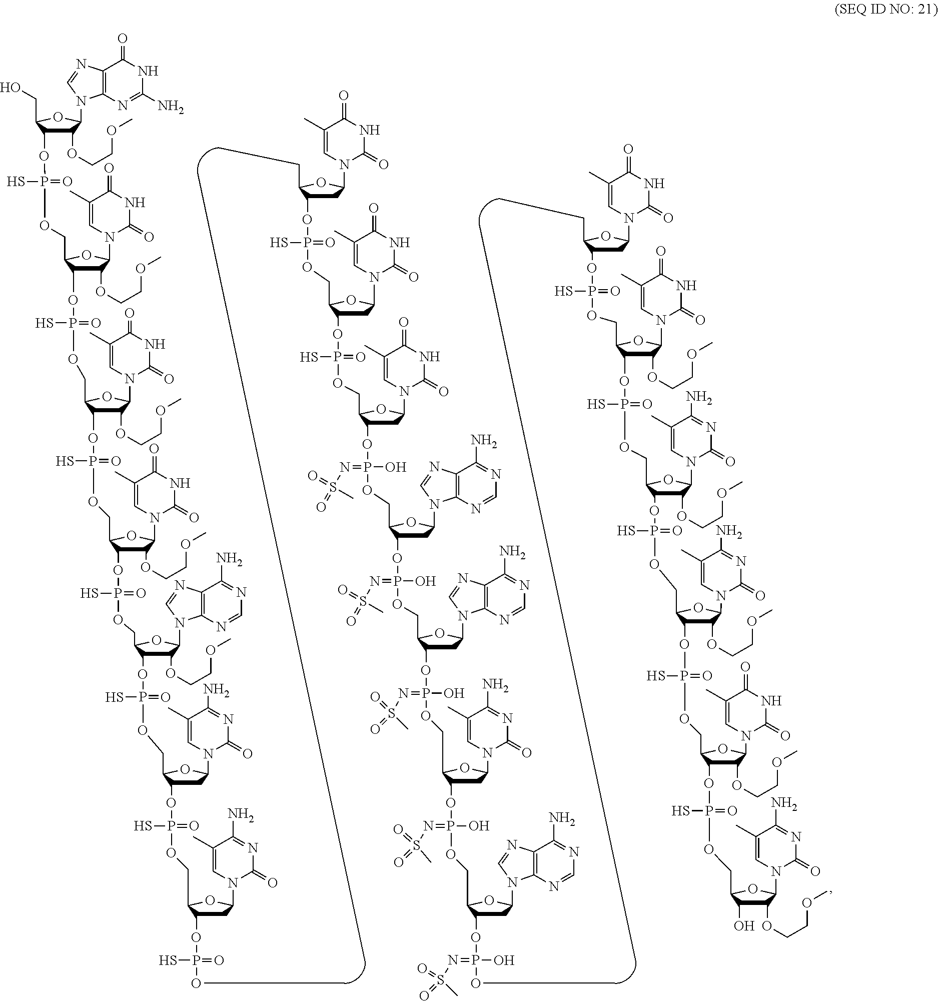

or a pharmaceutically acceptable salt thereof.
Embodiment 42. The modified oligonucleotide of embodiment 41, which is a pharmaceutically acceptable salt comprising one or more cations selected from sodium, potassium, calcium and magnesium.
Embodiment 43. The modified oligonucleotide of embodiment 42, which is the sodium salt or the potassium salt.
Embodiment 44. A modified oligonucleotide according to the following chemical structure:

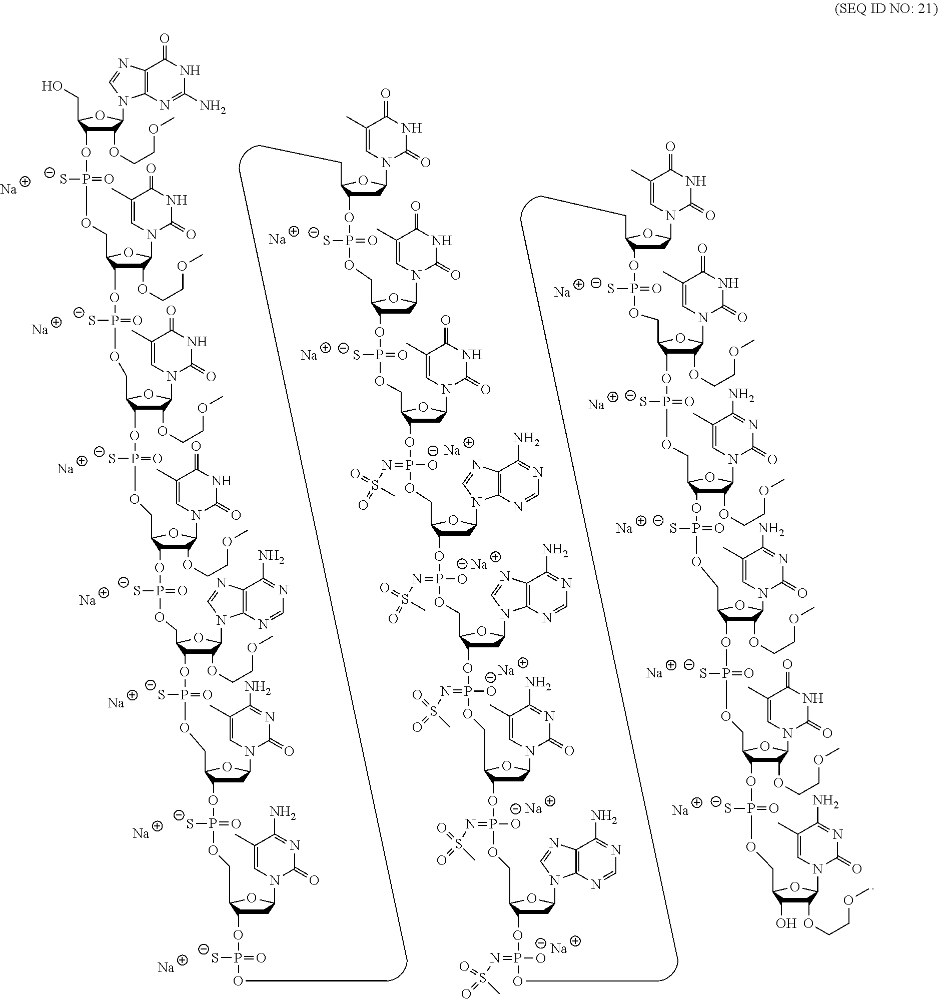

Embodiment 45. A modified oligonucleotide according to the following chemical structure:

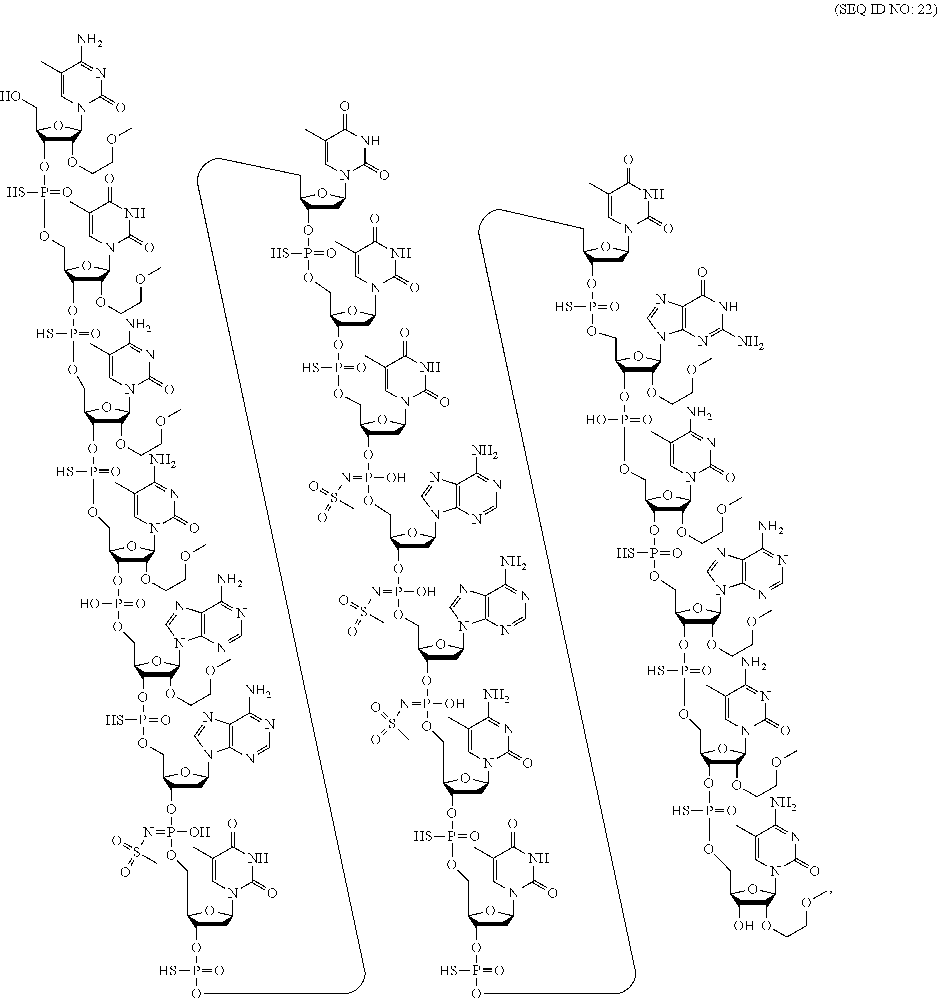

or a pharmaceutically acceptable salt thereof.
Embodiment 46. The modified oligonucleotide of embodiment 45, which is a pharmaceutically acceptable salt comprising one or more cations selected from sodium, potassium, calcium and magnesium.
Embodiment 47. The modified oligonucleotide of embodiment 46, which is the sodium salt or the potassium salt.
Embodiment 48. A modified oligonucleotide according to the following chemical structure:

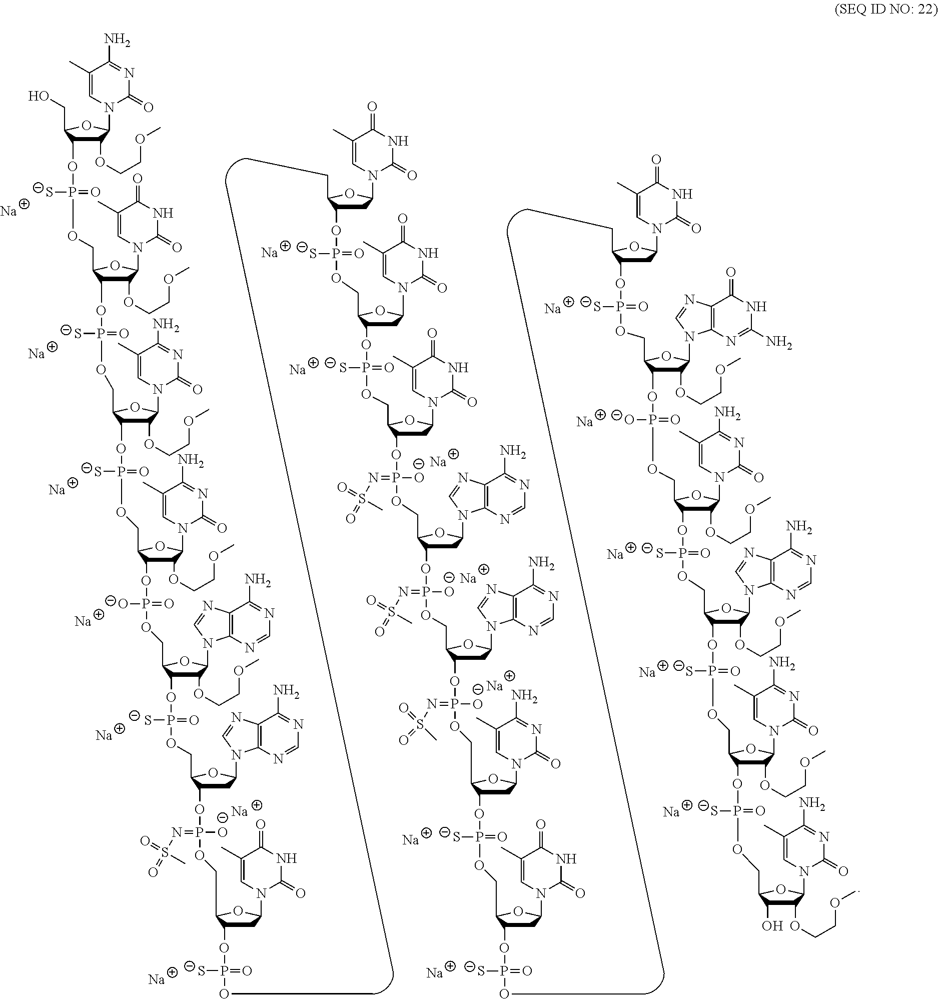

Embodiment 49. A modified oligonucleotide according to the following chemical structure:

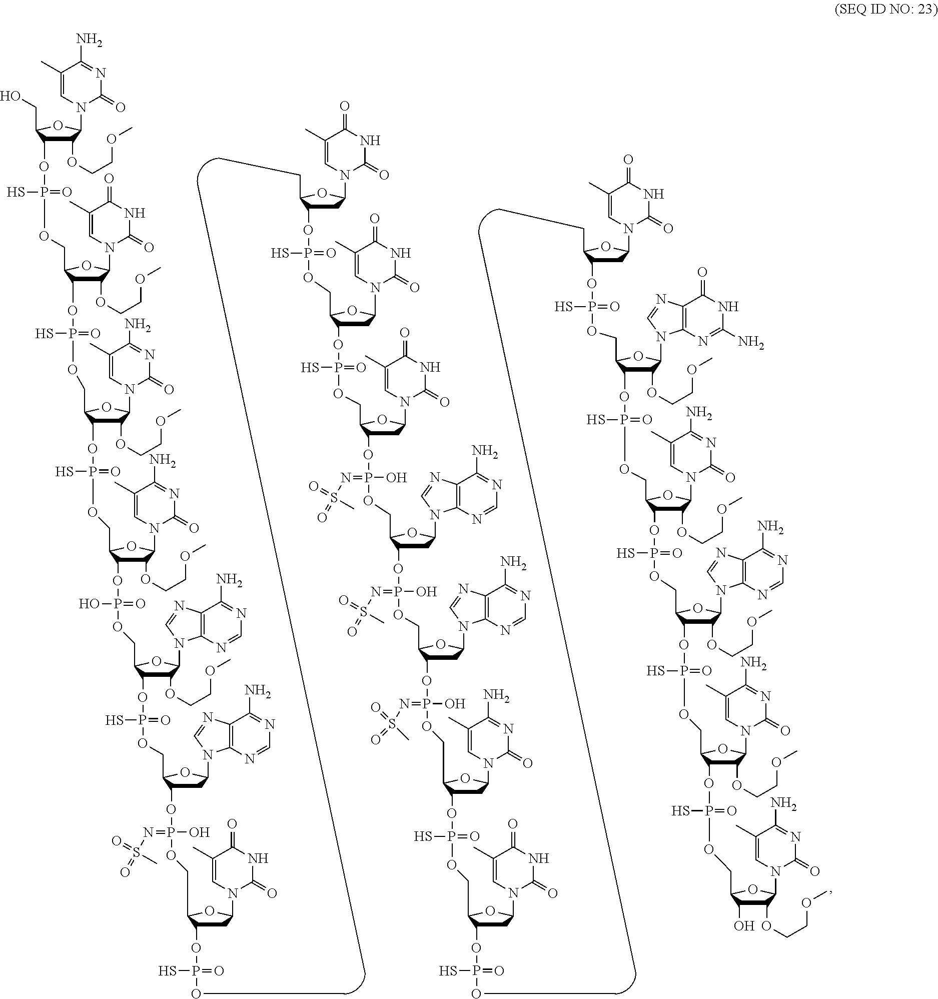

or a pharmaceutically acceptable salt thereof.
Embodiment 50. The modified oligonucleotide of embodiment 49, which is a pharmaceutically acceptable salt comprising one or more cations selected from sodium, potassium, calcium and magnesium.
Embodiment 51. The modified oligonucleotide of embodiment 50, which is the sodium salt or the potassium salt.
Embodiment 52. A modified oligonucleotide according to the following chemical structure:

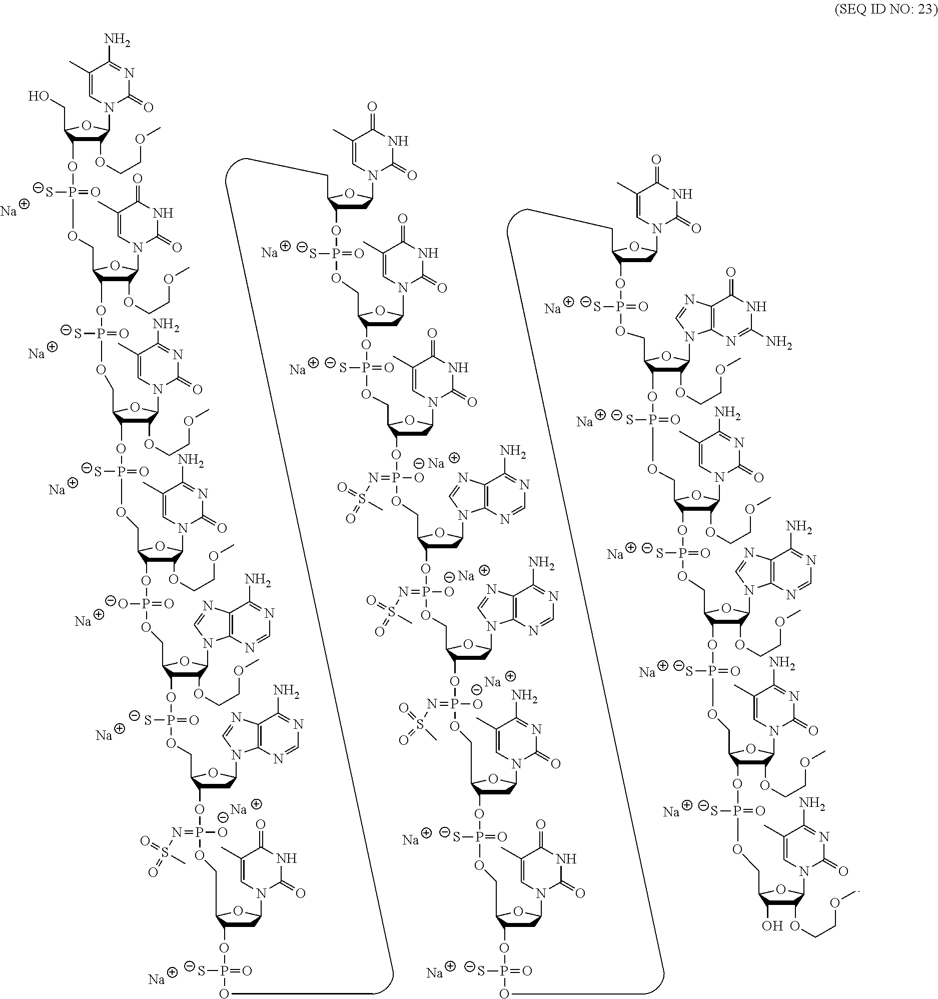

Embodiment 53. A modified oligonucleotide according to the following chemical structure:

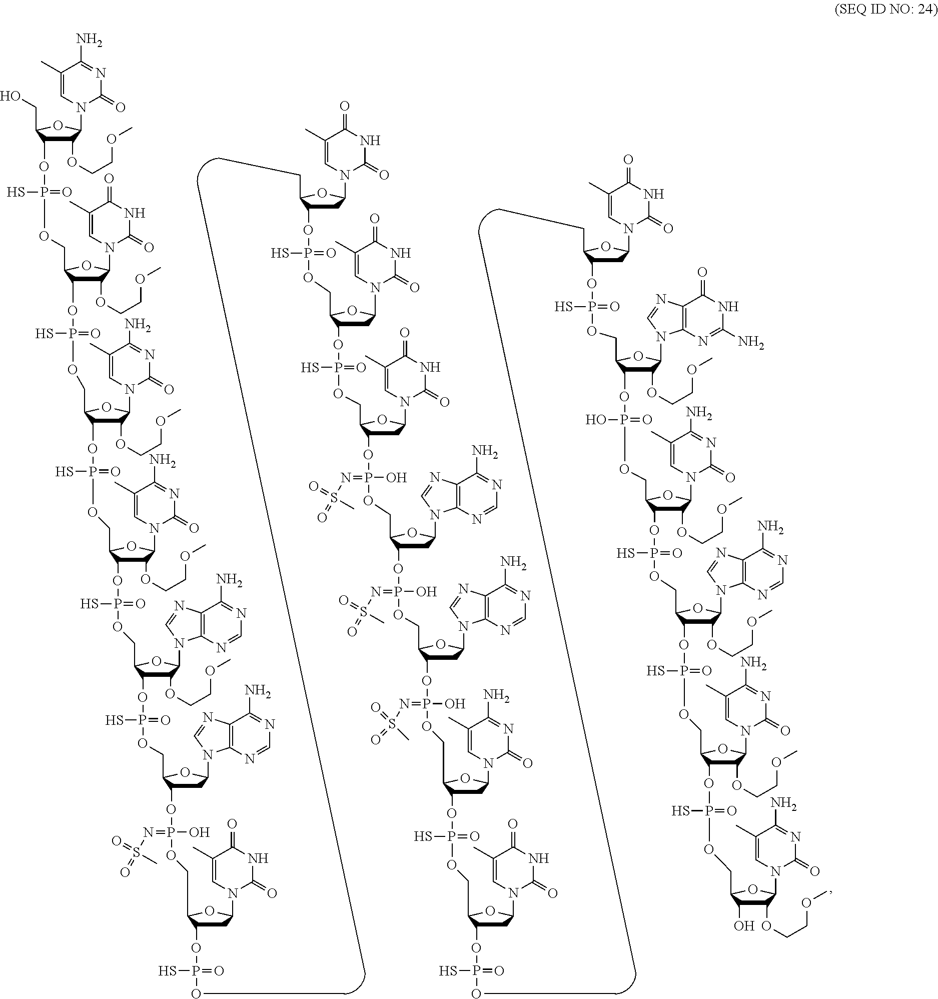

or a pharmaceutically acceptable salt thereof.
Embodiment 54. The modified oligonucleotide of embodiment 53, which is a pharmaceutically acceptable salt comprising one or more cations selected from sodium, potassium, calcium and magnesium.
Embodiment 55. The modified oligonucleotide of embodiment 54, which is the sodium salt or the potassium salt.
Embodiment 56. A modified oligonucleotide according to the following chemical structure:

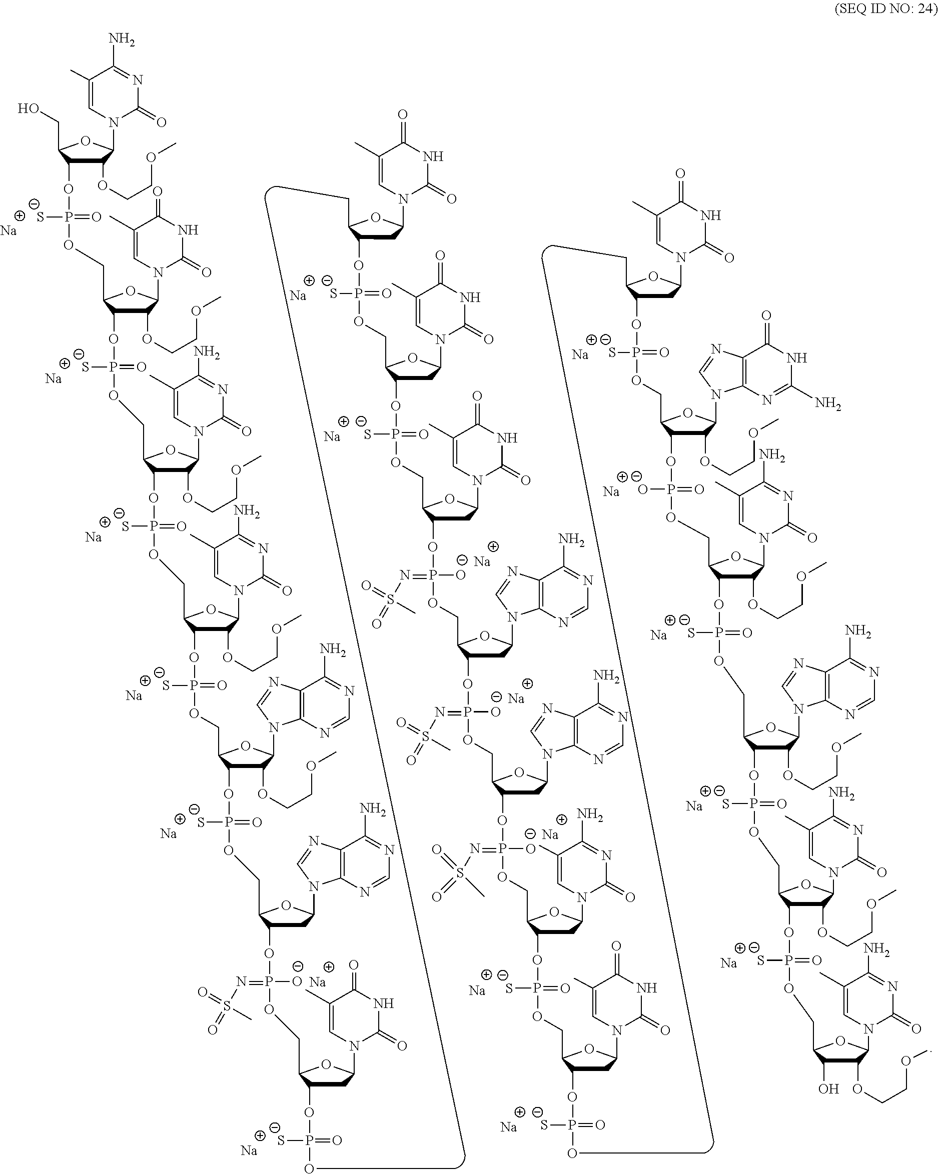

Embodiment 57. An oligomeric compound comprising a modified oligonucleotide according to the following chemical notation:

(SEQ ID NO: 19)
^{m}CₑₛTₑₛ^{m}Cₑₛ^{m}CₑₒAₑₛA_{dz}T_{ds}T_{ds}T_{ds}T_{ds}A_{dz}A_{dz}^{m}C_{ds}T_{ds}T_{ds}
Gₑₒ^{m}CₑₛAₑₛ^{m}Cₑₛ^{m}Cₑ,

wherein:
   A=an adenine nucleobase,
   ^{m}C=5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage,
   o=a pbsphodiester internucleoside linkage, and
   z=a mesyl phosphoramidate internucleoside linkage, and
   wherein the oligomeric compound optionally comprises a conjugate group or a terminal group.
Embodiment 58. An oligomeric compound comprising a modified oligonucleotide according to the following chemical notation:

(SEQ ID NO: 20)
GₑₛTₑₛTₑₒTₑₒAₑₛ^{m}C_{ds}^{m}C_{ds}T_{ds}T_{ds}T_{dz}A_{dz}A_{dz}^{m}C_{ds}A_{dz}T_{ds}
Tₑₒ^{m}Cₑₛ^{m}CₑₛTₑₛ^{m}Cₑ,

wherein:
   A=an adenine nucleobase,
   ^{m}C=a 5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage,
   o=a pbsphodiester internucleoside linkage, and
   z=a mesyl phosphoramidate internucleoside linkage, and
   wherein the oligomeric compound optionally comprises a conjugate group or a terminal group.
Embodiment 59. An oligomeric compound comprising a modified oligonucleotide according to the following chemical notation:

(SEQ ID NO: 21)
GₑₛTₑₛTₑₛTₑₛAₑₛ^{m}C_{ds}^{m}C_{ds}T_{ds}T_{ds}T_{dz}A_{dz}A_{dz}^{m}C_{dz}A_{dz}T_{ds}
Tₑₛ^{m}Cₑₛ^{m}CₑₛTₑₛ^{m}Cₑ,

wherein:
   A=an adenine nucleobase,
   ^{m}C=a 5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage, and
   z=a mesyl phosphoramidate internucleoside linkage, and
   wherein the oligomeric compound optionally comprises a conjugate group or a terminal group.
Embodiment 60. An oligomeric compound comprising a modified oligonucleotide according to the following chemical notation:

(SEQ ID NO: 22)
^{m}CₑₛTₑₛ^{m}Cₑₛ^{m}CₑₒAₑₛA_{dz}T_{ds}T_{ds}T_{ds}T_{dz}A_{dz}A_{dz}^{m}C_{ds}T_{ds}T_{ds}
Gₑₒ^{m}CₑₛAₑₛ^{m}Cₑₛ^{m}Cₑ,

wherein:
   A=an adenine nucleobase, PGP-35,DNA
   ^{m}C=a 5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage,
   o=a phosphodiester internucleoside linkage, and
   z=a mesyl phosphoramidate internucleoside linkage, and
   wherein the oligomeric compound optionally comprises a conjugate group or a terminal group.
Embodiment 61. An oligomeric compound comprising a modified oligonucleotide according to the following chemical notation:

(SEQ ID NO: 23)
^{m}CₑₛTₑₛ^{m}Cₑₛ^{m}CₑₒAₑₛA_{dz}T_{ds}T_{ds}T_{ds}T_{dz}A_{dz}A_{dz}^{m}C_{ds}T_{ds}T_{ds}
Gₑₛ^{m}CₑₛAₑₛ^{m}Cₑₛ^{m}Cₑ,

wherein:
   A=an adenine nucleobase,
   ^{m}C=a 5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage,
   o=a phosphodiester internucleoside linkage, and
   z=a mesyl phosphoramidate internucleoside linkage, and
   wherein the oligomeric compound optionally comprises a conjugate group or a terminal group.
Embodiment 62. An oligomeric compound comprising a modified oligonucleotide according to the following chemical notation:

(SEQ ID NO: 24)
^{m}CₑₛTₑₛ^{m}Cₑₛ^{m}CₑₛAₑₛA_{dz}T_{ds}T_{ds}T_{ds}T_{dz}A_{dz}A_{dz}^{m}C_{ds}T_{ds}T_{ds}
Gₑₒ^{m}CₑₛAₑₛ^{m}Cₑₛ^{m}Cₑ,

wherein:
   A=an adenine nucleobase,
   =a 5-methylcytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage,
   o=a phosphodiester internucleoside linkage, and
   z=a mesyl phosphoramidate internucleoside linkage, and
   wherein the oligomeric compound optionally comprises a conjugate group or a terminal group.
Embodiment 63. A population of oligomeric compounds of any of embodiments 1-27 or 52-57 or a population of modified oligonucleotides of any of embodiments 28-51, wherein all of the phosphorothioate internucleoside linkages of the modified oligonucleotide are stereorandom.
Embodiment 64. A pharmaceutical composition comprising an oligomeric compound of any of embodiments 1-32 or 57-62, or a modified oligonucleotide of any of embodiments 33-56, or a population of embodiment 63, and a pharmaceutically acceptable diluent.
Embodiment 65. The pharmaceutical composition of embodiment 64, wherein the pharmaceutically acceptable diluent is artificial cerebrospinal fluid or phosphate-buffered saline.
Embodiment 66. The pharmaceutical composition of any of embodiments 64-65, wherein the pharmaceutical composition consists essentially of the modified oligonucleotide, the oligomeric compound, or the population and artificial cerebrospinal fluid.
Embodiment 67. The pharmaceutical composition of any of embodiments 64-66, wherein the pharmaceutical composition consists essentially of the modified oligonucleotide, the oligomeric compound, or the population and phosphate-buffered saline.
Embodiment 68. A method comprising administering to a subject an oligomeric compound of any of embodiments 1-32 or 57-62, a modified oligonucleotide of any of embodiments 33-56, a population of embodiment 63, or a pharmaceutical composition of any of embodiments 64-67.
Embodiment 69. The method of embodiment 68, wherein the subject has or is at risk of developing a disease or disorder associated with APP.
Embodiment 70. The method of embodiment 68 or embodiment 69, wherein administering the oligomeric compound, the modified oligonucleotide, the population, or the pharmaceutical composition ameliorates at least one symptom or hallmark of a disease or disorder associated with APP.
Embodiment 71. The method of any of embodiments 68-70, wherein administering the modified oligonucleotide, the oligomeric compound, the population, or the pharmaceutical composition reduces or slows progression of cognitive impairment, reduces or slows decline in memory, reduces or slows decline in language skills, improves behavioral and psychological symptoms, reduces apathy, improves motivation, reduces gait disturbances, reduces seizures, reduces or slows progressive dementia, and/or reduces abnormal amyloid deposits.
Embodiment 72. The method of any of embodiments 68-71, wherein APP protein levels in the subject are reduced.
Embodiment 73. The method of any of embodiments 69-72, wherein the disease or disorder associated with APP is sporadic Alzheimer's Disease, genetic/familial Alzheimer's Disease, Alzheimer's Disease in a Down Syndrome patient, or Cerebral Amyloid Angiopathy.
Embodiment 74. A method of treating a disease or disorder associated with APP comprising administering to a subject having or at risk for developing a disease or disorder associated with APP a therapeutically effective amount of an oligomeric compound of any of embodiments 1-32 or 57-62, a modified oligonucleotide of any of embodiments 33-56, a population of embodiment 63, or a pharmaceutical composition of any of embodiments 64-67, thereby treating the disease or disorder associated with APP.
Embodiment 75. The method of embodiment 73, wherein administering the modified oligonucleotide, the oligomeric compound, the population, or the pharmaceutical composition ameliorates at least one symptom or hallmark of the disease or disorder associated with APP.
Embodiment 76. The method of any of embodiments 73-74, wherein administering the modified oligonucleotide, the oligomeric compound, the population, or the pharmaceutical composition reduces or slows progression of cognitive impairment, reduces or slows decline in memory, reduces or slows decline in language skills, improves behavioral and psychological symptoms, reduces apathy, improves motivation, reduces gait disturbances, reduces seizures, reduces or slows progressive dementia, and/or reduces abnormal amyloid deposits.
Embodiment 77. The method of any of embodiments 74-76, wherein APP protein levels in the subject are reduced.
Embodiment 78. The method of any of embodiments 74-77, wherein the disease or disorder associated with APP is sporadic Alzheimer's Disease, genetic/familial Alzheimer's Disease, Alzheimer's Disease in a Down Syndrome patient, or Cerebral Amyloid Angiopathy.
Embodiment 79. The method of any of embodiments 74-78, wherein the subject is Inman.
Embodiment 80. A method of reducing expression of APP in a cell comprising contacting the cell with an oligomeric compound of any of embodiments 1-32 or 56-62, a modified oligonucleotide of any of embodiments 33-56, a population of embodiment 63, or a pharmaceutical composition of any of embodiments 62-65.
Embodiment 81. The method of embodiment 80, wherein the cell is a cortical brain cell or a hippocampal cell.
Embodiment 82. The method of embodiment 80 or embodiment 81, wherein the cell is a human cell.
Embodiment 83. Use of an oligomeric compound of any of embodiments 1-32 or 57-62, a modified oligonucleotide of any of embodiments 33-56, a population of embodiment 63, or a pharmaceutical composition of any of embodiments 64-67 for treating a disease or disorder associated with APP.
Embodiment 84. Use of an oligomeric compound of any of embodiments 1-32 or 57-62, a modified oligonucleotide of any of embodiments 33-56, a population of embodiment 63, or a pharmaceutical composition of any of embodiments 64-67 in the manufacture of a medicament for treating a disease or disorder associated with APP.
Embodiment 85. The use of any of embodiments 83-84, wherein the disease or disorder is associated with an elevated level of APP.
Embodiment 86. The use of any of embodiments 83-85, wherein the disease or disorder associated with APP is sporadic Alzheimer's Disease, genetic/familial Alzheimer's Disease, Alzheimer's Disease in a Down Syndrome patient, or Cerebral Amyloid Angiopathy.

### Certain Oligomeric Agents and Oligomeric Compounds

Certain embodiments provide oligomeric agents targeted to an APP nucleic acid. In certain embodiments, an APP nucleic acid has the sequence set forth in SEQ ID NO: 1 (the cDNA of Ensembl transcript ENST00000346798.7 from version 94: October 2018) or SEQ ID NO: 2 (GENBANK Accession No. NC_000021.9 truncated sated from nucleotides 25878001 to 26174000). In certain embodiments, the oligomeric agent is a single-stranded oligomeric compound. In certain embodiments, the oligomeric agent is an oligomeric duplex.

Certain embodiments provide an oligomeric compound comprising a modified oligonucleotide consisting of 8 to 80 linked nucleosides, wherein the nucleobase sequence of the modified oligonucleotide is at least 80% complementary to an equal length portion of a APP nucleic acid, and wherein at least one internucleoside linkage of the modified oligonucleotide is a mesyl phosphoramidate internucleoside linkage. In certain embodiments, the APP nucleic acid has the nucleobase sequence of SEQ ID NO: 1 or SEQ ID NO: 2. In certain embodiments, the nucleobase sequence of the modified oligonucleotide is at least 85%, at least 90%, at least 95%, or 100% complementary to an equal length portion of the APP nucleic acid. In any of the oligomeric compounds provided herein, the nucleobase sequence of the modified oligonucleotide is at least 85%, at least 90%, at least 95%, or 100% complementary to an equal length portion of an APP nucleic acid, wherein the APP nucleic acid has the nucleobase sequence of SEQ ID NO: 1 or SEQ ID NO: 2.

Certain embodiments provide an oligomeric compound comprising a modified oligonucleotide consisting of 8 to 80 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases of the nucleobase sequence of any of SEQ ID NOS: 15-18. In certain embodiments, the modified oligonucleotide has a nucleobase sequence comprising the nucleobase sequence of any of SEQ ID NOS: 15-18.

Certain embodiments provide an oligomeric compound comprising a modified oligonucleotide consisting of 8 to 80 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases of the nucleobase sequence of SEQ ID NO: 15 or SEQ ID NO: 18. In certain embodiments, the modified oligonucleotide has a nucleobase sequence comprising the nucleobase sequence of SEQ ID NO: 15 or SEQ ID NO: 18.

Certain embodiments provide an oligomeric compound comprising a modified oligonucleotide consisting of 20 to 80 linked nucleosides and has a nucleobase sequence comprising the nucleobase sequence of any of SEQ ID NOS: 15-18. Certain embodiments provide an oligomeric compound comprising a modified oligonucleotide having a nucleobase sequence consisting of any of SEQ ID NOS: 15-18.

Certain embodiments provide an oligomeric compound comprising a modified oligonucleotide consisting of 20 to 80 linked nucleosides and has a nucleobase sequence comprising the nucleobase sequence of SEQ ID NO: 15 or SEQ ID NO: 18. Certain embodiments provide an oligomeric compound comprising a modified oligonucleotide having a nucleobase sequence consisting of SEQ ID NO: 15 or SEQ ID NO: 18.

Certain embodiments provide an oligomeric compound comprising a modified oligonucleotide consisting of 20 to 80 linked nucleosides, wherein the nucleobase sequence of the modified oligonucleotide comprises the nucleobase sequence of SEQ ID NO: 15. Certain embodiments provide an oligomeric compound comprising a modified oligonucleotide consisting of 20 linked nucleosides, wherein the nucleobase sequence of the modified oligonucleotide consists of the nucleobase sequence of SEQ ID NO: 15.

Certain embodiments provide an oligomeric compound comprising a modified oligonucleotide consisting of 20 to 80 linked nucleosides, wherein the nucleobase sequence of the modified oligonucleotide comprises the nucleobase sequence of SEQ ID NO: 16. Certain embodiments provide an oligomeric compound comprising a modified oligonucleotide consisting of 20 linked nucleosides, wherein the nucleobase sequence of the modified oligonucleotide consists of the nucleobase sequence of SEQ ID NO: 16.

Certain embodiments provide an oligomeric compound comprising a modified oligonucleotide consisting of 20 to 80 linked nucleosides, wherein the nucleobase sequence of the modified oligonucleotide comprises the nucleobase sequence of SEQ ID NO: 17. Certain embodiments provide an oligomeric compound comprising a modified oligonucleotide consisting of 20 linked nucleosides, wherein the nucleobase sequence of the modified oligonucleotide consists of the nucleobase sequence of SEQ ID NO: 17.

Certain embodiments provide an oligomeric compound comprising a modified oligonucleotide consisting of 20 to 80 linked nucleosides, wherein the nucleobase sequence of the modified oligonucleotide comprises the nucleobase sequence of SEQ ID NO: 18. Certain embodiments provide an oligomeric compound comprising a modified oligonucleotide consisting of 20 linked nucleosides, wherein the nucleobase sequence of the modified oligonucleotide consists of the nucleobase sequence of SEQ ID NO: 18.

In any of the oligomeric compounds provided herein, the modified oligonucleotide can consist of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 22, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides. In certain embodiments, the modified oligonucleotide consists of 20 linked nucleosides.

In any of the oligomeric compounds provided herein, the modified oligonucleotide comprises at least one modified internucleoside linkage, wherein the at least one modified internucleoside linkage of the modified oligonucleotide is a mesyl phosphoramidate internucleoside linkage. In any of the oligomeric compounds provided herein, at least 2, at least 3, at least 4, at least 5, at least 6, at least 7, or at least 8 internucleoside linkages of the modified oligonucleotide are mesyl phosphoramidate internucleoside linkages. In certain embodiments, the modified oligonucleotide comprises 1, 2, 3, 4, 5, 6, or 1-6 mesyl phosphoramidate internucleoside linkages. In any of the oligomeric compounds provided herein, the modified oligonucleotide has an internucleoside linkage motif comprising at least one mesyl phosphoramidate internucleoside linkage. In certain embodiments, the modified oligonucleotide comprises an internucleoside linkage motif comprising at least 2, at least 3, at least 4, at least 5, at least 6, at least 7, or at least 8 mesyl phosphoramidate internucleoside linkages. In certain embodiments, the modified oligonucleotide comprises an internucleoside linkage motif comprising 1, 2, 3, 4, 5, 6, or 1-6 mesyl phosphoramidate internucleoside linkages. In certain embodiments, the modified oligonucleotide comprises an internucleoside linkage motif comprising 2-5 mesyl phosphoramidate internucleoside linkages. In certain embodiments, the modified oligonucleotide comprises an internucleoside linkage motif comprising 2 mesyl phosphoramidate internucleoside linkages. In certain embodiments, the modified oligonucleotide comprises an internucleoside linkage motif comprising 3 mesyl phosphoramidate internucleoside linkages. In certain embodiments, the modified oligonucleotide comprises an internucleoside linkage motif comprising 4 mesyl phosphoramidate internucleoside linkages. In certain embodiments, the modified oligonucleotide comprises an internucleoside linkage motif comprising 5 mesyl phosphoramidate internucleoside linkages.

In any of the oligomeric compounds provided herein, at least one internucleoside linkage of the modified oligonucleotide may be a phosphorothioate internucleoside linkage. In certain embodiments, the modified oligonucleotide has an internucleoside linkage motif comprising at least one mesyl phosphoramidate internucleoside linkage and at least one phosphorothioate internucleoside linkage. In certain embodiments, at least 9, at least 10, at least 11, at least 12, at least 13, at least 14, at least 15, at least 16, at least 17, or at least 18 internucleoside linkages of the modified oligonucleotide are phosphorothioate internucleoside linkages. In certain embodiments, the modified oligonucleotide has an internucleoside linkage motif comprising 9-18 phosphorothioate internucleoside linkages. In certain embodiments, the modified oligonucleotide has an internucleoside linkage motif comprising 10-14 phosphorothioate internucleoside linkages. In certain embodiments, the modified oligonucleotide has an internucleoside linkage motif comprising 14 phosphorothioate internucleoside linkages.

In certain embodiments, at least one internucleoside linkage of the modified oligonucleotide can be a phosphodiester internucleoside linkage. In certain embodiments, the modified oligonucleotide comprises at least one a mesyl phosphoramidate internucleoside linkage and at least one phosphodiester internucleoside linkage. In certain embodiments, the modified oligonucleotide has an internucleoside linkage motif comprising at least one phosphodiester internucleoside linkage. In certain embodiments, the modified oligonucleotide has an internucleoside linkage motif comprising at least one, at least 2, at least 3, at least 4, at least 5, or at least 6 phosphodiester internucleoside linkages. In certain embodiments, the modified oligonucleotide has an internucleoside linkage motif comprising 1-3 phosphodiester internucleoside linkages. In certain embodiments, the modified oligonucleotide has an internucleoside linkage motif comprising 1 phosphodiester internucleoside linkage. In certain embodiments, the modified oligonucleotide has an internucleoside linkage motif comprising 2 phosphodiester internucleoside linkages.

In certain embodiments, the modified oligonucleotide comprises at least one mesyl phosphoramidate internucleoside linkage, and at least one of a phosphorothioate internucleoside linkage and a phosphodiester internucleoside linkage. In certain embodiments, the modified oligonucleotide comprises at least one mesyl phosphoramidate internucleoside linkage, at least one phosphorothioate internucleoside linkage, and at least one phosphodiester internucleoside linkage.

In any of the oligomeric compounds provided herein, at least one nucleoside of the modified oligonucleotide comprises a modified sugar moiety. In certain embodiments, the modified sugar moiety comprises a non-bicyclic sugar moiety, such as a 2′-MOE sugar moiety or 2′—OMe sugar moiety. In certain embodiments, the modified sugar moiety is a 2′-MOE sugar moiety.

In any of the oligomeric compounds provided herein, at least one nucleobase of the modified oligonucleotide can be a modified nucleobase, such as 5-methylcytosine. In certain embodiments, each cytosine is 5-methylcytosine. In any of the oligomeric compounds provided herein, the modified oligonucleotide can comprise a deoxy region consisting of 5-12 contiguous 2′-deoxynucleosides. In certain embodiments, each nucleoside of the deoxy region is a 2′-β-D-deoxynucleoside. In certain embodiments, the deoxy region consists of 6, 7, 8, 9, 10, or 6-10 linked nucleosides. In certain embodiments, each nucleoside immediately adjacent to the deoxy region comprises a modified sugar moiety. In certain embodiments, the deoxy region is flanked on the 5′-side by a 5′-external region consisting of 1-6 linked 5′-external region nucleosides and on the 3′-side by a 3′-external region consisting of 1-6 linked 3′-external region nucleosides; wherein the 3′-most nucleoside of the 5′-external region comprises a modified sugar moiety; and the 5′-most nucleoside of the 3′-external region comprises a modified sugar moiety. In certain embodiments, each nucleoside of the 3′-external region comprises a modified sugar moiety. In certain embodiments, each nucleoside of the 5′-external region comprises a modified sugar moiety. In certain embodiments, each of the 5′ external region nucleosides and each of the 3′ external region nucleosides is a 2′-MOE nucleoside. In certain embodiments, the modified oligonucleotide has a 5′ external region consisting of 5 linked nucleosides, a deoxy region consisting of 10 linked nucleosides, and a 3′ external region consisting of 5 linked nucleosides, wherein each of the 5′ external region nucleosides and each of the 3′ external region nucleosides is a 2′-MOE nucleoside.

### 1. Compound No. 1620705

Compound No. 1620705 is characterized as a 5-10-5 MOE gapmer consisting of 20 linked nucleosides and having a nucleobase sequence of (from 5′ to 3′) CTCCAATTITAACTTGCACC (SEQ ID NO: 18), wherein each of nucleosides 1-5 and 16-20 (from 5′ to 3′) are 2′-MOE nucleosides and each of nucleosides 6-15 are 2′-β-D-deoxynucleosides, wherein the internucleoside linkages between nucleosides 4 to 5 and 16 to 17 are phosphodiester internucleoside linkages, the internucleoside linkages between nucleosides 1 to 2, 2 to 3, 3 to 4, 5 to 6, 7 to 8, 8 to 9, 9 to 10, 10 to 11, 13 to 14, 14 to 15, 15 to 16, 17 to 18, 18 to 19, and 19 to 20 are phosphorothioate internucleoside linkages, and the internucleoside linkages between nucleosides 6 to 7, 11 to 12, and 12 to 13 are mesyl phosphoramidate linkages, and wherein each cytosine is a 5-methylcytosine.

Compound No. 1620705 is represented by the following chemical notation (5′ to 3′):
^{m}CₑₛTₑₛ^{m}Cₑₛ^{m}CₑₒAₑₛA_{dz}T_{ds}T_{ds}T_{ds}T_{ds}A_{dz}A_{dz}^{m}C_{ds}T_{ds}T_{ds}Gₑₒ^{m}CₑₛAₑₛ^{m}Cₑₛ^{m}Cₑ (SEQ ID NO: 19), wherein,
A=an adenine nucleobase,
^{m}C=a 5-methylcytosine nucleobase,
G=a guanine nucleobase,
T=a thymine nucleobase,
e=a 2′ MOE sugar moiety,
d=a 2′-β-D deoxyribosyl sugar moiety,
s=a phosphorothioate internucleoside linkage,
o=a pbsphodiester internucleoside linkage, and
z=a mesyl phosphoramidate internucleoside linkage, and the compound does not include a conjugate group or terminal group.

Compound No. 1620705 is represented by the following chemical structure:

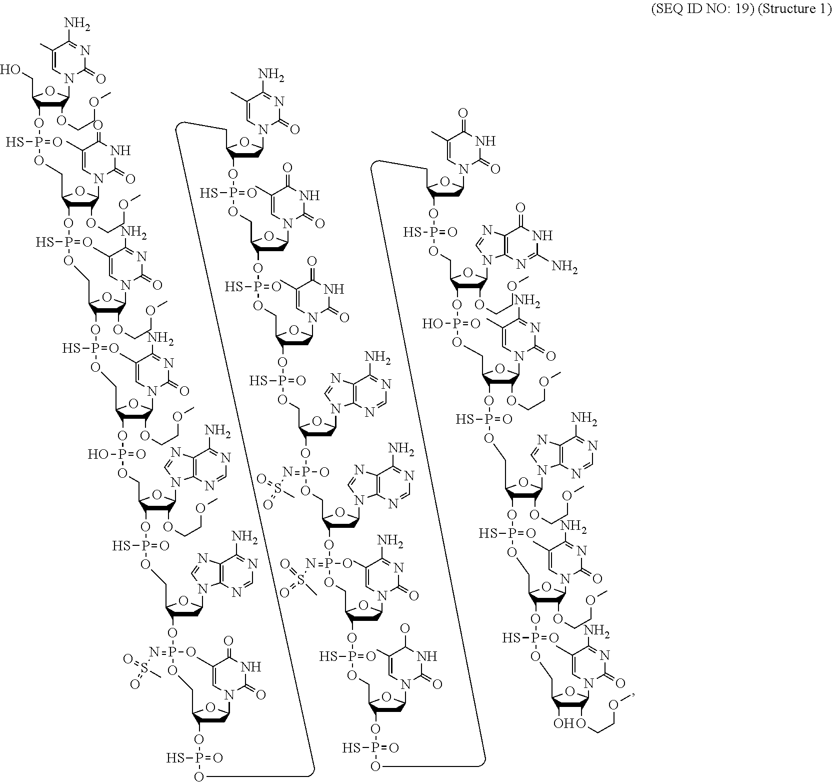

or a pharmaceutically acceptable salt thereof.

In certain embodiments, Compound 1620705 is a pharmaceutically acceptable salt comprising one or more cations selected from sodium, potassium, calcium, and magnesium. In certain embodiments, Compound 1620705 is a sodium salt or a potassium salt.

The sodium salt of Compound No. 1620705 is represented by the following chemical structure:

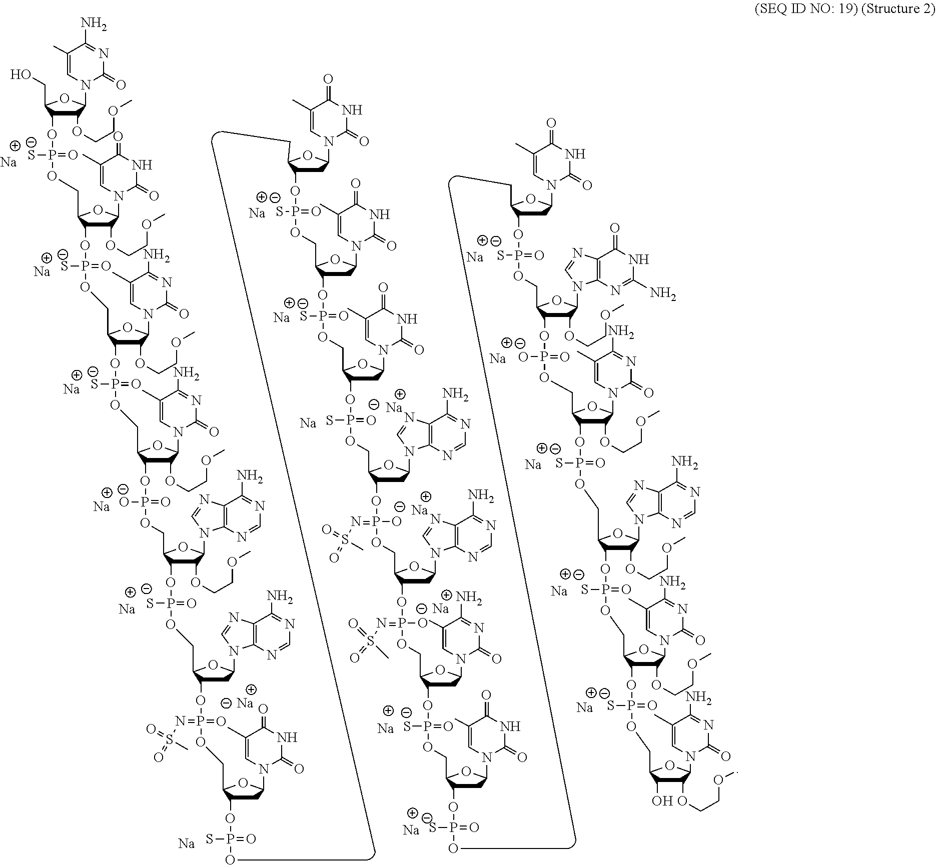

2. Compound No. 1681029

Compound No. 1681029 is characterized as a 5-10-5 MOE gapmer consisting of 20 linked nucleosides and having a nucleobase sequence of (from 5′ to 3′) GTTTACCTTTAACATTCCTC (SEQ ID NO: 15), wherein each of nucleosides 1-5 and 16-20 (from 5′ to 3′) are 2′-MOE nucleosides and each of nucleosides 6-15 are 2′-β-D-deoxynucleosides, wherein the internucleoside linkages between nucleosides 3 to 4, 4 to 5, and 16 to 17 are phosphodiester internucleoside linkages, the internucleoside linkages between nucleosides 1 to 2, 2 to 3, 5 to 6, 6 to 7, 7 to 8, 8 to 9, 9 to 10, 13 to 14, 15 to 16, 17 to 18, 18 to 19, and 19 to 20 are phosphorothioate internucleoside linkages, and the internucleoside linkages between nucleosides 10 to 11, 11 to 12, 12 to 13, and 14 to 15 are mesyl phosphoramidate internucleoside linkages, and wherein each cytosine is a 5-methylcytosine.

Compound No. 1681029 is represented by the following chemical notation (5′ to 3′):
GₑₛTₑₛTₑₒTₑₒAₑₛ^{m}C_{ds}^{m}C_{ds}T_{ds}T_{dz}A_{dz}A_{dz}^{m}C_{ds}A_{dz}T_{ds}Tₑₒ^{m}Cₑₛ^{m}CₑₛTₑₛ^{m}Cₑ (SEQ ID NO: 20), wherein,
A=an unmodified adenine nucleobase,
^{m}C=a 5-methylcytosine nucleobase,
G=an unmodified guanine nucleobase,
T=an unmodified thymine nucleobase,
e=a 2′-MOE sugar moiety,
d=a 2′-β-D-deoxyribosyl sugar moiety,
s=a phosphorothioate internucleoside linkage,
o=a pbsphodiester internucleoside linkage, and
z=a mesyl phosphoramidate internucleoside linkage; and the compound does not include a conjugate or a terminal group.

Compound No. 1681029 is represented by the following chemical structure:

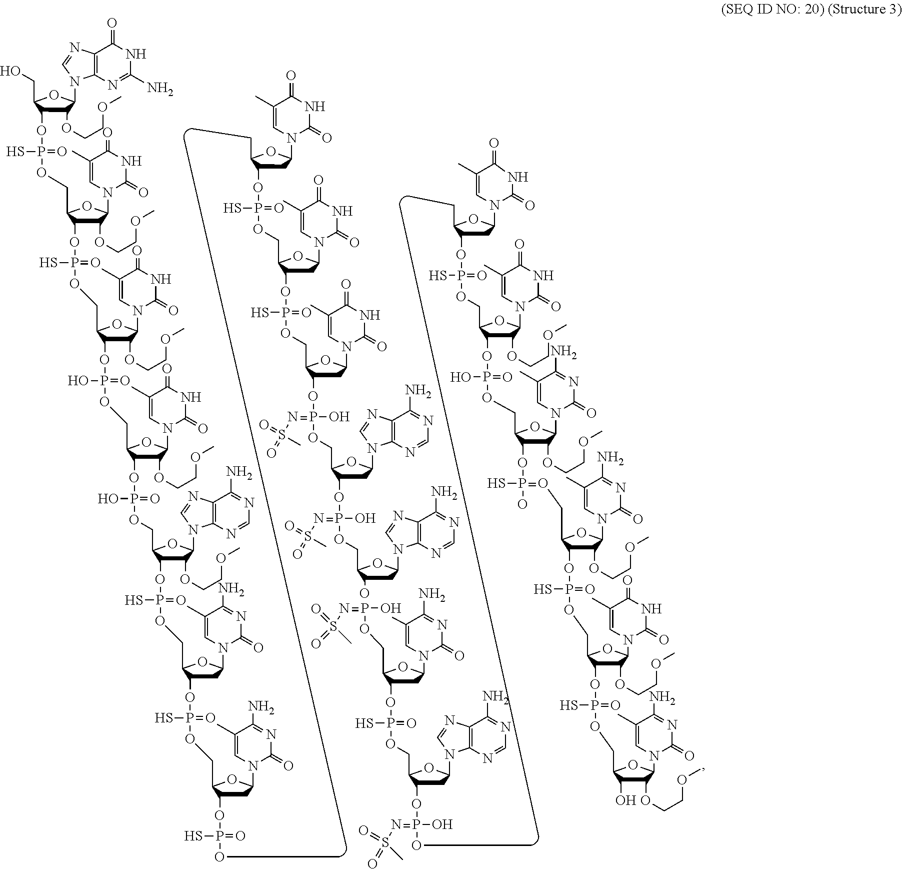

or a pharmaceutically acceptable salt thereof.

In certain embodiments, Compound 1621029 is a pharmaceutically acceptable salt comprising one or more cations selected from sodium, potassium, calcium, and magnesium. In certain embodiments, Compound 1621029 is a sodium salt or a potassium salt.

The sodium salt of Compound No. 1621029 is represented by the following chemical structure:

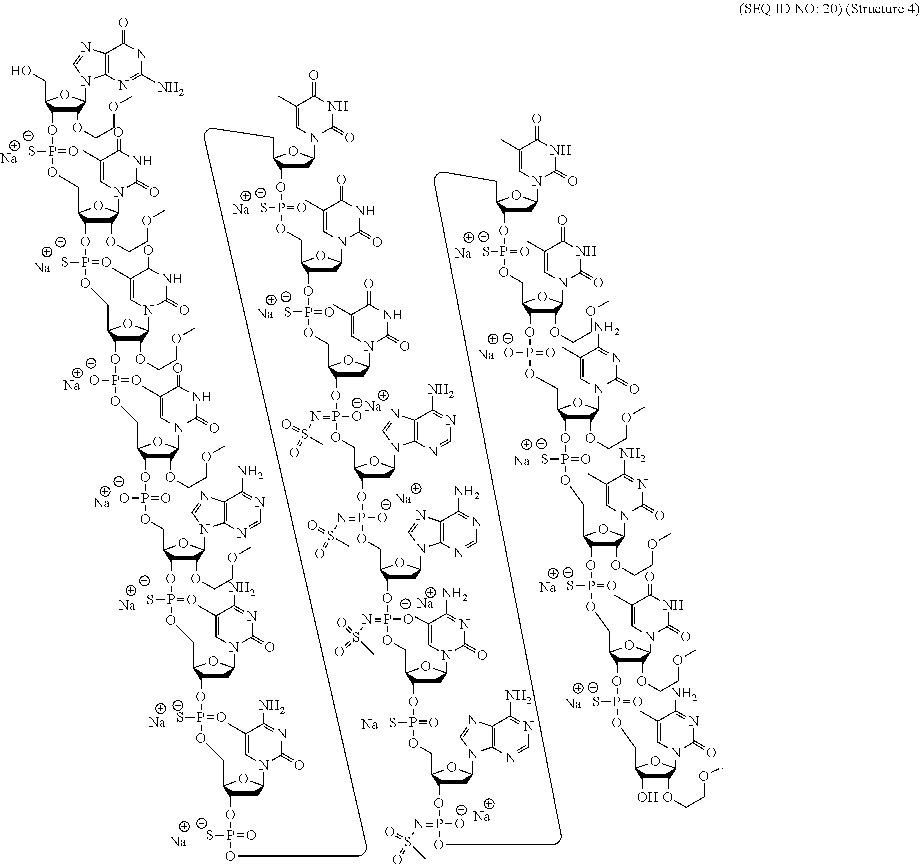

3. Compound No. 1681031

Compound No. 1681031 is characterized as a 5-10-5 MOE gapmer consisting of 20 linked nucleosides and having a nucleobase sequence of (from 5′ to 3′) GTTTACCMAACATTCCTC (SEQ ID NO: 15), wherein each of nucleosides 1-5 and 16-20 (from 5′ to 3′) are 2′-MOE nucleosides and each of nucleosides 6-15 are 2′-β-D-deoxynucleosides, wherein the internucleoside linkages between nucleosides 1 to 2, 2 to 3, 3 to 4, 4 to 5, 5 to 6, 6 to 7, 7 to 8, 8 to 9, 9 to 10, 15 to 16, 16 to 17, 17 to 18, 18 to 19, and 19 to 20 are phosphorothioate internucleoside linkages and the internucleoside linkages between nucleosides 10 to 11, 11 to 12, 12 to 13, 13 to 14, and 14 to 15 are mesyl phosphoramidate internucleoside linkages, and wherein each cytosine is a 5-methylcytosine.

Compound No. 1681031 is represented by the following chemical notation (5′ to 3′):
GₑₛTₑₛTₑₛTₑₛAₑₛ^{m}C_{ds}^{m}C_{ds}T_{ds}T_{ds}T_{dz}A_{dz}C_{dz}A_{dz}T_{ds}Tₑₛ^{m}Cₑₛ^{m}CₑₛTₑₛ^{m}Cₑ (SEQ ID NO: 21), wherein,
A=an adenine nucleobase,
^{m}C=a 5-methylcytosine nucleobase,
G=a guanine nucleobase,
T=a thymine nucleobase,
e=a 2′ MOE sugar moiety,
d=a 2′-β-D deoxyribosyl sugar moiety,
s=a phosphorothioate internucleoside linkage, and
z=a mesyl phosphoramidate internucleoside linkage, and the compound does not include a conjugate group or terminal group.

Compound No. 1681031 is represented by the following chemical structure:

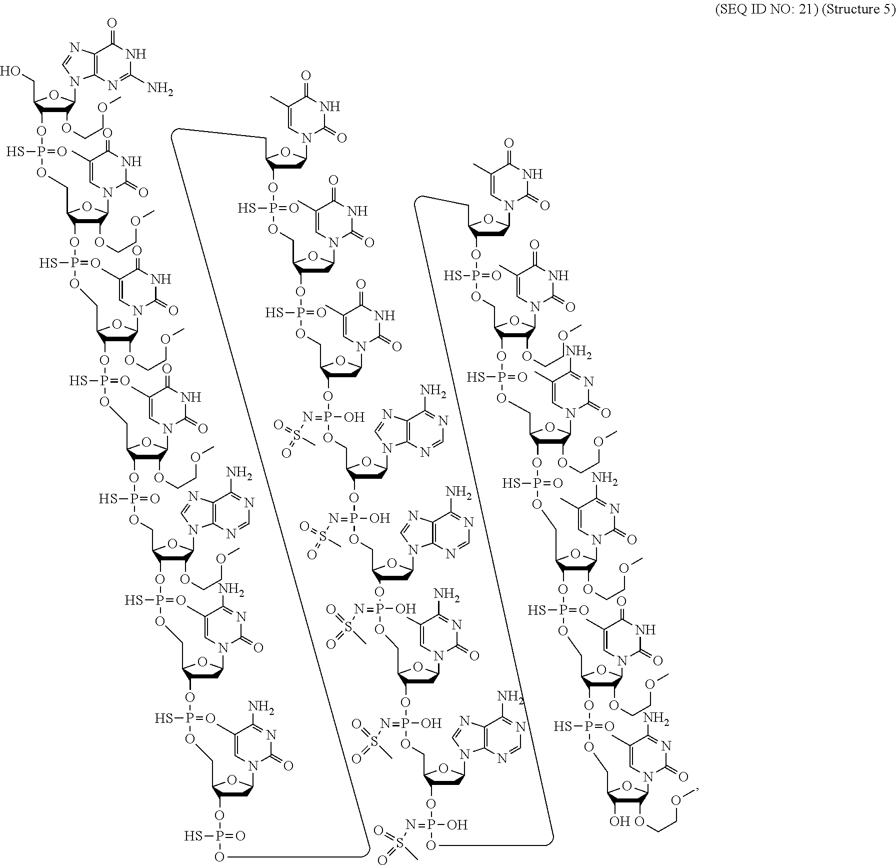

or a pharmaceutically acceptable salt thereof.

In certain embodiments, Compound 1681031 is a pharmaceutically acceptable salt comprising one or more cations selected from sodium, potassium, calcium, and magnesium. In certain embodiments, Compound 1681031 is a sodium salt or a potassium salt.

The sodium salt of Compound No. 1681031 is represented by the following chemical structure:

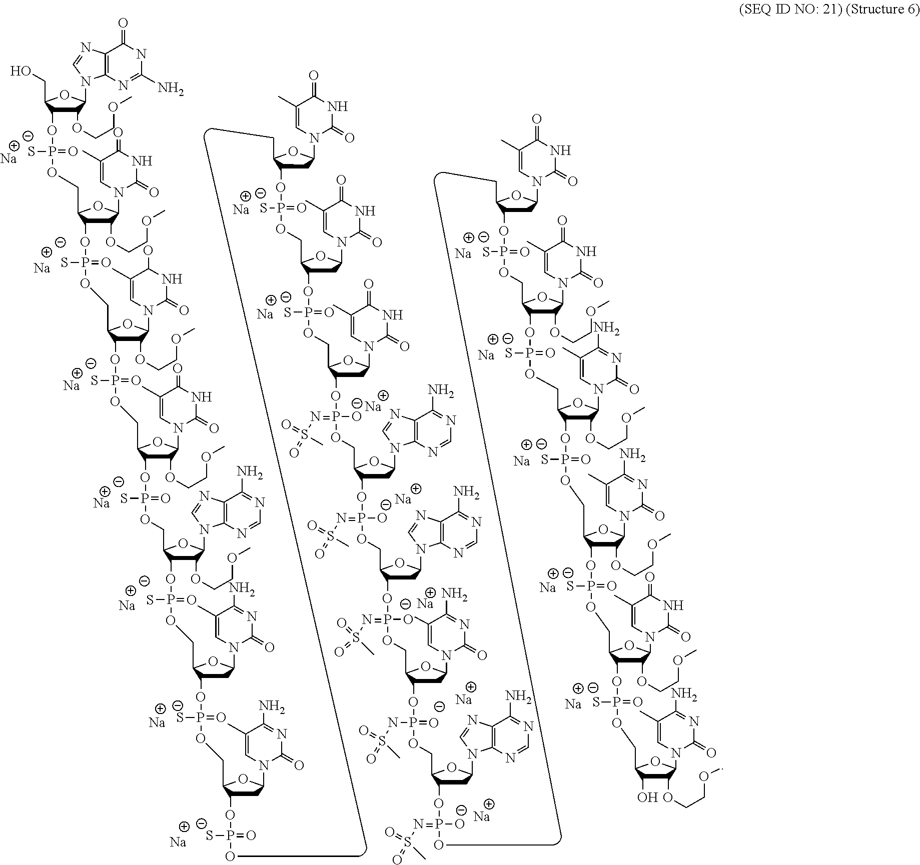

4. Compound No. 1683009

Compound No. 1683009 is characterized as a 5-10-5 MOE gapmer consisting of 20 linked nucleosides and having a nucleobase sequence of (from 5′ to 3′) CTCCAATITTAACTTGCACC (SEQ ID NO: 18), wherein each of nucleosides 1-5 and 16-20 (from 5′ to 3′) are 2′-MOE nucleosides and each of nucleosides 6-15 are 2′-β-D-deoxynucleosides, wherein the internucleoside linkages between nucleosides 4 to 5 and 16 to 17 are phosphodiester internucleoside linkages, the internucleoside linkages between nucleosides 1 to 2, 2 to 3, 3 to 4, 5 to 6, 7 to 8, 8 to 9, 9 to 10, 13 to 14, 14 to 15, 15 to 16, 17 to 18, 18 to 19, and 19 to 20 are phosphorothioate internucleoside linkages, and the internucleoside linkages between nucleosides 6 to 7, 10 to 11, 11 to 12, and 12 to 13 are mesyl phosphoramidate internucleoside linkages, and wherein each cytosine is a 5-methylcytosine.

Compound No. 1683009 is represented by the following chemical notation (5′ to 3′):
^{m}CₑₛTₑₛ^{m}Cₑₛ^{m}CₑₒAₑₛA_{dz}T_{ds}T_{ds}T_{ds}T_{dz}A_{dz}A_{dz}^{m}C_{ds}T_{ds}T_{ds}Gₑₒ^{m}CₑₛAₑₛ^{m}Cₑₛ^{m}Cₑ (SEQ ID NO: 22), wherein,
A=an adenine nucleobase,
^{m}C=a 5-methylcytosine nucleobase,
G=a guanine nucleobase,
T=a thymine nucleobase,
e=a 2′ MOE sugar moiety,
d=a 2′-β-D deoxyribosyl sugar moiety,
s=a phosphorothioate internucleoside linkage,
o=a phosphodiester internucleoside linkage, and
z=a mesyl phosphoramidate internucleoside linkage, and the compound does not include a conjugate group or terminal group.

Compound No. 1683009 is represented by the following chemical structure:

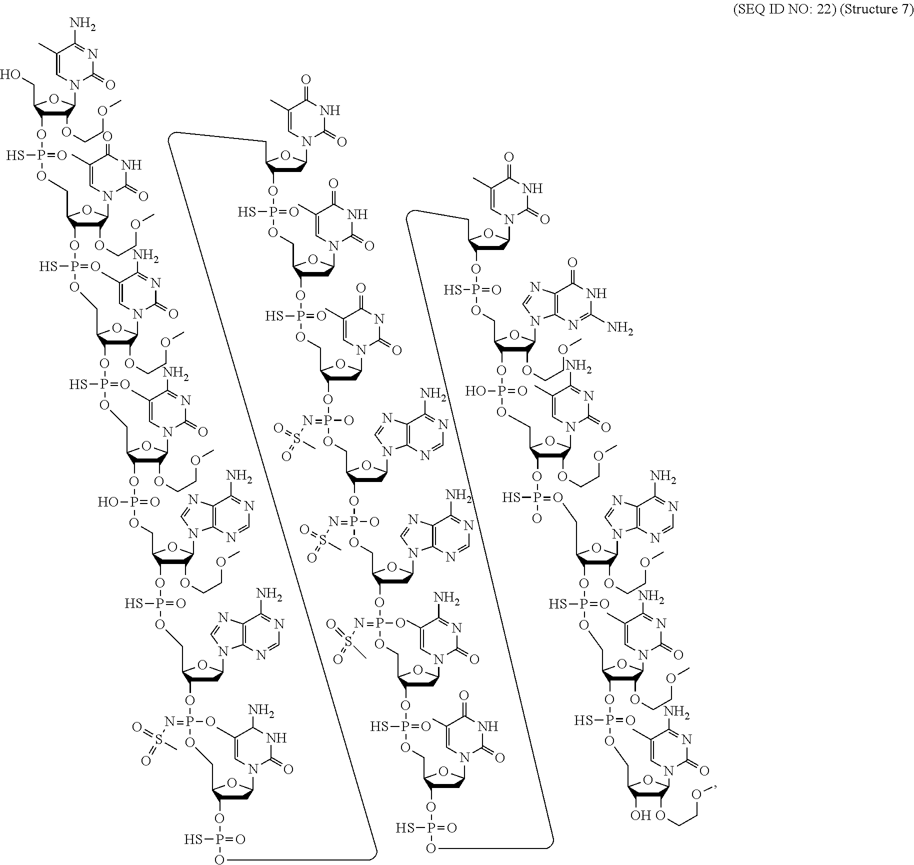

or a pharmaceutically acceptable salt thereof.

In certain embodiments, Compound 1683009 is a pharmaceutically acceptable salt comprising one or more cations selected from sodium, potassium, calcium, and magnesium. In certain embodiments, Compound 1683009 is a sodium salt or a potassium salt.

The sodium salt of Compound No. 1683009 is represented by the following chemical structure:

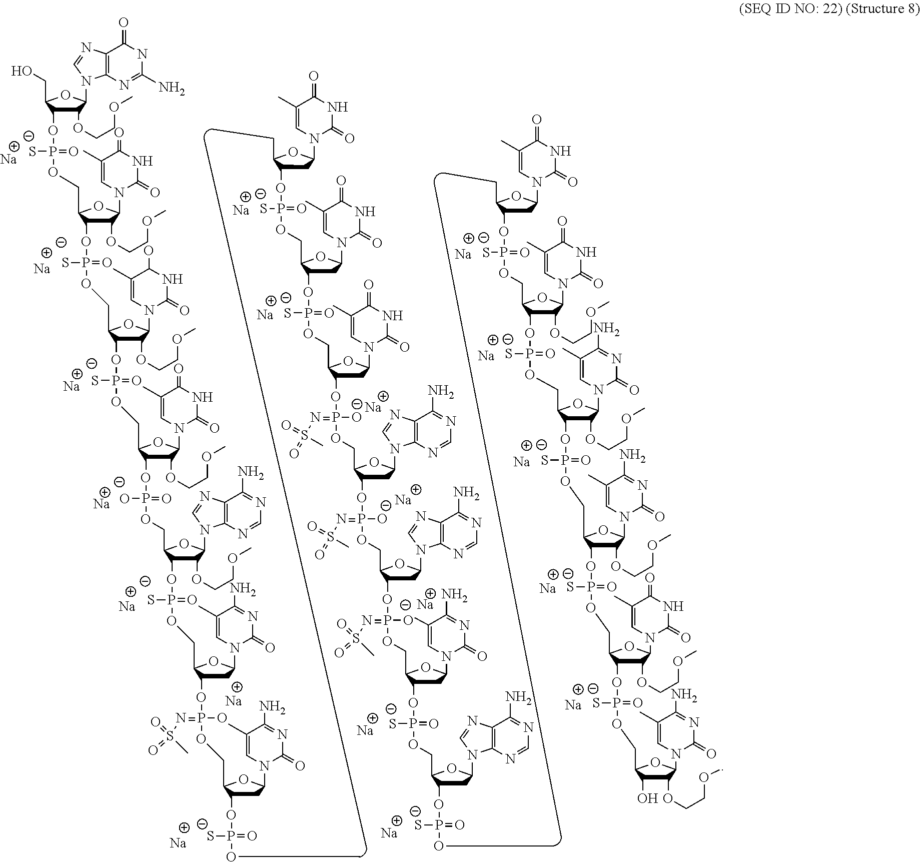

5. Compound No. 1683010

Compound No. 1683010 is characterized as a 5-10-5 MOE gapmer consisting of 20 linked nucleosides and having a nucleobase sequence of (from 5′ to 3′) CTCCAATTITAACTTGCACC (SEQ ID NO: 18), wherein each of nucleosides 1-5 and 16-20 (from 5′ to 3′) are 2′-MOE nucleosides and each of nucleosides 6-15 are 2′-β-D-deoxynucleosides, wherein the internucleoside linkage between nucleosides 4 to 5 is a phosphodiester internucleoside linkage, the internucleoside linkages between nucleosides 1 to 2, 2 to 3, 3 to 4, 5 to 6, 7 to 8, 8 to 9, 9 to 10, 13 to 14, 14 to 15, 15 to 16, 16 to 17, 17 to 18, 18 to 19, and 19 to 20 are phosphorothioate internucleoside linkages, and the internucleoside linkages between nucleosides 6 to 7, 10 to 11, 11 to 12, and 12 to 13 are mesyl phosphoramidate internucleoside linkages, and wherein each cytosine is a 5-methylcytosine.

Compound No. 1683010 is represented by the following chemical notation (5′ to 3′):
^{m}CₑₛTₑₛ^{m}Cₑₛ^{m}CₑₒAₑₛT_{ds}T_{ds}T_{ds}T_{dz}A_{dz}A_{dz}^{m}C_{ds}T_{ds}T_{ds}Gₑₛ^{m}CₑₛAₑₛ^{m}Cₑₛ^{m}Cₑ (SEQ ID NO: 23), wherein,
A=an adenine nucleobase,
^{m}C=a 5-methylcytosine nucleobase,
G=a guanine nucleobase,
T=a thymine nucleobase,
e=a 2′ MOE sugar moiety,
d=a 2′-β-D deoxyribosyl sugar moiety,
s=a phosphorothioate internucleoside linkage,
o=a pbsphodiester internucleoside linkage, and
z=a mesyl phosphoramidate internucleoside linkage, and the compound does not include a conjugate group or terminal group.

Compound No. 1683010 is represented by the following chemical structure:

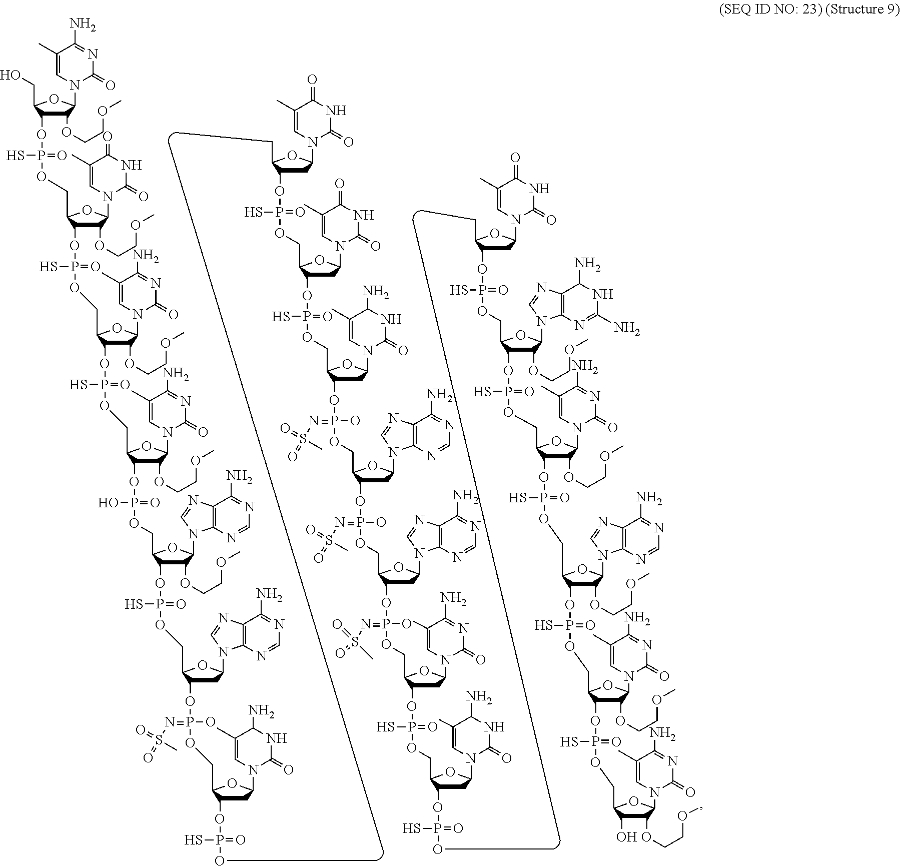

or a pharmaceutically acceptable salt thereof.

In certain embodiments, Compound 1683010 is a pharmaceutically acceptable salt comprising one or more cations selected from sodium, potassium, calcium, and magnesium. In certain embodiments, Compound 1683010 is a sodium salt or a potassium salt.

The sodium salt of Compound No. 1683010 is represented by the following chemical structure:

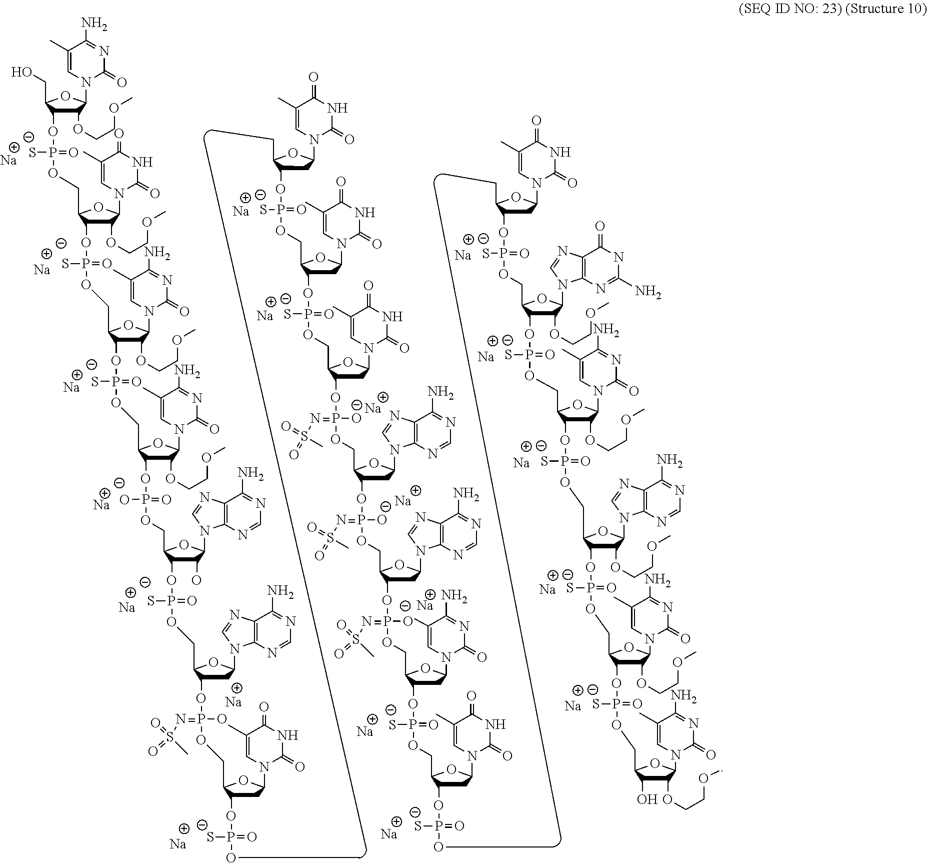

6. Compound No. 1683011

Compound No. 1683011 is characterized as a 5-10-5 MOE gapmer consisting of 20 linked nucleosides and having a nucleobase sequence of (from 5′ to 3′) CTCCAATITTAACTTGCACC (SEQ ID NO: 18), wherein each of nucleosides 1-5 and 16-20 (from 5′ to 3′) are 2′-MOE nucleosides and each of nucleosides 6-15 are 2′-β-D-deoxynucleosides, wherein the internucleoside linkage between nucleosides 16 to 17 is a phosphodiester internucleoside linkage, the internucleoside linkages between nucleosides 1 to 2, 2 to 3, 3 to 4, 4 to 5, 5 to 6, 7 to 8, 8 to 9, 9 to 10, 13 to 14, 14 to 15, 15 to 16, 17 to 18, 18 to 19, and 19 to 20 are phosphorothioate internucleoside linkages, and the internucleoside linkages between nucleosides 6 to 7, 10 to 11, 11 to 12, and 12 to 13 are mesyl phosphoramidate internucleoside linkage, and wherein each cytosine is a 5-methylcytosine.

Compound No. 1683011 is represented by the following chemical notation (5′ to 3′):
^{m}CₑₛTₑₛ^{m}Cₑₛ^{m}CₑₛAₑₛA_{dz}T_{ds}T_{ds}T_{ds}T_{dz}A_{dz}A_{dz}^{m}C_{ds}T_{ds}T_{ds}Gₑₒ^{m}CₑₛAₑₛ^{m}Cₑₛ^{m}Cₑ (SEQ ID NO: 24), wherein,
A=an adenine nucleobase,
^{m}C=a 5-methylcytosine nucleobase,
G=a guanine nucleobase,
T=a thymine nucleobase,
e=a 2′ MOE sugar moiety,
d=a 2′-β-D deoxyribosyl sugar moiety,
s=a phosphorothioate internucleoside linkage,
o=a phosphodiester internucleoside linkage, and
z=a mesyl phosphoramidate internucleoside linkage, and the compound does not include a conjugate group or terminal group.

Compound No. 1683011 is represented by the following chemical structure:

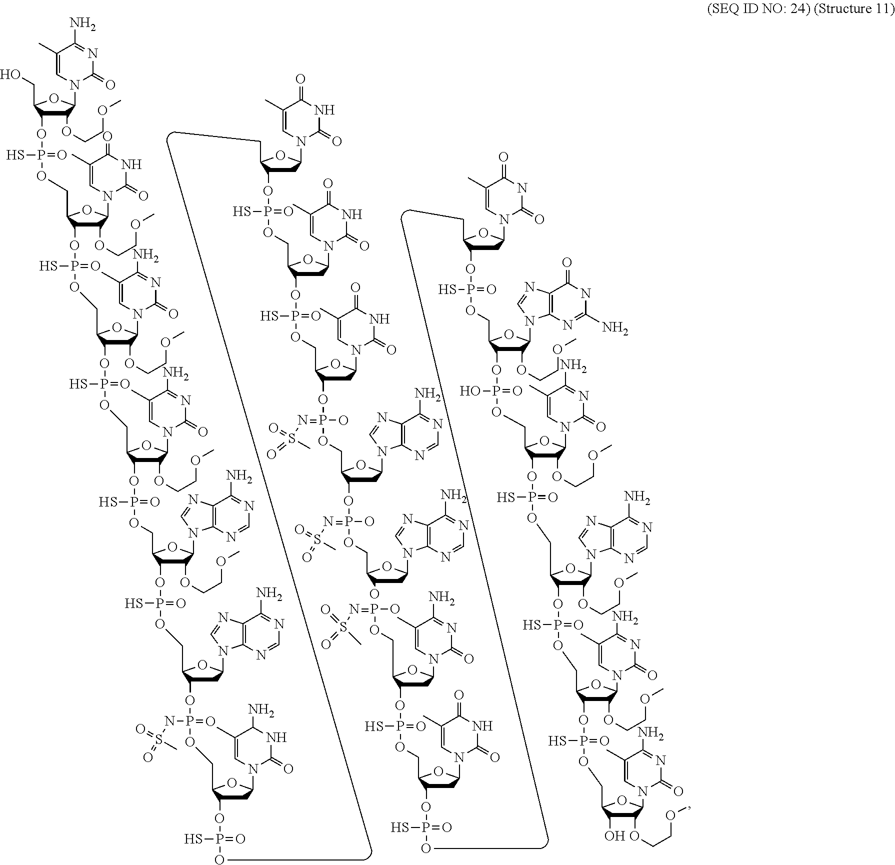

or a pharmaceutically acceptable salt thereof.

In certain embodiments, Compound 1683011 is a pharmaceutically acceptable salt comprising one or more cations selected from sodium, potassium, calcium, and magnesium. In certain embodiments, Compound 1683011 is a sodium salt or a potassium salt.

The sodium salt of Compound No. 1683011 is represented by the following chemical structure:

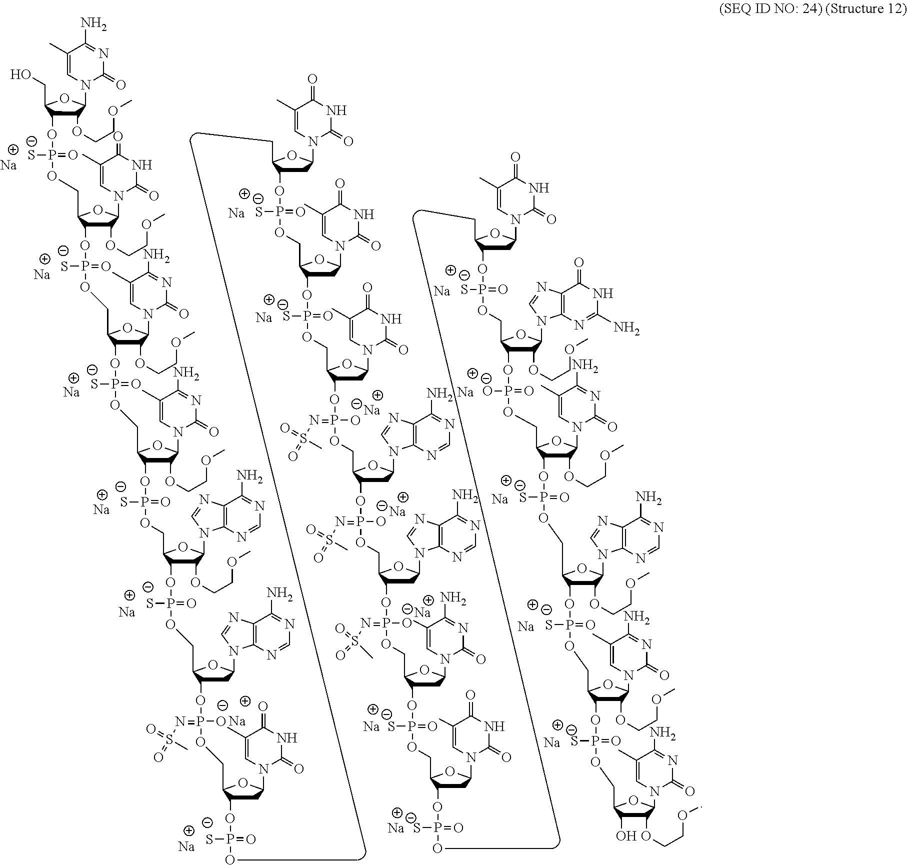

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage. Certain modified nucleosides and modified internucleoside linkages suitable for use in modified oligonucleotides are described below.

### A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into antisense oligonucleotides.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including, but not limited to, substituents at the 2′, 3′, 4′, and/or 5′ positions. In certain embodiments, the furanosyl sugar moiety is a ribosyl sugar moiety. In certain embodiments, one or more acyclic substituent of non-bicyclic modified sugar moieties is branched.

In certain embodiments, non-bicyclic modified sugar moieties comprise a substituent group at the 2′-position. Examples of substituent groups suitable for the 2′-position of modified sugar moieties include but are not limited to: F, OCH₃ (“OMe” or “O-methyl”), and OCH₂CH₂OCH₃ (“MOE” or “O-methoxyethyl”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), or 2′—O(CH₂)₂O(CH₂)₂N(CH₃)₂ (“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087.

Synthetic methods for some of these 2′-substituent groups can be found in, e.g., Cook et al., U.S. Pat. No. 6,531,584; and Cook et al., U.S. Pat. No. 5,859,221. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(RₘRₙ)), where each Rₘ and Rₙ is, independently, H, an amino protecting group, or substituted or unsubstituted C₁-C₁₀ alkyl.

In certain embodiments, a 2′-substituted sugar moiety of a modified nucleoside comprises 2′-substituent group selected from: F, OCF₃, OCH₃, OCH₂CH₂OCH₃, O(CH₂)₂SCH₃, O(CH₂)₂ON(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted sugar moiety of a modified nucleoside comprises 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2020/072991. A 2′-modified sugar moiety has an additional stereocenter at the 2′-position relative to a 2′-deoxyfuranosyl sugar moiety, therefore, such sugar moieties have a total of sixteen possible isomeric configurations. Modified furanosyl sugar moieties described herein are in the β-D-ribosyl isomeric configuration unless otherwise specified.

In certain embodiments, non-bicyclic modified sugar moieties are stereoisomers of DNA, such as 2′-β-D-deoxyxylosyl sugar moiety:

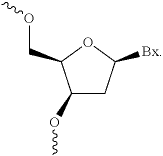

In certain embodiments, a non-bicyclic modified nucleoside comprises a 2′-α-L-deoxyribosyl sugar moiety:

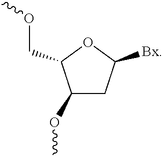

In certain embodiments, non-bicyclic modified sugar moieties comprise a substituent group at the 3′-position. Examples of substituent groups suitable for the 3′-position of modified sugar moieties include, but are not limited to, alkoxy (e.g., methoxy), alkyl (e.g., methyl, ethyl).

In certain embodiments, non-bicyclic modified sugar moieties comprise a substituent group at the 4′-position. Examples of substituent groups suitable for the 4′-position of modified sugar moieties include, but are not limited to, alkoxy (e.g., methoxy), alkyl, and those described in Manoharan et al., WO 2015/106128.

In certain embodiments, non-bicyclic modified sugar moieties comprise a substituent group at the 5′-position. Examples of substituent groups suitable for the 5′-position of modified sugar moieties include, but are not limited to, vinyl, alkoxy (e.g., methoxy), and alkyl (e.g., methyl (R or S), ethyl).

In certain embodiments, non-bicyclic modified sugar moieties comprise more than one non-bridging sugar substituent, for example, 2′—F-5′-methyl sugar moieties, such as described in Migawa et al., US 2010/0190837, or alternative 2′- and 5′-modified sugar moieties as described in Rajeev et al., US 2013/0203836.

In naturally occurring nucleic acids, sugars are linked to one another 3′ to 5′. In certain embodiments, oligonucleotides include one or more nucleoside or sugar moiety linked at an alternative position, for example at the 2′ position or inverted 5′ to 3′. For example, where the linkage is at the 2′ position, the 2′-substituent groups may instead be at the 3′-position.

Certain modified sugar moieties are bicyclic sugar moieties and comprise a substituent that bridges two atoms of the furanosyl ring to form a second ring, resulting in a bicyclic sugar moiety. In certain embodiments, the bicyclic sugar moiety comprises a bridge between the 4′ and the 2′ furanose ring atoms. Examples of such 4′ to 2′ bridging sugar substituents include, but are not limited to: 4′-CH₂-2′, 4′—(CH₂)₃-2′, 4′—(CH₂)₃-2′, 4′-CH₂O-2′ (“LNA”), 4′-CH₂—S-2′, 4′—(CH₂)₂—O-2′ (“ENA”), 4′-CH(CH₃)—O-2′ (referred to as “constrained ethyl” or “cEt” when in the S configuration), 4′-CH₂O—CH₂-2′, 4′-CH₂—N(R)-2′, 4′—CH(CH₂OCH₃)—O-2′ (“constrained MOE” or “cMOE”) and analogs thereof, 4′-C(CH₃)(CH₃)—O-2′ and analogs thereof, 4′-CH₂—N(OCH₃)-2′ and analogs thereof, 4′-CH₂—O—N(CH₃)-2′, 4′-CH₂—C(H)(CH₃)-2′, 4′-CH₂—C(═CH₂)-2′ and analogs thereof), 4′-C(RₐR_{b})—N(R)—O-2′, 4′—C(RₐR_{b})—O—N(R)-2′, 4′-CH₂—O—N(R)-2′, and 4′-CH₂N(R)—O-2′, wherein each R, Rₐ, and R_{b} is, independently, H, a protecting group, or C₁-C₁₂ alkyl. Representative U.S. patents that teach the preparation of such bicyclic sugar moieties include, but are not limited to: Imanishi et al., U.S. Pat. No. 7,427,672; Swayze et al., U.S. Pat. No. 7,741,457, and Swayze et al., U.S. Pat. No. 8,022,193; Seth et al., U.S. 8,278,283; Prakash et al., U.S. Pat. No. 8,278,425; Seth et al., U.S. Pat. No. 8,278,426).

In certain embodiments, such 4′ to 2′ bridges independently comprise from 1 to 4 linked groups independently selected from: —[C(RₐR_{b})]ₙ—, —[C(RₐR_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(β)₂-J₁), or sulfoxyl (S(═O)-J₁); and each J₁ and J₂ is, independently, H, C₁-C₁₂ alkyl, substituted C₁-C₁₂ alkyl, C₁-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Wan, et al., J. Medicinal Chemistry, 2016, 59, 9645-9667; Wengel et al., U.S. Pat. No. 8,080,644; Ramasany et al., U.S. Pat. No. 6,525,191; Seth et al., U.S. Pat. No. 7,547,684; and Seth et al., U.S. Pat. No. 7,666,854.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

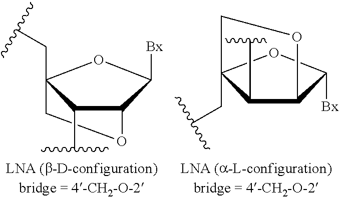

α-L-methyleneoxy (4′-CH₂O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mal Cane Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include, but are not limited to, hexitol nucleic acid (“HNA”), anitol nucleic acid (“ANA”), manitol nucleic acid (“MNA”), fluoro HNA:

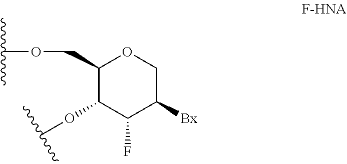

(“F-HNA”, see e.g., Egli, et. al., J Am Chem (2011)133(41): 16642-16649, Swayze et al., U.S. Pat. No. 8,088,904; and Swayze et al., U.S. Pat. No. 8,440,803). F-HNA can also be referred to as a F-THP or 3′-fluoro tetrahydropyran or 3′—FHNA, and nucleosides comprising additional modified THP compounds having the formula:

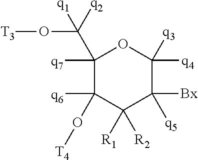

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently hydrogen, halogen, substituted or unsubstituted alkoxy, NJ₁J₂, SJ₁, N₃, OC(═X)J₁, OC(═X)NJ₁J₂, NJ₃C(═X)NJ₁J₂, or CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported. As used here, the term “morpholino” means a sugar surrogate having the following structure:

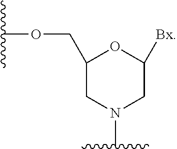

In certain embodiments, morpholinos may be modified, for example, by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid), and nucleosides and oligonucleotides described in Manoharan et al., U.S. Pat. No. 10,913,767. Representative U.S. patents that teach the preparation of PNA compounds include, but are not limited to, U.S. Pat. Nos. 5,539,082; 5,714,331; and 5,719,262.

In certain embodiments, sugar surrogates are the “unlocked” sugar structure of UNA (unlocked nucleic acid) nucleosides. UNA is a nucleoside wherein any of the bonds of the sugar moiety has been removed, forming an unlocked sugar surrogate. A representative U.S. publication that teaches the preparation of UNA includes, but is not limited to, US Patent Publication No 2011/0313020.

In certain embodiments, sugar surrogates are the glycerol as found in GNA (glycol nucleic acid) nucleosides as depicted below:

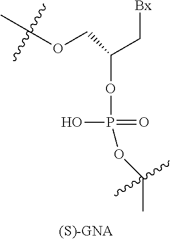

where Bx represents any nucleobase.

Many other bicyclic and tricyclic sugar and sugar surrogates are known in the art that can be used in modified nucleosides.

### 2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside. In certain embodiments, modified oligonucleotides comprise one or more inosine nucleosides (i.e., nucleosides comprising a hypoxanthine nucleobase). An “unmodified nucleobase” is adenine (A), thymine (T), cytosine (C), uracil (U), or guanine (G). A modified nucleobase is a group of atoms other than unmodified A, T, C, U, or G capable of pairing with at least one other nucleobase. A 5-methylcytosine is an example of a modified nucleobase. A universal base is a modified nucleobase that can pair with any one of the five unmodified nucleobases.

In certain embodiments, modified adenine has structure (I):

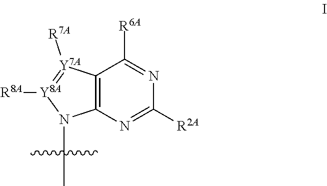

wherein: R^{2A} is H, C₁-C₆ alkyl, substituted C₁-C₆ alkyl, C₁-C₆ thioalkyl, or substituted C₁-C₆ thioalkyl, C₁-C₆ alkyloxy, or substituted C₁-C₆ alkyloxy; R^{6A} is H, N(R^{a}R^{b}), oxo, acetyl, formyl, or O-phenyl; Y^{7A} is N and R^{7A} is absent or is C₁-C₆ alkyl; or Y^{7A} is C and R^{7A} is H, C₁-C₆ alkyl, or CN(R^{a}R^{b}); Y^{8A} is N and R^{8A} is absent, or Y^{8A} is C and R^{8A} is H, a halogen, OH, C₁-C₆ alkyl, or substituted C₁-C₆ alkyl; R^{a} and R^{b} are independently H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where Y^{7A} is N; Y^{8A} is C, R^{8A} is H, R^{2A} is H, and R^{6A} is NH₂ (unmodified adenine).

In certain embodiments, modified guanine has structure (II):

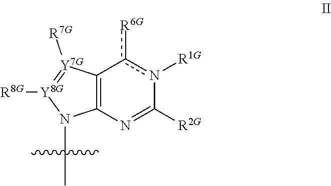

wherein: R^{2G} is N(R^{a}R^{b}). R^{6G} is oxo, and R^{1G} is H, or R^{6G} is O—C₁-C₆ alkyl or S—C₁-C₆ alkyl and R^{1G} is absent; Y^{7G} is N and R^{7A} is absent or is C₁-C₆ alkyl; or Y^{7G} is C and R^{7G} is H, C₁-C₆ alkyl, or CN(R^{a}R^{b}); Y^{8G} is N and R^{8G} is absent, or Y^{8G} is C and R^{8G} is H, a halogen, OH, C₁-C₆ alkyl, or substituted C₁-C₆ alkyl; R^{a} and R^{b} are independently H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where Y^{7G} is N; Y^{8G} is C, R^{8G} is H, R^{2G} is NH₂, and R^{6G} is ═O (unmodified guanosine).

In certain embodiments, modified thymine or modified uracil has structure (III):

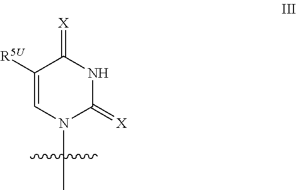

wherein: each X is independently O or S and R^{5U} is H, OH, halogen, O—C₁-C₁₂ alkyl, O—C₁-C₁₂ substituted alkyl, C₁-C₁₂ alkyl, substituted C₁-C₁₂ alkyl, C₁-C₁₂ alkenyl, or substituted C₁-C₁₂ alkenyl; wherein if each X is O, R^{5U} is not H or CH₃ (unmodified uracil and unmodified thymine, respectively).

In certain embodiments, modified cytosine has structure (IV):

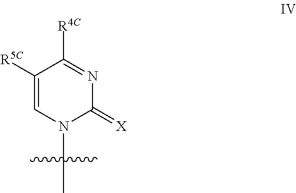

wherein: X is O or S; R^{4C} is N(R^{a})(R^{b}); R^{5C} is H, OH, halogen, O—C₁-C₁₂ alkyl, O—C₁-C₁₂ substituted alkyl, C₁-C₁₂ alkyl, substituted C₁-C₁₂ alkyl, C₁-C₁₂ alkenyl, or substituted C₁-C₁₂ alkenyl; Rₐ and R_{b} are independently H, C₁-C₆ alkyl, substituted C₁-C₆ alkyl, C₁-C₆ alkenyl, substituted C₁-C₆ alkenyl, acetyl, formyl, or together form a 5-7-membered heterocycle; excluding where X is O, R^{4C} is NH₂ and R^{5C} is H (wed cytosine).

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 5-methylcytosine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo (particularly 5-bromo), 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases, as well as other modified nucleobases include without limitation, Rogers et al., U.S. Pat. No. 5,134,066; Benner et al., U.S. Pat. No. 5,432,272; Matteucci et al., U.S. Pat. No. 5,502,177; Froehler et al., U.S. Pat. No. 5,594,121; and Cook et al., U.S. Pat. No. 5,681,941.

In certain embodiments, at least one nucleobase of a modified oligonucleotide is a modified nucleobase selected from modified adenine (A) having a structure represented by structure I, modified guanine (G) having a structure represented by structure II, modified thymine (T) or modified uracil (U) having a structure represented by structure III, and modified cytosine (C) having a structure represented by structure IV.

In certain embodiments, each nucleobase of a modified oligonucleotide is selected from unmodified A, unmodified G, unmodified C, unmodified T, unmodified U, and ^{m}C. 5-methylcytosine is a modified nucleobase having structure IV, where X is O, R^{4C} is NH₂, and R^{5C} is CH₃.

In certain embodiments, each nucleobase of a modified oligonucleotide is selected from unmodified A, unmodified G, unmodified C, unmodified T, unmodified U, ^{m}C, or hypoxanthine. Hypoxanthine is a modified adenine, where Y^{7A} is N and R^{7A} is absent, Y^{8A} is C, R^{8A} is H, R^{2A} is H, and R^{6A} is oxo.

In certain embodiments, there are no modified nucleobases in a modified oligonucleotide and each nucleobase of a modified oligonucleotide is selected from unmodified A, unmodified G, unmodified C, unmodified T, and unmodified U.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include, but are not limited to, phosphodiesters, which contain a phosphodiester bond (“P═O”) (also referred to as unmodified or naturally occurring linkages), phosphotriesters, methylphosphonates, phosphoramidates, phosphorothioates (“P═S”), and phosphorodithioates (“HS—P═S”). Representative non-phosphorus containing internucleoside linking groups include, but am not limited to, methylenemethylimino (—CH₂—N(CH₃)—O—CH₂—), thiodiester, thionocarbamate (—O—C(═O)(NH)—S—); siloxane (—O—SiH₂—O—); and N,N-dimethylhrydrazine (—CH₂N(CH₃)—N(CH₃)—). Modified internucleoside linkages, compared to naturally occurring phosphodiester internucleoside linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO 2021/030778. In certain embodiments, a modified internucleoside linkage comprises the formula:

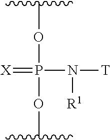

wherein independently for each such internucleoside linking group of a modified oligonucleotide:
X is O or S;
R₁ is H, C₁-C₆ alkyl, or substituted C₁-C₆ alkyl; and
T is SO₂R₂, C(═O)R₃, or P(═O)R₄R₅, wherein:
R₂ is an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, or a conjugate group; R₃ is an aryl, a substituted aryl, CH₃, N(CH₃)₂, OCH₃ or a conjugate group;
R₄ is OCH₃, OH, C₁-C₆ alkyl, substituted C₁-C₆ alkyl or a conjugate group; and
R₅ is OCH₃, OH, C₁-C₆ alkyl, or substituted C₁-C₆ alkyl.

In certain embodiments, a modified internucleoside linkage comprises a mesyl phosphoramidate linking group which has the formula:

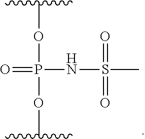 [SMILES: *OP(=O)(NS(C)(=O)=O)O*]

The mesyl phosphoramidate internucleoside linkage comprises a chiral center. In certain embodiments, modified oligonucleotides comprise (Rp) and/or (Sp) mesyl phosphoramidates, which are shown in the following formulas, respectively, wherein “B” indicates a nucleobase:

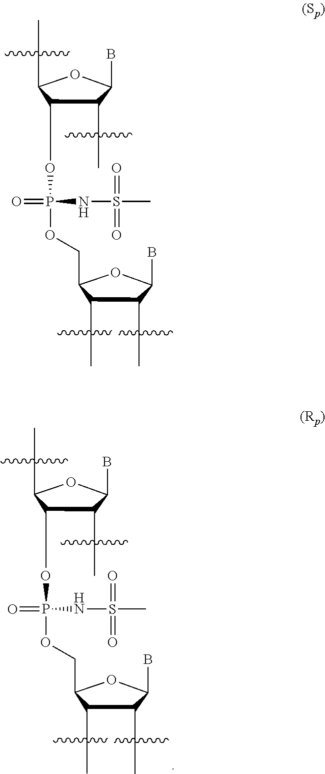

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising such internucleoside linkages in particular stenochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stenochemical configuration of each internucleoside linkage having a chiral center. Nonetheless, each individual internucleoside linkage having a chiral center of each individual oligonucleotide molecule has a defined stereoconfiguration. In certain embodiments, populations of modified oligonucleotides are enriched for modified oligonucleotides comprising one or more particular phosphorothioate and/or mesyl phosphoramidate internucleoside linkages, each independently in a particular, independently selected stenochemical configuration. In certain embodiments, the particular configuration of the particular phosphorothioate and/or mesyl phosphoramidate linkage is present in at least 65% of the molecules in the population. In certain embodiments, the particular configuration of the particular phosphorothioate and/or mesyl phosphoramidate linkage is present in at least 70% of the molecules in the population. In certain embodiments, the particular configuration of the particular phosphorothioate and/or mesyl phosphoramidate linkage is present in at least 80% of the molecules in the population. In certain embodiments, the particular configuration of the particular phosphorothioate and/or mesyl phosphoramidate linkage is present in at least 90% of the molecules in the population. In certain embodiments, the particular configuration of the particular phosphorothioate and/or mesyl phosphoramidate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate and/or mesyl phosphoramidate in the (Sp) configuration. In certain embodiments, a population of modified oligonucleotides is enriched for modified oligonucleotides having at least one phosphorothioate and/or mesyl phosphoramidate in the (Rp) configuration. In certain embodiments, modified oligonucleotides comprising (Rp) and/or (Sp) phosphorothioates comprise one or more of the following formulas, respectively, wherein “B” indicates a nucleobase:

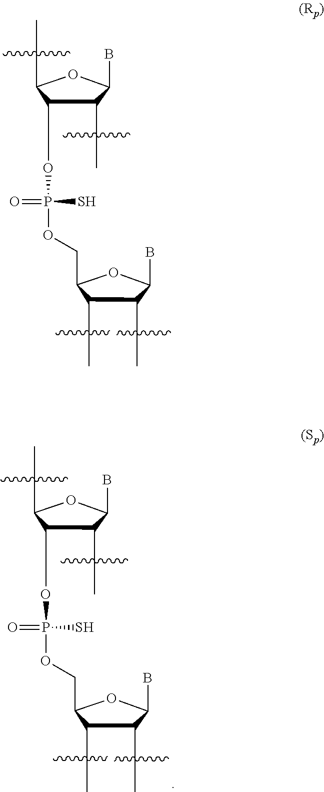

Unless otherwise indicated, internucleoside linkages having chiral centers of modified oligonucleotides described herein can be stereorandom or in a particular stereochemical configuration.

Neutral internucleoside linkages include, without limitation, phosphotriesters, methylphosphonates, M II (3′-CH₂—N(CH₃)—O-5′), amide-3 (3′-CH₂—C(═O)—N(H)—5′), amide-4 (3′—CH₂N(H)—C(═O)—5′), formacetyl (3′—O—CH₂—O—5′), methoxypropyl (MOP), and thioformacetal (3′—S—CH₂O—5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

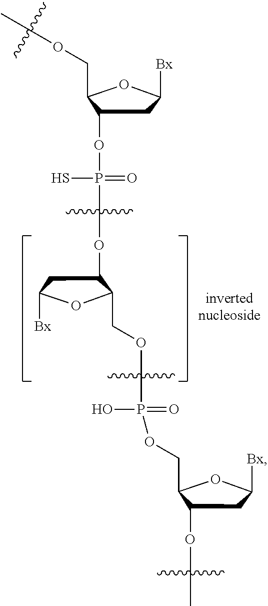

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, nucleic acids can be linked 2′ to 5′ rather than the standard 3′ to 5′ linkage. Such a linkage is illustrated below.

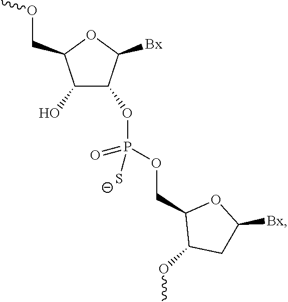

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise a deoxy region. In certain embodiments, each nucleoside of the deoxy region is a 2′β-D-deoxynucleoside. In certain embodiments, the deoxy region consists of 5-12 linked nucleosides. In certain embodiments, the deoxy region consists of 6, 7, 8, 9, 10, or 6-10 linked nucleosides. In certain embodiments, at least one nucleoside within the deoxy region comprises a modified sugar moiety. In certain embodiments, exactly one nucleoside within the deoxy region comprises a modified sugar moiety. In certain embodiments, two or three nucleosides within the deoxy region comprise a modified sugar moiety.

In certain embodiments, the deoxy region is flanked on the 5′-side by a 5′-region consisting of linked 5′-region nucleosides and on the 3′-side by a 3′-region consisting of linked 3′-region nucleosides; wherein the 3′-most nucleoside of the 5′-region is a modified nucleoside and the 5′-most nucleoside of the 3′-region is a modified nucleoside. At least one nucleoside of the 5′-region comprises a modified sugar moiety; and at least one nucleoside of the 3′-region comprises a modified sugar moiety. The three regions (the 5′-region, the deoxy region, and the 3′-region) form a contiguous sequence of nucleosides. In certain embodiments, the sugar moiety of the 3′-most nucleoside of the 5′-region and the sugar moiety of the 5′-most nucleoside of the 3′-region each differ from the sugar moiety of the respective adjacent nucleoside of the deoxy region, thus defining the boundary between the 5′-region, the deoxy region, and the 3′-region. In certain embodiments, each nucleoside of the 5′-region and each nucleoside of the 3′-region comprises a modified sugar moiety. In certain embodiments, the nucleosides within the 5′-region comprise the same sugar modification. In certain embodiments, the nucleosides within the 5′-region comprise two or more different sugar modifications. In certain embodiments, the nucleosides within the 3′-region comprise the same sugar modification. In certain embodiments, the nucleosides within the 3′-region comprise two or more different sugar modifications.

In certain embodiments, the 5′-region and the 3′-region of a modified oligonucleotide each comprises 1-8 nucleosides. In certain embodiments, the 5′-region comprises 1-7 nucleosides. In certain embodiments, the 5′-region comprises 1-6 nucleosides. In certain embodiments, the 5′-region comprises 1, 2, 3, 4, 5, 6, 7, or 8 nucleosides. In certain embodiments, the 3′-region comprises 1-7 nucleosides. In certain embodiments, the 3′-region comprises 1-6 nucleosides. In certain embodiments, the 3′-region comprises 1, 2, 3, 4, 5, 6, 7, or 8 nucleosides.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, one nucleoside of the gap comprises a modified sugar moiety and each remaining nucleoside of the gap comprises a 2′-deoxyribosyl sugar moiety. In certain embodiments, at least one nucleoside of the gap of a gapmer comprises a 2′-MOE sugar moiety.

Herein, the lengths (number of nucleosides) of the three regions of a gapmer may be provided using the notation [#of nucleosides in the 5′-wing]−[#of nucleosides in the gap]−[#of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing and the gap nucleosides comprise 2′-β-D-deoxyribosyl sugar moieties. Thus, a 5-10-5 MOE gapmer consists of 5 linked 2′-MOE nucleosides in the 5′-wing, 10 linked 2′-β-D-deoxynucleosides in the gap, and 5 linked 2′-MOE nucleosides in the 3′-wing. A 5-8-5 gapmer consists of 5 linked nucleosides comprising a modified sugar moiety in the 5′-wing, 8 linked 2′-β-D-deoxynucleosides in the gap, and 5 linked nucleosides comprising a modified sugar moiety in the 3′-wing. A 5-8-5 mixed gapmer has at least two different modified sugar moieties in the 5′- and/or the 3′-wing.

In certain embodiments, modified oligonucleotides disclosed herein are modified by a specific sugar modification. In certain embodiments, modified oligonucleotides are 5-10-5 MOE gapmers.

In certain embodiments, modified oligonucleotides disclosed herein are modified by two or more sugar modifications. In certain embodiments, modified oligonucleotides are 5-10-5 mixed gapmers, wherein each nucleoside within the 5′ and the 3′ wings comprises a 2′-MOE sugar moiety, and each nucleoside within the gap comprises a sugar moiety selected from a 2′-β-D-deoxyxylosyl sugar moiety, a 2′-α-L-deoxyribosyl sugar moiety, and 2′-β-D-deoxyribosyl sugar moiety. In certain embodiments, modified oligonucleotides are 5-10-5 mixed gapmers, wherein each nucleoside within the 5′ and the 3′ wings comprises a 2′-MOE sugar moiety, and each nucleoside within the gap comprises 2′-β-D-deoxyribosyl sugar moieties.

In certain embodiments, modified oligonucleotides have a sugar motif selected from 5′ to 3′: eeeeeddddddddddeeeee; wherein each “d” represents a 2′-β-D-deoxyribosyl sugar moiety, and each “e” represents a 2′-MOE sugar moiety.

In certain embodiments, each nucleobase of a modified oligonucleotide disclosed herein is selected from A, G, C, T, U, and ^{m}C.

In certain embodiments, each nucleobase of a modified oligonucleotide of disclosed herein is selected from A, G, T, and ^{m}C (i.e., unmodified purines and 5-methyl pyrimidines).

### 2. Certain Nucleobase Motifs

In certain embodiments, oligonucleotides comprise modified and/or unmodified nucleobases arranged along the oligonucleotide or region thereof in a defined pattern or motif. In certain embodiments, at least one nucleobase is modified. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases. In certain embodiments, each nucleobase is independently selected from 5-methylcytosine, unmodified cytosine, unmodified thymine, unmodified uracil, unmodified adenine, unmodified guanine, and unmodified hypoxanthine.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl sugar moiety. In certain embodiments, the modified nucleobase is selected from a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, at least one internucleoside linking group is a phosphodiester internucleoside linkage (P═O). In certain embodiments, at least one internucleoside linking group of a modified oligonucleotide is a phosphorothioate internucleoside linkage (P═S). In certain embodiments, at least one internucleoside linking group of a modified oligonucleotide is a mesyl phosphoramidate internucleoside linkage. In certain embodiments, each internucleoside linkage of a modified oligonucleotide is independently selected from a phosphorothioate internucleoside linkage, a phosphodiester internucleoside linkage, and a mesyl phosphoramidate internucleoside linkage. In certain embodiments, each internucleoside linkage of a modified oligonucleotide is independently selected from a phosphorothioate internucleoside linkage and a mesyl phosphoramidate internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, each mesyl phosphoramidate internucleoside linkage is independently selected from a stereorandom mesyl phosphoramidate, (Sp) mesyl phosphoramidate, and (Rp) mesyl phosphoramidate.

In certain embodiments, the sugar motif of a modified oligonucleotide is a gapmer and the internucleoside linkages within the gap are all modified. In certain such embodiments, the the sugar motif of a modified oligonucleotide is a gapmer and the internucleoside linkages within the gap are all modified, wherein at least one, at least 2, at least 3, or at least 4 internucleoside linkages within the gap is a mesyl phosphoramidate linkage and the remaining internucleoside linkages are phosphorothioate internucleoside linkages. In certain such embodiments, 1, 2, 3, 4, or 1-4 internucleoside linkages within the gap is a mesyl phosphoramidate linkage. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages and mesyl phosphoramidate linkages. In certain such embodiments, all of the phosphorothioate internucleoside linkages and mesyl phosphoramidate internucleoside linkages are stereorandom. In certain embodiments, all of the phosphorothioate internucleoside linkages and mesyl phosphoramidate internucleoside linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, modified nucleotides have an internucleoside linkage motif of sooosssssszzszsooss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of sooosssssszszssooss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of sooosszsssszsssooss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of soooszsssszzsssooss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of sooosszzssszsssooss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of sooossssszszssooss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of sooossssszzzzssooss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of sooosszssszzsssooss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of soooszssszzzsssooss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of sssosssssszzszsosss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of ssoosssssszszssosss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of ssoosszsssszsssosss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of sssoszsssszzsssosss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of ssoossssszzzszsosss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of ssssssssszzzzzzsssss, wherein each “s” represents a phosphorothioate internucleoside linkage and each “z” represents a mesyl phosphoramidate internucleoside linkage. In certain embodiments, modified nucleotides have an internucleoside linkage motif of sssoszssszzzsssosss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of sssoszssszzzsssssss, wherein each “s” represents a phosphorothioate internucleoside linkage, each “o” represents a phosphodiester internucleoside linkage, and each “z” represents a mesyl phosphoramidate internucleoside linkage. In certain embodiments, modified nucleotides have an internucleoside linkage motif of ssssszssszzzsssosss, wherein each “s” represents a phosphorothioate internucleoside linkage, each “o” represents a phosphodiester internucleoside linkage, and each “z” represents a mesyl phosphoramidate internucleoside linkage.

In certain embodiments the modified oligonucleotide comprises an internucleoside linkage motif (5′ to 3′) selected from sooosssssszzszsooss, sooosssssszszssooss, sooosszsssszsssooss, soooszsssszzsssooss, sooosszzssszsssooss, sooossssszzzszsooss, sooossssszzzzssooss, sooosszssszzsssooss, soooszssszzzsssooss, sssosssssszzszsosss, ssoossssszszssosss, ssoosszsssszsssosss, sssoszsssszzsssosss, ssoossssszzzszsosss, ssssssssszzzzzsssss, sssoszssszzzsssosss, sssoszssszzzsssssss, and ssssszssszzzsssosss, wherein each “s” represents a phosphorothioate internucleoside linkage, each “o” represents a phosphodiester internucleoside linkage, and each “z” represents a mesyl phosphoramidate internucleoside linkage. In certain embodiments, the modified oligonucleotide comprises an internucleoside linkage motif (5′ to 3′) selected from sssoszsssszzsssosss, ssoossssszzzszsosss, ssssszssszzzsssossss, sssoszssszzzsssosss, sssoszssszzzsssssss, and ssssszssszzzsssosss, wherein each “s” represents a phosphorothioate internucleoside linkage, each “o” represents a phosphodiester internucleoside linkage, and each “z” represents a mesyl phosphoramidate internucleoside linkage. In certain embodiments, the modified oligonucleotide comprises an internucleoside linkage motif (5′ to 3′) of sssoszsssszzsssosss, wherein each “s” represents a phosphorothioate internucleoside linkage, each “o” represents a phosphodiester internucleoside linkage, and each “z” represents a mesyl phosphoramidate internucleoside linkage.

### . Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y am each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides (including modified oligonucleotides) consist of 16 linked nucleosides. In certain embodiments, oligonucleotides (including modified oligonucleotides) consist of 17 linked nucleosides. In certain embodiments, oligonucleotides (including modified oligonucleotides) consist of 18 linked nucleosides. In certain embodiments, oligonucleotides (including modified oligonucleotides) consist of 19 linked nucleosides. In certain embodiments, oligonucleotides (including modified oligonucleotides) consist of 20 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one particular phosphorothioate internucleoside linkage and/or mesyl phosphoramidate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides (or portions thereof) have a nucleobase sequence that is complementary to a second oligonucleotide or an identified reference nucleic acid (or portion thereof), such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid (or portion thereof), such as a target nucleic acid.

### II. Certain Oligomeric Compounds

In certain embodiments, provided herein are oligomeric compounds, which consist of an oligonucleotide and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups are attached to either or both ends of an oligonucleotide (such conjugate groups are also terminal groups). In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can alter one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a catbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesteral moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds. In certain embodiments, a conjugate group is a lipid having the following structure:

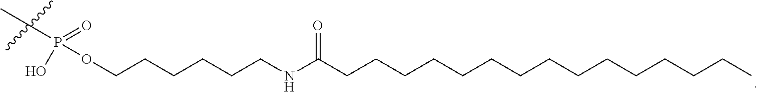 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluorescein, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indomethacin, a barbiturate, a cephalosporin, a sulfa drug, an antidiabetic, an antibacterial or an antibiotic.

In certain embodiments, conjugate moieties are selected from any of a C₂₂ alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, CO alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, CO alkenyl, or C5 alkenyl.

In certain embodiments, conjugate moieties are selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include, but are not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

In certain embodiments, a conjugate group comprises a cell-targeting moiety. In certain embodiments, a conjugate group has the general formula:

in certain embodiments, a conjugate group comprises a cell-targeting moiety. In certain embodiments, a conjugate group has the formula:

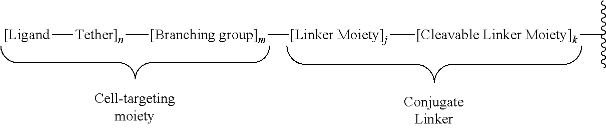

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

In certain embodiments, conjugate groups comprise cell-targeting moieties that have affinities for transferrin receptor (TfR) (also referred to herein as TfR1 and CD71). In certain embodiments, a conjugate group described herein comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the anti-TfR1 antibody or fragment thereof can be any known in the art including but not limited to those described in WO 1991/004753; WO 2013/103800; WO 2014/144060; WO 2016/081643; WO 2016/179257; WO 2016/207240; WO 2017/221883; WO 2018/129384; WO 2018/124121; WO 2019/151539; WO 2020/132584; WO 2020/028864; U.S. Pat. Nos. 7,208,174; 9,034,329; and 10,550,188. In certain embodiments, a fragment of an anti-TfR1 antibody is F(ab′)₂, Fab, Fab′, Fv, or scFv.

In certain embodiments, the conjugate group comprises a protein or peptide capable of binding TfR 1. In certain embodiments, the protein or peptide capable of binding TfR1 can be any known in the art including but not limited to those described in WO 2019/140050; WO 2020/037150; WO 2020/124032; and U.S. Pat. No. 10,138,483.

In certain embodiments, the conjugate group comprises an aptamer capable of binding TfR1. In certain embodiments, the aptamer capable of binding TfR 1 can be any known in the art including but not limited to those described in WO 2013/163303; WO 2019/033051; and WO 2020/245198.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic sugar moieties and/or inverted nucleosides. In certain embodiments, an terminal group comprises an inverted abasic sugar moiety. In certain embodiments, the inverted abasic sugar moiety may be further attached to a conjugate group. In certain embodiments, terminal groups comprise one or more 2′-linked nucleosides or sugar moieties. In certain such embodiments, the 2′-linked group is an abasic sugar moiety. Such terminal abasic sugar moieties can be attached to either or both ends of an oligonucleotide.

### III. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard in vivo assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA or dsRNAi) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed dimly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

### IV. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from a mature mRNA and a pre-mRNA, including intronic, exonic and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron. In certain embodiments, the target nucleic acid is a non-coding RNA. In certain embodiments, the target non-coding RNA is selected from a long non-coding RNA, a short non-coding RNA, an intronic RNA molecule.

### A. Complementarity/Mismatches to the Target Nucleic Acid and Duplex Complementarity

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved.

In certain embodiments, a mismatch is specifically positioned within an oligonucleotide having a gapmer motif.

In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### B. App

In certain embodiments, oligomeric compounds comprise or consist of an oligonucleotide comprising a region that is complementary to a target nucleic acid, wherein the target nucleic acid is APP. In certain embodiments, APP nucleic acid has the sequence set forth SEQ ID NO: 1 (the cDNA of Ensembl transcript ENST00100346798.7 from version 94: October 2018) or the complement of SEQ ID NO: 2 (GENBANK Accession No. NC_000021.9 truncated from nucleotides 25878001 to 26174000). In certain embodiments, APP nucleic acid has the sequence set forth in any of known splice variants of APP, including but not limited to SEQ ID NO: 3 (the cDNA of Ensembl transcript ENST00000357903.7 from version 94: October 2018), SEQ ID NO: 4 (the cDNA of Ensembl transcript ENST00000348990.9 from version 94: October 2018), SEQ ID NO: 5 (the cDNA of Ensembl transcript ENST00000440126.7 from version 94: October 2018), SEQ ID NO: 6 (the cDNA of Ensembl transcript ENST00000354192.7 from version 94: October 2018), SEQ ID NO: 7 (the cDNA of Ensembl transcript ENST00000358918.7 from version 94: October 2018), and/or SEQ ID NO: 8 (GENBANK Accession No. NM_201414.2). In certain embodiments, contacting a cell with an oligomeric compound described herein complementary to SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8 reduces the amount of APP RNA, and in certain embodiments reduces the amount of APP protein. In certain embodiments, the oligomeric compound consists of a modified oligonucleotide. In certain embodiments, contacting a cell with an oligomeric compound described herein complementary to SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8 results in reduced aggregation of β-amyloid. In certain embodiments, the oligomeric compound consists of a modified oligonucleotide. In certain embodiments, the oligomeric compound consists of a modified oligonucleotide and a conjugate group.

In certain embodiments, contacting a cell with an oligomeric compound described herein complementary to SEQ ID NO: 1 or SEQ ID NO: 2 reduces the amount of APP RNA in the cell. In certain embodiments, contacting a cell with an oligomeric compound described herein complementary to SEQ ID NO: 1 or SEQ ID NO: 2 reduces the amount of APP protein in a cell. In certain embodiments, the cell is in vitro. In certain embodiments, contacting a cell in a subject with an oligomeric compound described herein complementary to SEQ ID NO: 1 or SEQ ID NO: 2 ameliorates one or more symptoms or hallmarks of a disease or disorder associated with APP. In certain embodiments, the disease or disorder associated with APP is any of sporadic Alzheimer's Disease, genetic/familial Alzheimer's Disease, Alzheimer's Disease in a Down Syndrome patient, and Cerebral Amyloid Angiopathy. In certain embodiments, the symptom or hallmark is any of cognitive impairment, including a decline in memory and language skills, behavioral and psychological symptoms such as apathy and lack of motivation, gait disturbances and seizures, progressive dementia, and/or abnormal amyloid deposits.

In certain embodiments, an oligomeric compound described herein complementary to SEQ ID NO: 1 or SEQ ID NO: 2 is capable of reducing the detectable amount of APP RNA in vitro by at least 10%, at least 20%, at least 30%, at least 40%, at least 50%, at least 60%, at least 70%, at least 80%, or at least 90% in the standard in viva assay. In certain embodiments, an oligomeric compound described herein complementary to SEQ ID NO: 1 or SEQ ID NO: 2 is capable of reducing the detectable amount of APP protein in vitro by at least 10%, at least 20%, at least 30%, at least 40%, at least 50%, at least 60%, at least 70%, at least 80%, or at least 90%. In certain embodiments, an oligomeric compound described herein complementary to SEQ ID NO: 1 or SEQ ID NO: 2, is capable of reducing the detectable amount of APP RNA in the CSF of a subject by at least 10%, at least 20%, at least 30%, at least 40%, at least 50%, at least 60%, at least 70%, at least 80%, or at least 90%. In certain embodiments, an oligomeric compound described herein complementary to SEQ ID NO: 1 or SEQ ID NO: 2, is capable of reducing the detectable amount of APP protein in the CSF of a subject by at least 10%, at least 20%, at least 30%, at least 40%, at least 50%, at least 60%, at least 70%, at least 80%, or at least 90%.

### C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments the pharmacologically relevant cell is an APP-expressing cell. In certain embodiments, the pharmacologically relevant tissues are the cells and tissues that comprise the central nervous system. Such tissues include the cortex, and the hippocampus. Such cells include cortical brain cells, hippocampal cells. In certain embodiments, such cells include cells within the limbic system, for example, cells within the hippocampus, the amygdala, and/or parahippocampal gyrus.

### V. Certain Methods and Uses

Certain embodiments provided herein relate to methods of reducing or inhibiting APP expression or activity, which can be useful for treating, preventing, or ameliorating a disease or disorder associated with overexpression of APP in a subject, by administration of an oligomeric agent, an oligomeric compound, an oligomeric duplex, or an antisense agent, any of which comprise a modified oligonucleotide having a nucleobase sequence complementary to a APP nucleic acid. In certain embodiments, the disease or disorder associated with APP is a neurodegenerative disease or disorder. In certain embodiments, the neurodegenerative disease or disorder is sporadic Alzheimer's Disease, genetic/familial Alzheimer's Disease, Alzheimer's Disease in a Down Syndrome patient, or Cerebral Amyloid Angiopathy.

In certain embodiments, a method comprises administering to a subject an oligomeric agent, an oligomeric compound, an oligomeric duplex, or an antisense agent, any of which having a nucleobase sequence complementary to a APP nucleic acid. In certain embodiments, the subject has or is at risk for developing sporadic Alzheimer's Disease, genetic/familial Alzheimer's Disease, Alzheimer's Disease in a Down Syndrome patient, or Cerebral Amyloid Angiopathy.

In certain embodiments, a method of treating a neurodegenerative disease or disorder associated with APP comprises administering to a subject a therapeutically effective amount of an oligomeric agent, an oligomeric compound, an oligomeric duplex, or an antisense agent, any of which having a nucleobase sequence complementary to an APP nucleic acid, thereby treating the subject. In certain embodiments, the subject has or is at risk for developing a neurodegenerative disease or disorder associated with APP. In certain embodiments, the disease or disorder is associated with an elevated level of APP in the subject.

In certain embodiments, the subject has or is at risk for developing sporadic Alzheimer's Disease, genetic/familial Alzheimer's Disease, Alzheimer's Disease in a Down Syndrome patient, or Cerebral Amyloid Angiopathy. In certain embodiments, at least one symptom or hallmark of the neurodegenerative disease or disorder associated with APP is ameliorated. Exemplary symptoms or hallmarks include, but are not limited to, cognitive impairment, including a decline in memory and language skills, behavioral and psychological symptoms such as apathy and lack of motivation, gait disturbances and seizures, progressive dementia, and abnormal amyloid deposits.

In certain embodiments, a method of reducing expression of APP nucleic acid, for example RNA, or reducing the expression of APP protein in a cell comprises administering to the subject an oligomeric agent, an oligomeric compound, an oligomeric duplex, or an antisense agent, any of which having a nucleobase sequence complementary to an APP nucleic acid, thereby inhibiting expression of APP nucleic acid in the subject. In certain embodiments, administering the oligomeric agent, the oligomeric compound, the oligomeric duplex, or the antisense agent inhibits expression of APP in the brain or the spinal cord of the subject. In certain embodiments, the subject has a neurological disease or condition associated with APP. In certain embodiments, the subject has or is at risk for developing sporadic Alzheimer's Disease, genetic/familial Alzheimer's Disease, Alzheimer's Disease in a Down Syndrome patient, and/or Cerebral Amyloid Angiopathy.

In certain embodiments, a method of inhibiting expression of APP nucleic acid in a cell comprises contacting the cell with an oligomeric agent, an oligomeric compound, an oligomeric duplex, or an antisense agent, any of which having a nucleobase sequence complementary to a APP nucleic acid, thereby inhibiting expression of APP nucleic acid in the cell. In certain embodiments, the cell is a human cell. In certain embodiments, the cell is a brain cell. In certain embodiments, the cell is a cortical brain cell or a hippocampal cell. In certain embodiments, the cell is obtained from a subject, e.g., a subject that has or is at risk for developing a disease or disorder associated with APP. In certain embodiments, the cell is in a subject having a disease or condition associated with APP such as sporadic Alzheimer's Disease, genetic/familial Alzheimer's Disease, Alzheimer's Disease in a Down Syndrome patient, or Cerebral Amyloid Angiopathy.

Certain embodiments are drawn to an oligomeric agent, an oligomeric compound, an oligomeric duplex, or an antisense agent, any of which having a nucleobase sequence complementary to an APP nucleic acid, for use in treating a disease or disorder associated with elevated APP signaling, or with over-expression of APP. In certain embodiments, the disease or disorder is sporadic Alzheimer's Disease, genetic/familial Alzheimer's Disease, Alzheimer's Disease in a Down Syndrome patient, or Cerebral Amyloid Angiopathy. In certain embodiments, an oligomeric compound, an oligomeric duplex, or an antisense agent is for use in improving a symptom or hallmark of a disease or disorder associated with sporadic Alzheimer's Disease, genetic/familial Alzheimer's Disease, Alzheimer's Disease in a Down Syndrome patient, or Cerebral Amyloid Angiopathy. In certain embodiments, the symptom or hallmark is selected from cognitive impairment, including a decline in memory and language skills, behavioral and psychological symptoms such as apathy and lack of motivation, gait disturbances and seizures, progressive dementia, and abnormal amyloid deposits.

In certain embodiments, an oligomeric agent, an oligomeric compound, a modified oligonucleotide, an oligomeric duplex, or an antisense agent is for use in reducing APP expression in a subject.

Certain embodiments are drawn to an oligomeric agent, an oligomeric compound, an oligomeric duplex, or an antisense agent, any of which comprising a modified oligonucleotide having a nucleobase sequence complementary to an APP nucleic acid, for the manufacture or preparation of a medicament for treating a disease or disorder associated with APP. In certain embodiments, the disease or disorder is sporadic Alzheimer's Disease, genetic/familial Alzheimer's Disease, Alzheimer's Disease in a Down Syndrome patient, or Cerebral Amyloid Angiopathy. In certain embodiments, an oligomeric agent, an oligomeric compound, an oligomeric duplex, or an antisense agent is for the manufacture or preparation of a medicament for improving symptoms or hallmarks associated with APP. In certain embodiments, the symptom or hallmark is selected from seizures, cognitive impairment, including a decline in memory and language skills, behavioral and psychological symptoms such as apathy and lack of motivation, gait disturbances and seizures, progressive dementia, and abnormal amyloid deposits. In certain embodiments, an oligomeric agent, an oligomeric compound, an oligomeric duplex, or an antisense agent is for the manufacture or preparation of a medicament for use in reducing APP expression in a subject.

In any of the methods or uses described herein, the oligomeric compound, oligomeric duplex, or antisense agent can be any described herein.

### VI. Certain Pharmaceutical Compositions

In certain embodiments, described herein are pharmaceutical compositions comprising one or more oligomeric agents, oligomeric compounds, oligomeric duplexes, or antisense agents. In certain embodiments, the one or more oligomeric agents, oligomeric compounds, oligomeric duplexes, or antisense agents each comprise a modified oligonucleotide. In certain embodiments, the one or more oligomeric agents, oligomeric compounds, oligomeric duplexes, or antisense agents each consists of a modified oligonucleotide. In certain embodiments, the pharmaceutical composition comprises a pharmaceutically acceptable diluent. In certain embodiments, a pharmaceutical composition comprises or consists of a sterile saline solution and one or more oligomeric compounds, oligomeric duplexes, or antisense agents. In certain embodiments, the sterile saline is pharmaceutical grade saline. In certain embodiments, a pharmaceutical composition comprises or consists of one or more oligomeric agents, oligomeric compounds, oligomeric duplexes, or antisense agents and sterile water. In certain embodiments, the sterile water is pharmaceutical grade water. In certain embodiments, a pharmaceutical composition comprises or consists of one or more oligomeric agents, oligomeric compounds, oligomeric duplexes, or antisense agents and phosphate-buffered saline (PBS). In certain embodiments, the sterile PBS is pharmaceutical grade PBS. In certain embodiments, a pharmaceutical composition comprises or consists of one or more oligomeric agents, oligomeric compounds, oligomeric duplexes, or antisense agents and artificial cerebrospinal fluid (“artificial CSF” or “aCSF”). In certain embodiments, the artificial cerebrospinal fluid is pharmaceutical grade artificial cerebrospinal fluid.

In certain embodiments, a pharmaceutical composition comprises an oligomeric compound and PBS. In certain embodiments, a pharmaceutical composition consists of an oligomeric compound and PBS. In certain embodiments, a pharmaceutical composition consists essentially of an oligomeric compound and PBS. In certain embodiments, the PBS is pharmaceutical grade. In certain embodiments, a pharmaceutical composition comprises a modified oligonucleotide and PBS. In certain embodiments, a pharmaceutical composition consists of a modified oligonucleotide and PBS. In certain embodiments, a pharmaceutical composition consists essentially of a modified oligonucleotide and PBS. In certain embodiments, the PBS is pharmaceutical grade.

In certain embodiments, a pharmaceutical composition comprises an oligomeric compound and artificial cerebrospinal fluid (aCSF). In certain embodiments, a pharmaceutical composition consists of an oligomeric compound and aCSF. In certain embodiments, a pharmaceutical composition consists essentially of an oligomeric compound and aCSF. In certain embodiments, the aCSF is pharmaceutical grade. In certain embodiments, a pharmaceutical composition comprises a modified oligonucleotide and aCSF. In certain embodiments, a pharmaceutical composition consists of a modified oligonucleotide and aCSF. In certain embodiments, a pharmaceutical composition consists essentially of a modified oligonucleotide and aCSF. In certain embodiments, the artificial cerebrospinal fluid is pharmaceutical grade. In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric agents, oligomeric compounds, oligomeric duplexes, or antisense agents and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose, and polyvurylpyrolidone.

In certain embodiments, oligomeric agents, oligomeric compounds, oligomeric duplexes, or antisense agents may be admixed with pharmaceutically acceptable active and/or inert substances for the preparation of pharmaceutical compositions or formulations. Compositions and methods for the formulation of pharmaceutical compositions depend on a number of criteria, including, but not limited to, mute of administration, extent of disease, or dose to be administered.

In certain embodiments, pharmaceutical compositions comprising an oligomeric agent, an oligomeric compound, oligomeric duplex, or antisense agent encompass any pharmaceutically acceptable salts of the oligomeric agent, the oligomeric compound, the oligomeric duplex, or the antisense agent, esters of the oligomeric agent, the oligomeric compound, the oligomeric duplex, or the antisense agent, or salts of such esters. In certain embodiments, pharmaceutical compositions comprising oligomeric agents, oligomeric compounds, oligomeric duplexes, or antisense agents, comprising one or more modified oligonucleotide, upon administration to a subject, including a Inman, are capable of providing (directly or indirectly) the biologically active metabolite or residue thereof. Accordingly, for example, the disclosure is also drawn to pharmaceutically acceptable salts of oligomeric agents, oligomeric compounds, oligomeric duplexes, or antisense agents, prodrugs thereof, pharmaceutically acceptable salts of such prodrugs, and other bioequivalents. Suitable pharmaceutically acceptable salts include, but are not limited to, sodium, potassium, calcium, and magnesium salts. In certain embodiments, prodrugs comprise one or more conjugate group attached to an oligonucleotide, wherein the conjugate group is cleaved by endogenous nucleases within the body.

In certain embodiments, oligomeric agents, oligomeric compounds, oligomeric duplexes, or antisense agents are lyophilized and isolated as sodium salts. In certain embodiments, the sodium salt of an oligomeric compound is mixed with a pharmaceutically acceptable diluent. In certain embodiments, the pharmaceutically acceptable diluent comprises sterile saline, sterile water, PBS, or aCSF. In certain embodiments, the sodium salt of an oligomeric agent, an oligomeric compound, oligomeric duplex, or antisense agent is mixed with PBS. In certain embodiments, the sodium salt of an oligomeric agent, an oligomeric compound, oligomeric duplex, or antisense agent is mixed with aCSF. In certain embodiments, the sodium salt of the oligomeric compound, oligomeric duplex, or antisense agent is a sodium salt of a modified oligonucleotide.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents disclosed herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” expressly includes all such forms that may be fully or partially protonated/de-protonated/in association with a cation. In certain instances, one or more specific cation is identified.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid.

For example, where a modified oligonucleotide or an oligomeric compound is in solution comprising sodium (e.g., saline), the modified oligonucleotide or oligomeric compound may be partially or fully de-protonated and in association with sodium ions. However, the mass of the protons are nevertheless counted toward the weight of the dose, and the mass of the sodium ions are not counted toward the weight of the dose. Thus, for example, a dose, or dosage unit, of 10 mg of a number of fully protonated molecules that weighs 10 mg. This would be equivalent to 10.57 mg of solvent-free, sodium acetate-free, anhydrous sodiated Compound No. 1620705. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

In certain embodiments, where a modified oligonucleotide or oligomeric compound is in a solution, such as aCSF, comprising sodium, potassium, calcium, and magnesium, the modified oligonucleotide or oligomeric compound may be partially or fully de-protonated and in association with sodium, potassium, calcium, and/or magnesium. However, the mass of the protons is nevertheless counted toward the weight of the dose, and the mass of the sodium, potassium, calcium, and magnesium ions is not counted toward the weight of the dose.

In certain embodiments, when an oligomeric compound comprises a conjugate group, the mass of the conjugate group may be included in calculating the dose of such oligomeric compound. If the conjugate group also has an acid, the conjugate group is likewise assumed to be fully protonated for the purpose of calculating dose.

### VII. Certain Comparator Compositions

Compound 1353884 is a comparator compound and is previously described in WO 2022/026589. Compound 1353884 consists of the sequence (from 5′ to 3′): GTTTACCTTTAACATTCCTC, designated herein as SEQ ID NO: 15. The sugar motif for Compound No. 1353884 is (from 5′ to 3′): eeeeeddddddddddeeeee; wherein each “d” represents a 2′-β-D-deoxyribosyl sugar moiety, and each “e” represents a ribo-2′-MOE sugar moiety. The internucleoside linkage motif for Compound No. 1353884 is (from 5′ to 3′): sooosssssssssssooss; wherein each “s” represents a phosphorothioate internucleoside linkage and each “o” represents a phosphodiester internucleoside linkage. Each cytosine nucleobase in Compound No. 1353884 is a 5-methylcytosine.

Compound 1398227 is a comparator compound and is previously described in WO 2022/026589. Compound 1398227 consists of the sequence (from 5′ to 3′): CTCCAATTTTAACTTGCACC, designated herein as SEQ ID NO: 18. The sugar motif for Compound No. 1398227 is (from 5′ to 3′): eeeeeddddddddddeeeee; wherein each “d” represents a 2′-β-D-deoxyribosyl sugar moiety, and each “e” represents a ribo-2′-MOE sugar moiety. The internucleoside linkage motif for Compound No. 1398227 is (from 5′ to 3′): sooosssssssssssooss; wherein each “s” represents a phosphorothioate internucleoside linkage and each “o” represents a phosphodiester internucleoside linkage. Each cytosine nucleobase in Compound No. 1398227 is a 5-methylcytosine.

In certain embodiments, compounds described herein are superior relative to compounds described in WO 2022/026589, because the compounds described herein demonstrate one or more improved properties such as duration of action and potency.

For example, Compound No. 1620705 demonstrated a longer duration of action in vivo as compared to Compound Nos. 1353884 and 1398227 in the assay shown in Example 7. In particular, at day 112 (16 weeks) post-dose, Compound No. 1620705 achieved a 88% reduction of human APP RNA in the cortex, whereas Compound No. 1353884 achieved a 54% reduction of human APP RNA in the cortex and Compound No. 1393227 achieved a 32% reduction of human APP RNA in the cortex. Further, at day 140 (20 weeks) post-dose, Compound No. 1620705 achieved a 60% reduction of human APP RNA in the cortex, whereas Compound No. 1353884 achieved a 43% reduction of human APP RNA in the cortex and Compound No. 1393227 achieved a 37% reduction of human APP RNA in the cortex. Therefore, Compound No. 1620705 exhibited a longer duration of action compared to Compound Nos. 1353884 and 1393227.

Also, Compound No. 1620705 demonstrated greater potency in vivo compared to Compound Nos. 1353884 and 1398227 in the assay shown in Example 4. In particular, in transgenic mice expressing the full length human APP gene (APP YAC transgenic mice), the half maximal effective dose (ED₅₀) of Compound No. 1620705 as measured in the spinal cord, cortex, and hippocampus of APP YAC transgenic mice at 4 weeks post-dose is 48 μg, 57 μg, and 62 μg, respectively. In comparison, the ED₅₀ of Compound No. 1353884 as measured in the spinal cord, cortex, and hippocampus of APP YAC transgenic mice at 4 weeks post-dose is 95 μg, 116 μg, and 76 μg, respectively, and the ED of Compound No. 1398227 as measured in the spinal cord, cortex, and hippocampus of APP YAC transgenic mice at 4 weeks post-dose is 72 μg, 190 μg, and 120 μg, respectively. Further, as shown in Examples 5 and 6, the ED₅₀ of Compound No. 1620705 as measured in the spinal cord, cortex, and hippocampus of APP YAC transgenic mice at 12 weeks post-dose is 89 μg, 57 μg, and 71 μg respectively, whereas the ED₅₀ of Compound No. 1398227 as measured in the spinal cord, cortex, and hippocampus of APP YAC transgenic mice at 12 weeks post-dose is 157 μg, 206 μg, and 210 μg, respectively. Therefore, Compound No. 1620705 exhibited improved potency compared to Compound Nos. 1353884 and 1393227 in these assays.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

The sequence listing accompanying this filing identifies each nucleic acid sequence as either “RNA” or “DNA” as required; however, one of skill in the art will readily appreciate that designation of “RNA” or “DNA” to describe modified oligonucleotides is, in certain instances, arbitrary. For example, an oligonucleotide comprising a nucleoside comprising a 2′—OH sugar moiety and a thymine base could be described as a DNA having a modified sugar (2′—OH in place of one 2′-H of DNA) or as an RNA having a modified base (thymine (5-methyl uracil)) in place of an uracil of RNA); and certain nucleic acid compounds described herein comprise one or more nucleosides comprising modified sugar moieties having 2′-substituent(s) that are neither OH nor H. One of skill in the art will readily appreciate that labeling such nucleic acid compounds “RNA” or “DNA” does not alter or limit the description of such nucleic acid compounds.

Herein, the description of compounds as having “the nucleobase sequence of” a SEQ ID NO. describes only the nucleobase sequence. Accordingly, absent additional description, such description of compounds by reference to a nucleobase sequence of a SEQ ID NO. does not limit sugar or internucleoside linkage modifications or presence or absence of additional substituents such as a conjugate group. Further, absent additional description, the nucleobases of a compound “having the nucleobase sequence of” a SEQ ID NO. include such compounds having modified forms of the identified nucleobases as described herein.

Herein, the description of compounds by chemical notation (subscripts and/or superscripts to indicate chemical modifications) without reference to a specific Compound No. include only each noted modification, but may include additional substituents, such as a conjugate group, unless otherwise indicated. For example, the chemical notation of “AₑₛTₖₒ^{m}C_{ez}G_{ds}C_{d}” indicates a compound wherein the first nucleoside comprises a 2′-MOE sugar moiety (indicated by the “e” subscript) and an unmodified adenine nucleobase linked to the second nucleoside via a phosphorothioate linkage (indicated by the “s” subscript); the second nucleoside comprises a cEt sugar moiety (indicated by the “k” subscript) and an unmodified thymine nucleobase linked to the third nucleoside via a phosphodiester linkage (indicated by the “o” subscript); the third nucleoside comprises a 2′-MOE sugar moiety and a 5-methyl modified cytosine nucleobase (indicated by the “m” superscript) linked to the fourth nucleoside via a mesylphosphoramidate linkage (indicated by the “z” subscript); the fourth nucleoside comprises a 2′-β-D-deoxyribosyl sugar moiety (indicated by the “d” subscript) and an unmodified guanine nucleobase linked to the fifth nucleoside with a phosphorothioate linkage; and the fifth nucleoside comprises a 2′-β-D-deoxyribosyl sugar moiety and an unmodified cytosine nucleobase; and the compound may include additional substituents, such as a conjugate group.

Herein, where a specific compound (e.g., with reference to a Compound No.) is described (as in the examples) by chemical notation, each nucleobase, sugar, and internucleoside linkage of such specific compound is modified only as indicated. Accordingly, in the context of a description of a specific compound having a particular Compound No., “AₑₛTₖₒ^{m}C_{ez}G_{ds}C_{d}” indicates a compound wherein the first nucleoside comprises a 2′-MOE sugar moiety (indicated by the “e” subscript) and an unmodified adenine nucleobase linked to the second nucleoside via a phosphorothioate linkage (indicated by the “s” subscript); the second nucleoside comprises a cEt sugar moiety (indicated by the “k” subscript) and an unmodified thymine nucleobase linked to the third nucleoside via a phosphodiester linkage (indicated by the “o” subscript); the third nucleoside comprises a 2′-MOE sugar moiety and a 5-methyl modified cytosine nucleobase (indicated by the “m” superscript) linked to the fourth nucleoside via a mesylphosphoramidate linkage (indicated by the “z” subscript); the fourth nucleoside comprises a 2′-β-D-deoxyribosyl sugar moiety (indicated by the “d” subscript) and an unmodified guanine nucleobase linked to the fifth nucleoside with a phosphorothioate linkage; and the fifth nucleoside comprises a 2′-β-D-deoxyribosyl sugar moiety and an unmodified cytosine nucleobase; and the compound does not include additional substituents.

Herein, sugar, internucleoside linkage, and nucleobase modifications may be indicated within a nucleotide or nucleobase sequence (e.g., by superscript or subscript, as shown above) or may be indicated in text accompanying a sequence (e.g., in separate text that appears within or above or below a table of compounds).

Where a specific compound is described herein by way of a drawn chemical structure, each nucleobase, sugar, and internucleoside linkage of such a specific compound includes only the modifications indicated in the drawn chemical structure. One of skill will appreciate, however, that drawn compounds may exist in equilibrium between tautomeric forms and/or as salts in equilibrium with protonated or ionic forms. Drawn structures are intended to capture all such forms of such compounds.

While effort has been made to accurately describe compounds in the accompanying sequence listing, should there be any discrepancies between a description in this specification and in the accompanying sequence listing, the description in the specification and not in the sequence listing is the accurate description.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### Examples

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Design of Modified Oligonucleotides Complementary to Human APP RNA

Modified oligonucleotides complementary to a human APP RNA were designed as described in the tables below.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 2 (the complement of GENBANK Accession No. NC 000021.9, truncated from nucleotides 25878001 to 26174000).

The modified oligonucleotides in the table below are 5-10-5 MOE gapmers with mixed internucleoside linkage motifs. The modified oligonucleotides in the table below are 20 nucleosides in length, wherein the sugar motif for the modified oligonucleotides is (from 5′ to 3′): eeeeeddddddddddeeeee; wherein each “e” represents a ribo-2′-MOE sugar moiety and each “d” represents a 2′-β-D-deoxyribosyl sugar moiety. The internucleoside linkage motifs for the modified oligonucleotides are presented in the column labeled “Internucleoside Linkages (5′ to 3′)” in the table below, wherein each “s” represents a phosphorothioate internucleoside linkage, each “z” represents a mesyl phosphoramidate internucleoside linkage, and each “o” represents a phosphodiester internucleoside linkage. Each cytosine residue is a 5-methylcytosine.

TABLE 1
5-10-5 MOE gapmers with mixed internucleoside linkages complementary to human APP

| | SEQ ID | SEQ ID | | | |
|---|---|---|---|---|---|
| | NO: 2 | NO: 2 | | | SEQ |
| Compound | Start | Stop | | Internucleoside | ID |
| No. | Site | Site | Sequence (5′ to 3′) | Linkages (5′ to 3′) | NO |
| 1620690 | 158795 | 158814 | GTTTACCTTTAACATTCCTC | sooosssssszzszsooss | 15 |
| 1620691 | 12586 | 12605 | GCATTCTCTTATATTCCTTA | sooosssssszszssooss | 16 |
| 1620692 | 159759 | 159778 | GTTCACAGTTTACCCCAAGC | sooosszsssszsssooss | 17 |
| 1620693 | 34556 | 34575 | CTCCAATTTTAACTTGCACC | soooszsssszzsssooss | 18 |
| 1620696 | 159759 | 159778 | GTTCACAGTTTACCCCAAGC | sooosszzssszsssooss | 17 |
| 1620698 | 158795 | 158814 | GTTTACCTTTAACATTCCTC | sooossssszzzszsooss | 15 |
| 1620699 | 12586 | 12605 | GCATTCTCTTATATTCCTTA | sooossssszzzzssooss | 16 |
| 1620700 | 159759 | 159778 | GTTCACAGTTTACCCCAAGC | sooosszssszzsssooss | 17 |
| 1620701 | 34556 | 34575 | CTCCAATTTTAACTTGCACC | soooszssszzzsssooss | 18 |
| 1620702 | 158795 | 158814 | GTTTACCTTTAACATTCCTC | sssosssssszzszsosss | 15 |
| 1620703 | 12586 | 12605 | GCATTCTCTTATATTCCTTA | ssoosssssszszssosss | 16 |
| 1620704 | 159759 | 159778 | GTTCACAGTTTACCCCAAGC | ssoosszsssszsssosss | 17 |
| 1620705 | 34556 | 34575 | CTCCAATTTTAACTTGCACC | sssoszsssszzsssosss | 18 |
| 1681029 | 158795 | 158814 | GTTTACCTTTAACATTCCTC | ssoossssszzzszsosss | 15 |
| 1681031 | 158795 | 158814 | GTTTACCTTTAACATTCCTC | ssssssssszzzzzsssss | 15 |
| 1683009 | 34556 | 34575 | CTCCAATTTTAACTTGCACC | sssoszssszzzsssosss | 18 |
| 1683010 | 34556 | 34575 | CTCCAATTTTAACTTGCACC | sssoszssszzzsssssss | 18 |
| 1683011 | 34556 | 34575 | CTCCAATTTTAACTTGCACC | ssssszssszzzsssosss | 18 |

### Example 2: Duration of Action Study of Modified Oligonucleotides Complementary to Human APP in APP YAC Transgenic Mice, Single Dose

Modified oligonucleotides described above were tested in APP YAC transgenic mice, expressing the full length human APP gene (Lamb B T et al., PubMed: 8220418, B6.129S2-Tg(APP)8.9Btla/J, Strain 005301 from Jackson Laboratory). The APP YAC mice were created using a 650 kb YAC transgene containing the entire human amyloid beta (A4) precursor protein (APP) gene, and approximately 250 kb of flanking sequence, was altered to include the Swiss mutation APPK670N/M671L associated with Familial Alzheimer's Disease. This transgene was injected into (129X1/SvJ×129S1/Sv)F₁-derived R1 embryonic stem (ES) cells. Founder animals (line R1.40) were backcrossed to C₅₇BL/6J for 21 generations.

The APP YAC transgenic mice were divided into groups of 3 mice each or as otherwise specified in the column labeled “No. of Animals” in the tables below. Each mouse received a single ICV bolus of 300 μg of modified oligonucleotide. A group of 3-4 mice received a single ICV bolus of PBS as a negative control.

Mice were sacrificed at various timepoints as indicated in the tables below and RNA was extracted from cortical brain tissue, spinal cord, and hippocampus for RTPCR analysis to measure amount of APP RNA using human primer probe set RTS35571 (forward sequence CCCACTTTGTGATTCCCTACC, designated herein as SEQ ID NO: 9; reverse sequence ATCCATCCTCTCCTGGTGTAA, designated herein as SEQ ID NO: 10; probe sequence TGATGCCCTTCTCGTTCCTGACAA, designated herein as SEQ ID NO: 11). Results are presented as percent human APP RNA relative to the amount of APP in PBS treated control animals, normalized to mouse PPIA (% control). Mouse PPIA was amplified using primer probe set m_cyclo24 (forward sequence TCGCCGCTTGCTGCA, designated herein as SEQ ID NO: 12; reverse sequence ATCGGCCGTGATGTCGA, designated herein as SEQ ID NO: 13; probe sequence CCATGGTCAACCCCACCGTGTTC, designated herein as SEQ ID NO: 14).

TABLE 2
Reduction of human APP RNA
in APP YAC transgenic mice

| | Timepoint | | | |
|---|---|---|---|---|
| | (Days | APP RNA (% control) | | |
| Compound | post- | Spinal | | Hippo- |
| No. | dose) | Cord | Cortex | campus |
| PBS | 14 | 100 | 100 | 100 |
| | 84 | 100 | 100 | 100 |
| 1620705 | 14 | 24 | 28 | 22 |
| | 84 | 25 | 23 | 31 |
| 1681031 | 14 | 71 | 76 | 58 |
| | 84 | 72 | 81 | 83 |
| 1683010 | 14 | 33 | 70 | 39 |
| | 84 | 42 | 34 | 50 |

TABLE 3
Reduction of human APP RNA in APP YAC transgenic mice

| | | Timepoint | APP RNA (% control) | | |
|---|---|---|---|---|---|
| Compound | No. of | (Days | Spinal | | Hippo- |
| No. | Animals | post-dose) | Cord | Cortex | campus |
| PBS | 4 | 28 | 100 | 100 | 100 |
| | 4 | 112 | 100 | 100 | 100 |
| 1620705 | 6 | 28 | 18 | 12 | 14 |
| | 6 | 112 | 26 | 11 | 18 |

TABLE 4
Reduction of human APP RNA
in APP YAC transgenic mice

| | Timepoint | APP RNA (% control) | | |
|---|---|---|---|---|
| Compound | (Days | Spinal | | Hippo- |
| No. | post-dose) | Cord | Cortex | campus |
| PBS | 56 | 100 | 100 | 100 |
| | 140 | 100 | 100 | 100 |
| 1620690 | 56 | 33 | 52 | 60 |
| | 140 | 47 | 78 | 79 |
| 1620691 | 56 | 24 | 43 | 48 |
| | 140 | 44 | 68 | 75 |
| 1620692 | 56 | 28 | 62 | 58 |
| | 140 | 46 | 73 | 67 |
| 1620693 | 56 | 39 | 32 | 56 |
| | 140 | 77 | 77 | 81 |
| 1620696 | 56 | 24 | 37 | 35 |
| | 140 | 38 | 61 | 67 |
| 1620698 | 56 | 40 | 51 | 47 |
| | 140 | 46 | 72 | 80 |
| 1620699 | 56 | 28 | 38 | 57 |
| | 140 | 32 | 63 | 78 |
| 1620700 | 56 | 30 | 43 | 50 |
| | 140 | 42 | 68 | 69 |
| 1620701 | 56 | 44 | 41 | 61 |
| | 140 | 41 | 66 | 82 |
| 1620702 | 56 | 34 | 59 | 66 |
| | 140 | 37 | 71 | 86 |
| 1620703 | 56 | 25 | 47 | 62 |
| | 140 | 36 | 75 | 81 |
| 1620704 | 56 | 27 | 54 | 52 |
| | 140 | 36 | 63 | 67 |
| 1620705 | 56 | 21‡ | 15‡ | 29‡ |
| | 140 | 54 | 40 | 46 |

‡indicates fewer than 3 samples available

### Example 3: Activity of Modified Oligonucleotides Complementary to Human APP in APP YAC Transgenic Mice

The APP YAC transgenic mouse model (described herein above) was used to test the activity of modified oligonucleotides described above.

The APP YAC transgenic mice were divided into groups of 4 mice each. Each mouse received a single ICV bolus of 300 μg of modified oligonucleotide. A group of 4 mice received a single ICV bolus of PBS as a negative control.

Twenty weeks post treatment, mice were sacrificed and RNA was extracted from cortical brain tissue, spinal cord, and hippocampus for RTPCR analysis to measure amount of APP RNA using human primer probe set RTS35571 (described herein above). Results are presented as percent human APP RNA relative to the amount of APP in PBS treated control animals, normalized to mouse PPIA (% control). Mouse PPIA was amplified using primer probe set m_cyclo24 (described herein above).

TABLE 5
Reduction of human APP RNA in APP YAC transgenic mice

| | APP RNA (% control) | | |
|---|---|---|---|
| Compound No. | Spinal Cord | Cortex | Hippocampus |
| PBS | 100‡ | 100‡ | 100‡ |
| 1681029 | 72 | 84 | 83 |
| 1681031 | 95 | 98 | 98 |
| 1683009 | 39 | 67 | 50 |
| 1683010 | 48 | 68 | 75 |
| 1683011 | 55 | 62 | 60 |

‡indicates fewer than 4 samples available

### Example 4: Potency of Modified Oligonucleotides Complementary to Human APP in APP YAC Transgenic Mice, 4-Weeks

Modified oligonucleotides described above were tested in the APP YAC transgenic mouse model described herein above.

### Treatment

The APP YAC transgenic mice were divided into groups of 4 mice each. Each mouse received a single ICV bolus of modified oligonucleotide at doses indicated in the tables below. A group of 4 mice received a single ICV bolus with PBS as a negative control.

### RNA Analysis

Four weeks post treatment, mice were sacrificed, and RNA was extracted from cortical brain tissue, spinal cord, and hippocampus for RTPCR analysis to measure amount of APP RNA using human primer probe set RTS35571 (described herein above). Results are presented as percent human APP RNA relative to the amount of APP in PBS treated control animals, normalized to mouse PPIA (% control). Mouse PPIA was amplified using primer probe set m_cyclo24 (described herein above).

The half maximal effective dose (ED50) of each modified oligonucleotide was calculated using GraphPad Prism 7 software (GraphPad Software, San Diego, CA). N. C. refers to values not calculated.

Compound 1353884 is a comparator compound and is previously described in WO 2022/026589. Compound 1353884 consists of the sequence (from 5′ to 3′): GTTTACCTTTAACATTCCTC, designated herein as SEQ ID NO: 15. The sugar motif for Compound No. 1353884 is (from 5′ to 3′): eeeeeddddddddddeeeee; wherein each “d” represents a 2′-β-D-deoxyribosyl sugar moiety, and each “e” represents a ribo-2′-MOE sugar moiety. The internucleoside linkage motif for Compound No. 1353884 is (from 5′ to 3′): sooosssssssssssooss; wherein each “s” represents a phosphorothioate internucleoside linkage and each “o” represents a phosphodiester internucleoside linkage. Each cytosine nucleobase in Compound No. 1353884 is a 5-methylcytosine.

Compound 1398227 is a comparator compound and is previously described in WO 2022/026589. Compound 1398227 consists of the sequence (from 5′ to 3′): CTCCAATITTAACTTGCACC, designated herein as SEQ ID NO: 18. The sugar motif for Compound No. 1398227 is (from 5′ to 3′): eeeeeddddddddddeeeee; wherein each “d” represents a 2′-β-D-deoxyribosyl sugar moiety, and each “e” represents a ribo-2′-MOE sugar moiety. The internucleoside linkage motif for Compound No. 1398227 is (from 5′ to 3′): sooosssssssssssooss; wherein each “s” represents a phosphorothioate internucleoside linkage and each “o” represents a phosphodiester internucleoside linkage. Each cytosine nucleobase in Compound No. 1398227 is a 5-methylcytosine.

TABLE 6
Dose-dependent percent reduction of human
APP mRNA in APP YAC transgenic mice

| Com- | | APP RNA (% control) | | | | | |
|---|---|---|---|---|---|---|---|
| pound | Dose | Spinal | ED50 | | ED50 | Hippo- | ED50 |
| No. | (μg) | Cord | (μg) | Cortex | (μg) | campus | (μg) |
| PBS | — | 100 | — | 100 | — | 100 | — |
| 1620705 | 10 | 85 | 48 | 90 | 57 | 91 | 62 |
| | 30 | 55 | | 65 | | 65 | |
| | 100 | 33 | | 35 | | 36 | |
| | 300 | 19 | | 16 | | 20 | |
| | 700 | 13 | | 7 | | 11 | |

TABLE 7
Dose-dependent percent reduction of human
APP mRNA in APP YAC transgenic mice

| Com- | | APP RNA (% control) | | | | | |
|---|---|---|---|---|---|---|---|
| pound | Dose | Spinal | ED50 | | ED50 | Hippo- | ED50 |
| No. | (μg) | Cord | (μg) | Cortex | (μg) | campus | (μg) |
| PBS | — | 100 | — | 100 | — | 100 | — |
| 1353884 | 10 | 88 | 95 | 94 | 116 | 94 | 76 |
| | 30 | 74 | | 82 | | 74 | |
| | 100 | 49 | | 57 | | 41 | |
| | 300 | 23 | | 21 | | 16 | |
| | 700 | 19 | | 13 | | 12 | |
| 1398227 | 10 | 80 | 72 | 96 | 190 | 95 | 120 |
| | 30 | 64 | | 82 | | 73 | |
| | 100 | 37 | | 74 | | 60 | |
| | 300 | 39 | | 37 | | 28 | |
| | 700 | 19 | | 13 | | 12 | |

### Example 5: Potency of Modified Oligonucleotides Complementary to Human APP in APP YAC Transgenic Mice, 12-Weeks

Modified oligonucleotides described above were tested in the APP YAC transgenic mouse model described herein above.

### Treatment

The APP YAC transgenic mice were divided into groups of 4 mice each. Each mouse received a single ICV bolus of modified oligonucleotide at doses indicated in the tables below. A group of 4 mice received a single ICV bolus with PBS as a negative control.

### RNA Analysis

Twelve weeks post treatment, mice were sacrificed, and RNA was extracted from cortical brain tissue, spinal cord, and hippocampus for RTPCR analysis to measure amount of APP RNA using human primer probe set RTS35571 (described herein above). Results are presented as percent human APP RNA relative to the amount of APP in PBS treated control animals, normalized to mouse PPIA (% control). Mouse PPIA was amplified using primer probe set m_cyclo24 (described herein above).

The half maximal effective dose (ED50) of each modified oligonucleotide was calculated using GraphPad Prism 7 software (GraphPad Software, San Diego, CA).

TABLE 8
Dose-dependent percent reduction of human
APP mRNA in APP YAC transgenic mice

| Com- | | APP RNA (% control) | | | | | |
|---|---|---|---|---|---|---|---|
| pound | Dose | Spinal | ED50 | | ED50 | Hippo- | ED50 |
| No. | (μg) | Cord | (μg) | Cortex | (μg) | campus | (μg) |
| PBS | — | 100 | — | 100 | — | 100 | — |
| 1620705 | 10 | 90 | 89 | 98 | 57 | 98 | 71 |
| | 30 | 68 | | 67 | | 78 | |
| | 100 | 44 | | 30 | | 31 | |
| | 300 | 28 | | 15 | | 20 | |
| | 700 | 21 | | 7 | | 13 | |

### Example 6: Potency of Modified Oligonucleotides Complementary to Human APP in APP YAC Transgenic Mice, 12-Weeks

Modified oligonucleotides described above were tested in the APP YAC transgenic mouse model described herein above.

### Treatment

The APP YAC transgenic mice were divided into groups of 3-4 mice each. Each mouse received a single ICV bolus of modified oligonucleotide at doses indicated in the tables below. A group of 4 mice received a single ICV bolus with PBS as a negative control.

### RNA Analysis

Twelve weeks post treatment, mice were sacrificed, and RNA was extracted from cortical brain tissue, spinal cord, and striatum for RTPCR analysis to measure amount of APP RNA using human primer probe set RTS35571 (described herein above). Results are presented as percent human APP RNA relative to the amount of APP RNA in PBS treated control animals, normalized to mouse PPIA (% control). Mouse PPIA was amplified using primer probe set m_cyclo24 (described herein above).

The half maximal effective dose (ED50) of each modified oligonucleotide was calculated using GraphPad Prism 10 software (GraphPad Software, San Diego, CA).

TABLE 9
Dose-dependent reduction of human
APP RNA in APP YAC transgenic mice

| Com- | | APP RNA (% control) | | | | | |
|---|---|---|---|---|---|---|---|
| pound | Dose | Spinal | ED50 | | ED50 | Hippo- | ED50 |
| No. | (μg) | Cord | (μg) | Cortex | (μg) | campus | (μg) |
| PBS | — | 100 | — | 100 | — | 100 | — |
| 1398227 | 10 | 86 | 157 | 104 | 206 | 97 | 210 |
| | 30 | 70 | | 99 | | 89 | |
| | 100 | 53 | | 65 | | 65 | |
| | 300 | 46 | | 41 | | 45 | |
| | 700 | 28 | | 19 | | 18 | |

### Example 7: Duration of Action Study of Modified Oligonucleotides Complementary to Human APP in APP YAC Transgenic Mice, Single Dose

Modified oligonucleotides described above were tested in APP YAC transgenic mice. The APP YAC transgenic mice were divided into groups of 3-6 mice each. Each mouse received a single ICV bolus of 300 μg of modified oligonucleotide. A group of 3-4 mice received a single ICV bolus with PBS as a negative control.

Mice were sacrificed at various timepoints as indicated in the tables below and RNA was extracted from cortical brain tissue for RTPCR analysis to measure amount of APP RNA using human primer probe set RTS35571 (described herein above). Results from several different experiments are combined and are presented in the table below as percent human APP RNA relative to the amount of APP in PBS treated control animals, normalized to mouse PPIA (% control). Mouse PPIA was amplified using primer probe set m_cyclo24 (described herein above).

TABLE 10
Reduction of human APP RNA in APP YAC transgenic mice

| | Timepoint | APP RNA (% control) |
|---|---|---|
| Compound No. | (Days post-dose) | in Cortex |
| 1353884 | 28 | 38 |
| | 56 | 29 |
| | 84 | 56 |
| | 112 | 46 |
| | 140 | 57 |
| 1398227 | 28 | 35 |
| | 56 | 34 |
| | 84 | 49 |
| | 112 | 68 |
| | 140 | 63 |
| 1620705 | 28 | 12 |
| | 56 | 15‡ |
| | 84 | 23 |
| | 112 | 12 |
| | 140 | 40 |

‡indicates fewer than 3 samples available

1. A modified oligonucleotide according to the following chemical structure: 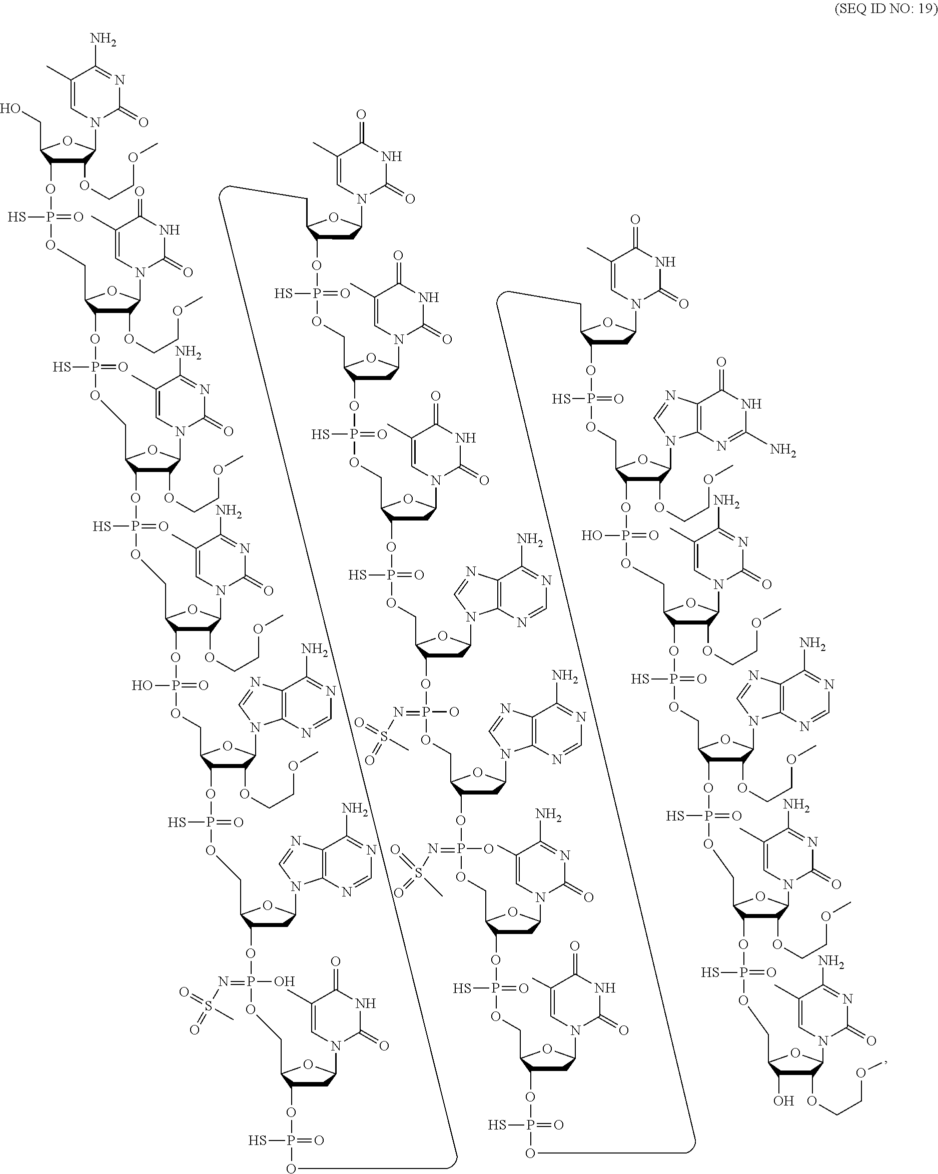
or a pharmaceutically acceptable salt thereof.

2. The modified oligonucleotide of claim 1, which is a pharmaceutically acceptable salt comprising one or more cations selected from sodium, potassium, calcium, and magnesium.

3. The modified oligonucleotide of claim 2, which is the sodium salt or the potassium salt.

4. An oligomeric compound comprising a modified oligonucleotide according to the following chemical structure:

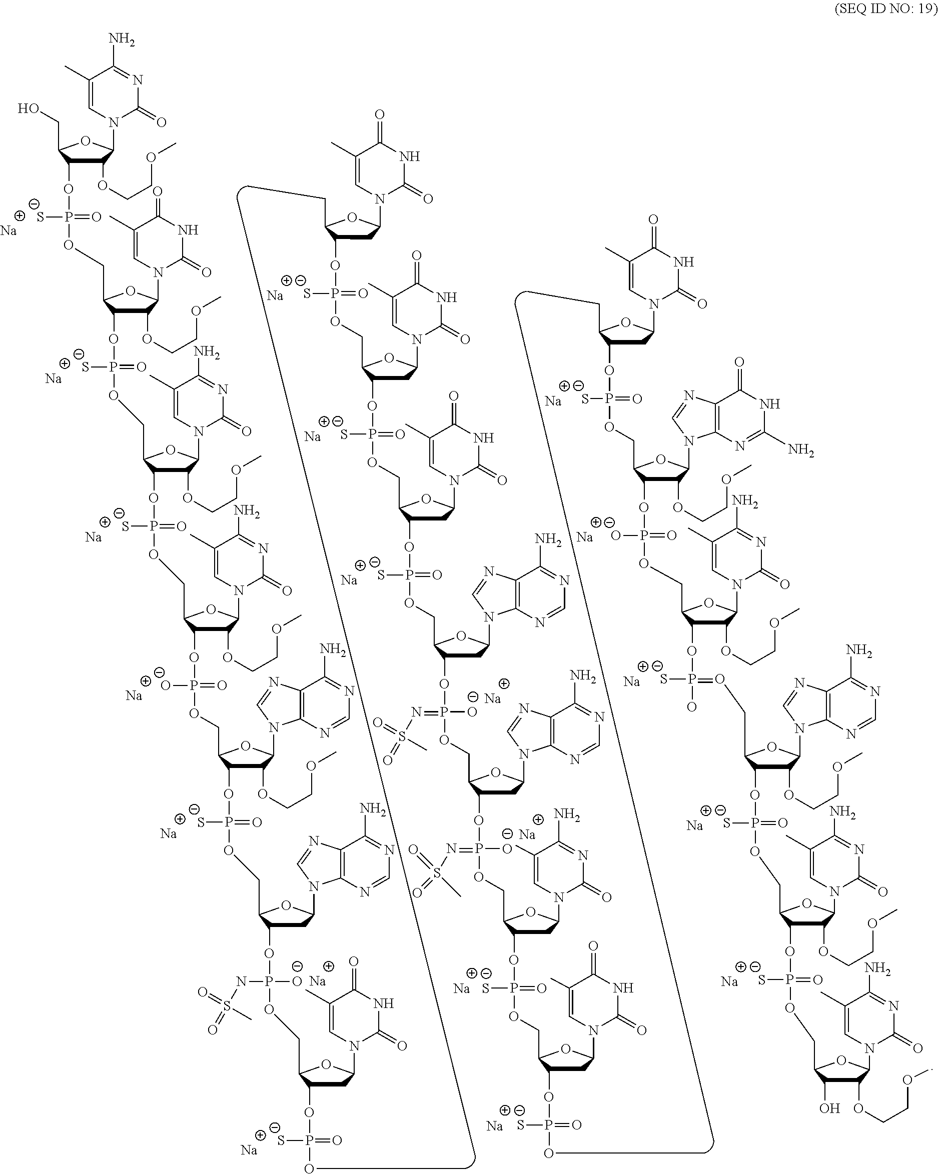

5. An oligomeric compound comprising a modified oligonucleotide according to the following chemical notation:
(SEQ ID NO: 19)
^{m}CₑₛTₑₛ^{m}Cₑₛ^{m}CₑₒAₑₛA_{dz}T_{ds}T_{ds}T_{ds}T_{ds}A_{dz}A_{dz}^{m}C_{ds}T_{ds}T_{ds}
Gₑₒ^{m}CₑₛAₑₛ^{m}Cₑₛ^{m}Cₑ,
wherein:
A=an adenine nucleobase,
^{m}C=a 5-methylcytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage,
o=a pbsphodiester internucleoside linkage, and
z=a mesyl phosphoramidate internucleoside linkage,
wherein the oligomeric compound optionally comprises a conjugate group or a terminal group.

6. A population of modified oligonucleotides of claim 1, wherein each of the phosphorothioate internucleoside linkages of the modified oligonucleotide are stereorandom.

7. A pharmaceutical composition comprising a modified oligonucleotide of claim 1 and a pharmaceutically acceptable diluent.

8. The pharmaceutical composition of claim 7, wherein the pharmaceutically acceptable diluent is artificial cerebrospinal fluid, phosphate-buffered saline, or water.

9. The pharmaceutical composition of claim 8, wherein the pharmaceutical composition consists essentially of the modified oligonucleotide and phosphate-buffered saline or artificial cerebrospinal fluid.

10. A population of modified oligonucleotides of claim 2, wherein all of the phosphorothioate internucleoside linkages of the modified oligonucleotide are stereorandom.

11. A pharmaceutical composition comprising a modified oligonucleotide of claim 2 and a pharmaceutically acceptable diluent.

12. The pharmaceutical composition of claim 11, wherein the pharmaceutically acceptable diluent is phosphate-buffered saline or artificial cerebrospinal fluid.

13. The pharmaceutical composition of claim 12, wherein the pharmaceutical composition consists essentially of the modified oligonucleotide and the phosphate-buffered saline or the artificial cerebrospinal fluid.

14. A population of modified oligonucleotides of claim 4, wherein all of the phosphorothioate internucleoside linkages of the modified oligonucleotide are stereorandom.

15. A pharmaceutical composition comprising a modified oligonucleotide of claim 4 and a pharmaceutically acceptable diluent.

16. The pharmaceutical composition of claim 15, wherein the pharmaceutically acceptable diluent is phosphate-buffered saline or artificial cerebrospinal fluid.

17. The pharmaceutical composition of claim 16, wherein the pharmaceutical composition consists essentially of the modified oligonucleotide and the phosphate-buffered saline or the artificial cerebrospinal fluid.

18. A population of oligomeric compounds of claim 5, wherein all of the phosphorothioate internucleoside linkages of the oligomeric compound are stereorandom.

19. A pharmaceutical composition comprising an oligomeric compound of claim 5 and a pharmaceutically acceptable diluent.

20. The pharmaceutical composition of claim 19, wherein the pharmaceutically acceptable diluent is phosphate-buffered saline or artificial cerebrospinal fluid.

21. The pharmaceutical composition of claim 20, wherein the pharmaceutical composition consists essentially of the oligomeric compound and the phosphate-buffered saline or the artificial cerebrospinal fluid.

22. A pharmaceutical composition comprising a population of claim 6 and a pharmaceutically acceptable diluent.

23. The pharmaceutical composition of claim 22, wherein the pharmaceutically acceptable diluent is phosphate-buffered saline or artificial cerebrospinal fluid.

24. A pharmaceutical composition comprising a population of claim 10 and a pharmaceutically acceptable diluent.

25. The pharmaceutical composition of claim 24, wherein the pharmaceutically acceptable diluent is phosphate-buffered saline or artificial cerebrospinal fluid.

26. A pharmaceutical composition comprising a population of claim 14 and a pharmaceutically acceptable diluent.

27. The pharmaceutical composition of claim 26, wherein the pharmaceutically acceptable diluent is phosphate-buffered saline or artificial cerebrospinal fluid.

28. A pharmaceutical composition comprising a population of claim 18 and a pharmaceutically acceptable diluent.

29. The pharmaceutical composition of claim 28, wherein the pharmaceutically acceptable diluent is phosphate-buffered saline or artificial cerebrospinal fluid.
